(19) United States
(12) Patent Application Publication
(10) Pub. No.: US 2003/0087847 A1
(43) Pub. Date: May 8, 2003

(54) Method and reagent for the inhibition of checkpoint kinase-1 (Chk1) enzyme

(75) Inventors: Thale Jarvis, Boulder, CO (US); James McSwiggen, Boulder, CO (US); Robert N. Booher, San Francisco, CA (US); Patricia S. Holman, San Francisco, CA (US); Ali R. Fattaey, San Francisco, CA (US)

Correspondence Address: MCDONNELL BOEHNEN HULBERT & BERGHOFF, 300 SOUTH WACKER DRIVE, SUITE 3200, CHICAGO, IL, 60606, US

(73) Assignee: Onyx Pharmaceuticals, Inc.

(21) Appl. No.: 09/776,474

(22) Filed: Feb. 2, 2001

Related U.S. Application Data
(60) Provisional application No. 60/179,983, filed on Feb. 3, 2000.

(51) Int. Cl. A61K 48/00; C07H 21/02; C12Q 1/68

(52) U.S. Cl. 514/44; 536/23.1; 435/6

(57) ABSTRACT

The present invention relates to nucleic acid molecules, including antisense and enzymatic nucleic acid molecules, such as hammerhead ribozymes, DNAzymes, and antisense, which modulate the expression of the Chk-1 gene.

### BACKGROUND OF THE INVENTION

This patent application claims priority from Jarvis et al., USSN (60/179,983), filed Feb. 3, 2000, entitled “METHOD AND REAGENT FOR THE INHIBITION OF CHECKPOINT KINASE-1 (CHK1) ENZYME”. This application is hereby incorporated by reference herein in its entirety including the drawings.

The present invention concerns compounds, compositions, and methods for the study, diagnosis, and treatment of conditions and diseases related to the expression of kinases which phosphorylate Cdc25 S216, such as Chk1 (checkpoint kinase 1) enzyme.

The following is a brief description of the current understanding of Chk1. The discussion is not meant to be complete and is provided only for understanding the invention that follows. The summary is not an admission that any of the work described below is prior art to the claimed invention.

Mammalian cells treated with agents that inhibit DNA replication or cause DNA damage undergo cell cycle arrest due to the presence of multiple checkpoint response mechanisms. Cancer cells frequently lack the p53-induced G1 DNA damage checkpoint response and instead arrest in G2 due to a checkpoint pathway directed towards preventing Cdc2 kinase activation. Inhibition of Cdc2 kinase activity is mediated by Wee1-like kinases, which phosphorylate key residues within the ATP-binding pocket of Cdc2 (accession No. X05360). Maintenance of this arrest also involves repressing Cdc25 function, the phosphatase that removes the Cdc2 inhibitory phosphorylations, by a mechanism involving the binding of 14-3-3 proteins to a phosphorylated serine residue (S216) in Cdc25. Multiple kinases, including Chk1 (accession No. AF016582), Chk2 (Cds1) (accession No. NM_{—}007194), and C-TAK1 (accession No. AL050393), can phosphorylate Cdc25 S216 (accession No. M34065) in-vitro. These kinases may function in the DNA replication and/or DNA damage checkpoint response in vivo.

Hoekstra et al, International PCT publication No. WO/9955844, describe, in general terms, a method for promoting differentiation of a differentiation-inhibited cell by introducing into a cell a first polynucleotide encoding an antisense polynucleotide that hybridizes to a second polynucleotide encoding a cell cycle checkpoint protein.

### SUMMARY OF THE INVENTION

The invention features novel nucleic acid-based techniques [e.g., enzymatic nucleic acid molecules (ribozymes), antisense nucleic acids, 2-5A antisense chimeras, triplex DNA, antisense nucleic acids containing RNA cleaving chemical groups] and methods for their use to modulate the expression of kinases which phosphorylate Cdc25 S216, such as Chk1 (checkpoint kinase 1) enzyme, Chk2 (Cds1) and C-TAK1.

The description below of the various aspects and embodiments is provided with reference to the exemplary gene Chk1. However, the various aspects and embodiments are also directed to each of the other genes which phosphorylate Cdc25S216. Those additional genes can be analyzed for target sites as described for Chk1. Further, the nucleic acid-based techniques, molecules, and compositions targeted to those genes can be performed as for Chk1. Thus, the inhibition and the effects of such inhibition of the other genes can be performed as described herein.

In a preferred embodiment, the invention features the use of one or more of the nucleic acid-based techniques independently or in combination to inhibit the expression of the genes encoding Chk1. Specifically, the invention features the use of nucleic acid-based techniques to specifically inhibit the expression of Chk1 gene.

In another preferred embodiment, the invention features the use of an enzymatic nucleic acid molecule, preferably in the hammerhead, NCH, G-cleaver, amberzyme, zinzyme and/or DNAzyme motif, to inhibit the expression of Chk1 gene.

By “inhibit” it is meant that the activity of Chk1 or level of RNAs or equivalent RNAs encoding one or more protein subunits of Chk1 is reduced below that observed in the absence of the nucleic acid molecules of the invention. In one embodiment, inhibition with enzymatic nucleic acid molecule preferably is below that level observed in the presence of an enzymatically inactive or attenuated molecule that is able to bind to the same site on the target RNA, but is unable to cleave that RNA. In another embodiment, inhibition with antisense oligonucleotides is preferably below that level observed in the presence of, for example, an oligonucleotide with scrambled sequence or with mismatches. In another embodiment, inhibition of Chk1 genes with the nucleic acid molecule of the instant invention is greater than in the presence of the nucleic acid molecule than in its absence.

By “enzymatic nucleic acid molecule” it is meant a nucleic acid molecule which has complementarity in a substrate-binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% may also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids may be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

By “nucleic acid molecule” as used herein is meant a molecule having nucleotides. The nucleic acid can be single, double, or multiple stranded and may comprise modified or unmodified nucleotides or non-nucleotides or various mixtures and combinations thereof.

By “enzymatic portion” or “catalytic domain” is meant that portion/region of the enzymatic nucleic acid molecule essential for cleavage of a nucleic acid substrate (for example, see FIGS. 1-5).

By “substrate binding arm” or “substrate binding domain” is meant that portion/region of a enzymatic nucleic acid which is able to interact, for example via complementarity (i.e., able to base-pair with), with a portion of its substrate. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). Examples of such arms are shown generally in FIGS. 1-5. That is, these arms contain sequences within a enzymatic nucleic acid which are intended to bring enzymatic nucleic acid and target RNA together through complementary base-pairing interactions. The enzymatic nucleic acid of the invention may have binding arms that are contiguous or non-contiguous and may be of varying lengths. The length of the binding arm(s) are preferably greater than or equal to four nucleotides and of sufficient length to stably interact with the target RNA; preferably 12-100 nucleotides; more preferably 14-24 nucleotides long (see for example Werner and Uhlenbeck, supra; Hamman et al., supra; Hampel et al., EP0360257; Berzal-Herrance et al., 1993, EMBO J., 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “Inozyme” or “NCH” motif is meant, an enzymatic nucleic acid molecule comprising a motif as is generally described as NCH Rz in FIG. 2. Inozymes possess endonuclease activity to cleave RNA substrates having a cleavage triplet NCH/, where N is a nucleotide, C is cytidine and H is adenosine, uridine or cytidine, and/represents the cleavage site. H is used interchangeably with X. Inozymes can also possess endonuclease activity to cleave RNA substrates having a cleavage triplet NCN/, where N is a nucleotide, C is cytidine, and/represents the cleavage site. “I” in FIG. 2 represents an Inosine nucleotide, preferably a ribo-Inosine or xylo-Inosine nucleoside.

By “G-cleaver” motif is meant, an enzymatic nucleic acid molecule comprising a motif as is generally described as G-cleaver in FIG. 2. G-cleavers possess endonuclease activity to cleave RNA substrates having a cleavage triplet NYN/, where N is a nucleotide, Y is uridine or cytidine and/represents the cleavage site. G-cleavers may be chemically modified as is generally shown in FIG. 2.

By “amberzyme” motif is meant, an enzymatic nucleic acid molecule comprising a motif as is generally described in FIG. 3. Amberzymes possess endonuclease activity to cleave RNA substrates having a cleavage triplet NG/N, where N is a nucleotide, G is guanosine, and/represents the cleavage site. Amberzymes may be chemically modified to increase nuclease stability through substitutions as are generally shown in FIG. 3. In addition, differing nucleoside and/or non-nucleoside linkers can be used to substitute the 5′-gaaa-3′ loops shown in the figure. Amberzymes represent a non-limiting example of an enzymatic nucleic acid molecule that does not require a ribonucleotide (2′-OH) group within its own nucleic acid sequence for activity.

By “zinzyme” motif is meant, an enzymatic nucleic acid molecule comprising a motif as is generally described in FIG. 4. Zinzymes possess endonuclease activity to cleave RNA substrates having a cleavage triplet including but not limited to YG/Y, where Y is uridine or cytidine, and G is guanosine and/represents the cleavage site. Zinzymes may be chemically modified to increase nuclease stability through substitutions as are generally shown in FIG. 4, including substituting 2′-O-methyl guanosine nucleotides for guanosine nucleotides. In addition, differing nucleotide and/or non-nucleotide linkers can be used to substitute the 5′-gaaa-2′ loop shown in the figure. Zinzymes represent a non-limiting example of an enzymatic nucleic acid molecule that does not require a ribonucleotide (2′-OH) group within its own nucleic acid sequence for activity.

By ‘DNAzyme’ is meant, an enzymatic nucleic acid molecule that does not require the presence of a 2′-OH group for its activity. In particular embodiments the enzymatic nucleic acid molecule may have an attached linker(s) or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is shown in FIG. 5 and is generally reviewed in Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. Additional DNAzyme motifs can be selected for using techniques similar to those described in these references, and hence, are within the scope of the present invention.

By “sufficient length” is meant an oligonucleotide of greater than or equal to 3 nucleotides that is of a length great enough to provide the intended function under the expected condition. For example, for binding arms of enzymatic nucleic acid “sufficient length” means that the binding arm sequence is long enough to provide stable binding to a target site under the expected binding conditions. Preferably, the binding arms are not so long as to prevent useful turnover.

By “stably interact” is meant interaction of the oligonucleotides with target nucleic acid (e.g., by forming hydrogen bonds with complementary nucleotides in the target under physiological conditions) that is sufficient to the intended purpose (e.g., cleavage of target RNA by an enzyme).

By “equivalent” RNA to Chk1 is meant to include those naturally occurring RNA molecules having homology (partial or complete) to Chk1 proteins or encoding for proteins with similar function as Chk1 in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

By “homology” is meant the nucleotide sequence of two or more nucleic acid molecules is partially or completely identical.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule may bind to substrate such that the substrate molecule forms a loop, and/or an antisense molecule may bind such that the antisense molecule forms a loop. Thus, the antisense molecule may be complementary to two (or even more) non-contiguous substrate sequences or two (or even more) non-contiguous sequence portions of an antisense molecule may be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol, 40, 1-49. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

By “RNase H activating region” is meant a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

By “2-5A antisense chimera” is meant an antisense oligonucleotide containing a 5′-phosphorylated 2′-5′-linked adenylate residue. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300; Silverman et al., 2000, Methods Enzymol., 313, 522-533; Player and Torrence, 1998, Pharmacol. Ther., 78, 55-113).

By “triplex forming oligonucleotides” is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504; Fox, 2000, Curr. Med. Chem., 7, 17-37; Praseuth et. al., 2000, Biochim. Biophys. Acta, 1489, 181-206).

By “gene” it is meant a nucleic acid that encodes an RNA, for example, nucleic acid sequences including but not limited to structural genes encoding a polypeptide.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

By “decoy RNA” is meant a RNA molecule that mimics the natural binding domain for a ligand. The decoy RNA therefore competes with natural binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is but a specific example and those in the art will recognize that other embodiments can be readily generated using techniques generally known in the art.

Several varieties of naturally occurring enzymatic RNAs are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

The enzymatic nucleic acid molecule that cleave the specified sites in Chk1 -specific RNAs represent a novel therapeutic approach to treat a variety of pathologic indications, including cancer.

In one of the preferred embodiments of the inventions described herein, the enzymatic nucleic acid molecule is formed in a hammerhead or hairpin motif, but may also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfuis, supra, Rossi et al., 1992, AIDS Research and Human Retroviruses 8, 183. Examples of hairpin motifs are described by Hampel et al., EP0360257, Hampel and Tritz, 1989 Biochemistry 28, 4929, Feldstein et al., 1989, Gene 82, 53, Haseloff and Gerlach, 1989, Gene, 82, 43, Hampel et al., 1990 Nucleic Acids Res. 18, 299; and Chowrira & McSwiggen, U.S. Pat. No. 5,631,359. The hepatitis delta virus motif is described by Perrotta and Been, 1992 Biochemistry 31, 16. The RNase P motif is described by Guerrier-Takada et al., 1983 Cell 35, 849; Forster and Altman, 1990, Science 249, 783; and Li and Altman, 1996, Nucleic Acids Res. 24, 835. The Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; and Guo and Collins, 1995, EMBO. J. 14, 363). Group II introns are described by Griffin et al., 1995, Chem. Biol. 2, 761; Michels and Pyle, 1995, Biochemistry 34, 2965; and Pyle et al., International PCT Publication No. WO 96/22689. The Group I intron is described by Cech et al., U.S. Pat. No. 4,987,071. DNAzymes are described by Usman et al., International PCT Publication No. WO 95/11304; Chartrand et al., 1995, NAR 23, 4092; Breaker et al., 1995, Chem. Bio. 2, 655; and Santoro et al., 1997, PNAS 94, 4262. NCH cleaving motifs are described in Ludwig & Sproat, International PCT Publication No. WO 98/58058; and G-cleavers are described in Kore et al., 1998, Nucleic Acids Research 26, 4116-4120 and Eckstein et al., International PCT Publication No. WO 99/16871. Additional motifs include the Aptazyme (Breaker et al., WO 98/43993), Amberzyme (Class I motif; FIG. 3; Beigelman et al., International PCT publication No. WO 99/55857) and Zinzyme (Beigelman et al., International PCT publication No. WO 99/55857), all these references are incorporated by reference herein in their totalities, including drawings and can also be used in the present invention. These specific motifs are not limiting in the invention. and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In preferred embodiments of the present invention, a nucleic acid molecule of the instant invention can be between 13 and 100 nucleotides in length. Exemplary enzymatic nucleic acid molecules of the invention are shown in Tables III-XIII. For example, enzymatic nucleic acid molecules of the invention are preferably between 15 and 50 nucleotides in length, more preferably between 25 and 40 nucleotides in length, e.g., 34, 36, or 38 nucleotides in length (for example see Jarvis et al., 1996, J. Biol. Chem., 271, 29107-29112). Exemplary DNAzymes of the invention are preferably between 15 and 40 nucleotides in length, more preferably between 25 and 35 nucleotides in length, e.g., 29, 30, 31, or 32 nucleotides in length (see for example Santoro et al., 1998, Biochemistry, 37, 13330-13342; Chartrand et al., 1995, Nucleic Acids Research, 23, 4092-4096). Exemplary antisense molecules of the invention are preferably between 15 and 75 nucleotides in length, more preferably between 20 and 35 nucleotides in length, e.g., 25, 26, 27, or 28 nucleotides in length (see for example Woolf et al., 1992, PNAS., 89, 7305-7309; Milner et al., 1997, Nature Biotechnology, 15, 537-541). Exemplary triplex forming oligonucleotide molecules of the invention are preferably between 10 and 40 nucleotides in length, more preferably between 12 and 25 nucleotides in length, e.g., 18, 19, 20, or 21 nucleotides in length (see for example Maher et al., 1990, Biochemistiy, 29, 8820-8826; Strobel and Dervan, 1990, Science, 249, 73-75). Those skilled in the art will recognize that all that is required is for the nucleic acid molecule are of length and conformation sufficient and suitable for the nucleic acid molecule to catalyze a reaction contemplated herein. The length of the nucleic acid molecules of the instant invention are not limiting within the general limits stated.

Preferably, a nucleic acid molecule that down regulates the replication of Chk1 comprises between 12 and 100 bases complementary to a RNA molecule of Chk1. Even more preferably, a nucleic acid molecule that down regulates the replication of Chk1 comprises between 14 and 24 bases complementary to a RNA molecule of Chk1.

In a preferred embodiment, the invention provides a method for producing a class of nucleic acid-based gene inhibiting agents which exhibit a high degree of specificity for the RNA of a desired target. For example, the enzymatic nucleic acid molecule is preferably targeted to a highly conserved sequence region of target RNAs encoding kinases which phosphorylate Cdc25 S216, such as Chk1 proteins (specifically Chk1 gene) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and/or RNA vectors that are delivered to specific cells.

In a preferred embodiment, the invention features the use of nucleic acid-based inhibitors of the invention to specifically target genes that share homology with the Chk1 gene.

As used in herein “cell” is used in its usual biological sense, and does not refer to an entire multicellular organism, e.g., specifically does not refer to a human. The cell may be present in an organism which may be a human but is preferably a non-human multicellular organism, e.g., birds, plants and mammals such as cows, sheep, apes, monkeys, swine, dogs, and cats. The cell may be prokaryotic (e.g., bacterial cell) or eukaryotic (e.g., mammalian or plant cell).

By “Chk1 proteins” is meant, a protein or a mutant protein derivative thereof, comprising phosphorylation activity, preferably to serine residue (S216), or its equivalent, in Cdc25 phosphatase.

By “highly conserved sequence region” is meant, a nucleotide sequence of one or more regions in a target gene does not vary significantly from one generation to the other or from one biological system to the other.

The nucleic acid-based inhibitors of Chk1 expression are useful for the prevention and/or treatment of diseases and conditions such as cancer, including cancer of the colon, rectum, lung, breast, prostate and any other diseases or conditions that are related to or will respond to the levels of Chk1 in a cell or tissue, alone or in combination with other therapies. In addition, Chk1 inhibition may be used as a therapeutic target for abrogating the G2 DNA damage checkpoint arrest; a situation that may selectively sensitize p53-deficient tumor cells to radiation or chemotherapy treatment.

By “related” is meant that the reduction of Chk1 expression (specifically Chk1 gene) RNA levels and thus reduction in the level of the respective protein will relieve, to some extent, the symptoms of the disease or condition.

The nucleic acid-based inhibitors of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers. In preferred embodiments, the enzymatic nucleic acid inhibitors comprise sequences, which are complementary to the substrate sequences in Tables III to VIII. Examples of such enzymatic nucleic acid molecules also are shown in Tables III to VIII. Examples of such enzymatic nucleic acid molecules consist essentially of sequences defined in these Tables.

In yet another embodiment, the invention features antisense nucleic acid molecules and 2-5A chimera including sequences complementary to the substrate sequences shown in Tables III to IX. Such nucleic acid molecules can include sequences as shown for the binding arms of the enzymatic nucleic acid molecules in Tables III to VIII and sequences shown as GeneBloc™ sequences in Table IX. Similarly, triplex molecules can be provided targeted to the corresponding DNA target regions, and containing the DNA equivalent of a target sequence or a sequence complementary to the specified target (substrate) sequence. Typically, antisense molecules will be complementary to a target sequence along a single contiguous sequence of the antisense molecule. However, in certain embodiments, an antisense molecule may bind to substrate such that the substrate molecule forms a loop, and/or an antisense molecule may bind such that the antisense molecule forms a loop. Thus, the antisense molecule may be complementary to two (or even more) non-contiguous substrate sequences or two (or even more) non-contiguous sequence portions of an antisense molecule may be complementary to a target sequence or both.

By “consists essentially of” is meant that the active nucleic acid molecule of the invention, for example an enzymatic nucleic acid molecule, contains an enzymatic center or core equivalent to those in the examples, and binding arms able to bind RNA such that cleavage at the target site occurs. Other sequences may be present which do not interfere with such cleavage. Thus, a core region may, for example, include one or more loop, stem-loop structure or linker, which does not prevent enzymatic activity. Thus, the underlined regions in the sequences in Tables III and IV can be such a loop, stem-loop, nucleotide linker, and/or non-nucleotide linker and can be represented generally as sequence “X”. For example, a core sequence for a hammerhead enzymatic nucleic acid can comprise a conserved sequence, such as 5′-CUGAUGAG-3′ and 5′-CGAA-3 ′ connected by a sequence X, where is 5′-GCCGUUAGGC-3′ (SEQ ID NO 3173) or any other stem II region known in the art or a nucleotide and/or non-nucleotide linker. Similarly, for other nucleic acid molecules of the instant invention, such as Inozyme, G-cleaver, amberzyme, zinzyme, DNAzyme, antisense, 2-5A antisense, triplex forming nucleic acid, and decoy nucleic acids, other sequences or non-nucleotide linkers may be present that do not interfere with the function of the nucleic acid molecule.

Sequence X may be a linker of ≧2 nucleotides in length, preferably 3, 4, 5, 6, 7, 8, 9, 10, 15, 20, 26, 30, where the nucleotides may preferably be internally base-paired to form a stem of preferably ≧2 base pairs. Alternatively or in addition, sequence X may be a non-nucleotide linker. In yet another embodiment, the nucleotide linker X can be a nucleic acid aptamer, such as an ATP aptamer, HIV Rev aptamer (RRE), HIV Tat aptamer (TAR) and others (for a review see Gold et al., 1995, Annu. Rev. Biochem., 64, 763; and Szostak & Ellington, 1993, in The RNA World, ed. Gesteland and Atkins, pp. 511, CSH Laboratory Press). A “nucleic acid aptamer” as used herein is meant to indicate a nucleic acid sequence capable of interacting with a ligand. The ligand can be any natural or a synthetic molecule, including but not limited to a resin, metabolites, nucleosides, nucleotides, drugs, toxins, transition state analogs, peptides, lipids, proteins, amino acids, nucleic acid molecules, hormones, carbohydrates, receptors, cells, viruses, bacteria and others.

In yet another embodiment, the non-nucleotide linker X is as defined herein. The term “non-nucleotide” as used herein include either abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. A “non-nucleotide” further means any group or compound which can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine. Thus, in a preferred embodiment, the invention features an enzymatic nucleic acid molecule having one or more non-nucleotide moieties, and having enzymatic activity to cleave an RNA or DNA molecule.

In another aspect of the invention, ribozymes or antisense molecules that cleave target RNA molecules and inhibit Chk1 (specifically Chk1 gene) activity are expressed from transcription units inserted into DNA or RNA vectors. The recombinant vectors are preferably DNA plasmids or viral vectors. Ribozyme or antisense expressing viral vectors could be constructed based on, but not limited to, adeno-associated virus, retrovirus, adenovirus, or alphavirus. Preferably, the recombinant vectors capable of expressing the ribozymes or antisense are delivered as described above, and persist in target cells. Alternatively, viral vectors may be used that provide for transient expression of ribozymes or antisense. Such vectors can be repeatedly administered as necessary. Once expressed, the ribozymes or antisense bind to the target RNA and inhibit its function or expression. Delivery of ribozyme or antisense expressing vectors can be systemic, such as by intravenous or intramuscular administration, by administration to target cells ex-planted from the patient followed by reintroduction into the patient, or by any other means that would allow for introduction into the desired target cell.

By “vectors” is meant any nucleic acid- and/or viral-based technique used to deliver a desired nucleic acid.

By “patient” is meant an organism, which is a donor or recipient of explanted cells or the cells themselves. “Patient” also refers to an organism to which the nucleic acid molecules of the invention can be administered. Preferably, a patient is a mammal or mammalian cells. More preferably, a patient is a human or human cells.

By “enhanced enzymatic activity” is meant to include activity measured in cells and/or in vivo where the activity is a reflection of both the catalytic activity and the stability of the nucleic acid molecules of the invention. In this invention, the product of these properties can beincreased in vivo compared to an all RNA enzymatic nucleic acid or all DNA enzyme. In some cases, the activity or stability of the nucleic acid molecule can bedecreased (i.e., less than ten-fold), but the overall activity of the nucleic acid molecule is enhanced, in vivo.

The nucleic acid molecules of the instant invention, individually, or in combination or in conjunction with other drugs, can be used to treat diseases or conditions discussed above. For example, to treat a disease or condition associated with the levels of Chk1, the patient may be treated, or other appropriate cells may be treated, as is evident to those skilled in the art, individually or in combination with one or more drugs under conditions suitable for the treatment.

In a further embodiment, the described molecules, such as antisense or ribozymes, can be used in combination with other known treatments to treat conditions or diseases discussed above. For example, the described molecules could be used in combination with one or more known therapeutic agents to treat cancer, including but not limited to cancer of the colon, rectum, lung, breast and prostate.

In another preferred embodiment, the invention features nucleic acid-based inhibitors (e.g., enzymatic nucleic acid molecules (ribozymes), antisense nucleic acids, 2-5A antisense chimeras, triplex DNA, antisense nucleic acids containing RNA cleaving chemical groups) and methods for their use to down regulate or inhibit the expression of genes (e.g., Chk1) capable of progression and/or maintenance of cancer.

In another aspect, the invention provides mammalian cells containing one or more nucleic acid molecules and/or expression vectors of this invention. The one or more nucleic acid molecules may independently be targeted to the same or different sites.

By “comprising” is meant including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present.

Other features and advantages of the invention will be apparent from the following description of the preferred embodiments thereof, and from the claims.

### DESCRIPTION OF THE PREFERRED EMBODIMENTS

First the drawings will be described briefly.

### DRAWINGS

FIG. 1 shows the secondary structure model for seven different classes of enzymatic nucleic acid molecules. Arrow indicates the site of cleavage. -------- indicate the target sequence. Lines interspersed with dots are meant to indicate tertiary interactions. - is meant to indicate base-paired interaction. Group I Intron: P1-P9.0 represent various stem-loop structures (Cech et al., 1994, Nature Struc. Bio., 1, 273). RNase P (MIRNA): EGS represents external guide sequence (Forster et al., 1990, Science, 249, 783; Pace et al., 1990, J. Biol. Chem., 265, 3587). Group II Intron: 5′SS means 5′ splice site; 3′SS means 3′-splice site; IBS means intron binding site; EBS means exon binding site (Pyle et al., 1994, Biochemistry, 33, 2716). VS RNA: I-VI are meant to indicate six stem-loop structures; shaded regions are meant to indicate tertiary interaction (Collins, International PCT Publication No. WO 96/19577). HDV Ribozyme: : I-IV are meant to indicate four stem-loop structures (Been et al., U.S. Pat. No. 5,625,047). Hammerhead Ribozyme: I-III are meant to indicate three stem-loop structures; stems I-III can be of any length and may be symmetrical or asymmetrical (Usman et al., 1996, Curr. Op. Struct. Bio., 1, 527). Hairpin Ribozyme: Helix 1, 4 and 5 can be of any length; Helix 2 is between 3 and 8 base-pairs long; Y is a pyrimidine; Helix 2 (H2) is provided with a least 4 base pairs (i.e., n is 1, 2, 3 or 4) and helix 5 can be optionally provided of length 2 or more bases (preferably 3 -20 bases, i.e., m is from 1-20 or more). Helix 2 and helix 5 may be covalently linked by one or more bases (i.e., r is ≧1 base). Helix 1, 4 or 5 may also be extended by 2 or more base pairs (e.g., 4-20 base pairs) to stabilize the ribozyme structure, and preferably is a protein binding site. In each instance, each N and N′ independently is any normal or modified base and each dash represents a potential base-pairing interaction. These nucleotides may be modified at the sugar, base or phosphate. Complete base-pairing is not required in the helices, but is preferred. Helix 1 and 4 can be of any size (i.e., o and p is each independently from 0 to any number, e.g., 20) as long as some base-pairing is maintained. Essential bases are shown as specific bases in the structure, but those in the art will recognize that one or more may be modified chemically (abasic, base, sugar and/or phosphate modifications) or replaced with another base without significant effect. Helix 4 can be formed from two separate molecules, i.e., without a connecting loop. The connecting loop when present may be a ribonucleotide with or without modifications to its base, sugar or phosphate. “q”≧ is 2 bases. The connecting loop can also be replaced with a non-nucleotide linker molecule. H refers to bases A, U, or C. Y refers to pyrimidine bases. “______” refers to a covalent bond. (Burke et al., 1996, Nucleic Acids & MoL Biol., 10, 129; Chowrira etal., U.S. Pat. No. 5,631,359).

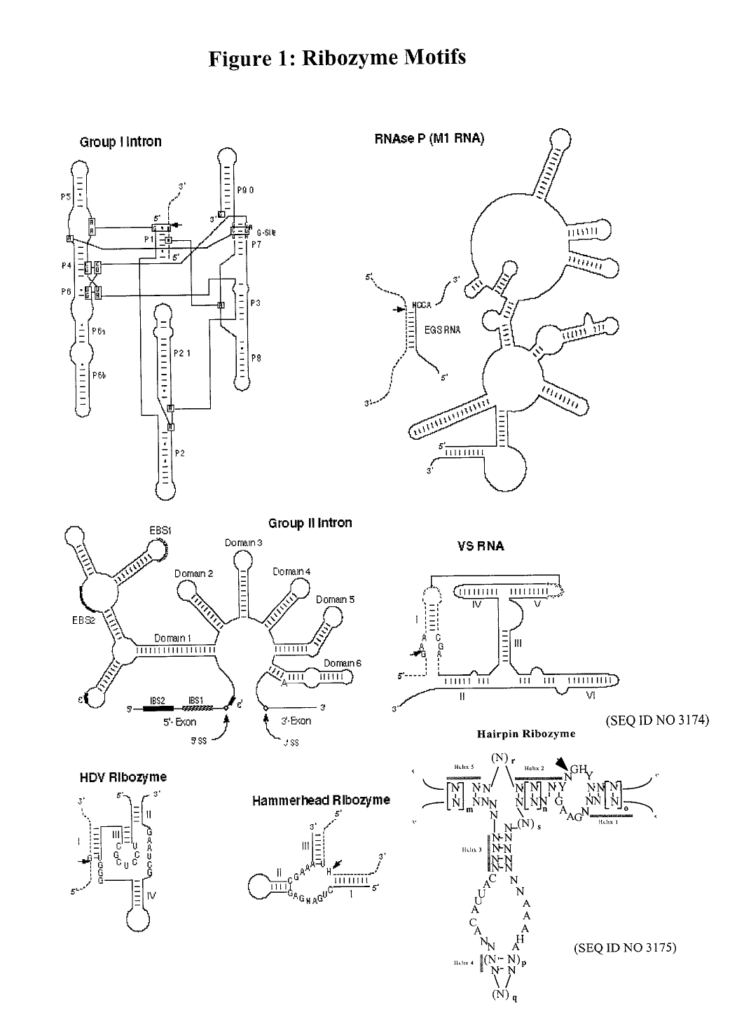

FIG. 2 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig & Sproat, International PCT Publication No. WO 98/58058); G-Cleaver, represents G-cleaver ribozyme motif (Kore et al., 1998, Nucleic Acids Research 26, 4116-4120). N or n, represent independently a nucleotide which may be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

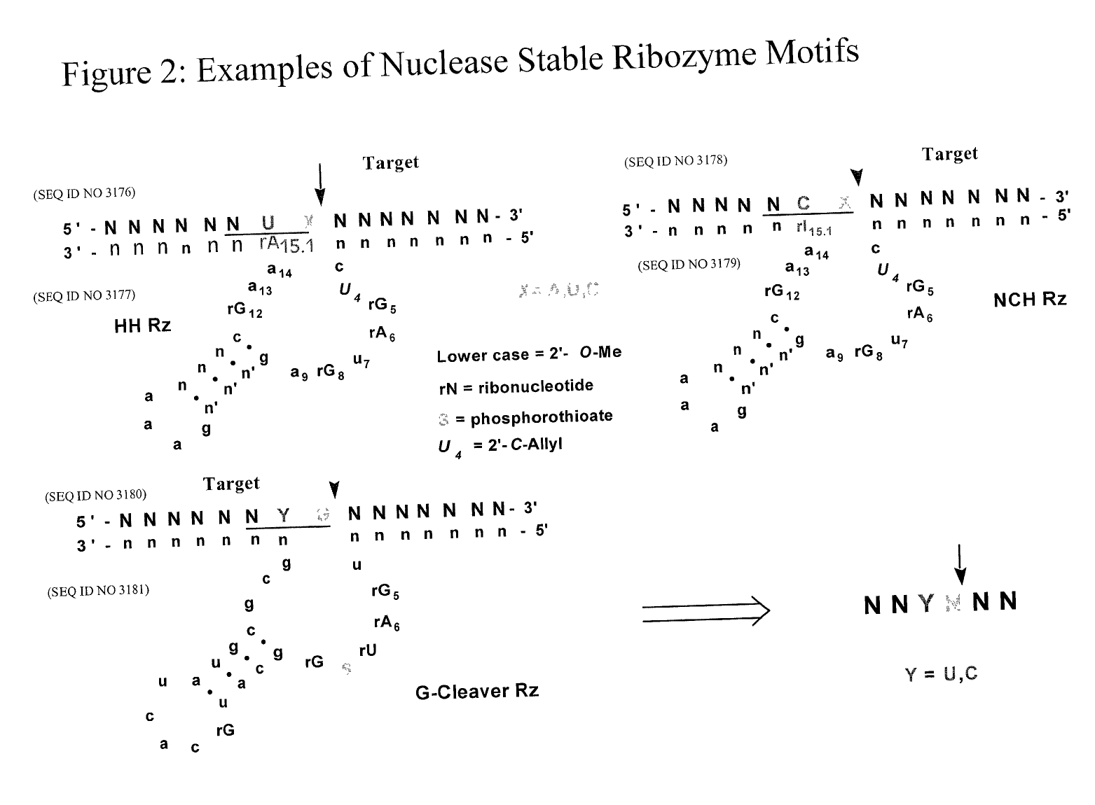

FIG. 3 shows an example of the Amberzyme ribozyme motif that is chemically stabilized (see, for example, Beigelman et al., International PCT publication No. WO 99/55857, incorporated by reference herein; also referred to as Class I Motif). The Amberzyme motif is a class of enzymatic nucleic molecules that do not require the presence of a ribonucleotide (2′-OH) group for its activity.

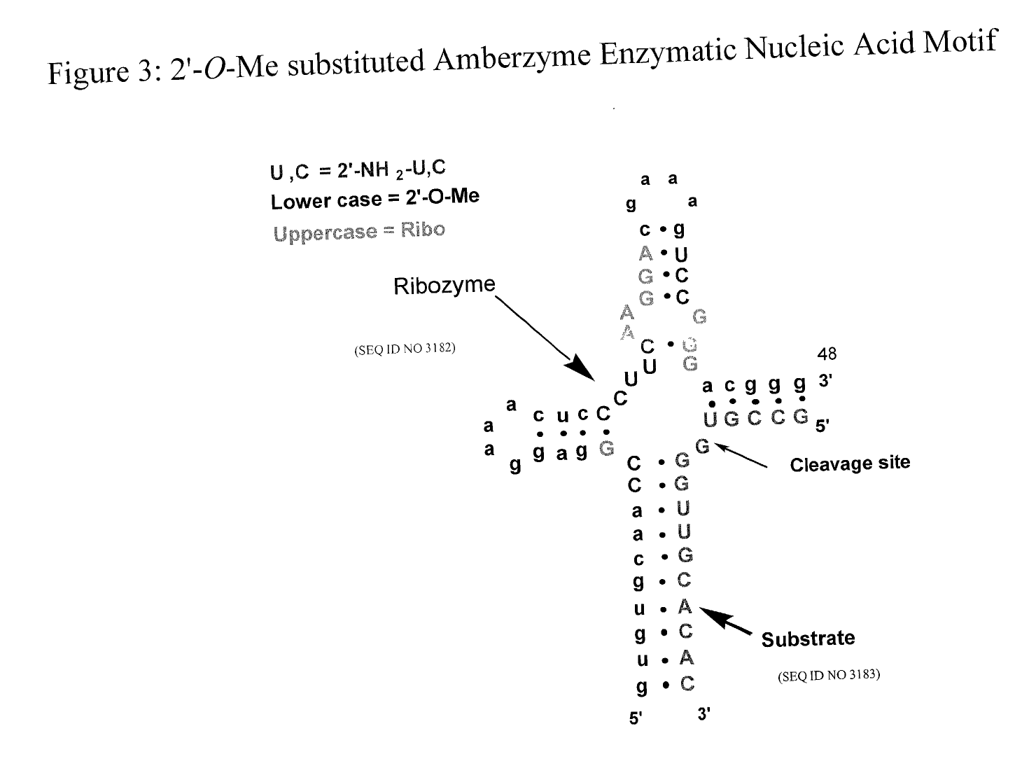

FIG. 4 shows an example of the Zinzyme A ribozyme motif that is chemically stabilized (Beigelman et al., International PCT publication No. WO 99/55857, incorporated by reference herein; also referred to as Class A or Class II Motif). The Zinzyme motif is a class of enzymatic nucleic molecules that do not require the presence of a ribonucleotide (2′-OH) group for its activity.

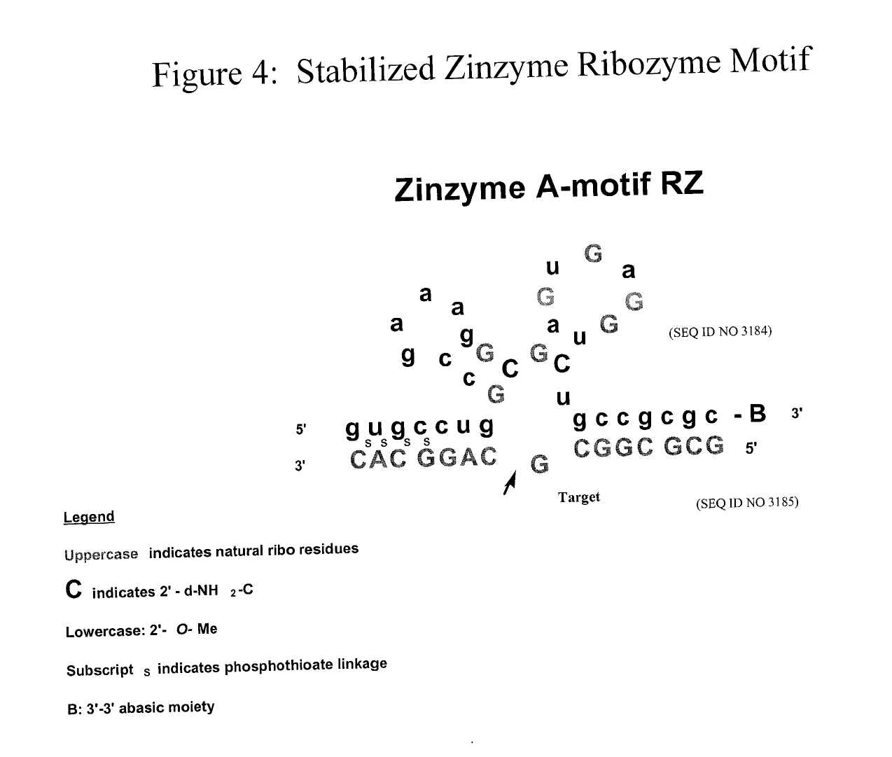

FIG. 5 shows an example of a DNAzyme motif described by Santoro et al., 1997, PNAS, 94, 4262.

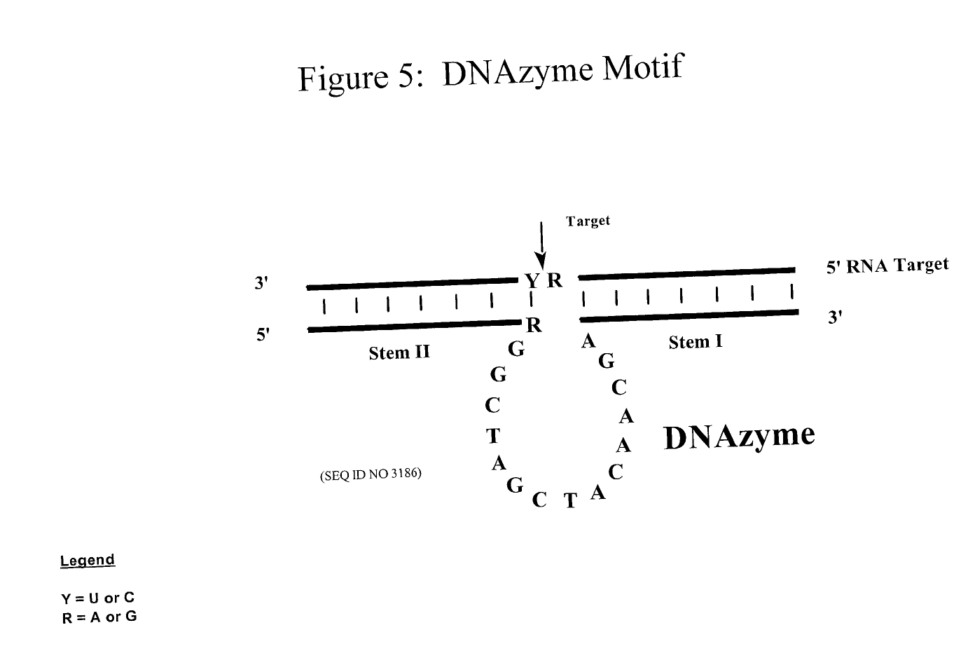

FIG. 6 shows a bar graph of a nucleic acid inhibitor (50 to 200 nM GeneBloc™ screen against Chk1 RNA in HeLa cells using 1.25 μg/ml GSV lipid with 24 hour sustained delivery in a 96-well format. Relative amounts of target RNA were measured normalized to actin using real-time PCR monitoring of amplification compared to mismatch nucleic acid and untreated controls. The sequences of GeneBloc™ reagents used in this experiment are shown in Table IX.

FIG. 7 shows a bar graph of a lipid optimization study utilizing lead nucleic acid inhibitors (GeneBlocs™) targeting Chk1 RNA in HeLa cells; 96-well plate format, 5000 cells/well, GSV lipid. Six different lipid concentrations are shown in conjunction with two different concentrations of the nucleic acid inhibitors.

FIG. 8 shows a bar graph displaying a time-course inhibition study of a lead nucleic acid inhibitor (GeneBloc™) targeting Chk1 RNA compared to a scrambled nucleic acid control, both at 5 and 100 nM concentrations; 96-well plate format, 5000 cells/well, 1.0 μg/ml GSV lipid.

FIG. 9 shows a bar graph representing inhibition of Chk1 RNA via primary lead (GeneBloc™) inhibition as described in FIG. 6, however utilizing a 6-well plate format with a cell density of 150,000 cells per well.

FIG. 10 shows a bar graph representing inhibition of Chk1 RNA via primary lead (GeneBloc™) inhibition in conjunction with +/− etoposide and nocodazole treatment; 50 nM GeneBloc™, 1.25 μg/ml GSV lipid, HeLa cells, 6-well plate format, 100,000 cells/well.

FIG. 11 shows a bar graph of a lipid optimization study utilizing a lead nucleic acid inhibitor (GeneBloc™) targeting Chk1 RNA in DLD-1 cells; 96-well plate format, 15,000 cells/well, GSV lipid. Four different lipid concentrations are shown in conjunction with two different concentrations of the nucleic acid inhibitor.

FIG. 12 shows a bar graph of a lipid optimization study utilizing a lead nucleic acid inhibitor (GeneBloc™) targeting Chk1 RNA in MCF-7 cells; 96-well plate format, 10,000 cells/well, GSV lipid. Four different lipid concentrations are shown in conjunction with two different concentrations of the nucleic acid inhibitor.

FIG. 13 shows a dose curve of primary and secondary nucleic acid inhibitor (GeneBloc™) leads targeting Chk1 RNA in HeLa cells using 1.25 μg/ml GSV lipid, 24 hr time-point, 96-well plate format with a density of 5000 cells/well.

### Mechanism of action of Nucleic Acid Molecules of the Invention

Antisense: Antisense molecules can be modified or unmodified RNA, DNA, or mixed polymer oligonucleotides which primarily function by specifically binding to matching sequences resulting in inhibition of peptide synthesis (Wu-Pong, November 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA may result in nuclease degradation of the heteroduplex (Wu-Pong, supra; Crooke, supra). To date, the only backbone modified DNA chemistry which will act as substrates for RNase H are phosphorothioates, phosphorodithioates, and borontrifluoridates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., USSN 60/101,174 which was filed on Sep. 21, 1998) all of these are incorporated by reference herein in their entirety.

In addition, antisense deoxyoligoribonucleotides can be used to target RNA by means of DNA-RNA interactions, thereby activating RNase H, which digests the target RNA in the duplex. Antisense DNA can be expressed via the use of a single stranded DNA intracellular expression vector or equivalents and variations thereof.

Triplex Forming Olihonucleotides (TFO): Single stranded DNA can be designed to bind to genomic DNA in a sequence specific manner. TFOs are comprised of pyrimidine-rich oligonucleotides which bind DNA helices through Hoogsteen Base-pairing (Wu-Pong, supra). The resulting triple helix composed of the DNA sense, DNA antisense, and TFO disrupts RNA synthesis by RNA polymerase. The TFO mechanism can result in gene expression or cell death since binding may be irreversible (Mukhopadhyay & Roth, supra).

2-5A Antisense Chimera: The 2-5A system is an interferon mediated mechanism for RNA degradation found in higher vertebrates (Mitra et al., 1996, Proc Nat Acad Sci USA 93, 6780-6785 ). Two types of enzymes, 2-5A synthetase and RNase L, are required for RNA cleavage. The 2-5A synthetases require double stranded RNA to form 2′-5′ oligoadenylates (2-5A). 2-5 A then acts as an allosteric effector for utilizing RNase L which has the ability to cleave single stranded RNA. The ability to form 2-5A structures with double stranded RNA makes this system particularly useful for inhibition of viral replication.

(2′-5′) oligoadenylate structures can be covalently linked to antisense molecules to form chimeric oligonucleotides capable of RNA cleavage (Torrence, supra). These molecules putatively bind and activate a 2-5A dependent RNase, the oligonucleotide/enzyme complex then binds to a target RNA molecule which can then be cleaved by the RNase enzyme.

Enzymatic Nucleic Acid: Seven basic varieties of naturally occurring enzymatic RNAs are presently known. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al.,1993, Science 261:1411-1418; Szostak, 1993, TIBS 17, 89-93; Kumar et al., 1995, FASEB J, 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention will block to some extent Chk1 protein expression and can be used to treat disease or diagnose disease associated with the levels of Chk1.

The enzymatic nature of a ribozyme has significant advantages, such as the concentration of ribozyme necessary to affect a therapeutic treatment is lower. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a ribozyme.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and achieve efficient cleavage in vitro (Zaug et al., 324, Nature 429 1986 ; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving ribozymes can be used as therapeutic agents for human disease (Usman & McSwiggen, 1995 Ann. Rep. Med. Chem. 30, 285-294; Christoffersen and Marr, 1995 J. Med. Chem. 38, 2023-2037). Ribozymes can be designed to cleave specific RNA targets within the background of cellular RNA. Such a cleavage event renders the RNA non-functional and abrogates protein expression from that RNA. In this manner, synthesis of a protein associated with a disease state can be selectively inhibited (Warashina et al., 1999, Chemistry and Biology, 6, 237-250).

The nucleic acid molecules of the instant invention are also referred to as GeneBloc™ reagents, which are essentially nucleic acid molecules (e.g.; ribozymes, antisense) capable of down-regulating gene expression.

GeneBlocs are modified oligonucleotides including ribozymes and modified antisense oligonucleotides that bind to and target specific mRNA molecules. Because GeneBlocs can be designed to target any specific mRNA, their potential applications are quite broad. Traditional antisense approaches have often relied heavily on the use of phosphorothioate modifications to enhance stability in biological samples, leading to a myriad of specificity problems stemming from non-specific protein binding and general cytotoxicity (Stein, 1995, Nature Medicine, 1, 1119). In contrast, GeneBlocs contain a number of modifications that confer nuclease resistance while making minimal use of phosphorothioate linkages, which reduces toxicity, increases binding affinity and minimizes non-specific effects compared with traditional antisense oligonucleotides. Similar reagents have recently been utilized successfully in various cell culture systems (Vassar, et al., 1999, Science, 286, 735) and in vivo (Jarvis et al., manuscript in preparation). In addition, novel cationic lipids can be utilized to enhance cellular uptake in the presence of serum. Since ribozymes and antisense oligonucleotides regulate gene expression at the RNA level, the ability to maintain a steady-state dose of GeneBloc over several days was important for target protein and phenotypic analysis. The advances in resistance to nuclease degradation and prolonged activity in vitro have supported the use of GeneBlocs in target validation applications.

### Target sites

Targets for useful ribozymes and antisense nucleic acids can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468. All of these publications are hereby incorporated by reference herein in their totality. Other examples include the following PCT applications, which concern inactivation of expression of disease-related genes: WO 95/23225, WO 95/13380, WO 94/02595, all of which are incorporated by reference herein. Rather than repeat the guidance provided in those documents here, specific examples of such methods are provided herein, not limiting to those in the art. Ribozymes and antisense to such targets are designed as described in those applications and synthesized to be tested in vitro and in vivo, as also described. The sequences of human Chk1 RNAs were screened for optimal enzymatic nucleic acid and antisense target sites using a computer-folding algorithm. Antisense, hammerhead, DNAzyme, NCH, amberzyme, zinzyme, or G-Cleaver ribozyme binding/cleavage sites were identified. These sites are shown in Tables III to VIII (all sequences are 5′ to 3′ in the tables; underlined regions can be any sequence or linker X, the actual sequence is not relevant here). The nucleotide base position is noted in the Tables as that site to be cleaved by the designated type of enzymatic nucleic acid molecule. While human sequences can be screened and enzymatic nucleic acid molecule and/or antisense thereafter designed, as discussed in Stinchcomb et al., WO 95/23225, mouse targeted ribozymes may be useful to test efficacy of action of the enzymatic nucleic acid molecule and/or antisense prior to testing in humans.

Antisense, hammerhead, DNAzyme, NCH, amberzyme, zinzyme or G-Cleaver ribozyme binding/cleavage sites were identified. The nucleic acid molecules are individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions such as between the binding arms and the catalytic core are eliminated from consideration. Varying binding arm lengths can be chosen to optimize activity.

Antisense, hammerhead, DNAzyme, NCH, amberzyme, zinzyme or G-Cleaver ribozyme binding/cleavage sites were identified and were designed to anneal to various sites in the RNA target. The binding arms are complementary to the target site sequences described above. The nucleic acid molecules were chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; Wincott et al., 1995 Nucleic Acids Res. 23, 2677-2684; and Caruthers et al., 1992, Methods in Enzymology 211,3-19.

### Synthesis of Nucleic acid Molecules

Synthesis of nucleic acids greater than 100 nucleotides in length is difficult using automated methods, and the therapeutic cost of such molecules is prohibitive. In this invention, small nucleic acid motifs (“small refers to nucleic acid motifs no more than 100 nucleotides in length, preferably no more than 80 nucleotides in length, and most preferably no more than 50 nucleotides in length; e.g., antisense oligonucleotides, hammerhead or the NCH ribozymes) are preferably used for exogenous delivery. The simple structure of these molecules increases the ability of the nucleic acid to invade targeted regions of RNA structure. Exemplary molecules of the instant invention are chemically synthesized, and others can similarly be synthesized.

Oligonucleotides (e.g.; antisense GeneBlocs™) are synthesized using protocols known in the art as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphorarnidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 sec coupling step for 2′-deoxy nucleotides. Table II outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by calorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the antisense oligonucleotides is performed as follows: the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H20/3:1:1, vortexed and the supernatant is then added to the first supernatant. The combined supernatants, containing the oligoribonucleotide, are dried to a white powder.

The method of synthesis used for normal RNA including certain enzymatic nucleic acid molecules follows the procedure as described in Usman et al., 1987, J. Am. Chem. Soc., 109, 7845; Scaringe et al., 1990, Nucleic Acids Res., 18, 5433; Wincott et al., 1995, Nucleic Acids Res. 23, 2677-2684 and Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H20/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA•3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA•3HF (0.1 mL) is added and the vial is heated at 65° C. for 15 min. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive hammerhead ribozymes or binding attenuated control (BAC) oligonucleotides) are synthesized by substituting a U for G₅ and a U for A₁₄ (numbering from Hertel, K. J., et al., 1992, Nucleic Acids Res., 20, 3252). Similarly, one or more nucleotide substitutions can be introduced in other enzymatic nucleic acid molecules to inactivate the molecule and such molecules can serve as a negative control.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the examples described above including but not limited to 96-well format, all that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes are purified by gel electrophoresis using general methods or are purified by high pressure liquid chromatography (HPLC; See Wincott et al., supra, the totality of which is hereby incorporated herein by reference) and are re-suspended in water.

The sequences of the ribozymes and antisense constructs that are chemically synthesized, useful in this study, are shown in Tables III to IX. Those in the art will recognize that these sequences are representative only of many more such sequences where the enzymatic portion of the ribozyme (all but the binding arms) is altered to affect activity. The ribozyme and antisense construct sequences listed in Tables III to IX may be formed of ribonucleotides or other nucleotides or non-nucleotides. Such ribozymes with enzymatic activity are equivalent to the ribozymes described specifically in the Tables.

### Optimizing Activity of the nucleic acid molecule of the invention.

Chemically synthesizing nucleic acid molecules with modifications (base, sugar and/or phosphate) that prevent their degradation by serum ribonucleases can increase their potency (see e.g., Eckstein et at., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; Rossi et al., International Publication No. WO 91/03162; Sproat, U.S. Pat. No. 5,334,711; and Burgin et al., supra); all of these describe various chemical modifications that can be made to the base, phosphate and/or sugar moieties of the nucleic acid molecules described herein. All these references are incorporated by reference herein. Modifications which enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry , 35, 14090). Sugar modifications of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci. , 1992, 17, 334-339; Usman et al. International Publication PCT No. WO 93/15187; Sproat, U.S. Pat. No. 5,334,711 and Beigelman et al, 1995, J. Biol. Chem., 270,25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., USSN 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated by reference herein in their totalities). Such publications describe general methods and strategies to determine the location of incorporation of sugar, base and/or phosphate modifications and the like into ribozymes without inhibiting catalysis. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications may cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications which maintain or enhance activity are provided. Such nucleic acid is also generally more resistant to nucleases than unmodified nucleic acid. Thus, in a cell and/or in vivo the activity may not be significantly lowered. Therapeutic nucleic acid molecules delivered exogenously must optimally be stable within cells until translation of the target RNA has been inhibited long enough to reduce the levels of the undesirable protein. This period of time varies between hours to days depending upon the disease state. Clearly, nucleic acid molecules must be resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein) have expanded the ability to modify nucleic acid molecules by introducing nucleotide modifications to enhance their nuclease stability as described above.

Use of the nucleic acid-based molecules of the present invention will lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients with nucleic acid molecules can also include combinations of different types of nucleic acid molecules.

Therapeutic nucleic acid molecules (e.g., enzymatic nucleic acid molecules and antisense nucleic acid molecules) delivered exogenously must optimally be stable within cells until translation of the target RNA has been inhibited long enough to reduce the levels of the undesirable protein. This period of time varies between hours to days depending upon the disease state. Clearly, these nucleic acid molecules must be resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

In yet another preferred embodiment, nucleic acid catalysts having chemical modifications which maintain or enhance enzymatic activity are provided. Such nucleic acid is also generally more resistant to nucleases than unmodified nucleic acid. Thus, in a cell and/or in vivo the activity may not be significantly lowered. As exemplified herein such ribozymes are useful in a cell and/or in vivo even if activity over all is reduced 10 fold (Burgin et al., 1996, Biochemistry, 35, 14090). Such ribozymes herein are said to “maintain” the enzymatic activity of an all RNA ribozyme.

In another aspect the nucleic acid molecules comprise a 5′ and/or a 3′-cap structure.

By “cap structure” is meant chemical modifications, which have been incorporated at either terminus of the oligonucleotide (see, for example, Wincott et al., WO 97/26270, incorporated by reference herein). These terminal modifications protect the nucleic acid molecule from exonuclease degradation, and may help in delivery and/or localization within a cell. The cap may be present at the 5′-terminus (5′-cap) or at the 3′-terminus (3′-cap) or may be present on both termini. In non-limiting examples the 5′-cap is selected from the group comprising inverted abasic residue (moiety), 4′,5′-methylene nucleotide; I-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofaranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

In yet another preferred embodiment, the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details, see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group may be substituted or unsubstituted. When substituted the substituted group(s) is preferably, hydroxyl, cyano, alkoxy, ═O, ═S, NO2 or N(CH3)2, amino, or SH. The term also includes alkenyl groups which are unsaturated hydrocarbon groups containing at least one carbon—carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group may be substituted or unsubstituted. When substituted the substituted group(s) is preferably, hydroxyl, cyano, alkoxy, ═O, ═S, NO2, halogen, N(CH3)2, amino, or SH. The term “alkyl” also includes alkynyl groups which have an unsaturated hydrocarbon group containing at least one carbon—carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group may be substituted or unsubstituted. When substituted the substituted group(s) is preferably, hydroxyl, cyano, alkoxy, ═O, ═S, NO2 or N(CH3)2, amino or SH.

Such alkyl groups may also include aryl, alkylaryl, carbocyclic aryl, heterocyclic aryl, amide and ester groups. An “aryl” group refers to an aromatic group which has at least one ring having a conjugated π electron system and includes carbocyclic aryl, heterocyclic aryl and biaryl groups, all of which may be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, -D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases may be used at any position, for example, within the catalytic core of an enzymatic nucleic acid molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2 -thiouridine, 5-carboxymethylaminomethyluridine, -D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases may be used at any position, for example, within the catalytic core of an enzymatic nucleic acid molecule and/or in the substrate-binding regions of the nucleic acid molecule.

In a preferred embodiment, the invention features modified ribozymes with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39.

By “abasic” is meant sugar moieties lacking a base or having other chemical groups in place of a base at the 1′ position, (for more details, see Wincott et al., International PCT publication No. WO 97/26270).

By “unmodified nucleoside” is meant one of the bases adenine, cytosine, guanine, thymine, uracil joined to the 1′ carbon of beta-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′—O—NH₂, which may be modified or unmodified. Such modified groups are described, for example, in Eckstein et al., U.S. Pat. No. 5,672,695 and Matulic-Adamic et al., WO 98/28317, respectively, which are both incorporated by reference herein in their entireties.

Various modifications to nucleic acid (e.g., antisense and ribozyme) structure can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

Use of these molecules will lead to better treatment of the disease progression by affording the possibility of combination therapies (e.g., multiple ribozymes targeted to different genes, ribozymes coupled with known small molecule inhibitors, or intermittent treatment with combinations of ribozymes (including different ribozyme motifs) and/or other chemical or biological molecules). The treatment of patients with nucleic acid molecules may also include combinations of different types of nucleic acid molecules. Therapies may be devised which include a mixture of ribozymes (including different ribozyme motifs), antisense and/or 2-5A chimera molecules to one or more targets to alleviate symptoms of a disease.

### Administration of Nucleic Acid Molecules

Methods for the delivery of nucleic acid molecules are described in Akhtar et al., 1992, Trends Cell Bio., 2, 139; and Delivery Strategies for Antisense Oligonucleotide Therapeutics, ed. Akhtar, 1995 which are both incorporated herein by reference. Sullivan et al., PCT WO 94/02595, further describes the general methods for delivery of enzymatic RNA molecules. These protocols may be utilized for the delivery of virtually any nucleic acid molecule. Nucleic acid molecules may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, nucleic acid molecules may be directly delivered ex vivo to cells or tissues with or without the aforementioned vehicles. Alternatively, the nucleic acid/vehicle combination is locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of nucleic acid delivery and administration are provided in Sullivan et al., supra, Draper et al., PCT W093/23569, Beigelman et al., PCT W099/05094, and Klimuk et al., PCT W099/04819 all of which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration; suppositories for rectal administration; sterile solutions; suspensions for injectable administration; and other compositions known in the art.

The present invention also includes pharmaceutically acceptable formulations of the compounds described. These formulations include salts of the above compounds, e.g., acid addition salts, including salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes that lead to systemic absorption include, without limitations: intravenous, subcutaneous, intraperitoneal, inhalation, oral, intrapulmonary and intramuscular. Each of these administration routes exposes the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

By pharmaceutically acceptable formulation is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: P-glycoprotein inhibitors (such as Pluronic P85) which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, DF et al, 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies for the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). All incorporated by reference herein. Such liposomes have been shown to accumulate selectively in tumors, presumably by extravasation and capture in the neovascularized target tissues (Lasic et al., Science 1995, 267, 1275-1276; Oku et al.,1995, Biochim. Biophys. Acta, 1238, 86-90). All incorporated by reference herein. The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of which are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the present invention may also be administered to a patient in combination with other therapeutic compounds to increase the overall therapeutic effect. The use of multiple compounds to treat an indication may increase the beneficial effects while reducing the presence of side effects.

Alternatively, certain of the nucleic acid molecules of the instant invention can be expressed within cells from eukaryotic promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol, 66, 1432-41; Weerasinghe et al., 1991, J. Virol., 65, 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Thompson et al., 1995, Nucleic Acids Res., 23, 2259; Good et al., 1997, Gene Therapy, 4, 45; all of the references are hereby incorporated in their totality by reference herein). Those skilled in the art realize that any nucleic acid can be expressed in eukaryotic cells from the appropriate DNA/RNA vector. The activity of such nucleic acids can be augmented by their release from the primary transcript by a ribozyme (Draper et al., PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-30; Ventura et al., 1993, Nucleic Acids Res., 21, 3249-55; Chowrira et al., 1994, J. Biol. Chem., 269, 25856; all of these references are hereby incorporated in their totalities by reference herein).

In another aspect of the invention, RNA molecules of the present invention are preferably expressed from transcription units (see, for example, Couture et al., 1996, TIG., 12, 510) inserted into DNA or RNA vectors. The recombinant vectors are preferably DNA plasmids or viral vectors. Ribozyme expressing viral vectors could be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors may be used that provide for transient expression of nucleic acid molecules. Such vectors might be repeatedly administered as necessary. Once expressed, the nucleic acid molecule binds to the target MRNA. Delivery of nucleic acid molecule expressing vectors could be systemic, such as by intravenous or intra-muscular administration, by administration to target cells ex-planted from the patient followed by reintroduction into the patient, or by any other means that would allow for introduction into the desired target cell (for a review, see Couture et al., 1996, TIG., 12, 510).

In one aspect, the invention features an expression vector comprising a nucleic acid sequence encoding at least one of the nucleic acid molecules disclosed in the instant invention. The nucleic acid sequence encoding the nucleic acid molecule of the instant invention is operable linked in a manner which allows expression of that nucleic acid molecule.

In another aspect, the invention features an expression vector comprising: a) a transcription initiation region (e.g., eukaryotic pol I,II or III initiation region); b) a transcription termination region (e.g., eukaryotic pol I,II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector may optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. U S A, 87, 6743-7; Gao and Huang 1993, Nucleic Acids Res.., 21, 2867-72; Lieber et al., 1993, Methods Enzymol., 217, 47-66; Zhou et al., 1990, Mol. Cell. Biol., 10, 4529-37). All of these references are incorporated by reference herein.

Several investigators have demonstrated that nucleic acid molecules, such as ribozymes expressed from such promoters can function in mammalian cells (e.g. Kashani-Sabet et al., 1992, Antisense Res. Dev., 2, 3-15; Ojwang et al., 1992, Proc. Natl. Acad. Sci. U S A, 89, 10802-6; Chen et al., 1992, Nucleic Acids Res., 20, 4581-9; Yu et al., 1993, Proc. Natl. Acad. Sci. U S A, 90, 6340-4; L'Huillier et al., 1992, EMBO J., 11, 4411-8; Lisziewicz et al., 1993, Proc. Natl. Acad. Sci. U. S. A, 90, 8000-4; Thompson et al., 1995, Nucleic Acids Res., 23, 2259; and Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; and Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review, see Couture and Stinchcomb, 1996, supra).

In yet another aspect, the invention features an expression vector comprising a nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule.

In another preferred embodiment, the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule.

In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule.

In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of Antisense, hammerhead, DNAzyme, NCH, Amberzyme, Zinzyme, or G-Cleaver ribozyme molecules and binding/cleavage sites within Chk1 RNA.

### Nucleic acid inhibition of Chk1 target RNA

Control of the cell cycle is one of the most highly orchestrated events in the cell. There is a great deal of interest in discovering the function of genes involved in mitotic checkpoint abrogation, since inhibition of these genes or activities of these gene products could sensitize cells to DNA damaging agents. In these studies, the cell cycle regulatory role of Chk1 (GeneBank Accession #AF016582 is investigated).

In the fission yeast Schizosaccharomyces pombe, DNA damage by gamma irradiation or a chemical agent such as etoposide leads to activation of Cbk1 by phosphorylation. Chk1, also known as p56chk1, is a Wee 1-like protein kinase, which phosphorylates and inactivates Cdc25. Cdc25 is a phosphatase that acts directly on Cdc2. Chk1 is required for the DNA damage checkpoint, whereas the rad gene products are required for both S—M and DNA damage checkpoints. Wee 1 is also phosphorylated by Chk1 in vitro, also suggesting that Wee 1 is regulated by Chk1 in vivo and the resulting G2 delay is the result of maintaining Y15 phosphorylation on Cdc2. In normal mammalian cells, DNA damage would lead to arrest at G1/S arrest via the p53 pathway, or G2/M arrest via the Cdc2/CyclinB pathway. Thus, p53- cells can remain viable following DNA damage because of the Cdc2/CyclinB arrest pathway. If the Cdc2/CyclinB mediated checkpoint is abrogated via inhibition of Wee1 and Myt1 by small molecule inhibitors in a p53- cell type, then viability is compromised. Chk1 has recently been cloned from mammalian cells. The Chk1 protein is modified in response to DNA damage, and has been shown to bind and phosphorylate Cdc25A, Cdc25B and Cdc25C. The phosphorylation of Cdc25C prevents activation of the Cdc2/CyclinB complex and blocks entry into mitosis, thereby validating the inhibition of Chk1 as a target for nucleic acid based therapeutics.

To address whether checkpoint kinases function redundantly during DNA replication and/or DNA damage checkpoint responses, applicant undertook an oligonucleotide-based approach to block Chk1 gene function in a human cell line. HeLa cells lacking Chk1 protein failed to maintain a G2 cell cycle arrest after etoposide or gamma radiation-induced DNA damaging treatments. Additionally, Chk1-defeicient cells failed to respond to the DNA replication inhibitor hydroxyurea. Based on these results, applicant concludes that the Chk1 kinase plays an essential role in both the DNA replication and DNA damage checkpoint responses. These results also suggest the neither Chk2 nor C-TAK1 kinases function in these checkpoint responses to a significant level, at least in HeLa cells. Thus, Chk1 is validated as an attractive therapeutic target for abrogating the G2 DNA damage checkpoint arrest; a situation that may selectively sensitize p53-deficient tumor cells to radiation or chemotherapy treatment.

### Example 1

### Identification of Potential Target Sites in Human Chk1 RNA

The sequence of human Chk1 is screened for accessible sites using a computer-folding algorithm. Regions of the RNA are identified that do not form secondary folding structures. These regions contain potential ribozyme and/or antisense binding/cleavage sites. The sequences of these binding/cleavage sites are shown in Tables III-IX.

### Example 2

### Selection of Enzymatic Nucleic Acid Cleavage Sites in Human Chk1 RNA

Ribozyme target sites are chosen by analyzing sequences of Human Chk1 (Genbank accession number: AF016582) and prioritizing the sites on the basis of folding. Ribozymes are designed that could bind each target and are individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the ribozyme sequences fold into the appropriate secondary structure. Those ribozymes with unfavorable intramolecular interactions between the binding arms and the catalytic core are eliminated from consideration. As noted below, varying binding arm lengths can be chosen to optimize activity. Generally, at least 5 bases on each arm are able to bind to, or otherwise interact with, the target RNA.

### Example 3

### Chemical Synthesis and Purification of Ribozymes and Antisense for Efficient Cleavage and/or blocking of Chk1 RNA

Ribozymes and antisense constructs are designed to anneal to various sites in the RNA message. The binding arms of the ribozymes are complementary to the target site sequences described above, while the antisense constructs are fully complimentary to the target site sequences described above. The ribozymes and antisense constructs were chemically synthesized. The method of synthesis used followed the procedure for normal RNA synthesis as described above and in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were typically >98%.

Ribozymes and antisense constructs are also synthesized from DNA templates using bacteriophage T7 RNA polymerase (Milligan and Uhlenbeck, 1989, Methods Enzymol. 180, 51). Ribozymes and antisense constructs are purified by gel electrophoresis using general methods or are purified by high pressure liquid chromatography (HPLC; see Wincott et al., supra; the totality of which is hereby incorporated herein by reference) and are resuspended in water. The sequences of the chemically synthesized ribozymes and antisense constructs used in this study are shown below in Table III-IX.

### Example 4

### Ribozyme Cleavage of Chk1 RNA Target in vitro

Ribozymes targeted to the human Chk1 RNA are designed and synthesized as described above. These ribozymes can be tested for cleavage activity in vitro, for example, using the following procedure. The target sequences and the nucleotide location within the Chk1 RNA are given in Tables III-IX.

Cleavage Reactions: Full-length or partially full-length, internally-labeled target RNA for ribozyme cleavage assay is prepared by in vitro transcription in the presence of [a-³²P] CTP, passed over a G 50 Sephadex® column by spin chromatography and used as substrate RNA without further purification. Alternately, substrates are 5′-³²P-end labeled using T4 polynucleotide kinase enzyme. Assays are performed by pre-warming a 2× concentration of purified ribozyme in ribozyme cleavage buffer (50 mM Tris-HCl, pH 7.5 at 37° C., 10 mM MgCl₂) and the cleavage reaction was initiated by adding the 2× ribozyme mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM ribozyme, i.e., ribozyme excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by ribozyme cleavage are visualized on an autoradiograph of the gel. The percentage of cleavage is determined by Phosphor Imager® quantitation of bands representing the intact substrate and the cleavage products.

### Example 5

### Nucleic acid inhibition of Chk1 target RNA in vivo

Antisense nucleic acid molecules (GeneBlocs™) targeted to the human Chk1 RNA are designed and synthesized as described above. These nucleic acid molecules can be tested for cleavage activity in vivo, for example, using the following procedure. The target sequences and the nucleotide location within the Chk1 RNA are given in Tables III-IX.

Two formats were used to test the efficacy of nucleic acid reagents (GeneBlocs™ targeting Chk1. First, the reagents were tested on asynchronous HeLa cells, to determine the extent of RNA and protein inhibition. To demonstrate whether cells bypass the G2/M checkpoint, HeLa cells (p53−) are treated with etoposide to damage the DNA. Nocodazole and the potential checkpoint inhibitor are added 16 hours later, when all the cells should be arrested in G2. Nocodazole blocks cells from leaving mitosis, so if they have abrogated the checkpoint, the cells will be blocked in mitosis and appear “rounded” in shape. Other surrogate mitotic markers include decreased phosphorylation of cdc-2 at Thr14 and Tyr15, phosphorylation of Myt-1, and phosphorylation of PP1. This study set out to determine whether inhibiting expression of the Chk1 gene would allow the G2/M checkpoint to be bypassed after DNA damage, as well as determining if the presence of p53 influences the DNA-damage checkpoint response.

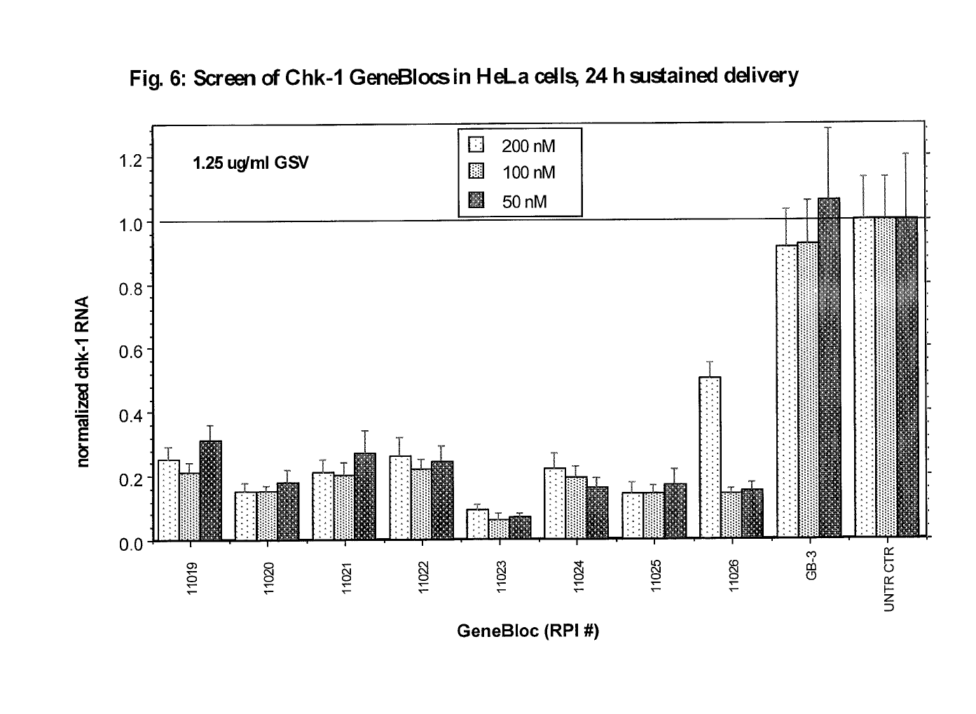

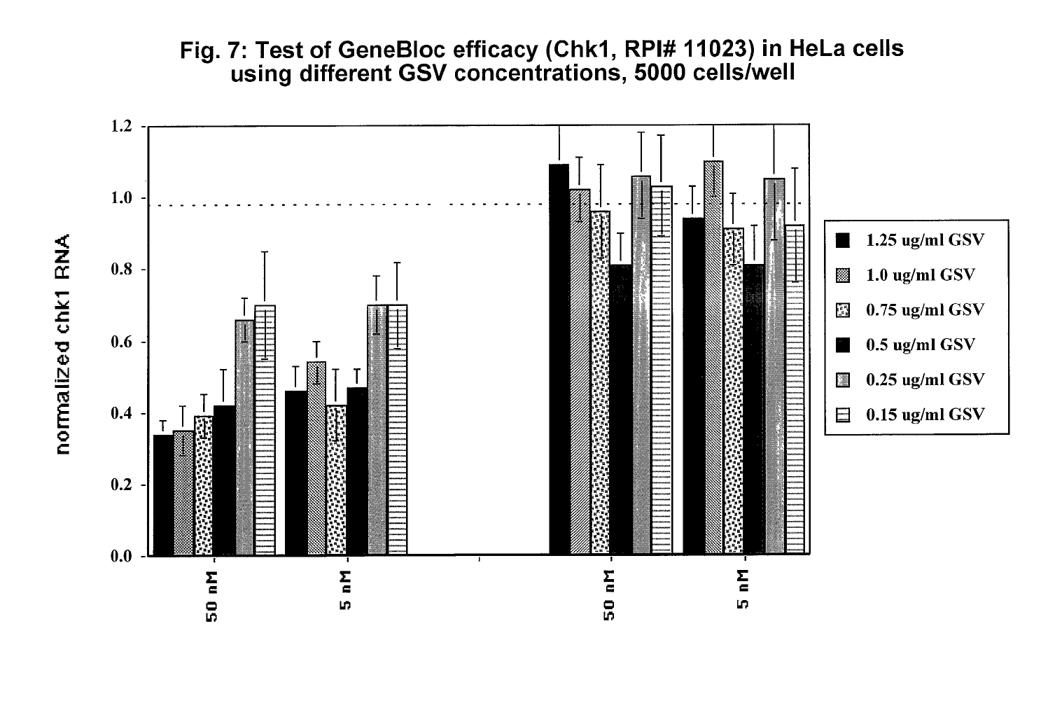

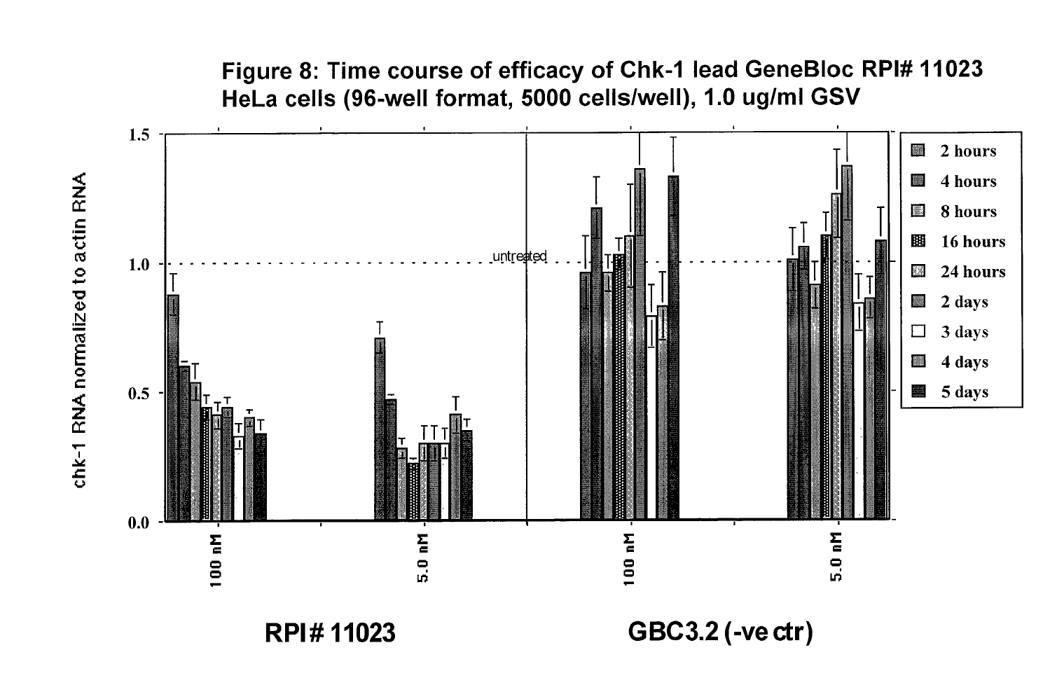

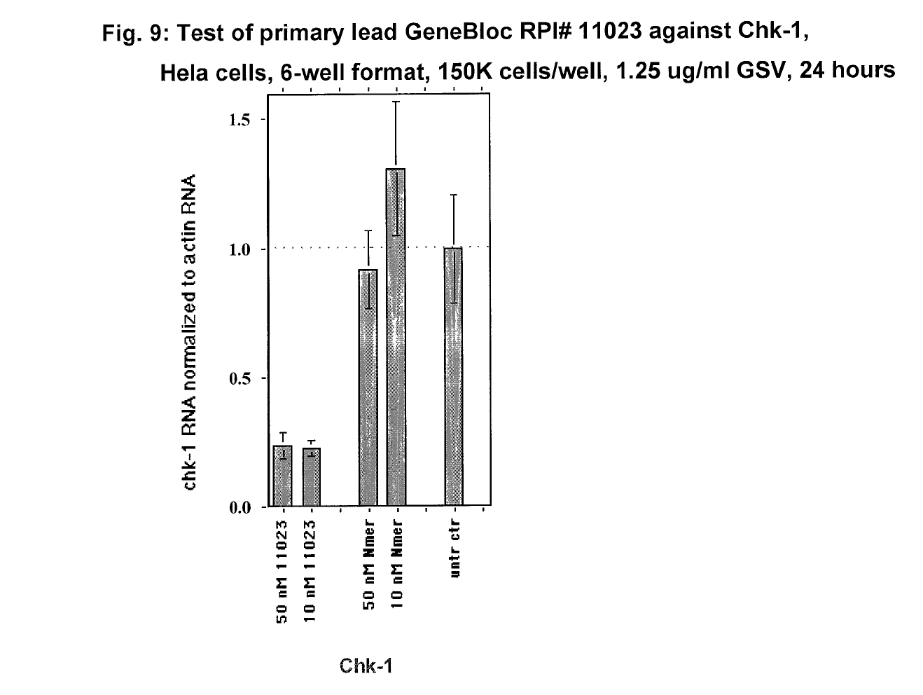

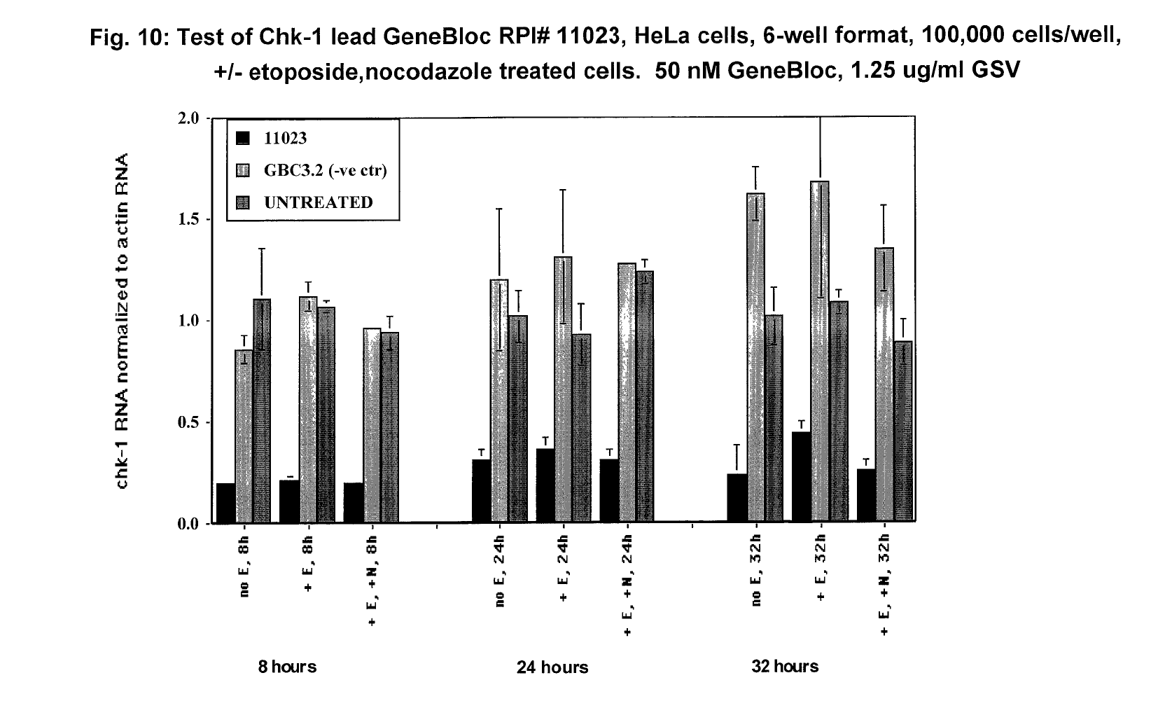

Eight GeneBloc™ reagents (e.g.; see Table IX) were selected against the Chk1 cDNA target. RNA inhibition was measured after delivery of these reagents by GSV lipid (Glenn Research) to HeLa cells. Relative amounts of target RNA were measured versus actin using real-time PCR monitoring of amplification (ABI 7700 Taqman®). The results are shown in FIG. 6. The comparison is made to a mixture of 5 oligonucleotide sequences made to unrelated targets (GB-3) or to a randomized oligonucleotide control with the same overall length and chemistry, but randomly substituted at each position (GBC3.2). Primary and secondary lead reagents were chosen for the target and optimization performed. The optimal GSV lipid concentration was chosen after screening for RNA inhibition with oligonucleotides at 5 and 50 nM (FIG. 7). After optimal lipid concentration was chosen, a RNA time-course of inhibition was performed with the lead nucleic acid molecule (GeneBloc™) (FIG. 8). In addition, a cell-plating format was tested for RNA inhibition. The use of a 96-well (5000 cells/well) versus six-well (150,000 cells/well) plating density made no difference in the extent of RNA inhibition (FIG. 9). The phenotypic assays require treatment with etoposide and nocodazole as described above, and RNA inhibition in this assay was also determined (FIG. 10). The various treatments had essentially no effect on RNA levels.

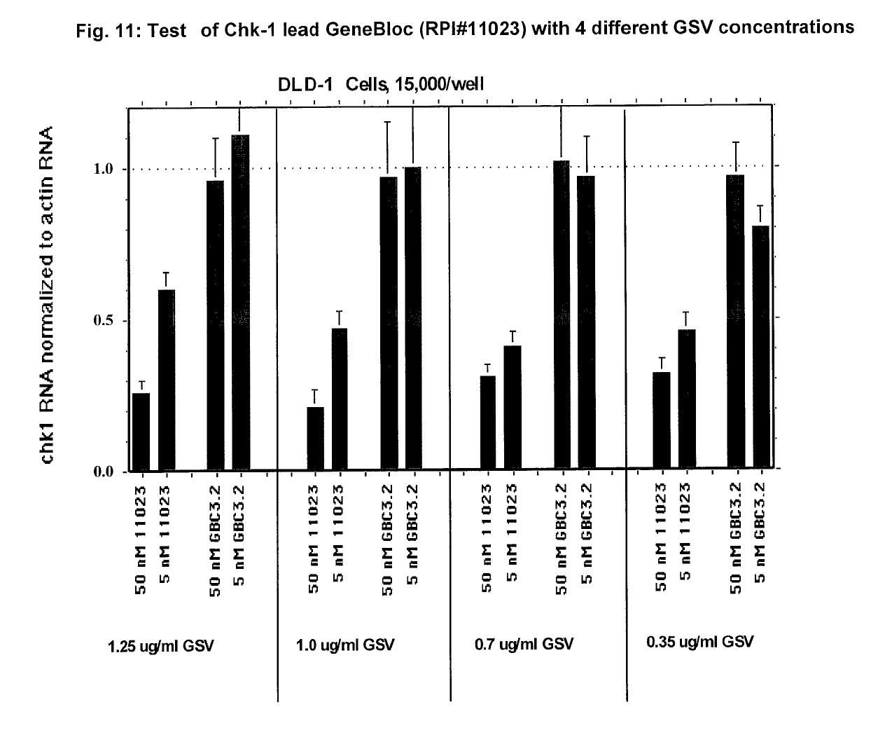

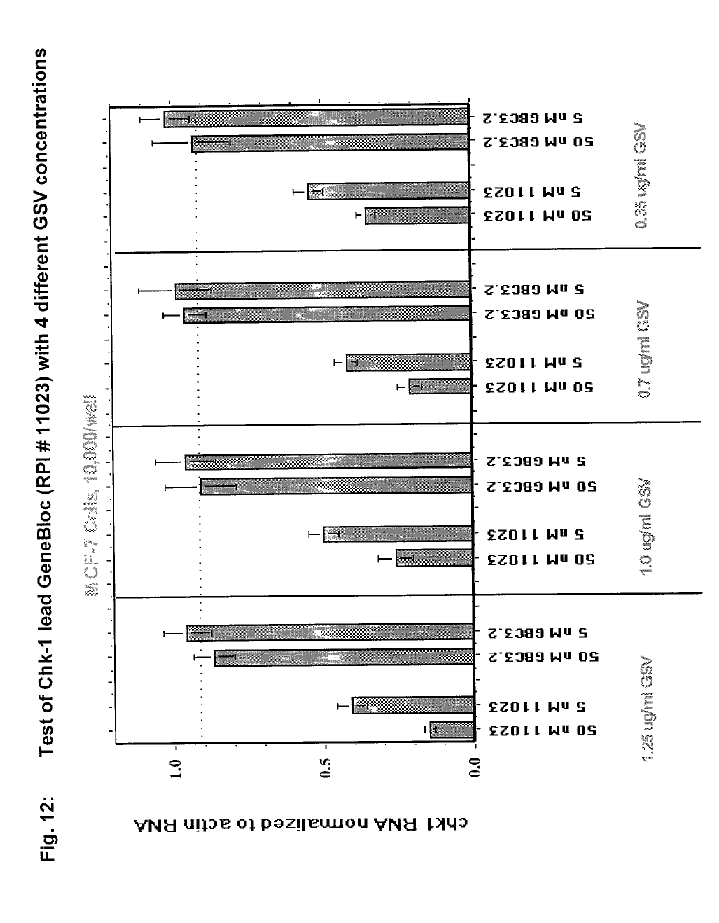

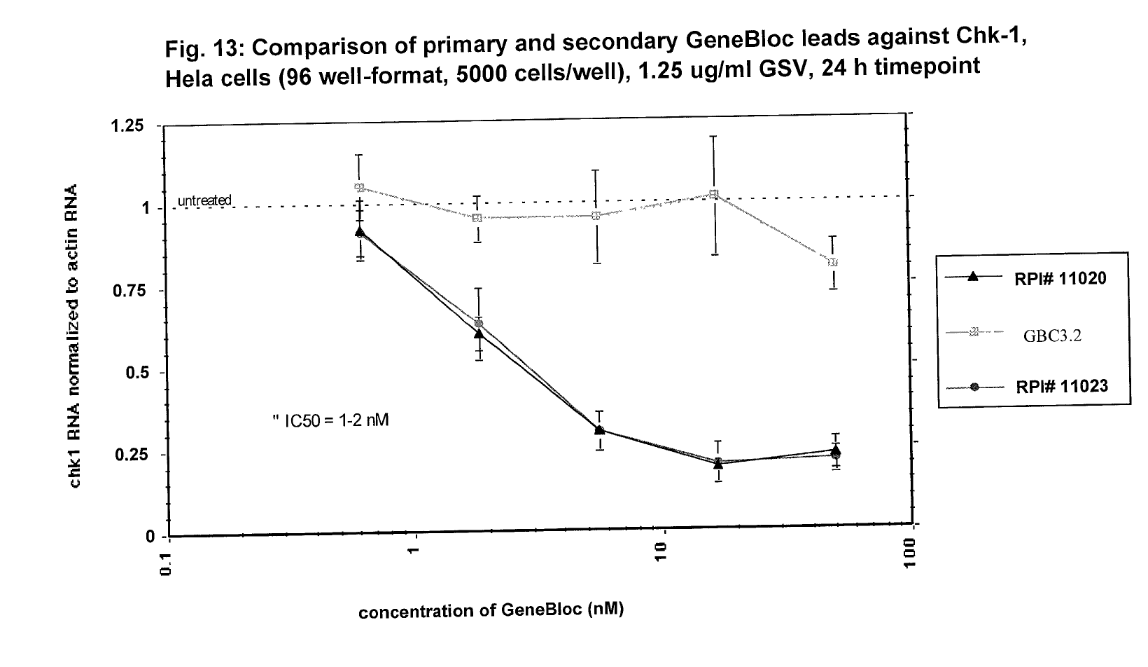

Optimization of delivery conditions were also performed in DLD-1 (p53−) (FIG. 11) and MCF-7 (FIG. 12) (p53+) cells. Similar levels of inhibition were observed when compared to HeLa cells at the optimal GSV concentration. Dose curves were also generated in HeLa cells with the two best lead nucleic acid molecules (FIG. 13). IC50 values for both leads were in the 1-2 nM range. Similar IC50s were observed in DLD-1 and MCF-7 cells. Protein levels were assessed at 8, 24 and 32 hours after nucleic acid administration, as well as one to five days post delivery. The target protein was significantly reduced (80-90%) by 24 hours after nucleic acid administration and remained low (undetectable by western blot) until at least day 5. Application of nucleic acid inhibitors in the checkpoint abrogation assay resulted in the “rounding up” phenotype for the Chk1 target. Also, there is an increase in Myt 1 phosphorylation and a large increase in PP1 phosphorylation. There also appear to be decreases in phosphorylation of the Y15 and T14 residues on Cdc2, although this is not complete. Most importantly, this evidence demonstrates the role of Chk1 in the G2/M checkpoint and suggests that inhibitors of Chk1 activity can be useful alone or in combination with DNA damaging agents in treatment of certain types of cancer.

### Indications

Particular degenerative and disease states that can be associated with Chk1 expression modulation include but are not limited to cancers of the colon, rectum, lung, breast and prostate

The present body of knowledge in Chk1 research indicates the need for methods to assay Chk1 activity and for compounds that can regulate Chk1 expression for research, diagnostic, and therapeutic use.

Radiation and chemotherapeutic treatments are non-limiting examples of methods that can be combined with or used in conjunction with the nucleic acid molecules (e.g. ribozymes and antisense molecules) of the instant invention. Those skilled in the art will recognize that other drug compounds and therapies can be similarly be readily combined with the nucleic acid molecules of the instant invention (e.g. ribozymes and antisense molecules) are hence within the scope of the instant invention.

### Diagnostic uses

The nucleic acid molecules of this invention (e.g., ribozymes) can be used as diagnostic tools to examine genetic drift and mutations within diseased cells or to detect the presence of Chk1 RNA in a cell. The close relationship between ribozyme activity and the structure of the target RNA allows the detection of mutations in any region of the molecule which alters the base-pairing and three-dimensional structure of the target RNA. By using multiple ribozymes described in this invention, one can map nucleotide changes which are important to RNA structure and function in vitro, as well as in cells and tissues. Cleavage of target RNAs with ribozymes can be used to inhibit gene expression and define the role (essentially) of specified gene products in the progression of disease. In this manner, other genetic targets can be defined as important mediators of the disease. These experiments will lead to better treatment of the disease progression by affording the possibility of combinational therapies (e.g., multiple ribozymes targeted to different genes, ribozymes coupled with known small molecule inhibitors, or intermittent treatment with combinations of ribozymes and/or other chemical or biological molecules). Other in vitro uses of ribozymes of this invention are well known in the art, and include detection of the presence of mRNAs associated with Chk1-related condition. Such RNA is detected by determining the presence of a cleavage product after treatment with a ribozyme using standard methodology.

In a specific example, ribozymes which can cleave only wild-type or mutant forms of the target RNA are used for the assay. The first ribozyme is used to identify wild-type RNA present in the sample and the second ribozyme is used to identify mutant RNA in the sample. As reaction controls, synthetic substrates of both wild-type and mutant RNA is cleaved by both ribozymes to demonstrate the relative ribozyme efficiencies in the reactions and the absence of cleavage of the “non-targeted” RNA species. The cleavage products from the synthetic substrates also serve to generate size markers for the analysis of wild-type and mutant RNAs in the sample population. Thus, each analysis requires two ribozymes, two substrates and one unknown sample, which is combined into six reactions. The presence of cleavage products is determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., Chk1) is adequate to establish risk. If probes of comparable specific activity are used for both transcripts, then a qualitative comparison of RNA levels will be adequate and will decrease the cost of the initial diagnosis. Higher mutant form to wild-type ratios are correlated with higher risk whether RNA levels are compared qualitatively or quantitatively.

### Additional Uses

Potential usefulness of sequence-specific enzymatic nucleic acid molecules of the instant invention might have many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs could be specifically cleaved to fragments of a size more useful for study. The ability to engineer sequence specificity of the enzymatic nucleic acid molecule is ideal for cleavage of RNAs of unknown sequence. Applicant has described the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of’ and “consisting of’ may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the claims that follow.

TABLE I
Characteristics of naturally occurring ribozymes

Group I Introns
Size: ˜150 to >1000 nucleotides.
Requires a U in the target sequence immediately 5′ of the cleavage site.
Binds 4-6 nucleotides at the 5′-side of the cleavage site.
Reaction mechanism: attack by the 3′-OH of guanosine to generate
cleavage products with 3′-OH and 5′-guanosine.
Additional protein cofactors required in some cases to help folding and
maintainance of the active structure.
Over 300 known members of this class. Found as an intervening sequence
in Tetrahymena thermophila rRNA, fungal mitochondria, chloroplasts,
phage T4, blue-green algae, and others.
Major structural features largely established through phylogenetic
comparisons, mutagenesis, and biochemical studies [^{i,ii}].
Complete kinetic framework established for one ribozyme [^{iii,iv,v,vi}].
Studies of ribozyme folding and substrate docking underway [^{vii,viii,ix}].
Chemical modification investigation of important residues well
established [^{x,xi}].
The small (4-6 nt) binding site may make this ribozyme too non-specific
for targeted RNA cleavage, however, the Tetrahymena group I intron has
been used to repair a “defective” beta-galactosidase message by
the ligation of new beta-galactosidase sequences onto the defective
message [^{xii}].
RNAse P RNA (M1 RNA)
Size: ˜290 to 400 nucleotides.
RNA portion of a ubiquitous ribonucleoprotein enzyme.
Cleaves tRNA precursors to form mature tRNA [^{xiii}].
Reaction mechanism: possible attack by M²⁺—OH to generate cleavage
products with 3′-OH and 5′-phosphate.
RNAse P is found throughout the prokaryotes and eukaryotes. The RNA
subunit has been sequenced from bacteria, yeast, rodents, and primates.
Recruitment of endogenous RNAse P for therapeutic applications is
possible through hybridization of an External Guide Sequence (EGS) to
the target RNA [^{xiv,xv}]
Important phosphate and 2′ OH contacts recently identified [^{xvi,xvii}]
Group II Introns
Size: >1000 nucleotides.
Trans cleavage of target RNAs recently demonstrated [^{xviii,xix}].
Sequence requirements not fully determined.
Reaction mechanism: 2′-OH of an internal adenosine generates cleavage
products with 3′-OH and a “lariat” RNA containing a 3′-5′ and a
2′-5′ branch point.
Only natural ribozyme with demonstrated participation in DNA cleavage
[^{xx,xxi}] in addition to RNA cleavage and ligation.
Major structural features largely established through phylogenetic
comparisons [^{xxii}].
Important 2′ OH contacts beginning to be identified [^{xxiii}]
Kinetic framework under development [^{xxiv}]
Neurospora VS RNA
Size: ˜144 nucleotides.
Trans cleavage of hairpin target RNAs recently demonstrated [^{xxv}].
Sequence requirements not fully determined.
Reaction mechanism: attack by 2′-OH 5′ to the scissile bond to generate
cleavage products with 2′,3′-cyclic phosphate and 5′-OH ends.
Binding sites and structural requirements not fully determined.
Only 1 known member of this class. Found in Neurospora VS RNA.
Hammerhead Ribozyme
(see text for references)
Size: ˜13 to 40 nucleotides.
Requires the target sequence UH immediately 5′ of the cleavage site.
Binds a variable number nucleotides on both sides of the cleavage site.
Reaction mechanism: attack by 2′-OH 5′ to the scissile bond to generate
cleavage products with 2′,3′-cyclic phosphate and 5′-OH ends.
14 known members of this class. Found in a number of plant pathogens
(virusoids) that use RNA as the infectious agent.
Essential structural features largely defined, including 2 crystal structures
[^{xxvi,xxvii}]
Minimal ligation activity demonstrated (for engineering through in vitro
selection) [^{xxviii}]
Complete kinetic framework established for two or more ribozymes [^{xxix}].
Chemical modification investigation of important residues well
established [^{xxx}].
Hairpin Ribozyme
Size: ˜50 nucleotides.
Requires the target sequence GUC immediately 3′ of the cleavage site.
Binds 4-6 nucleotides at the 5′-side of the cleavage site and a variable
number to the 3′-side of the cleavage site.
Reaction mechanism: attack by 2′-OH 5′ to the scissile bond to generate
cleavage products with 2′,3′-cyclic phosphate and 5′-OH ends.
3 known members of this class. Found in three plant pathogen (satellite
RNAs of the tobacco ringspot virus, arabis mosaic virus and chicory
yellow mottle virus) which uses RNA as the infectious agent.
Essential structural features largely defined [^{xxxi,xxxii,xxxiii,xxxiv}]
Ligation activity (in addition to cleavage activity) makes ribozyme
amenable to engineering through in vitro selection [^{xxxv}]
Complete kinetic framework established for one ribozyme [^{xxxvi}].
Chemical modification investigation of important residues
begun [^{xxxvii,xxxviii}].
Hepatitis Delta Virus (HDV) Ribozyme
Size: ˜60 nucleotides.
Trans cleavage of target RNAs demonstrated [^{xxxix}].
Binding sites and structural requirements not fully determined, although no
sequences 5′ of cleavage site are required. Folded ribozyme contains a
pseudoknot structure [^{xl}].
Reaction mechanism: attack by 2′-OH 5′ to the scissile bond to
generate cleavage products with 2′,3′-cyclic phosphate and 5′-OH ends.
Only 2 known members of this class. Found in human HDV.
Circular form of HDV is active and shows increased nuclease stability [^{xli}]
ⁱMichel, Francois; Westhof, Eric. Slippery substrates. Nat. Struct Biol. (1994), 1(1), 5-7.
ⁱⁱLisacek, Frederique; Diaz, Yolande; Michel, Francois. Automatic identification of group I intron cores in genomic DNA sequences. J. Mol. Biol. (1994), 235(4), 1206-17.
ⁱⁱⁱHerschlag, Daniel; Cech, Thomas R. Catalysis of RNA cleavage by the Tetrahymena thermophila ribozyme. 1. Kinetic description of the reaction of an RNA substrate complementary to the active site. Biochemistry (1990), 29(44), 10159-71.
^{iv}Herschlag, Daniel; Cech, Thomas R. Catalysis of RNA cleavage by the Tetrahymena thermophila ribozyme. 2. Kinetic description of the reaction of an RNA substrate that forms a mismatch at the active site. Biochemistry (1990), 29(44), 10172-80.
^{v}Knitt, Deborah S.; Herschlag, Daniel. pH Dependencies of the Tetrahymena Ribozyme Reveal an Unconventional Origin of an Apparent pKa. Biochemistry (1996), 35(5), 1560-70.
^{vi}Bevilacqua, Philip C.; Sugimoto, Naoki; Turner, Douglas H. A mechanistic framework for the second step of splicing catalyzed by the Tetrahymena ribozyme. Biochemistry (1996), 35(2), 648-58.
^{vii}Li, Yi; Bevilacqua, Philip C.; Mathews, David; Turner, Douglas H. Thermodynamic and activation parameters for binding of a pyrene-labeled substrate by the Tetrahymena ribozyme: docking is not diffusion-controlled and is driven by a favorable entropy change. Biochemistry (1995), 34(44), 14394-9.
^{viii}Banerjee, Aloke Raj; Turner, Douglas H. The time dependence of chemical modification reveals slow steps in the folding of a group I ribozyme. Biochemistry (1995), 34(19), 6504-12.
^{ix}Zarrinkar, Patrick P.; Williamson, James R. The P9.1-P9.2 peripheral extension helps guide folding of the Tetrahymena ribozyme. Nucleic Acids Res. (1996), 24(5), 854-8.
^{x}Strobel, Scott A.; Cech, Thomas R. Minor groove recognition of the conserved G.cntdot.U pair at the Tetrahymena ribozyme reaction site. Science (Washington, D.C.) (1995), 267(5198), 675-9.
^{xi}Strobel, Scott A.; Cech, Thomas R. Exocyclic Amine of the Conserved G.cntdot.U Pair at the Cleavage Site of the Tetrahymena Ribozyme Contributes to 5′-Splice Site Selection and Transition State Stabilization. Biochemistry (1996), 35(4), 1201-11.
^{xii}Sullenger, Bruce A.; Cech, Thomas R. Ribozyme-mediated repair of defective mRNA by targeted trans-splicing. Nature (London) (1994), 371 (6498), 619-22.
^{xiii}Robertson, H.D.; Altman, S.; Smith, J. D. J. Biol. Chem., 247, 5243-5251 (1972).
^{xiv}Forster, Anthony C.; Altman, Sidney. External guide sequences for an RNA enzyme. Science (Washington, D.C., 1883-) (1990), 249(4970), 783-6.
^{xv}Yuan, Y.; Hwang, E. S.; Altman, S. Targeted cleavage of mRNA by human RNase P. Proc. Natl. Acad. Sci. USA (1992) 89, 8006-10.
^{xvi}Harris, Michael E.; Pace, Norman R. Identification of phosphates involved in catalysis by the ribozyme RNase P RNA. RNA (1995), 1(2), 210-18.
^{xvii}Pan, Tao; Loria, Andrew; Zhong, Kun. Probing of tertiary interactions in RNA: 2′-hydroxyl-base contacts between the RNase P RNA and pre-tRNA. Proc. Natl. Acad. Sci. U.S.A. (1995), 92(26), 12510-14.
^{xviii}Pyle, Anna Marie; Green, Justin B. Building a Kinetic Framework for Group II Intron Ribozyme Activity: Quantitation of Interdomain Binding and Reaction Rate. Biochemistry (1994), 33(9), 2716-25.
^{xix}Michels, William J. Jr.; Pyle, Anna Marie. Conversion of a Group II Intron into a New Multiple-Turnover Ribozyme that Selectively Cleaves Oligonucleotides: Elucidation of Reaction Mechanism and Structure/Function Relationships. Biochemistry (1995), 34(9), 2965-77.
^{xx}Zimmerly, Steven; Guo, Huatao; Eskes, Robert; Yang, Jian; Perlman, Philip S.; Lambowitz, Alan M. A group II intron RNA is a catalytic component of a DNA endonuclease involved in intron mobility. Cell (Cambridge, Mass.) (1995), 83(4), 529-38.
^{xxi}Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2′-hydroxyl groups. Chem. Biol. (1995), 2(11), 761-70.
^{xxii}Michel, Francois; Ferat, Jean Luc. Structure and activities of group II introns. Annu. Rev. Biochem. (1995), 64, 435-61.
^{xxiii}Abramovitz, Dana L.; Friedman, Richard A.; Pyle, Anna Marie. Catalytic role of 2′-hydroxyl groups within a group II intron active site. Science (Washington, D.C.) (1996), 271(5254), 1410-13.
^{xxiv}Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256(1), 31-49.
^{xxv}Guo, Hans C. T.; Collins, Richard A. Efficient trans-cleavage of a stem-loop RNA substrate by a ribozyme derived from Neurospora VS RNA. EMBO J. (1995), 14(2), 368-76.
^{xxvi}Scott, W. G., Finch, J. T., Aaron, K. The crystal structure of an all RNA hammerhead ribozyme: A proposed mechanism for RNA catalytic cleavage. Cell, (1995), 81, 991-1002.
^{xxvii}McKay, Structure and function of the hammerhead ribozyme: an unfinished story. RNA, (1996), 2, 395-403.
^{xxviii}Long, D., Uhlenbeck, O., Hertel, K. Ligation with hammerhead ribozymes. U.S. Pat. No. 5,633,133.
^{xxix}Hertel, K. J., Herschlag, D., Uhlenbeck, O. A kinetic and thermodynamic framework for the hammerhead ribozyme reaction. Biochemistry, (1994) 33, 3374-3385. Beigelman, L., et al., Chemical modifications of hammerhead ribozymes. J. Biol. Chem., (1995) 270, 25702-25708.
^{xxx}Beigelman, L., et al., Chemical modifications of hammerhead ribozymes. J. Biol. Chem., (1995) 270, 25702-25708.
^{xxxi}Hampel, Arnold; Tritz, Richard; Hicks, Margaret; Cruz, Philip. ‘Hairpin’ catalytic RNA model: evidence for helixes and sequence requirement for substrate RNA. Nucleic Acids Res. (1990), 18(2), 299-304.
^{xxxii}Chowrira, Bharat M.; Berzal-Herranz, Alfredo; Burke, John M. Novel guanosine requirement for catalysis by the hairpin ribozyme. Nature (London) (1991), 354(6351), 320-2.
^{xxxiii}Berzal-Herranz, Alfredo; Joseph, Simpson; Chowrira, Bharat M.; Butcher, Samuel E.; Burke, John M. Essential nucleotide sequences and secondary structure elements of the hairpin ribozyme. EMBO J. (1993), 12(6), 2567-73.
^{xxxiv}Joseph, Simpson; Berzal-Herranz, Alfredo; Chowrira, Bharat M.; Butcher, Samuel E. Substrate selection rules for the hairpin ribozyme determined by in vitro selection, mutation, and analysis of mismatched substrates. Genes Dev. (1993), 7(1), 130-8.
^{xxxv}Berzal-Herranz, Alfredo; Joseph, Simpson; Burke, John M. In vitro selection of active hairpin ribozymes by sequential RNA-catalyzed cleavage and ligation reactions. Genes Dev. (1992), 6(1), 129-34.
^{xxxvi}Hegg, Lisa A.; Fedor, Martha J. Kinetics and Thermodynamics of Intermolecular Catalysis by Hairpin Ribozymes. Biochemistry (1995), 34(48), 15813-28.
^{xxxvii}Grasby, Jane A.; Mersmann, Karin; Singh, Mohinder; Gait, Michael J. Purine Functional Groups in Essential Residues of the Hairpin Ribozyme Required for Catalytic Cleavage of RNA. Biochemistry (1995), 34(12), 4068-76.
^{xxxviii}Schmidt, Sabine; Beigelman, Leonid; Karpeisky, Alexander; Usman, Nassim; Sorensen, Ulrik S.; Gait, Michael J. Base and sugar requirements for RNA cleavage of essential nucleoside residues in internal loop B of the hairpin ribozyme: implications for secondary structure. Nucleic Acids Res. (1996), 24(4), 573-81.
^{xxxix}Perrotta, Anne T.; Been, Michael D. Cleavage of oligoribonucleotides by a ribozyme derived from the hepatitis delta virus RNA sequence. Biochemistry (1992), 31(1), 16-21.
^{xl}Perrotta, Anne T.; Been, Michael D. A pseudoknot-like structure required for efficient self-cleavage of hepatitis delta virus RNA. Nature (London) (1991), 350(6317), 434-6.
^{xli}Puttaraju, M.; Perrotta, Anne T.; Been, Michael D. A circular trans-acting hepatitis delta virus ribozyme. Nucleic Acids Res. (1993), 21(18), 4253-8.

TABLE II
A. 2.5 μmol Synthesis Cycle ABI 394 Instrument

| Reagent | Equivalents | Amount | Wait Time* DNA | Wait Time* 2’-O-methyl | Wait Time* RNA |
|---|---|---|---|---|---|
| Phosphoramidites | 6.5 | 163 μL | 45 sec | 2.5 min | 7.5 min |
| S-Ethyl Tetrazole | 23.8 | 238 μL | 45 sec | 2.5 min | 7.5 min |
| Acetic Anhydride | 100 | 233 μL | 5 sec | 5 sec | 5 sec |
| N-Methyl | 186 | 233 μL | 5 sec | 5 sec | 5 sec |
| Imidazole | | | | | |
| TCA | 176 | 2.3 μL | 21 sec | 21 sec | 21 sec |
| Iodine | 11.2 | 1.7 μL | 45 sec | 45 sec | 45 sec |
| Beaucage | 12.9 | 645 μL | 100 sec | 300 sec | 300 sec |
| Acetonitrile | NA | 6.67 μL | NA | NA | NA |

B. 0.2 μmol Synthesis Cycle ABI 394 Instrument

| | | | | | |
|---|---|---|---|---|---|
| Phosphoramidites | 15 | 31 μL | 45 sec | 233 sec | 465 sec |
| S-Ethyl Tetrazole | 38.7 | 31 μL | 45 sec | 233 min | 465 sec |
| Acetic Anhydride | 655 | 124 μL | 5 sec | 5 sec | 5 sec |
| N-Methyl | 1245 | 124 μL | 5 sec | 5 sec | 5 sec |
| Imidazole | | | | | |
| TCA | 700 | 732 μL | 10 sec | 10 sec | 10 sec |
| Iodine | 20.6 | 244 μL | 15 sec | 15 sec | 15 sec |
| Beaucage | 7.7 | 232 μL | 100 sec | 300 sec | 300 sec |
| Acetonitrile | NA | 2.64 μL | NA | NA | NA |

C. 0.2 μmol Synthesis Cycle 96 well Instrument

| | Equivalents: DNA/ | Amount: DNA/2’-O- | | Wait Time* 2’-O- | |
|---|---|---|---|---|---|
| Reagent | 2’-O-methyl/Ribo | methyl/Ribo | Wait Time* DNA | methyl | Wait Time* Ribo |
| Phosphoramidites | 22/33/66 | 40/60/120 μL | 60 sec | 180 sec | 360 sec |
| S-Ethyl Tetrazole | 70/105/210 | 40/60/120 μL | 60 sec | 180 min | 360 sec |
| Acetic Anhydride | 265/265/265 | 50/50/50 μL | 10 sec | 10 sec | 10 sec |
| N-Methyl | 502/502/502 | 50/50/50 μL | 10 sec | 10 sec | 10 sec |
| Imidazole | | | | | |
| TCA | 238/475/475 | 250/500/500μL | 15 sec | 15 sec | 15 sec |
| Iodine | 6.8/6.8/6.8 | 80/80/80 μL | 30 sec | 30 sec | 30 sec |
| Beaucage | 34/51/51 | 80/120/120 | 100 sec | 200 sec | 200 sec |
| Acetonitrile | NA | 1150/1150/1150 μL | NA | NA | NA |

*Wait time does not include contact time during delivery.

TABLE III
Human Chk1 Hammerhead Ribozyme and Substrate Sequence

| | | | | Rz Seq |
|---|---|---|---|---|
| Pos | Substrate | Seq ID | Ribozyme | ID |
| 12 | CGGACAGU C CGCCGAGG | 1 | CCUCGGCG CUGAUGAG GCCGUUAGGC CGAA ACUGUCCG | 1423 |
| 25 | GAGGUGCU C GGUGGAGU | 2 | ACUCCACC CUGAUGAG GCCGUUAGGC CGAA AGCACCUC | 1424 |
| 34 | GGUGGAGU C AUGGCAGU | 3 | ACUGCCAU CUGAUGAG GCCGUUAGGC CGAA ACUCCACC | 1425 |
| 48 | AGUGCCCU U UGUGGAAG | 4 | CUUCCACA CUGAUGAG GCCGUUAGGC CGAA AGGGCACU | 1426 |
| 49 | GUGCCCUU U GUGGAAGA | 5 | UCUUCCAC CUGAUGAG GCCGUUAGGC CGAA AAGGGCAC | 1427 |
| 66 | CUGGGACU U GGUGCAAA | 6 | UUUGCACC CUGAUGAG GCCGUUAGGC CGAA AGUCCCAG | 1428 |
| 93 | AGGUGCCU A UGGAGAAG | 7 | CUUCUCCA CUGAUGAG GCCGUUAGGC CGAA AGGCACCU | 1429 |
| 103 | GGAGAAGU U CAACUUGC | 8 | GCAAGUUG CUGAUGAG GCCGUUAGGC CGAA ACUUCUCC | 1430 |
| 104 | GAGAAGUU C AACUUGCU | 9 | AGCAAGUU CUGAUGAG GCCGUUAGGC CGAA AACUUCUC | 1431 |
| 109 | GUUCAACU U GCUGUGAA | 10 | UUCACAGC CUGAUGAG GCCGUUAGGC CGAA AGUUGAAC | 1432 |
| 119 | CUGUGAAU A GAGUAACU | 11 | AGUUACUC CUGAUGAG GCCGUUAGGC CGAA AUUCACAG | 1433 |
| 123 | AAUAGAGU A ACUGAAGA | 12 | UCUUCAGU CUGAUGAG GCCGUUAGGC CGAA ACUCUAUU | 1434 |
| 139 | GAAGCAGU C GCAGUGAA | 13 | UUCACUGC CUGAUGAG GCCGUUAGGC CGAA ACUGCUUC | 1435 |
| 151 | GUGAAGAU U GUAGAUAU | 14 | AUAUCUAC CUGAUGAG GCCGUUAGGC CGAA AUCUUCAC | 1436 |
| 154 | AAGAUUGU A GAUAUGAA | 15 | UUCAUAUC CUGAUGAG GCCGUUAGGC CGAA ACAAUCUU | 1437 |
| 158 | UUGUAGAU A UGAAGCGU | 16 | ACGCUUCA CUGAUGAG GCCGUUAGGC CGAA AUCUACAA | 1438 |
| 172 | CGUGCCGU A GACUGUCC | 17 | GGACAGUC CUGAUGAG GCCGUUAGGC CGAA ACGGCACG | 1439 |
| 179 | UAGACUGU C CAGAAAAU | 18 | AUUUUCUG CUGAUGAG GCCGUUAGGC CGAA ACAGUCUA | 1440 |
| 188 | CAGAAAAU A UUAAGAAA | 19 | UUUCUUAA CUGAUGAG GCCGUUAGGC CGAA AUUUUCUG | 1441 |
| 190 | GAAAAUAU U AAGAAAGA | 20 | UCUUUCUU CUGAUGAG GCCGUUAGGC CGAA AUAUUUUC | 1442 |
| 191 | AAAAUAUU A AGAAAGAG | 21 | CUCUUUCU CUGAUGAG GCCGUUAGGC CGAA AAUAUUUU | 1443 |
| 202 | AAAGAGAU C UGUAUCAA | 22 | UUGAUACA CUGAUGAG GCCGUUAGGC CGAA AUCUCUUU | 1444 |
| 206 | AGAUCUGU A UCAAUAAA | 23 | UUUAUUGA CUGAUGAG GCCGUUAGGC CGAA ACAGAUCU | 1445 |
| 208 | AUCUGUAU C AAUAAAAU | 24 | AUUUUAUU CUGAUGAG GCCGUUAGGC CGAA AUACAGAU | 1446 |
| 212 | GUAUCAAU A AAAUGCUA | 25 | UAGCAUUU CUGAUGAG GCCGUUAGGC CGAA AUUGAUAC | 1447 |
| 220 | AAAAUGCU A AAUCAUGA | 26 | UCAUGAUU CUGAUGAG GCCGUUAGGC CGAA AGCAUUUU | 1448 |
| 224 | UGCUAAAU C AUGAAAAU | 27 | AUUUUCAU CUGAUGAG GCCGUUAGGC CGAA AUUUAGCA | 1449 |
| 235 | GAAAAUGU A GUAAAAUU | 28 | AAUUUUAC CUGAUGAG GCCGUUAGGC CGAA ACAUUUUC | 1450 |
| 238 | AAUGUAGU A AAAUUCUA | 29 | UAGAAUUU CUGAUGAG GCCGUUAGGC CGAA ACUACAUU | 1451 |
| 243 | AGUAAAAU U CUAUGGUC | 30 | GACCAUAG CUGAUGAG GCCGUUAGGC CGAA AUUUUACU | 1452 |
| 244 | GUAAAAUU C UAUGGUCA | 31 | UGACCAUA CUGAUGAG GCCGUUAGGC CGAA AAUUUUAC | 1453 |
| 246 | AAAAUUCU A UGGUCACA | 32 | UGUGACCA CUGAUGAG GCCGUUAGGC CGAA AGAAUUUU | 1454 |
| 251 | UCUAUGGU C ACAGGAGA | 33 | UCUCCUGU CUGAUGAG GCCGUUAGGC CGAA ACCAUAGA | 1455 |
| 269 | AAGGCAAU A UCCAAUAU | 34 | AUAUUGGA CUGAUGAG GCCGUUAGGC CGAA AUUGCCUU | 1456 |
| 271 | GGCAAUAU C CAAUAUUU | 35 | AAAUAUUG CUGAUGAG GCCGUUAGGC CGAA AUAUUGCC | 1457 |
| 276 | UAUCCAAU A UUUAUUUC | 36 | GAAAUAAA CUGAUGAG GCCGUUAGGC CGAA AUUGGAUA | 1458 |
| 278 | UCCAAUAU U UAUUUCUG | 37 | CAGAAAUA CUGAUGAG GCCGUUAGGC CGAA AUAUUGGA | 1459 |
| 279 | CCAAUAUU U AUUUCUGG | 38 | CCAGAAAU CUGAUGAG GCCGUUAGGC CGAA AAUAUUGG | 1460 |
| 280 | CAAUAUUU A UUUCUGGA | 39 | UCCAGAAA CUGAUGAG GCCGUUAGGC CGAA AAAUAUUG | 1461 |
| 282 | AUAUUUAU U UCUGGAGU | 40 | ACUCCAGA CUGAUGAG GCCGUUAGGC CGAA AUAAAUAU | 1462 |
| 283 | UAUUUAUU U CUGGAGUA | 41 | UACUCCAG CUGAUGAG GCCGUUAGGC CGAA AAUAAAUA | 1463 |
| 284 | AUUUAUUU C UGGAGUAC | 42 | GUACUCCA CUGAUGAG GCCGUUAGGC CGAA AAAUAAAU | 1464 |
| 291 | UCUGGAGU A CUGAUGUG | 43 | CACUACAG CUGAUGAG GCCGUUAGGC CGAA ACUCCAGA | 1465 |
| 296 | AGUACUGU A GUGGAGGA | 44 | UCCUCCAC CUGAUGAG GCCGUUAGGC CGAA ACAGUACU | 1466 |
| 310 | GGAGAGCU U UUUGACAG | 45 | CUGUCAAA CUGAUGAG GCCGUUAGGC CGAA AGCUCUCC | 1467 |
| 311 | GAGAGCUU U UUGACAGA | 46 | UCUGUCAA CUGAUGAG GCCGUUAGGC CGAA AAGCUCUC | 1468 |
| 312 | AGAGCUUU U UGACAGAA | 47 | UUCUGUCA CUGAUGAG GCCGUUAGGC CGAA AAAGCUCU | 1469 |
| 313 | GAGCUUUU U GACAGAAU | 48 | AUUCUGUC CUGAUGAG GCCGUUAGGC CGAA AAAAGCUC | 1470 |
| 322 | GACAGAAU A GAGCCAGA | 49 | UCUGGCUC CUGAUGAG GCCGUUAGGC CGAA AUUCUGUC | 1471 |
| 334 | CCAGACAU A GGCAUGCC | 50 | GGCAUGCC CUGAUGAG GCCGUUAGGC CGAA AUGUCUGG | 1472 |
| 356 | CAGAUGCU C AGAGAUUC | 51 | GAAUCUCU CUGAUGAG GCCGUUAGGC CGAA AGCAUCUG | 1473 |
| 363 | UCAGAGAU U CUUCCAUC | 52 | GAUGGAAG CUGAUGAG GCCGUUAGGC CGAA AUCUCUGA | 1474 |
| 364 | CAGAGAUU C UUCCAUCA | 53 | UGAUGGAA CUGAUGAG GCCGUUAGGC CGAA AAUCUCUG | 1475 |
| 366 | GAGAUUCU U CCAUCAAC | 54 | GUUGAUGG CUGAUGAG GCCGUUAGGC CGAA AGAAUCUC | 1476 |
| 367 | AGAUUCUU C CAUCAACU | 55 | AGUUGAUG CUGAUGAG GCCGUUAGGC CGAA AAGAAUCU | 1477 |
| 371 | UCUUCCAU C AACUCAUG | 56 | CAUGAGUU CUGAUGAG GCCGUUAGGC CGAA AUGGAAGA | 1478 |
| 376 | CAUCAACU C AUGGCAGG | 57 | CCUGCCAU CUGAUGAG GCCGUUAGGC CGAA AGUUGAUG | 1479 |
| 391 | GGGGUGGU U UAUCUGCA | 58 | UGCAGAUA CUGAUGAG GCCGUUAGGC CGAA ACCACCCC | 1480 |
| 392 | GGGUGGUU U AUCUGCAU | 59 | AUGCAGAU CUGAUGAG GCCGUUAGGC CGAA AACCACCC | 1481 |
| 393 | GGUGGUUU A UCUGCAUG | 60 | CAUGCAGA CUGAUGAG GCCGUUAGGC CGAA AAACCACC | 1482 |
| 395 | UGGUUUAU C UGCAUGGU | 61 | ACCAUGCA CUGAUGAG GCCGUUAGGC CGAA AUAAACCA | 1483 |
| 404 | UGCAUGGU A UUGGAAUA | 62 | UAUUCCAA CUGAUGAG GCCGUUAGGC CGAA ACCAUGCA | 1484 |
| 406 | CAUGGUAU U GGAAUAAC | 63 | GUUAUUCC CUGAUGAG GCCGUUAGGC CGAA AUACCAUG | 1485 |
| 412 | AUUGGAAU A ACUCACAG | 64 | CUGUGAGU CUGAUGAG GCCGUUAGGC CGAA AUUCCAAU | 1486 |
| 416 | GAAUAACU C ACAGGGAU | 65 | AUCCCUGU CUGAUGAG GCCGUUAGGC CGAA AGUUAUUC | 1487 |
| 425 | ACAGGGAU A UUAAACCA | 66 | UGGUUUAA CUGAUGAG GCCGUUAGGC CGAA AUCCCUGU | 1488 |
| 427 | AGGGAUAU U AAACCAGA | 67 | UCUGGUUU CUGAUGAG GCCGUUAGGC CGAA AUAUCCCU | 1489 |
| 428 | GGGAUAUU A AACCAGAA | 68 | UUCUGGUU CUGAUGAG GCCGUUAGGC CGAA AAUAUCCC | 1490 |
| 440 | CAGAAAAU C UUCUGUUG | 69 | CAACAGAA CUGAUGAG GCCGUUAGGC CGAA AUUUUCUG | 1491 |
| 442 | GAAAAUCU U CUGUUGGA | 70 | UCCAACAG CUGAUGAG GCCGUUAGGC CGAA AGAUUUUC | 1492 |
| 443 | AAAAUCUU C UGUUGGAU | 71 | AUCCAACA CUGAUGAG GCCGUUAGGC CGAA AAGAUUUU | 1493 |
| 447 | UCUUCUGU U GGAUGAAA | 72 | UUUCAUCC CUGAUGAG GCCGUUAGGC CGAA ACAGAAGA | 1494 |
| 461 | AAAGGGAU A ACCUCAAA | 73 | UUUGAGGU CUGAUGAG GCCGUUAGGC CGAA AUCCCUUU | 1495 |
| 466 | GAUAACCU C AAAAUCUC | 74 | GAGAUUUU CUGAUGAG GCCGUUAGGC CGAA AGGUUAUC | 1496 |
| 472 | CUCAAAAU C UCAGACUU | 75 | AAGUCUGA CUGAUGAG GCCGUUAGGC CGAA AUUUUGAG | 1497 |
| 474 | CAAAAUCU C AGACUUUG | 76 | CAAAGUCU CUGAUGAG GCCGUUAGGC CGAA AGAUUUUG | 1498 |
| 480 | CUCAGACU U UGGCUUGG | 77 | CCAAGCCA CUGAUGAG GCCGUUAGGC CGAA AGUCUGAG | 1499 |
| 481 | UCAGACUU U GGCUUGGC | 78 | GCCAAGCC CUGAUGAG GCCGUUAGGC CGAA AAGUCUGA | 1500 |
| 486 | CUUUGGCU U GGCAACAG | 79 | CUGUUGCC CUGAUGAG GCCGUUAGGC CGAA AGCCAAAG | 1501 |
| 496 | GCAACAGU A UUUCGGUA | 80 | UACCGAAA CUGAUGAG GCCGUUAGGC CGAA ACUGUUGC | 1502 |
| 498 | AACAGUAU U UCGGUAUA | 81 | UAUACCGA CUGAUGAG GCCGUUAGGC CGAA AUACUGUU | 1503 |
| 499 | ACAGUAUU U CGGUAUAA | 82 | UUAUACCG CUGAUGAG GCCGUUAGGC CGAA AAUACUGU | 1504 |
| 500 | CAGUAUUU C GGUAUAAU | 83 | AUUAUACC CUGAUGAG GCCGUUAGGC CGAA AAAUACUG | 1505 |
| 504 | AUUUCGGU A UAAUAAUC | 84 | GAUUAUUA CUGAUGAG GCCGUUAGGC CGAA ACCGAAAU | 1506 |
| 506 | UUCGGUAU A AUAAUCGU | 85 | ACGAUUAU CUGAUGAG GCCGUUAGGC CGAA AUACCGAA | 1507 |
| 509 | GGUAUAAU A AUCGUGAG | 86 | CUCACGAU CUGAUGAG GCCGUUAGGC CGAA AUUAUACC | 1508 |
| 512 | AUAAUAAU C GUGAGCGU | 87 | ACGCUCAC CUGAUGAG GCCGUUAGGC CGAA AUUAUUAU | 1509 |
| 521 | GUGAGCGU U UGUUGAAC | 88 | GUUCAACA CUGAUGAG GCCGUUAGGC CGAA ACGCUCAC | 1510 |
| 522 | UGAGCGUU U GUUGAACA | 89 | UGUUCAAC CUGAUGAG GCCGUUAGGC CGAA AACGCUCA | 1511 |
| 525 | GCGUUUGU U GAACAAGA | 90 | UCUUGUUC CUGAUGAG GCCGUUAGGC CGAA ACAAACGC | 1512 |
| 542 | UGUGUGGU A CUUUACCA | 91 | UGGUAAAG CUGAUGAG GCCGUUAGGC CGAA ACCACACA | 1513 |
| 545 | GUGGUACU U UACCAUAU | 92 | AUAUGGUA CUGAUGAG GCCGUUAGGC CGAA AGUACCAC | 1514 |
| 546 | UGGUACUU U ACCAUAUG | 93 | CAUAUGGU CUGAUGAG GCCGUUAGGC CGAA AAGUACCA | 1515 |
| 547 | GGUACUUU A CCAUAUGU | 94 | ACAUAUGG CUGAUGAG GCCGUUAGGC CGAA AAAGUACC | 1516 |
| 552 | UUUACCAU A UGUUGCUC | 95 | GAGCAACA CUGAUGAG GCCGUUAGGC CGAA AUGGUAAA | 1517 |
| 556 | CCAUAUGU U GCUCCAGA | 96 | UCUGGAGC CUGAUGAG GCCGUUAGGC CGAA ACAUAUGG | 1518 |
| 560 | AUGUUGCU C CAGAACUU | 97 | AAGUUCUG CUGAUGAG GCCGUUAGGC CGAA AGCAACAU | 1519 |
| 568 | CCAGAACU U CUGAAGAG | 98 | CUCUUCAG CUGAUGAG GCCGUUAGGC CGAA AGUUCUGG | 1520 |
| 569 | CAGAACUU C UGAAGAGA | 99 | UCUCUUCA CUGAUGAG GCCGUUAGGC CGAA AAGUUCUG | 1521 |
| 585 | AAGAGAAU U UCAUGCAG | 100 | CUGCAUGA CUGAUGAG GCCGUUAGGC CGAA AUUCUCUU | 1522 |
| 586 | AGAGAAUU U CAUGCAGA | 101 | UCUGCAUG CUGAUGAG GCCGUUAGGC CGAA AAUUCUCU | 1523 |
| 587 | GAGAAUUU C AUGCAGAA | 102 | UUCUGCAU CUGAUGAG GCCGUUAGGC CGAA AAAUUCUC | 1524 |
| 601 | GAACCAGU U GAUGUUUG | 103 | CAAACAUC CUGAUGAG GCCGUUAGGC CGAA ACUGGUUC | 1525 |
| 607 | GUUGAUGU U UGGUCCUG | 104 | CAGGACCA CUGAUGAG GCCGUUAGGC CGAA ACAUCAAC | 1526 |
| 608 | UUGAUGUU U GGUCCUGU | 105 | ACAGGACC CUGAUGAG GCCGUUAGGC CGAA AACAUCAA | 1527 |
| 612 | UGUUUGGU C CUGUGGAA | 106 | UUCCACAG CUGAUGAG GCCGUUAGGC CGAA ACCAAACA | 1528 |
| 622 | UGUGGAAU A GUACUUAC | 107 | GUAAGUAC CUGAUGAG GCCGUUAGGC CGAA AUUCCACA | 1529 |
| 625 | GGAAUAGU A CUUACUGC | 108 | GCAGUAAG CUGAUGAG GCCGUUAGGC CGAA ACUAUUCC | 1530 |
| 628 | AUAGUACU U ACUGCAAU | 109 | AUUGCAGU CUGAUGAG GCCGUUAGGC CGAA AGUACUAU | 1531 |
| 629 | UAGUACUU A CUGCAAUG | 110 | CAUUGCAG CUGAUGAG GCCGUUAGGC CGAA AAGUACUA | 1532 |
| 640 | GCAAUGCU C GCUGGAGA | 111 | UCUCCAGC CUGAUGAG GCCGUUAGGC CGAA AGCAUUGC | 1533 |
| 651 | UGGAGAAU U GCCAUGGG | 112 | CCCAUGGC CUGAUGAG GCCGUUAGGC CGAA AUUCUCCA | 1534 |
| 680 | ACAGCUGU C AGGAGUAU | 113 | AUACUCCU CUGAUGAG GCCGUUAGGC CGAA ACAGCUGU | 1535 |
| 687 | UCAGGAGU A UUCUGACU | 114 | AGUCAGAA CUGAUGAG GCCGUUAGGC CGAA ACUCCUGA | 1536 |
| 689 | AGGAGUAU U CUGACUGG | 115 | CCAGUCAG CUGAUGAG GCCGUUAGGC CGAA AUACUCCU | 1537 |
| 690 | GGAGUAUU C UGACUGGA | 116 | UCCAGUCA CUGAUGAG GCCGUUAGGC CGAA AAUACUCC | 1538 |
| 714 | AAAAACAU A CCUCAACC | 117 | GGUUGAGG CUGAUGAG GCCGUUAGGC CGAA AUGUUUUU | 1539 |
| 718 | ACAUACCU C AACCCUUG | 118 | CAAGGGUU CUGAUGAG GCCGUUAGGC CGAA AGGUAUGU | 1540 |
| 725 | UCAACCCU U GGAAAAAA | 119 | UUUUUUCC CUGAUGAG GCCGUUAGGC CGAA AGGGUUGA | 1541 |
| 736 | AAAAAAAU C GAUUCUGC | 120 | GCAGAAUC CUGAUGAG GCCGUUAGGC CGAA AUUUUUUU | 1542 |
| 740 | AAAUCGAU U CUGCUCCU | 121 | AGGAGCAG CUGAUGAG GCCGUUAGGC CGAA AUCGAUUU | 1543 |
| 741 | AAUCGAUU C UGCUCCUC | 122 | GAGGAGCA CUGAUGAG GCCGUUAGGC CGAA AAUCGAUU | 1544 |
| 746 | AUUCUGCU C CUCUAGCU | 123 | AGCUAGAG CUGAUGAG GCCGUUAGGC CGAA AGCAGAAU | 1545 |
| 749 | CUGCUCCU C AUGCUCUG | 124 | CAGAGCUA CUGAUGAG GCCGUUAGGC CGAA AGGAGCAG | 1546 |
| 751 | GCUCCUCU A GCUCUGCU | 125 | AGCAGAGC CUGAUGAG GCCGUUAGGC CGAA AGAGGAGC | 1547 |
| 755 | CUCUAGCU C UGCUGCAU | 126 | AUGCAGCA CUGAUGAG GCCGUUAGGC CGAA AGCUAGAG | 1548 |
| 764 | UGCUGCAU A AAAUCUUA | 127 | UAAGAUUU CUGAUGAG GCCGUUAGGC CGAA AUGCAGCA | 1549 |
| 769 | CAUAAAAU C UUAGUUGA | 128 | UCAACUAA CUGAUGAG GCCGUUAGGC CGAA AUUUUAUG | 1550 |
| 771 | UAAAAUCU U AGUUGAGA | 129 | UCUCAACU CUGAUGAG GCCGUUAGGC CGAA AGAUUUUA | 1551 |
| 772 | AAAAUCUU A GUUGAGAA | 130 | UUCUCAAC CUGAUGAG GCCGUUAGGC CGAA AAGAUUUU | 1552 |
| 775 | AUCUUAGU U GAGAAUCC | 131 | GGAUUCUC CUGAUGAG GCCGUUAGGC CGAA ACUAAGAU | 1553 |
| 782 | UUGAGAAU C CAUCAGCA | 132 | UGCUGAUG CUGAUGAG GCCGUUAGGC CGAA AUUCUCAA | 1554 |
| 786 | GAAUCCAU C AGCAAGAA | 133 | UUCUUGCU CUGAUGAG GCCGUUAGGC CGAA AUGGAUUC | 1555 |
| 796 | GCAAGAAU U ACCAUUCC | 134 | GGAAUGGU CUGAUGAG GCCGUUAGGC CGAA AUUCUUGC | 1556 |
| 797 | CAAGAAUU A CCAUUCCA | 135 | UGGAAUGG CUGAUGAG GCCGUUAGGC CGAA AAUUCUUG | 1557 |
| 802 | AUUACCAU U CCAGACAU | 136 | AUGUCUGG CUGAUGAG GCCGUUAGGC CGAA AUGGUAAU | 1558 |
| 803 | UUACCAUU C CAGACAUC | 137 | GAUGUCUG CUGAUGAG GCCGUUAGGC CGAA AAUGGUAA | 1559 |
| 811 | CCAGACAU C AAAAAAGA | 138 | UCUUUUUU CUGAUGAG GCCGUUAGGC CGAA AUGUCUGG | 1560 |
| 821 | AAAAAGAU A GAUGGUAC | 139 | GUACCAUC CUGAUGAG GCCGUUAGGC CGAA AUCUUUUU | 1561 |
| 828 | UAGAUGGU A CAACAAAC | 140 | GUUUGUUG CUGAUGAG GCCGUUAGGC CGAA ACCAUCUA | 1562 |
| 841 | AAACCCCU C AAGAAAGG | 141 | CCUUUCUU CUGAUGAG GCCGUUAGGC CGAA AGGGGUUU | 1563 |
| 868 | CCCCGAGU C ACUUCAGG | 142 | CCUGAAGU CUGAUGAG GCCGUUAGGC CGAA ACUCGGGG | 1564 |
| 872 | GAGUCACU U CAGGUGGU | 143 | ACCACCUG CUGAUGAG GCCGUUAGGC CGAA AGUGACUC | 1565 |
| 873 | AGUCACUU C AGGUGGUG | 144 | CACCACCU CUGAUGAG GCCGUUAGGC CGAA AAGUGACU | 1566 |
| 885 | UGGUGUGU C AGAGUCUC | 145 | GAGACUCU CUGAUGAG GCCGUUAGGC CGAA ACACACCA | 1567 |
| 891 | GUCAGAGU C UCCCAGUG | 146 | CACUGGGA CUGAUGAG GCCGUUAGGC CGAA ACUCUGAC | 1568 |
| 893 | CAGAGUCU C CCAGUGGA | 147 | UCCACUGG CUGAUGAG GCCGUUAGGC CGAA AGACUCUG | 1569 |
| 903 | CAGUGGAU U UUCUAAGC | 148 | GCUUAGAA CUGAUGAG GCCGUUAGGC CGAA AUCCACUG | 1570 |
| 904 | AGUGGAUU U UCUAAGCA | 149 | UGCUUAGA CUGAUGAG GCCGUUAGGC CGAA AAUCCACU | 1571 |
| 905 | GUGGAUUU U CUAAGCAC | 150 | GUGCUUAG CUGAUGAG GCCGUUAGGC CGAA AAAUCCAC | 1572 |
| 906 | UGGAUUUU C UAAGCACA | 151 | UGUGCUUA CUGAUGAG GCCGUUAGGC CGAA AAAAUCCA | 1573 |
| 908 | GAUUUUCU A AGCACAUU | 152 | AAUGUGCU CUGAUGAG GCCGUUAGGC CGAA AGAAAAUC | 1574 |
| 916 | AAGCACAU U CAAUCCAA | 153 | UUGGAUUG CUGAUGAG GCCGUUAGGC CGAA AUGUGCUU | 1575 |
| 917 | AGCACAUU C AAUCCAAU | 154 | AUUGGAUU CUGAUGAG GCCGUUAGGC CGAA AAUGUGCU | 1576 |
| 921 | CAUUCAAU C CAAUUUGG | 155 | CCAAAUUG CUGAUGAG GCCGUUAGGC CGAA AUUGAAUG | 1577 |
| 926 | AAUCCAAU U UGGACUUC | 156 | GAAGUCCA CUGAUGAG GCCGUUAGGC CGAA AUUGGAUU | 1578 |
| 927 | AUCCAAUU U GGACUUCU | 157 | AGAAGUCC CUGAUGAG GCCGUUAGGC CGAA AAUUGGAU | 1579 |
| 933 | UUUGGACU U CUCUCCAG | 158 | CUGGAGAG CUGAUGAG GCCGUUAGGC CGAA AGUCCAAA | 1580 |
| 934 | UUGGACUU C UCUCCAGU | 159 | ACUGGAGA CUGAUGAG GCCGUUAGGC CGAA AAGUCCAA | 1581 |
| 936 | GGACUUCU C UCCAGUAA | 160 | UUACUGGA CUGAUGAG GCCGUUAGGC CGAA AGAAGUCC | 1582 |
| 938 | ACUUCUCU C CAGUAAAC | 161 | GUUUACUG CUGAUGAG GCCGUUAGGC CGAA AGAGAAGU | 1583 |
| 943 | UCUCCAGU A AACAGUGC | 162 | GCACUGUU CUGAUGAG GCCGUUAGGC CGAA ACUGGAGA | 1584 |
| 953 | ACAGUGCU U CUAGUGAA | 163 | UUCACUAG CUGAUGAG GCCGUUAGGC CGAA AGCACUGU | 1585 |
| 954 | CAGUGCUU C UAGUGAAG | 164 | CUUCACUA CUGAUGAG GCCGUUAGGC CGAA AAGCACUG | 1586 |
| 956 | GUGCUUCU A GUGAAGAA | 165 | UUCUUCAC CUGAUGAG GCCGUUAGGC CGAA AGAAGCAC | 1587 |
| 975 | UGUGAAGU A CUCCAGUU | 166 | AACUGGAG CUGAUGAG GCCGUUAGGC CGAA ACUUCACA | 1588 |
| 978 | GAAGUACU C CAGUUCUC | 167 | GAGAACUG CUGAUGAG GCCGUUAGGC CGAA AGUACUUC | 1589 |
| 983 | ACUCCAGU U CUCAGCCA | 168 | UGGCUGAG CUGAUGAG GCCGUUAGGC CGAA ACUGGAGU | 1590 |
| 984 | CUCCAGUU C UCAGCCAG | 169 | CUGGCUGA CUGAUGAG GCCGUUAGGC CGAA AACUGGAG | 1591 |
| 986 | CCAGUUCU C AGCCAGAA | 170 | UUCUGGCU CUGAUGAG GCCGUUAGGC CGAA AGAACUGG | 1592 |
| 1007 | GCACAGGU C UUUCCUUA | 171 | UAAGGAAA CUGAUGAG GCCGUUAGGC CGAA ACCUGUGC | 1593 |
| 1009 | ACAGGUCU U UCCUUAUG | 172 | CAUAAGGA CUGAUGAG GCCGUUAGGC CGAA AGACCUGU | 1594 |
| 1010 | CAGGUCUU U CCUUAUGG | 173 | CCAUAAGG CUGAUGAG GCCGUUAGGC CGAA AAGACCUG | 1595 |
| 1011 | AGGUCUUU C CUUAUGGG | 174 | CCCAUAAG CUGAUGAG GCCGUUAGGC CGAA AAAGACCU | 1596 |
| 1014 | UCUUUCCU U AUGGGAUA | 175 | UAUCCCAU CUGAUGAG GCCGUUAGGC CGAA AGGAAAGA | 1597 |
| 1015 | CUUUCCUU A UGGGAUAC | 176 | GUAUCCCA CUGAUGAG GCCGUUAGGC CGAA AAGGAAAG | 1598 |
| 1022 | UAUGGGAU A CCAGCCCC | 177 | GGGGCUGG CUGAUGAG GCCGUUAGGC CGAA AUCCCAUA | 1599 |
| 1032 | CAGCCCCU C AUACAUUG | 178 | CAAUGUAU CUGAUGAG GCCGUUAGGC CGAA AGGGGCUG | 1600 |
| 1035 | CCCCUCAU A CAUUGAUA | 179 | UAUCAAUG CUGAUGAG GCCGUUAGGC CGAA AUGAGGGG | 1601 |
| 1039 | UCAUACAU U GAUAAAUU | 180 | AAUUUAUC CUGAUGAG GCCGUUAGGC CGAA AUGUAUGA | 1602 |
| 1043 | ACAUUGAU A AAUUGGUA | 181 | UACCAAUU CUGAUGAG GCCGUUAGGC CGAA AUCAAUGU | 1603 |
| 1047 | UGAUAAAU U GGUACAAG | 182 | CUUGUACC CUGAUGAG GCCGUUAGGC CGAA AUUUAUCA | 1604 |
| 1051 | AAAUUGGU A CAAGGGAU | 183 | AUCCCUUG CUGAUGAG GCCGUUAGGC CGAA ACCAAUUU | 1605 |
| 1060 | CAAGGGAU C AGCUUUUC | 184 | GAAAAGCU CUGAUGAG GCCGUUAGGC CGAA AUCCCUUG | 1606 |
| 1065 | GAUCAGCU U UUCCCAGC | 185 | GCUGGGAA CUGAUGAG GCCGUUAGGC CGAA AGCUGAUC | 1607 |
| 1066 | AUCAGCUU U UCCCAGCC | 186 | GGCUGGGA CUGAUGAG GCCGUUAGGC CGAA AAGCUGAU | 1608 |
| 1067 | UCAGCUUU U CCCAGCCC | 187 | GGGCUGGG CUGAUGAG GCCGUUAGGC CGAA AAAGCUGA | 1609 |
| 1068 | CAGCUUUU C CCAGCCCA | 188 | UGGGCUGG CUGAUGAG GCCGUUAGGC CGAA AAAAGCUG | 1610 |
| 1082 | CCACAUGU C CUGAUCAU | 189 | AUGAUCAG CUGAUGAG GCCGUUAGGC CGAA ACAUGUGG | 1611 |
| 1088 | GUCCUGAU C AUAUGCUU | 190 | AAGCAUAU CUGAUGAG GCCGUUAGGC CGAA AUCAGGAC | 1612 |
| 1091 | CUGAUCAU A UGCUUUUG | 191 | CAAAAGCA CUGAUGAG GCCGUUAGGC CGAA AUGAUCAG | 1613 |
| 1096 | CAUAUGCU U UUGAAUAG | 192 | CUAUUCAA CUGAUGAG GCCGUUAGGC CGAA AGCAUAUG | 1614 |
| 1097 | AUAUGCUU U UGAAUAGU | 193 | ACUAUUCA CUGAUGAG GCCGUUAGGC CGAA AAGCAUAU | 1615 |
| 1098 | UAUGCUUU U GAAUAGUC | 194 | GACUAUUC CUGAUGAG GCCGUUAGGC CGAA AAAGCAUA | 1616 |
| 1103 | UUUUGAAU A GUCAGUUA | 195 | UAACUGAC CUGAUGAG GCCGUUAGGC CGAA AUUCAAAA | 1617 |
| 1106 | UGAAUAGU C AGUUACUU | 196 | AAGUAACU CUGAUGAG GCCGUUAGGC CGAA ACUAUUCA | 1618 |
| 1110 | UAGUCAGU U ACUUGGCA | 197 | UGCCAAGU CUGAUGAG GCCGUUAGGC CGAA ACUGACUA | 1619 |
| 1111 | AGUCAGUU A CUUGGCAC | 198 | GUGCCAAG CUGAUGAG GCCGUUAGGC CGAA AACUGACU | 1620 |
| 1114 | CAGUUACU U GGCACCCC | 199 | GGGGUGCC CUGAUGAG GCCGUUAGGC CGAA AGUAACUG | 1621 |
| 1128 | CCCAGGAU C CUCACAGA | 200 | UCUGUGAG CUGAUGAG GCCGUUAGGC CGAA AUCCUGGG | 1622 |
| 1131 | AGGAUCCU C ACAGAACC | 201 | GGUUCUGU CUGAUGAG GCCGUUAGGC CGAA AGGAUCCU | 1623 |
| 1152 | GCAGCGGU U GGUCAAAA | 202 | UUUUGACC CUGAUGAG GCCGUUAGGC CGAA ACCGCUGC | 1624 |
| 1156 | CGGUUGGU C AAAAGAAU | 203 | AUUCUUUU CUGAUGAG GCCGUUAGGC CGAA ACCAACCG | 1625 |
| 1173 | GACACGAU U CUUUACCA | 204 | UGGUAAAG CUGAUGAG GCCGUUAGGC CGAA AUCGUGUC | 1626 |
| 1174 | ACACGAUU C UUUACCAA | 205 | UUGGUAAA CUGAUGAG GCCGUUAGGC CGAA AAUCGUGU | 1627 |
| 1176 | ACGAUUCU U UACCAAAU | 206 | AUUUGGUA CUGAUGAG GCCGUUAGGC CGAA AGAAUCGU | 1628 |
| 1177 | CGAUUCUU U ACCAAAUU | 207 | AAUUUGGU CUGAUGAG GCCGUUAGGC CGAA AAGAAUCG | 1629 |
| 1178 | GAUUCUUU A CCAAAUUG | 208 | CAAUUUGG CUGAUGAG GCCGUUAGGC CGAA AAAGAAUC | 1630 |
| 1185 | UACCAAAU U GGAUGCAG | 209 | CUGCAUCC CUGAUGAG GCCGUUAGGC CGAA AUUUGGUA | 1631 |
| 1200 | AGACAAAU C UUAUCAAU | 210 | AUUGAUAA CUGAUGAG GCCGUUAGGC CGAA AUUUGUCU | 1632 |
| 1202 | ACAAAUCU U AUCAAUGC | 211 | GCAUUGAU CUGAUGAG GCCGUUAGGC CGAA AGAUUUGU | 1633 |
| 1203 | CAAAUCUU A UCAAUGCC | 212 | GGCAUUGA CUGAUGAG GCCGUUAGGC CGAA AAGAUUUG | 1634 |
| 1205 | AAUCUUAU C AAUGCCUG | 213 | CAGGCAUU CUGAUGAG GCCGUUAGGC CGAA AUAAGAUU | 1635 |
| 1223 | AAGAGACU U GUGAGAAG | 214 | CUUCUCAC CUGAUGAG GCCGUUAGGC CGAA AGUCUCUU | 1636 |
| 1233 | UGAGAAGU U GGGCUAUC | 215 | GAUAGCCC CUGAUGAG GCCGUUAGGC CGAA ACUUCUCA | 1637 |
| 1239 | GUUGGGCU A UCAAUGGA | 216 | UCCAUUGA CUGAUGAG GCCGUUAGGC CGAA AGCCCAAC | 1638 |
| 1241 | UGGGCUAU C AAUGGAAG | 217 | CUUCCAUU CUGAUGAG GCCGUUAGGC CGAA AUAGCCCA | 1639 |
| 1256 | AGAAAAGU U GUAUGAAU | 218 | AUUCAUAC CUGAUGAG GCCGUUAGGC CGAA ACUUUUCU | 1640 |
| 1259 | AAAGUUGU A UGAAUCAG | 219 | CUGAUUCA CUGAUGAG GCCGUUAGGC CGAA ACAACUUU | 1641 |
| 1265 | GUAUGAAU C AGGUUACU | 220 | AGUAACCU CUGAUGAG GCCGUUAGGC CGAA AUUCAUAC | 1642 |
| 1270 | AAUCAGGU U ACUAUAUC | 221 | GAUAUAGU CUGAUGAG GCCGUUAGGC CGAA ACCUGAUU | 1643 |
| 1271 | AUCAGGUU A CUAUAUCA | 222 | UGAUAUAG CUGAUGAG GCCGUUAGGC CGAA AACCUGAU | 1644 |
| 1274 | AGGUUACU A UAUCAACA | 223 | UGUUGAUA CUGAUGAG GCCGUUAGGC CGAA AGUAACCU | 1645 |
| 1276 | GUUACUAU A UCAACAAC | 224 | GUUGUUGA CUGAUGAG GCCGUUAGGC CGAA AUAGUAAC | 1646 |
| 1278 | UACUAUAU C AACAACUG | 225 | CAGUUGUU CUGAUGAG GCCGUUAGGC CGAA AUAUAGUA | 1647 |
| 1289 | CAACUGAU A GGAGAAAC | 226 | GUUUCUCC CUGAUGAG GCCGUUAGGC CGAA AUCAGUUG | 1648 |
| 1301 | GAAACAAU A AACUCAUU | 227 | AAUGAGUU CUGAUGAG GCCGUUAGGC CGAA AUUGUUUC | 1649 |
| 1306 | AAUAAACU C AUUUUCAA | 228 | UUGAAAAU CUGAUGAG GCCGUUAGGC CGAA AGUUUAUU | 1650 |
| 1309 | AAACUCAU U UUCAAAGU | 229 | ACUUUGAA CUGAUGAG GCCGUUAGGC CGAA AUGAGUUU | 1651 |
| 1310 | AACUCAUU U UCAAAGUG | 230 | CACUUUGA CUGAUGAG GCCGUUAGGC CGAA AAUGAGUU | 1652 |
| 1311 | ACUCAUUU U CAAAGUGA | 231 | UCACUUUG CUGAUGAG GCCGUUAGGC CGAA AAAUGAGU | 1653 |
| 1312 | CUCAUUUU C AAAGUGAA | 232 | UUCACUUU CUGAUGAG GCCGUUAGGC CGAA AAAAUGAG | 1654 |
| 1322 | AAGUGAAU U UGUUAGAA | 233 | UUCUAACA CUGAUGAG GCCGUUAGGC CGAA AUUCACUU | 1655 |
| 1323 | AGUGAAUU U GUUAGAAA | 234 | UUUCUAAC CUGAUGAG GCCGUUAGGC CGAA AAUUCACU | 1656 |
| 1326 | GAAUUUGU U AGAAAUGG | 235 | CCAUUUCU CUGAUGAG GCCGUUAGGC CGAA ACAAAUUC | 1657 |
| 1327 | AAUUUGUU A GAAAUGGA | 236 | UCCAUUUC CUGAUGAG GCCGUUAGGC CGAA AACAAAUU | 1658 |
| 1340 | UGGAUGAU A AAAUAUUG | 237 | CAAUAUUU CUGAUGAG GCCGUUAGGC CGAA AUCAUCCA | 1659 |
| 1345 | GAUAAAAU A UUGGUUGA | 238 | UCAACCAA CUGAUGAG GCCGUUAGGC CGAA AUUUUAUC | 1660 |
| 1347 | UAAAAUAU U GGUUGACU | 239 | AGUCAACC CUGAUGAG GCCGUUAGGC CGAA AUAUUUUA | 1661 |
| 1351 | AUAUUGGU U GACUUCCG | 240 | CGGAAGUC CUGAUGAG GCCGUUAGGC CGAA ACCAAUAU | 1662 |
| 1356 | GGUUGACU U CCGGCUUU | 241 | AAAGCCGG CUGAUGAG GCCGUUAGGC CGAA AGUCAACC | 1663 |
| 1357 | GUUGACUU C CGGCUUUC | 242 | GAAAGCCG CUGAUGAG GCCGUUAGGC CGAA AAGUCAAC | 1664 |
| 1363 | UUCCGGCU U UCUAAGGG | 243 | CCCUUAGA CUGAUGAG GCCGUUAGGC CGAA AGCCGGAA | 1665 |
| 1364 | UCCGGCUU U CUAAGGGU | 244 | ACCCUUAG CUGAUGAG GCCGUUAGGC CGAA AAGCCGGA | 1666 |
| 1365 | CCGGCUUU C UAAGGGUG | 245 | CACCCUUA CUGAUGAG GCCGUUAGGC CGAA AAAGCCGG | 1667 |
| 1367 | GGCUUUCU A AGGGUGAU | 246 | AUCACCCU CUGAUGAG GCCGUUAGGC CGAA AGAAAGCC | 1668 |
| 1380 | UGAUGGAU U GGAGUUCA | 247 | UGAACUCC CUGAUGAG GCCGUUAGGC CGAA AUCCAUCA | 1669 |
| 1386 | AUUGGAGU U CAAGAGAC | 248 | GUCUCUUG CUGAUGAG GCCGUUAGGC CGAA ACUCCAAU | 1670 |
| 1387 | UUGGAGUU C AAGAGACA | 249 | UGUCUCUU CUGAUGAG GCCGUUAGGC CGAA AACUCCAA | 1671 |
| 1398 | GAGACACU U CCUGAAGA | 250 | UCUUCAGG CUGAUGAG GCCGUUAGGC CGAA AGUGUCUC | 1672 |
| 1399 | AGACACUU C CUGAAGAU | 251 | AUCUUCAG CUGAUGAG GCCGUUAGGC CGAA AAGUGUCU | 1673 |
| 1408 | CUGAAGAU U AAAGGGAA | 252 | UUCCCUUU CUGAUGAG GCCGUUAGGC CGAA AUCUUCAG | 1674 |
| 1409 | UGAAGAUU A AAGGGAAG | 253 | CUUCCCUU CUGAUGAG GCCGUUAGGC CGAA AAUCUUCA | 1675 |
| 1423 | AAGCUGAU U GAUAUUGU | 254 | ACAAUAUC CUGAUGAG GCCGUUAGGC CGAA AUCAGCUU | 1676 |
| 1427 | UGAUUGAU A UUGUGAGC | 255 | GCUCACAA CUGAUGAG GCCGUUAGGC CGAA AUCAAUCA | 1677 |
| 1429 | AUUGAUAU U GUGAGCAG | 256 | CUGCUCAC CUGAUGAG GCCGUUAGGC CGAA AUAUCAAU | 1678 |
| 1447 | CAGAAGGU U UGGCUUCC | 257 | GGAAGCCA CUGAUGAG GCCGUUAGGC CGAA ACCUUCUG | 1679 |
| 1448 | AGAAGGUU U GGCUUCCU | 258 | AGGAAGCC CUGAUGAG GCCGUUAGGC CGAA AACCUUCU | 1680 |
| 1453 | GUUUGGCU U CCUGCCAC | 259 | GUGGCAGG CUGAUGAG GCCGUUAGGC CGAA AGCCAAAC | 1681 |
| 1454 | UUUGGCUU C CUGCCACA | 260 | UGUGGCAG CUGAUGAG GCCGUUAGGC CGAA AAGCCAAA | 1682 |
| 1467 | CACAUGAU C GGACCAUC | 261 | GAUGGUCC CUGAUGAG GCCGUUAGGC CGAA AUCAUGUG | 1683 |
| 1475 | CGGACCAU C GGCUCUGG | 262 | CCAGAGCC CUGAUGAG GCCGUUAGGC CGAA AUGGUCCG | 1684 |
| 1480 | CAUCGGCU C UGGGGAAU | 263 | AUUCCCCA CUGAUGAG GCCGUUAGGC CGAA AGCCGAUG | 1685 |
| 1489 | UGGGGAAU C CUGGUGAA | 264 | UUCACCAG CUGAUGAG GCCGUUAGGC CGAA AUUCCCCA | 1686 |
| 1499 | UGGUGAAU A UAGUGCUG | 265 | CAGCACUA CUGAUGAG GCCGUUAGGC CGAA AUUCACCA | 1687 |
| 1501 | GUGAAUAU A GUGCUGCU | 266 | AGCAGCAC CUGAUGAG GCCGUUAGGC CGAA AUAUUCAC | 1688 |
| 1510 | GUGCUGCU A UGUUGACA | 267 | UGUCAACA CUGAUGAG GCCGUUAGGC CGAA AGCAGCAC | 1689 |
| 1514 | UGCUAUGU U GACAUUAU | 268 | AUAAUGUC CUGAUGAG GCCGUUAGGC CGAA ACAUAGCA | 1690 |
| 1520 | GUUGACAU U AUUCUUCC | 269 | GGAAGAAU CUGAUGAG GCCGUUAGGC CGAA AUGUCAAC | 1691 |
| 1521 | UUGACAUU A UUCUUCCU | 270 | AGGAAGAA CUGAUGAG GCCGUUAGGC CGAA AAUGUCAA | 1692 |
| 1523 | GACAUUAU U CUUCCUAG | 271 | CUAGGAAG CUGAUGAG GCCGUUAGGC CGAA AUAAUGUC | 1693 |
| 1524 | ACAUUAUU C UUCCUAGA | 272 | UCUAGGAA CUGAUGAG GCCGUUAGGC CGAA AAUAAUGU | 1694 |
| 1526 | AUUAUUCU U CCUAGAGA | 273 | UCUCUAGG CUGAUGAG GCCGUUAGGC CGAA AGAAUAAU | 1695 |
| 1527 | UUAUUCUU C CUAGAGAA | 274 | UUCUCUAG CUGAUGAG GCCGUUAGGC CGAA AAGAAUAA | 1696 |
| 1530 | UUCUUCCU A GAGAAGAU | 275 | AUCUUCUC CUGAUGAG GCCGUUAGGC CGAA AGGAAGAA | 1697 |
| 1539 | GAGAAGAU U AUCCUGUC | 276 | GACAGGAU CUGAUGAG GCCGUUAGGC CGAA AUCUUCUC | 1698 |
| 1540 | AGAAGAUU A UCCUGUCC | 277 | GGACAGGA CUGAUGAG GCCGUUAGGC CGAA AAUCUUCU | 1699 |
| 1542 | AAGAUUAU C CUGUCCUG | 278 | CAGGACAG CUGAUGAG GCCGUUAGGC CGAA AUAAUCUU | 1700 |
| 1547 | UAUCCUGU C CUGCAAAC | 279 | GUUUGCAG CUGAUGAG GCCGUUAGGC CGAA ACAGGAUA | 1701 |
| 1563 | CUGCAAAU A GUAGUUCC | 280 | GGAACUAC CUGAUGAG GCCGUUAGGC CGAA AUUUGCAG | 1702 |
| 1566 | CAAAUAGU A GUUCCUGA | 281 | UCAGGAAC CUGAUGAG GCCGUUAGGC CGAA ACUAUUUG | 1703 |
| 1569 | AUAGUAGU U CCUGAAGU | 282 | ACUUCAGG CUGAUGAG GCCGUUAGGC CGAA ACUACUAU | 1704 |
| 1570 | UAGUAGUU C CUGAAGUG | 283 | CACUUCAG CUGAUGAG GCCGUUAGGC CGAA AACUACUA | 1705 |
| 1580 | UGAAGUGU U CACUUCCC | 284 | GGGAAGUG CUGAUGAG GCCGUUAGGC CGAA ACACUUCA | 1706 |
| 1581 | GAAGUGUU C ACUUCCCU | 285 | AGGGAAGU CUGAUGAG GCCGUUAGGC CGAA AACACUUC | 1707 |
| 1585 | UGUUCACU U CCCUGUUU | 286 | AAACAGGG CUGAUGAG GCCGUUAGGC CGAA AGUGAACA | 1708 |
| 1586 | GUUCACUU C CCUGUUUA | 287 | UAAACAGG CUGAUGAG GCCGUUAGGC CGAA AAGUGAAC | 1709 |
| 1592 | UUCCCUGU U UAUCCAAA | 288 | UUUGGAUA CUGAUGAG GCCGUUAGGC CGAA ACAGGGAA | 1710 |
| 1593 | UCCCUGUU U AUCCAAAC | 289 | GUUUGGAU CUGAUGAG GCCGUUAGGC CGAA AACAGGGA | 1711 |
| 1594 | CCCUGUUU A UCCAAACA | 290 | UGUUUGGA CUGAUGAG GCCGUUAGGC CGAA CUGAUGAG | 1712 |
| 1596 | CUGUUUAU C CAAACAUC | 291 | GAUGUUUG CUGAUGAG GCCGUUAGGC CGAA AUAAACAG | 1713 |
| 1604 | CCAAACAU C UUCCAAUU | 292 | AAUUGGAA CUGAUGAG GCCGUUAGGC CGAA AUGUUUGG | 1714 |
| 1606 | AAACAUCU U CCAAUUUA | 293 | UAAAUUGG CUGAUGAG GCCGUUAGGC CGAA AGAUGUUU | 1715 |
| 1607 | AACAUCUU C CAAUUUAU | 294 | AUAAAUUG CUGAUGAG GCCGUUAGGC CGAA AAGAUGUU | 1716 |
| 1612 | CUUCCAAU U UAUUUUGU | 295 | ACAAAAUA CUGAUGAG GCCGUUAGGC CGAA AUUGGAAG | 1717 |
| 1613 | UUCCAAUU U AUUUUGUU | 296 | AACAAAAU CUGAUGAG GCCGUUAGGC CGAA AAUUGGAA | 1718 |
| 1614 | UCCAAUUU A UUUUGUUU | 297 | AAACAAAA CUGAUGAG GCCGUUAGGC CGAA AAAUUGGA | 1719 |
| 1616 | CAAUUUAU U UUGUUUGU | 298 | ACAAACAA CUGAUGAG GCCGUUAGGC CGAA AUAAAUUG | 1720 |
| 1617 | AAUUUAUU U UGUUUGUU | 299 | AACAAACA CUGAUGAG GCCGUUAGGC CGAA AAUAAAUU | 1721 |
| 1618 | AUUUAUUU U GUUUGUUC | 300 | GAACAAAC CUGAUGAG GCCGUUAGGC CGAA AAAUAAAU | 1722 |
| 1621 | UAUUUUGU U UGUUCGGC | 301 | GCCGAACA CUGAUGAG GCCGUUAGGC CGAA ACAAAAUA | 1723 |
| 1622 | AUUUUGUU U GUUCGGCA | 302 | UGCCGAAC CUGAUGAG GCCGUUAGGC CGAA AACAAAAU | 1724 |
| 1625 | UUGUUUGU U CGGCAUAC | 303 | GUAUGCCG CUGAUGAG GCCGUUAGGC CGAA ACAAACAA | 1725 |
| 1626 | UGUUUGUU C GGCAUACA | 304 | UGUAUGCC CUGAUGAG GCCGUUAGGC CGAA AACAAACA | 1726 |
| 1632 | UUCGGCAU A CAAAUAAU | 305 | AUUAUUUG CUGAUGAG GCCGUUAGGC CGAA AUGCCGAA | 1727 |
| 1638 | AUACAAAU A AUACCUAU | 306 | AUAGGUAU CUGAUGAG GCCGUUAGGC CGAA AUUUGUAU | 1728 |
| 1641 | CAAAUAAU A CCUAUAUC | 307 | GAUAUAGG CUGAUGAG GCCGUUAGGC CGAA AUUAUUUG | 1729 |
| 1645 | UAAUACCU A UAUCUUAA | 308 | UUAAGAUA CUGAUGAG GCCGUUAGGC CGAA AGGUAUUA | 1730 |
| 1647 | AUACCUAU A UCUUAAUU | 309 | AAUUAAGA CUGAUGAG GCCGUUAGGC CGAA AUAGGUAU | 1731 |
| 1649 | ACCUAUAU C UUAAUUGU | 310 | ACAAUUAA CUGAUGAG GCCGUUAGGC CGAA AUAUAGGU | 1732 |
| 1651 | CUAUAUCU U AAUUGUAA | 311 | UUACAAUU CUGAUGAG GCCGUUAGGC CGAA AGAUAUAG | 1733 |
| 1652 | UAUAUCUU A AUUGUAAG | 312 | CUUACAAU CUGAUGAG GCCGUUAGGC CGAA AAGAUAUA | 1734 |
| 1655 | AUCUUAAU U GUAAGCAA | 313 | UUGCUUAC CUGAUGAG GCCGUUAGGC CGAA AUUAAGAU | 1735 |
| 1658 | UUAAUUGU A AGCAAAAC | 314 | GUUUUGCU CUGAUGAG GCCGUUAGGC CGAA ACAAUUAA | 1736 |
| 1668 | GCAAAACU U UGGGGAAA | 315 | UUUCCCCA CUGAUGAG GCCGUUAGGC CGAA AGUUUUGC | 1737 |
| 1669 | CAAAACUU U GGGGAAAG | 316 | CUUUCCCC CUGAUGAG GCCGUUAGGC CGAA AAGUUUUG | 1738 |
| 1685 | GGAUGAAU A GAAUUCAU | 317 | AUGAAUUC CUGAUGAG GCCGUUAGGC CGAA AUUCAUCC | 1739 |
| 1690 | AAUAGAAU U CAUUUGAU | 318 | AUCAAAUG CUGAUGAG GCCGUUAGGC CGAA AUUCUAUU | 1740 |
| 1691 | AUAGAAUU C AUUUGAUU | 319 | AAUCAAAU CUGAUGAG GCCGUUAGGC CGAA AAUUCUAU | 1741 |
| 1694 | GAAUUCAU U UGAUUAUU | 320 | AAUAAUCA CUGAUGAG GCCGUUAGGC CGAA AUGAAUUC | 1742 |
| 1695 | AAUUCAUU U GAUUAUUU | 321 | AAAUAAUC CUGAUGAG GCCGUUAGGC CGAA AAUGAAUU | 1743 |
| 1699 | CAUUUGAU U AUUUCUUC | 322 | GAAGAAAU CUGAUGAG GCCGUUAGGC CGAA AUCAAAUG | 1744 |
| 1700 | AUUUGAUU A UUUCUUCA | 323 | UGAAGAAA CUGAUGAG GCCGUUAGGC CGAA AAUCAAAU | 1745 |
| 1702 | UUGAUUAU U UCUUCAUG | 324 | CAUGAAGA CUGAUGAG GCCGUUAGGC CGAA AUAAUCAA | 1746 |
| 1703 | UGAUUAUU U CUUCAUGU | 325 | ACAUGAAG CUGAUGAG GCCGUUAGGC CGAA AAUAAUCA | 1747 |
| 1704 | GAUUAUUU C UUCAUGUG | 326 | CACAUGAA CUGAUGAG GCCGUUAGGC CGAA AAAUAAUC | 1748 |
| 1706 | UUAUUUCU U CAUGUGUG | 327 | CACACAUG CUGAUGAG GCCGUUAGGC CGAA AGAAAUAA | 1749 |
| 1707 | UAUUUCUU C AUGUGUGU | 328 | ACACACAU CUGAUGAG GCCGUUAGGC CGAA AAGAAAUA | 1750 |
| 1716 | AUGUGUGU U AUGUAUCU | 329 | AGAUACUA CUGAUGAG GCCGUUAGGC CGAA ACACACAU | 1751 |
| 1717 | UGUGUGUU U AGUAUCUG | 330 | CAGAUACU CUGAUGAG GCCGUUAGGC CGAA AACACACA | 1752 |
| 1718 | GUGUGUUU A GUAUCUGA | 331 | UCAGAUAC CUGAUGAG GCCGUUAGGC CGAA AAACACAC | 1753 |
| 1721 | UGUUUAGU A UCUGAAUU | 332 | AAUUCAGA CUGAUGAG GCCGUUAGGC CGAA ACUAAACA | 1754 |
| 1723 | UUUAGUAU C UGAAUUUG | 333 | CAAAUUCA CUGAUGAG GCCGUUAGGC CGAA AUACUAAA | 1755 |
| 1729 | AUCUGAAU U UGAAACUC | 334 | GAGUUUCA CUGAUGAG GCCGUUAGGC CGAA AUUCAGAU | 1756 |
| 1730 | UCUGAAUU U GAAACUCA | 335 | UGAGUUUC CUGAUGAG GCCGUUAGGC CGAA AAUUCAGA | 1757 |
| 1737 | UUGAAACU C AUCUGGUG | 336 | CACCAGAU CUGAUGAG GCCGUUAGGC CGAA AGUUUCAA | 1758 |
| 1740 | AAACUCAU C UGGUGGAA | 337 | UUCCACCA CUGAUGAG GCCGUUAGGC CGAA AUGAGUUU | 1759 |
| 1756 | AACCAAGU U UCAGGGGA | 338 | UCCCCUGA CUGAUGAG GCCGUUAGGC CGAA ACUUGGUU | 1760 |
| 1757 | ACCAAGUU U CAGGGGAC | 339 | GUCCCCUG CUGAUGAG GCCGUUAGGC CGAA AACUUGGU | 1761 |
| 1758 | CCAAGUUU C AGGGGACA | 340 | UGUCCCCU CUGAUGAG GCCGUUAGGC CGAA AAACUUGG | 1762 |
| 1772 | ACAUGAGU U UUCCAGCU | 341 | AGCUGGAA CUGAUGAG GCCGUUAGGC CGAA ACUCAUGU | 1763 |
| 1773 | CAUGAGUU U UCCAGCUU | 342 | AAGCUGGA CUGAUGAG GCCGUUAGGC CGAA AACUCAUG | 1764 |
| 1774 | AUGAGUUU U CCAGCUUU | 343 | AAAGCUGG CUGAUGAG GCCGUUAGGC CGAA AAACUCAU | 1765 |
| 1775 | UGAGUUUU C CAGCUUUU | 344 | AAAAGCUG CUGAUGAG GCCGUUAGGC CGAA AAAACUCA | 1766 |
| 1781 | UUCCAGCU U UUAUACAC | 345 | GUGUAUAA CUGAUGAG GCCGUUAGGC CGAA AGCUGGAA | 1767 |
| 1782 | UCCAGCUU U UAUACACA | 346 | UGUGUAUA CUGAUGAG GCCGUUAGGC CGAA AAGCUGGA | 1768 |
| 1783 | CCAGCUUU U AUACACAC | 347 | GUGUGUAU CUGAUGAG GCCGUUAGGC CGAA AAAGCUGG | 1769 |
| 1784 | CAGCUUUU A UACACACG | 348 | CGUGUGUA CUGAUGAG GCCGUUAGGC CGAA AAAAGCUG | 1770 |
| 1786 | GCUUUUAU A CACACGUA | 349 | UACGUGUG CUGAUGAG GCCGUUAGGC CGAA AUAAAAGC | 1771 |
| 1794 | ACACACGU A UCUCAUUU | 350 | AAAUGAGA CUGAUGAG GCCGUUAGGC CGAA ACGUGUGU | 1772 |
| 1796 | ACACGUAU C UCAUUUUU | 351 | AAAAAUGA CUGAUGAG GCCGUUAGGC CGAA AUACGUGU | 1773 |
| 1798 | ACGUAUCU C AUUUUUAU | 352 | AUAAAAAU CUGAUGAG GCCGUUAGGC CGAA AGAUACGU | 1774 |
| 1801 | UAUCUCAU U UUUAUCAA | 353 | UUGAUAAA CUGAUGAG GCCGUUAGGC CGAA AUGAGAUA | 1775 |
| 1802 | AUCUCAUU U UUAUCAAA | 354 | UUUGAUAA CUGAUGAG GCCGUUAGGC CGAA AAUGAGAU | 1776 |
| 1803 | UCUCAUUU U UAUCAAAA | 355 | UUUUGAUA CUGAUGAG GCCGUUAGGC CGAA AAAUGAGA | 1777 |
| 1804 | CUCAUUUU U AUCAAAAC | 356 | GUUUUGAU CUGAUGAG GCCGUUAGGC CGAA AAAAUGAG | 1778 |
| 1805 | UCAUUUUU A UCAAAACA | 357 | UGUUUUGA CUGAUGAG GCCGUUAGGC CGAA AAAAAUGA | 1779 |
| 1807 | AUUUUUAU C AAAACAUU | 358 | AAUGUUUU CUGAUGAG GCCGUUAGGC CGAA AUAAAAAU | 1780 |

Input Sequence = AF016582 Cut Site = UH/.
Stem Length = 8. Core Sequence = CUGAUGAG GCCGUUAGGC CGAA
AF016582 (Homo sapiens checkpoint kinase Chk1 (CHK1) mRNA; 1821 bp)

Underlined region can be any X sequence or linker as previously defined herein.

TABLE IV
Human Chk1 NCH Ribozyme and Substrate Sequence

| | | | | Rz Seq |
|---|---|---|---|---|
| Pos | Substrate | Seq ID | Ribozyme | ID |
| 9 | GGCCGGAC A GUCCGCCG | 359 | CGGCGGAC CUGAUGAG GCCGUUAGGC CGAA IUCCGGCC | 1781 |
| 13 | GGACAGUC C GCCGAGGU | 360 | ACCUCGGC CUGAUGAG GCCGUUAGGC CGAA IACUGUCC | 1782 |
| 16 | CAGUCCGC C GAGGUGCU | 361 | AGCACCUC CUGAUGAG GCCGUUAGGC CGAA ICGGACUG | 1783 |
| 24 | CGAGGUGC U CGGUGGAG | 362 | CUCCACCG CUGAUGAG GCCGUUAGGC CGAA ICACCUCG | 1784 |
| 35 | GUGGAGUC A UGGCAGUG | 363 | CACUGCCA CUGAUGAG GCCGUUAGGC CGAA IACUCCAC | 1785 |
| 40 | GUCAUGGC A GUGCCCUU | 364 | AAGGGCAC CUGAUGAG GCCGUUAGGC CGAA ICCAUGAC | 1786 |
| 45 | GGCAGUGC C CUUUGUGG | 365 | CCACAAAG CUGAUGAG GCCGUUAGGC CGAA ICACUGCC | 1787 |
| 46 | GCAGUGCC C UUUGUGGA | 366 | UCCACAAA CUGAUGAG GCCGUUAGGC CGAA IGCACUGC | 1788 |
| 47 | CAGUGCCC U UUGUGGAA | 367 | UUCCACAA CUGAUGAG GCCGUUAGGC CGAA IGGCACUG | 1789 |
| 59 | UGGAAGAC U GGGACUUG | 368 | CAAGUCCC CUGAUGAG GCCGUUAGGC CGAA IUCUUCCA | 1790 |
| 65 | ACUGGGAC U UGGUGCAA | 369 | UUGCACCA CUGAUGAG GCCGUUAGGC CGAA IUCCCAGU | 1791 |
| 72 | CUUGGUGC A AACCCUGG | 370 | CCAGGGUU CUGAUGAG GCCGUUAGGC CGAA ICACCAAG | 1792 |
| 76 | GUGCAAAC C CUGGGAGA | 371 | UCUCCCAG CUGAUGAG GCCGUUAGGC CGAA IUUUGCAC | 1793 |
| 77 | UGCAAACC C UGGGAGAA | 372 | UUCUCCCA CUGAUGAG GCCGUUAGGC CGAA IGUUUGCA | 1794 |
| 78 | GCAAACCC U GGGAGAAG | 373 | CUUCUCCC CUGAUGAG GCCGUUAGGC CGAA IGGUUUGC | 1795 |
| 91 | GAAGGUGC C UAUGGAGA | 374 | UCUCCAUA CUGAUGAG GCCGUUAGGC CGAA ICACCUUC | 1796 |
| 92 | AAGGUGCC U AUGGAGAA | 375 | UUCUCCAU CUGAUGAG GCCGUUAGGC CGAA IGCACCUU | 1797 |
| 105 | AGAAGUUC A ACUUGCUG | 376 | CAGCAAGU CUGAUGAG GCCGUUAGGC CGAA IAACUUCU | 1798 |
| 108 | AGUUCAAC U UGCUGUGA | 377 | UCACAGCA CUGAUGAG GCCGUUAGGC CGAA IUUGAACU | 1799 |
| 112 | CAACUUGC U GUGAAUAG | 378 | CUAUUCAC CUGAUGAG GCCGUUAGGC CGAA ICAAGUUG | 1800 |
| 127 | AGAGUAAC U GAAGAAGC | 379 | GCUUCUUC CUGAUGAG GCCGUUAGGC CGAA IUUACUCU | 1801 |
| 136 | GAAGAAGC A GUCGCAGU | 380 | ACUGCGAC CUGAUGAG GCCGUUAGGC CGAA ICUUCUUC | 1802 |
| 142 | GCAGUCGC A GUGAAGAU | 381 | AUCUUCAC CUGAUGAG GCCGUUAGGC CGAA ICGACUGC | 1803 |
| 169 | AAGCGUGC C GUAGACUG | 382 | CAGUCUAC CUGAUGAG GCCGUUAGGC CGAA ICACGCUU | 1804 |
| 176 | CCGUAGAC U GUCCAGAA | 383 | UUCUGGAC CUGAUGAG GCCGUUAGGC CGAA IUCUACGG | 1805 |
| 180 | AGACUGUC C AGAAAAUA | 384 | UAUUUUCU CUGAUGAG GCCGUUAGGC CGAA IACAGUCU | 1806 |
| 181 | GACUGUCC A GAAAAUAU | 385 | AUAUUUUC CUGAUGAG GCCGUUAGGC CGAA IGACAGUC | 1807 |
| 203 | AAGAGAUC U GUAUCAAU | 386 | AUUGAUAC CUGAUGAG GCCGUUAGGC CGAA IAUCUCUU | 1808 |
| 209 | UCUGUAUC A AUAAAAUG | 387 | CAUUUUAU CUGAUGAG GCCGUUAGGC CGAA IAUACAGA | 1809 |
| 219 | UAAAAUGC U AAAUCAUG | 388 | CAUGAUUU CUGAUGAG GCCGUUAGGC CGAA ICAUUUUA | 1810 |
| 225 | GCUAAAUC A UGAAAAUG | 389 | CAUUUUCA CUGAUGAG GCCGUUAGGC CGAA IAUUUAGC | 1811 |
| 245 | UAAAAUUC U AUGGUCAC | 390 | GUGACCAU CUGAUGAG GCCGUUAGGC CGAA IAAUUUUA | 1812 |
| 252 | CUAUGGUC A CAGGAGAG | 391 | CUCUCCUG CUGAUGAG GCCGUUAGGC CGAA IACCAUAG | 1813 |
| 254 | AUGGUCAC A GGAGAGAA | 392 | UUCUCUCC CUGAUGAG GCCGUUAGGC CGAA IUGACCAU | 1814 |
| 266 | GAGAAGGC A AUAUCCAA | 393 | UUGGAUAU CUGAUGAG GCCGUUAGGC CGAA ICCUUCUC | 1815 |
| 272 | GCAAUAUC C AAUAUUUA | 394 | UAAAUAUU CUGAUGAG GCCGUUAGGC CGAA IAUAUUGC | 1816 |
| 273 | CAAUAUCC A AUAUUUAU | 395 | AUAAAUAU CUGAUGAG GCCGUUAGGC CGAA IGAUAUUG | 1817 |
| 285 | UUUAUUUC U GGAGUACU | 396 | AGUACUCC CUGAUGAG GCCGUUAGGC CGAA IAAAUAAA | 1818 |
| 293 | UGGAGUAC U GUAGUGGA | 397 | UCCACUAC CUGAUGAG GCCGUUAGGC CGAA IUACUCCA | 1819 |
| 309 | AGGAGAGC U UUUUGACA | 398 | UGUCAAAA CUGAUGAG GCCGUUAGGC CGAA ICUCUCCU | 1820 |
| 317 | UUUUUGAC A GAAUAGAG | 399 | CUCUAUUC CUGAUGAG GCCGUUAGGC CGAA IUCAAAAA | 1821 |
| 327 | AAUAGAGC C AGACAUAG | 400 | CUAUGUCU CUGAUGAG GCCGUUAGGC CGAA ICUCUAUU | 1822 |
| 328 | AUAGAGCC A GACAUAGG | 401 | CCUAUGUC CUGAUGAG GCCGUUAGGC CGAA IGCUCUAU | 1823 |
| 332 | AGCCAGAC A UAGGCAUG | 402 | CAUGCCUA CUGAUGAG GCCGUUAGGC CGAA IUCUGGCU | 1824 |
| 338 | ACAUAGGC A UGCCUGAA | 403 | UUCAGGCA CUGAUGAG GCCGUUAGGC CGAA ICCUAUGU | 1825 |
| 342 | AGGCAUGC C UGAACCAG | 404 | CUGGUUCA CUGAUGAG GCCGUUAGGC CGAA ICAUGCCU | 1826 |
| 343 | GGCAUGCC U GAACCAGA | 405 | UCUGGUUC CUGAUGAG GCCGUUAGGC CGAA IGCAUGCC | 1827 |
| 348 | GCCUGAAC C AGAUGCUC | 406 | GAGCAUCU CUGAUGAG GCCGUUAGGC CGAA IUUCAGGC | 1828 |
| 349 | CCUGAACC A GAUGCUCA | 407 | UGAGCAUC CUGAUGAG GCCGUUAGGC CGAA IGUUCAGG | 1829 |
| 355 | CCAGAUGC U CAGAGAUU | 408 | AAUCUCUG CUGAUGAG GCCGUUAGGC CGAA ICAUCUGG | 1830 |
| 357 | AGAUGCUC A GAGAUUCU | 409 | AGAAUCUC CUGAUGAG GCCGUUAGGC CGAA IAGCAUCU | 1831 |
| 365 | AGAGAUUC U UCCAUCAA | 410 | UUGAUGGA CUGAUGAG GCCGUUAGGC CGAA IAAUCUCU | 1832 |
| 368 | GAUUCUUC C AUCAACUC | 411 | GAGUUGAU CUGAUGAG GCCGUUAGGC CGAA IAAGAAUC | 1833 |
| 369 | AUUCUUCC A UCAACUCA | 412 | UGAGUUGA CUGAUGAG GCCGUUAGGC CGAA IGAAGAAU | 1834 |
| 372 | CUUCCAUC A ACUCAUGG | 413 | CCAUGAGU CUGAUGAG GCCGUUAGGC CGAA IAUGGAAG | 1835 |
| 375 | CCAUCAAC U CAUGGCAG | 414 | CUGCCAUG CUGAUGAG GCCGUUAGGC CGAA IUUGAUGG | 1836 |
| 377 | AUCAACUC A UGGCAGGG | 415 | CCCUGCCA CUGAUGAG GCCGUUAGGC CGAA IAGUUGAU | 1837 |
| 382 | CUCAUGGC A GGGGUGGU | 416 | ACCACCCC CUGAUGAG GCCGUUAGGC CGAA ICCAUGAG | 1838 |
| 396 | GGUUUAUC U GCAUGGUA | 417 | UACCAUGC CUGAUGAG GCCGUUAGGC CGAA IAUAAACC | 1839 |
| 399 | UUAUCUGC A UGGUAUUG | 418 | CAAUACCA CUGAUGAG GCCGUUAGGC CGAA ICAGAUAA | 1840 |
| 415 | GGAAUAAC U CACAGGGA | 419 | UCCCUGUG CUGAUGAG GCCGUUAGGC CGAA IUUAUUCC | 1841 |
| 417 | AAUAACUC A CAGGGAUA | 420 | UAUCCCUG CUGAUGAG GCCGUUAGGC CGAA IAGUUAUU | 1842 |
| 419 | UAACUCAC A GGGAUAUU | 421 | AAUAUCCC CUGAUGAG GCCGUUAGGC CGAA IUGAGUUA | 1843 |
| 432 | UAUUAAAC C AGAAAAUC | 422 | GAUUUUCU CUGAUGAG GCCGUUAGGC CGAA IUUUAAUA | 1844 |
| 433 | AUUAAACC A GAAAAUCU | 423 | AGAUUUUC CUGAUGAG GCCGUUAGGC CGAA IGUUUAAU | 1845 |
| 441 | AGAAAAUC U UCUGUUGG | 424 | CCAACAGA CUGAUGAG GCCGUUAGGC CGAA IAUUUUCU | 1846 |
| 444 | AAAUCUUC U GUUGGAUG | 425 | CAUCCAAC CUGAUGAG GCCGUUAGGC CGAA IAAGAUUU | 1847 |
| 464 | GGGAUAAC C UCAAAAUC | 426 | GAUUUUGA CUGAUGAG GCCGUUAGGC CGAA IUUAUCCC | 1848 |
| 465 | GGAUAACC U CAAAAUCU | 427 | AGAUUUUG CUGAUGAG GCCGUUAGGC CGAA IGUUAUCC | 1849 |
| 467 | AUAACCUC A AAAUCUCA | 428 | UGAGAUUU CUGAUGAG GCCGUUAGGC CGAA IAGGUUAU | 1850 |
| 473 | UCAAAAUC U CAGACUUU | 429 | AAAGUCUG CUGAUGAG GCCGUUAGGC CGAA IAUUUUGA | 1851 |
| 475 | AAAAUCUC A GACUUUGG | 430 | CCAAAGUC CUGAUGAG GCCGUUAGGC CGAA IAGAUUUU | 1852 |
| 479 | UCUCAGAC U UUGGCUUG | 431 | CAAGCCAA CUGAUGAG GCCGUUAGGC CGAA IUCUGAGA | 1853 |
| 485 | ACUUUGGC U UGGCAACA | 432 | UGUUGCCA CUGAUGAG GCCGUUAGGC CGAA ICCAAAGU | 1854 |
| 490 | GGCUUGGC A ACAGUAUU | 433 | AAUACUGU CUGAUGAG GCCGUUAGGC CGAA ICCAAGCC | 1855 |
| 493 | UUGGCAAC A GUAUUUCG | 434 | CGAAAUAC CUGAUGAG GCCGUUAGGC CGAA IUUGCCAA | 1856 |
| 530 | UGUUGAAC A AGAUGUGU | 435 | ACACAUCU CUGAUGAG GCCGUUAGGC CGAA IUUCAACA | 1857 |
| 544 | UGUGGUAC U UUACCAUA | 436 | UAUGGUAA CUGAUGAG GCCGUUAGGC CGAA IUACCACA | 1858 |
| 549 | UACUUUAC C AUAUGUUG | 437 | CAACAUAU CUGAUGAG GCCGUUAGGC CGAA IUAAAGUA | 1859 |
| 550 | ACUUUACC A UAUGUUGC | 438 | GCAACAUA CUGAUGAG GCCGUUAGGC CGAA IGUAAAGU | 1860 |
| 559 | UAUGUUGC U CCAGAACU | 439 | AGUUCUGG CUGAUGAG GCCGUUAGGC CGAA ICAACAUA | 1861 |
| 561 | UGUUGCUC C AGAACUUC | 440 | GAAGUUCU CUGAUGAG GCCGUUAGGC CGAA IAGCAACA | 1862 |
| 562 | GUUGCUCC A GAACUUCU | 441 | AGAAGUUC CUGAUGAG GCCGUUAGGC CGAA IGAGCAAC | 1863 |
| 567 | UCCAGAAC U UCUGAAGA | 442 | UCUUCAGA CUGAUGAG GCCGUUAGGC CGAA IUUCUGGA | 1864 |
| 570 | AGAACUUC U GAAGAGAA | 443 | UUCUCUUC CUGAUGAG GCCGUUAGGC CGAA IAAGUUCU | 1865 |
| 588 | AGAAUUUC A UGCAGAAC | 444 | GUUCUGCA CUGAUGAG GCCGUUAGGC CGAA IAAAUUCU | 1866 |
| 592 | UUUCAUGC A GAACCAGU | 445 | ACUGGUUC CUGAUGAG GCCGUUAGGC CGAA ICAUGAAA | 1867 |
| 597 | UGCAGAAC C AGUUGAUG | 446 | CAUCAACU CUGAUGAG GCCGUUAGGC CGAA IUUCUGCA | 1868 |
| 598 | GCAGAACC A GUUGAUGU | 447 | ACAUCAAC CUGAUGAG GCCGUUAGGC CGAA IGUUCUGC | 1869 |
| 613 | GUUUGGUC C UGUGGAAU | 448 | AUUCCACA CUGAUGAG GCCGUUAGGC CGAA IACCAAAC | 1870 |
| 614 | UUUGGUCC U GUGGAAUA | 449 | UAUUCCAC CUGAUGAG GCCGUUAGGC CGAA IGACCAAA | 1871 |
| 627 | AAUAGUAC U UACUGCAA | 450 | UUGCAGUA CUGAUGAG GCCGUUAGGC CGAA IUACUAUU | 1872 |
| 631 | GUACUUAC U GCAAUGCU | 451 | AGCAUUGC CUGAUGAG GCCGUUAGGC CGAA IUAAGUAC | 1873 |
| 634 | CUUACUGC A AUGCUCGC | 452 | GCGAGCAU CUGAUGAG GCCGUUAGGC CGAA ICAGUAAG | 1874 |
| 639 | UGCAAUGC U CGCUGGAG | 453 | CUCCAGCG CUGAUGAG GCCGUUAGGC CGAA ICAUUGCA | 1875 |
| 643 | AUGCUCGC U GGAGAAUU | 454 | AAUUCUCC CUGAUGAG GCCGUUAGGC CGAA ICGAGCAU | 1876 |
| 654 | AGAAUUGC C AUGGGACC | 455 | GGUCCCAU CUGAUGAG GCCGUUAGGC CGAA ICAAUUCU | 1877 |
| 655 | GAAUUGCC A UGGGACCA | 456 | UGGUCCCA CUGAUGAG GCCGUUAGGC CGAA IGCAAUUC | 1878 |
| 662 | CAUGGGAC C AACCCAGU | 457 | ACUGGGUU CUGAUGAG GCCGUUAGGC CGAA IUCCCAUG | 1879 |
| 663 | AUGGGACC A ACCCAGUG | 458 | CACUGGGU CUGAUGAG GCCGUUAGGC CGAA IGUCCCAU | 1880 |
| 666 | GGACCAAC C CAGUGACA | 459 | UGUCACUG CUGAUGAG GCCGUUAGGC CGAA IUUGGUCC | 1881 |
| 667 | GACCAACC C AGUGACAG | 460 | CUGUCACU CUGAUGAG GCCGUUAGGC CGAA IGUUGGUC | 1882 |
| 668 | ACCAACCC A GUGACAGC | 461 | GCUGUCAC CUGAUGAG GCCGUUAGGC CGAA IGGUUGGU | 1883 |
| 674 | CCAGUGAC A GCUGUCAG | 462 | CUGACAGC CUGAUGAG GCCGUUAGGC CGAA IUCACUGG | 1884 |
| 677 | GUGACAGC U GACAGGAG | 463 | CUCCUGAC CUGAUGAG GCCGUUAGGC CGAA ICUGUCAC | 1885 |
| 681 | CAGCUGUC A GGAGUAUU | 464 | AAUACUCC CUGAUGAG GCCGUUAGGC CGAA IACAGCUG | 1886 |
| 691 | GAGUAUUC U GACUGGAA | 465 | UUCCAGUC CUGAUGAG GCCGUUAGGC CGAA IAAUACUC | 1887 |
| 695 | AUUCUGAC U GGAAAGAA | 466 | UUCUUUCC CUGAUGAG GCCGUUAGGC CGAA IUCAGAAU | 1888 |
| 712 | AAAAAAAC A UACCUCAA | 467 | UUGAGGUA CUGAUGAG GCCGUUAGGC CGAA IUUUUUUU | 1889 |
| 716 | AAACAUAC C UCAACCCU | 468 | AGGGUUGA CUGAUGAG GCCGUUAGGC CGAA IUAUGUUU | 1890 |
| 717 | AACAUACC U CAACCCUU | 469 | AAGGGUUG CUGAUGAG GCCGUUAGGC CGAA IGUAUGUU | 1891 |
| 719 | CAUACCUC A ACCCUUGG | 470 | CCAAGGGU CUGAUGAG GCCGUUAGGC CGAA IAGGUAUG | 1892 |
| 722 | ACCUCAAC C CUUGGAAA | 471 | UUUCCAAG CUGAUGAG GCCGUUAGGC CGAA IUUGAGGU | 1893 |
| 723 | CCUCAACC C UUGGAAAA | 472 | UUUUCCAA CUGAUGAG GCCGUUAGGC CGAA IGUUGAGG | 1894 |
| 724 | CUCAACCC U UGGAAAAA | 473 | UUUUUCCA CUGAUGAG GCCGUUAGGC CGAA IGGUUGAG | 1895 |
| 742 | AUCGAUUC U GCUCCUCU | 474 | AGAGGAGC CUGAUGAG GCCGUUAGGC CGAA IAAUCGAU | 1896 |
| 745 | GAUUCUGC U CCUCUAGC | 475 | GCUAGAGG CUGAUGAG GCCGUUAGGC CGAA ICAGAAUC | 1897 |
| 747 | UUCUGCUC C UCUAGCUC | 476 | GAGCUAGA CUGAUGAG GCCGUUAGGC CGAA IAGCAGAA | 1898 |
| 748 | UCUGCUCC U CUAGCUCU | 477 | AGAGCUAG CUGAUGAG GCCGUUAGGC CGAA IGAGCAGA | 1899 |
| 750 | UGCUCCUC U AGCUCUGC | 478 | GCAGAGCU CUGAUGAG GCCGUUAGGC CGAA IAGGAGCA | 1900 |
| 754 | CCUCUAGC U CUGCUGCA | 479 | UGCAGCAG CUGAUGAG GCCGUUAGGC CGAA ICUAGAGG | 1901 |
| 756 | UCUAGCUC U GCUGCAUA | 480 | UAUGCAGC CUGAUGAG GCCGUUAGGC CGAA IAGCUAGA | 1902 |
| 759 | AGCUCUGC U GCAUAAAA | 481 | UUUUAUGC CUGAUGAG GCCGUUAGGC CGAA ICAGAGCU | 1903 |
| 762 | UCUGCUGC A UAAAAUCU | 482 | AGAUUUUA CUGAUGAG GCCGUUAGGC CGAA ICAGCAGA | 1904 |
| 770 | AUAAAAUC U UAGUUGAG | 483 | CUCAACUA CUGAUGAG GCCGUUAGGC CGAA IAUUUUAU | 1905 |
| 783 | UGAGAAUC C AUCAGCAA | 484 | UUGCUGAU CUGAUGAG GCCGUUAGGC CGAA IAUUCUCA | 1906 |
| 784 | GAGAAUCC A UCAGCAAG | 485 | CUUGCUGA CUGAUGAG GCCGUUAGGC CGAA IGAUUCUC | 1907 |
| 787 | AAUCCAUC A GCAAGAAU | 486 | AUUCUUGC CUGAUGAG GCCGUUAGGC CGAA IAUGGAUU | 1908 |
| 790 | CCAUCAGC A AGAAUUAC | 487 | GUAAUUCU CUGAUGAG GCCGUUAGGC CGAA ICUGAUGG | 1909 |
| 799 | AGAAUUAC C AUUCCAGA | 488 | UCUGGAAU CUGAUGAG GCCGUUAGGC CGAA IUAAUUCU | 1910 |
| 800 | GAAUUACC A UUCCAGAC | 489 | GUCUGGAA CUGAUGAG GCCGUUAGGC CGAA IGUAAUUC | 1911 |
| 804 | UACCAUUC C AGACAUCA | 490 | UGAUGUCU CUGAUGAG GCCGUUAGGC CGAA IAAUGGUA | 1912 |
| 805 | ACCAUUCC A GACAUCAA | 491 | UUGAUGUC CUGAUGAG GCCGUUAGGC CGAA IGAAUGGU | 1913 |
| 809 | UUCCAGAC A UCAAAAAA | 492 | UUUUUUGA CUGAUGAG GCCGUUAGGC CGAA IUCUGGAA | 1914 |
| 812 | CAGACAUC A AAAAAGAU | 493 | AUCUUUUU CUGAUGAG GCCGUUAGGC CGAA IAUGUCUG | 1915 |
| 830 | GAUGGUAC A ACAAACCC | 494 | GGGUUUGU CUGAUGAG GCCGUUAGGC CGAA IUACCAUC | 1916 |
| 833 | GGUACAAC A AACCCCUC | 495 | GAGGGGUU CUGAUGAG GCCGUUAGGC CGAA IUUGUACC | 1917 |
| 837 | CAACAAAC C CCUCAAGA | 496 | UCUUGAGG CUGAUGAG GCCGUUAGGC CGAA IUUUGUUG | 1918 |
| 838 | AACAAACC C CUCAAGAA | 497 | UUCUUGAG CUGAUGAG GCCGUUAGGC CGAA IGUUUGUU | 1919 |
| 839 | ACAAACCC C UCAAGAAA | 498 | UUUCUUGA CUGAUGAG GCCGUUAGGC CGAA IGGUUUGU | 1920 |
| 840 | CAAACCCC U CAAGAAAG | 499 | CUUUCUUG CUGAUGAG GCCGUUAGGC CGAA IGGGUUUG | 1921 |
| 842 | AACCCCUC A AGAAAGGG | 500 | CCCUUUCU CUGAUGAG GCCGUUAGGC CGAA IAGGGGUU | 1922 |
| 853 | AAAGGGGC A AAAAGGCC | 501 | GGCCUUUU CUGAUGAG GCCGUUAGGC CGAA ICCCCUUU | 1923 |
| 861 | AAAAAGGC C CCGAGUCA | 502 | UGACUCGG CUGAUGAG GCCGUUAGGC CGAA ICCUUUUU | 1924 |
| 862 | AAAAGGCC C CGAGUCAC | 503 | GUGACUCG CUGAUGAG GCCGUUAGGC CGAA IGCCUUUU | 1925 |
| 863 | AAAGGCCC C GAGUCACU | 504 | AGUGACUC CUGAUGAG GCCGUUAGGC CGAA IGGCCUUU | 1926 |
| 869 | CCCGAGUC A CUUCAGGU | 505 | ACCUGAAG CUGAUGAG GCCGUUAGGC CGAA IACUCGGG | 1927 |
| 871 | CGAGUCAC U UCAGGUGG | 506 | CCACCUGA CUGAUGAG GCCGUUAGGC CGAA IUGACUCG | 1928 |
| 874 | GUCACUUC A GGUGGUGU | 507 | ACACCACC CUGAUGAG GCCGUUAGGC CGAA IAAGUGAC | 1929 |
| 886 | GGUGUGUC A GAGUCUCC | 508 | GGAGACUC CUGAUGAG GCCGUUAGGC CGAA IACACACC | 1930 |
| 892 | UCAGAGUC U CCCAGUGG | 509 | CCACUGGG CUGAUGAG GCCGUUAGGC CGAA IACUCUGA | 1931 |
| 894 | AGAGUCUC C CAGUGGAU | 510 | AUCCACUG CUGAUGAG GCCGUUAGGC CGAA IAGACUCU | 1932 |
| 895 | GAGUCUCC C AGUGGAUU | 511 | AAUCCACU CUGAUGAG GCCGUUAGGC CGAA IGAGACUC | 1933 |
| 896 | AGUCUCCC A GUGGAUUU | 512 | AAAUCCAC CUGAUGAG GCCGUUAGGC CGAA IGGAGACU | 1934 |
| 907 | GGAUUUUC U AAGCACAU | 513 | AUGUGCUU CUGAUGAG GCCGUUAGGC CGAA IAAAAUCC | 1935 |
| 912 | UUCUAAGC A CAUUCAAU | 514 | AUUGAAUG CUGAUGAG GCCGUUAGGC CGAA ICUUAGAA | 1936 |
| 914 | CUAAGCAC A UUCAAUCC | 515 | GGAUUGAA CUGAUGAG GCCGUUAGGC CGAA IUGCUUAG | 1937 |
| 918 | GCACAUUC A AUCCAAUU | 516 | AAUUGGAU CUGAUGAG GCCGUUAGGC CGAA IAAUGUGC | 1938 |
| 922 | AUUCAAUC C AAUUUGGA | 517 | UCCAAAUU CUGAUGAG GCCGUUAGGC CGAA IAUUGAAU | 1939 |
| 923 | UUCAAUCC A AUUUGGAC | 518 | GUCCAAAU CUGAUGAG GCCGUUAGGC CGAA IGAUUGAA | 1940 |
| 932 | AUUUGGAC U UCUCUCCA | 519 | UGGAGAGA CUGAUGAG GCCGUUAGGC CGAA IUCCAAAU | 1941 |
| 935 | UGGACUUC U CUCCAGUA | 520 | UACUGGAG CUGAUGAG GCCGUUAGGC CGAA IAAGUCCA | 1942 |
| 937 | GACUUCUC U CCAGUAAA | 521 | UUUACUGG CUGAUGAG GCCGUUAGGC CGAA IAGAAGUC | 1943 |
| 939 | CUUCUCUC C AGUAAACA | 522 | UGUUUACU CUGAUGAG GCCGUUAGGC CGAA IAGAGAAG | 1944 |
| 940 | UUCUCUCC A GUAAACAG | 523 | CUGUUUAC CUGAUGAG GCCGUUAGGC CGAA IGAGAGAA | 1945 |
| 947 | CAGUAAAC A GUGCUUCU | 524 | AGAAGCAC CUGAUGAG GCCGUUAGGC CGAA IUUUACUG | 1946 |
| 952 | AACAGUGC U UCUAGUGA | 525 | UCACUAGA CUGAUGAG GCCGUUAGGC CGAA ICACUGUU | 1947 |
| 955 | AGUGCUUC U AGUGAAGA | 526 | UCUUCACU CUGAUGAG GCCGUUAGGC CGAA IAAGCACU | 1948 |
| 977 | UGAAGUAC U CCAGUUCU | 527 | AGAACUGG CUGAUGAG GCCGUUAGGC CGAA IUACUUCA | 1949 |
| 979 | AAGUACUC C AGUUCUCA | 528 | UGAGAACU CUGAUGAG GCCGUUAGGC CGAA IAGUACUU | 1950 |
| 980 | AGUACUCC A GUUCUCAG | 529 | CUGAGAAC CUGAUGAG GCCGUUAGGC CGAA IGAGUACU | 1951 |
| 985 | UCCAGUUC U CAGCCAGA | 530 | UCUGGCUG CUGAUGAG GCCGUUAGGC CGAA IAACUGGA | 1952 |
| 987 | CAGUUCUC A GCCAGAAC | 531 | GUUCUGGC CUGAUGAG GCCGUUAGGC CGAA IAGAACUG | 1953 |
| 990 | UUCUCAGC C AGAACCCC | 532 | GGGGUUCU CUGAUGAG GCCGUUAGGC CGAA ICUGAGAA | 1954 |
| 991 | UCUCAGCC A GAACCCCG | 533 | CGGGGUUC CUGAUGAG GCCGUUAGGC CGAA IGCUGAGA | 1955 |
| 996 | GCCAGAAC C CCGCACAG | 534 | CUGUGCGG CUGAUGAG GCCGUUAGGC CGAA IUUCUGGC | 1956 |
| 997 | CCAGAACC C CGCACAGG | 535 | CCUGUGCG CUGAUGAG GCCGUUAGGC CGAA IGUUCUGG | 1957 |
| 998 | CAGAACCC C GCACAGGU | 536 | ACCUGUGC CUGAUGAG GCCGUUAGGC CGAA IGGUUCUG | 1958 |
| 1001 | AACCCCGC A CAGGUCUU | 537 | AAGACCUG CUGAUGAG GCCGUUAGGC CGAA ICGGGGUU | 1959 |
| 1003 | CCCCGCAC A GGUCUUUC | 538 | GAAAGACC CUGAUGAG GCCGUUAGGC CGAA IUGCGGGG | 1960 |
| 1008 | CACAGGUC U UUCCUUAU | 539 | AUAAGGAA CUGAUGAG GCCGUUAGGC CGAA IACCUGUG | 1961 |
| 1012 | GGUCUUUC C UUAUGGGA | 540 | UCCCAUAA CUGAUGAG GCCGUUAGGC CGAA IAAAGACC | 1962 |
| 1013 | GUCUUUCC U UAUGGGAU | 541 | AUCCCAUA CUGAUGAG GCCGUUAGGC CGAA IGAAAGAC | 1963 |
| 1024 | UGGGAUAC C AGCCCCUC | 542 | GAGGGGCU CUGAUGAG GCCGUUAGGC CGAA IUAUCCCA | 1964 |
| 1025 | GGGAUACC A GCCCCUCA | 543 | UGAGGGGC CUGAUGAG GCCGUUAGGC CGAA IGUAUCCC | 1965 |
| 1028 | AUACCAGC C CCUCAUAC | 544 | GUAUGAGG CUGAUGAG GCCGUUAGGC CGAA ICUGGUAU | 1966 |
| 1029 | UACCAGCC C CUCAUACA | 545 | UGUAUGAG CUGAUGAG GCCGUUAGGC CGAA IGCUGGUA | 1967 |
| 1030 | ACCAGCCC C UCAUACAU | 546 | AUGUAUGA CUGAUGAG GCCGUUAGGC CGAA IGGCUGGU | 1968 |
| 1031 | CCAGCCCC U CAUACAUU | 547 | AAUGUAUG CUGAUGAG GCCGUUAGGC CGAA IGGGCUGG | 1969 |
| 1033 | AGCCCCUC A UACAUUGA | 548 | UCAAUGUA CUGAUGAG GCCGUUAGGC CGAA IAGGGGCU | 1970 |
| 1037 | CCUCAUAC A UUGAUAAA | 549 | UUUAUCAA CUGAUGAG GCCGUUAGGC CGAA IUAUGAGG | 1971 |
| 1053 | AUUGGUAC A AGGGAUCA | 550 | UGAUCCCU CUGAUGAG GCCGUUAGGC CGAA IUACCAAU | 1972 |
| 1061 | AAGGGAUC A GCUUUUCC | 551 | GGAAAAGC CUGAUGAG GCCGUUAGGC CGAA IAUCCCUU | 1973 |
| 1064 | GGAUCAGC U UUUCCCAG | 552 | CUGGGAAA CUGAUGAG GCCGUUAGGC CGAA ICUGAUCC | 1974 |
| 1069 | AGCUUUUC C CAGCCCAC | 553 | GUGGGCUG CUGAUGAG GCCGUUAGGC CGAA IAAAAGCU | 1975 |
| 1070 | GCUUUUCC C AGCCCACA | 554 | UGUGGGCU CUGAUGAG GCCGUUAGGC CGAA IGAAAAGC | 1976 |
| 1071 | CUUUUCCC A GCCCACAU | 555 | AUGUGGGC CUGAUGAG GCCGUUAGGC CGAA IGGAAAAG | 1977 |
| 1074 | UUCCCAGC C CACAUGUC | 556 | GACAUGUG CUGAUGAG GCCGUUAGGC CGAA ICUGGGAA | 1978 |
| 1075 | UCCCAGCC C ACAUGUCC | 557 | GGACAUGU CUGAUGAG GCCGUUAGGC CGAA IGCUGGGA | 1979 |
| 1076 | CCCAGCCC A CAUGUCCU | 558 | AGGACAUG CUGAUGAG GCCGUUAGGC CGAA IGGCUGGG | 1980 |
| 1078 | CAGCCCAC A UGUCCUGA | 559 | UCAGGACA CUGAUGAG GCCGUUAGGC CGAA IUGGGCUG | 1981 |
| 1083 | CACAUGUC C UGAUCAUA | 560 | UAUGAUCA CUGAUGAG GCCGUUAGGC CGAA IACAUGUG | 1982 |
| 1084 | ACAUGUCC U GAUCAUAU | 561 | AUAUGAUC CUGAUGAG GCCGUUAGGC CGAA IGACAUGU | 1983 |
| 1089 | UCCUGAUC A UAUGCUUU | 562 | AAAGCAUA CUGAUGAG GCCGUUAGGC CGAA IAUCAGGA | 1984 |
| 1095 | UCAUAUGC U UUUGAAUA | 563 | UAUUCAAA CUGAUGAG GCCGUUAGGC CGAA ICAUAUGA | 1985 |
| 1107 | GAAUAGUC A GUUACUUG | 564 | CAAGUAAC CUGAUGAG GCCGUUAGGC CGAA IACUAUUC | 1986 |
| 1113 | UCAGUUAC U UGGCACCC | 565 | GGGUGCCA CUGAUGAG GCCGUUAGGC CGAA IUAACUGA | 1987 |
| 1118 | UACUUGGC A CCCCAGGA | 566 | UCCUGGGG CUGAUGAG GCCGUUAGGC CGAA ICCAAGUA | 1988 |
| 1120 | CUUGGCAC C CCAGGAUC | 567 | GAUCCUGG CUGAUGAG GCCGUUAGGC CGAA IUGCCAAG | 1989 |
| 1121 | UUGGCACC C CAGGAUCC | 568 | GGAUCCUG CUGAUGAG GCCGUUAGGC CGAA IGUGCCAA | 1990 |
| 1122 | UGGCACCC C AGGAUCCU | 569 | AGGAUCCU CUGAUGAG GCCGUUAGGC CGAA IGGUGCCA | 1991 |
| 1123 | GGCACCCC A GGAUCCUC | 570 | GAGGAUCC CUGAUGAG GCCGUUAGGC CGAA IGGGUGCC | 1992 |
| 1129 | CCAGGAUC C UCACAGAA | 571 | UUCUGUGA CUGAUGAG GCCGUUAGGC CGAA IAUCCUGG | 1993 |
| 1130 | CAGGAUCC U CACAGAAC | 572 | GUUCUGUG CUGAUGAG GCCGUUAGGC CGAA IGAUCCUG | 1994 |
| 1132 | GGAUCCUC A CAGAACCC | 573 | GGGUUCUG CUGAUGAG GCCGUUAGGC CGAA IAGGAUCC | 1995 |
| 1134 | AUCCUCAC A GAACCCCU | 574 | AGGGGUUC CUGAUGAG GCCGUUAGGC CGAA IUGAGGAU | 1996 |
| 1139 | CACAGAAC C CCUGGCAG | 575 | CUGCCAGG CUGAUGAG GCCGUUAGGC CGAA IUUCUGUG | 1997 |
| 1140 | ACAGAACC C CUGGCAGC | 576 | GCUGCCAG CUGAUGAG GCCGUUAGGC CGAA IGUUCUGU | 1998 |
| 1141 | CAGAACCC C UGGCAGCG | 577 | CGCUGCCA CUGAUGAG GCCGUUAGGC CGAA IGGUUCUG | 1999 |
| 1142 | AGAACCCC U GGCAGCGG | 578 | CCGCUGCC CUGAUGAG GCCGUUAGGC CGAA IGGGUUCU | 2000 |
| 1146 | CCCCUGGC A GCGGUUGG | 579 | CCAACCGC CUGAUGAG GCCGUUAGGC CGAA ICCAGGGG | 2001 |
| 1157 | GGUUGGUC A AAAGAAUG | 580 | CAUUCUUU CUGAUGAG GCCGUUAGGC CGAA IACCAACC | 2002 |
| 1168 | AGAAUGAC A CGAUUCUU | 581 | AAGAAUCG CUGAUGAG GCCGUUAGGC CGAA IUCAUUCU | 2003 |
| 1175 | CACGAUUC U UUACCAAA | 582 | UUUGGUAA CUGAUGAG GCCGUUAGGC CGAA IAAUCGUG | 2004 |
| 1180 | UUCUUUAC C AAAUUGGA | 583 | UCCAAUUU CUGAUGAG GCCGUUAGGC CGAA IUAAAGAA | 2005 |
| 1181 | UCUUUACC A AAUUGGAU | 584 | AUCCAAUU CUGAUGAG GCCGUUAGGC CGAA IGUAAAGA | 2006 |
| 1192 | UUGGAUGC A GACAAAUC | 585 | GAUUUGUC CUGAUGAG GCCGUUAGGC CGAA ICAUCCAA | 2007 |
| 1196 | AUGCAGAC A AAUCUUAU | 586 | AUAAGAUU CUGAUGAG GCCGUUAGGC CGAA IUCUGCAU | 2008 |
| 1201 | GACAAAUC U UAUCAAUG | 587 | CAUUGAUA CUGAUGAG GCCGUUAGGC CGAA IAUUUGUC | 2009 |
| 1206 | AUCUUAUC A AUGCCUGA | 588 | UCAGGCAU CUGAUGAG GCCGUUAGGC CGAA IAUAAGAU | 2010 |
| 1211 | AUCAAUGC C UGAAAGAG | 589 | CUCUUUCA CUGAUGAG GCCGUUAGGC CGAA ICAUUGAU | 2011 |
| 1212 | UCAAUGCC U GAAAGAGA | 590 | UCUCUUUC CUGAUGAG GCCGUUAGGC CGAA IGCAUUGA | 2012 |
| 1222 | AAAGAGAC U UGUGAGAA | 591 | UUCUCACA CUGAUGAG GCCGUUAGGC CGAA IUCUCUUU | 2013 |
| 1238 | AGUUGGGC U AUCAAUGG | 592 | CCAUUGAU CUGAUGAG GCCGUUAGGC CGAA ICCCAACU | 2014 |
| 1242 | GGGCUAUC A AUGGAAGA | 593 | UCUUCCAU CUGAUGAG GCCGUUAGGC CGAA IAUAGCCC | 2015 |
| 1266 | UAUGAAUC A GGUUACUA | 594 | UAGUAACC CUGAUGAG GCCGUUAGGC CGAA IAUUCAUA | 2016 |
| 1273 | CAGGUUAC U AUAUCAAC | 595 | GUUGAUAU CUGAUGAG GCCGUUAGGC CGAA IUAACCUG | 2017 |
| 1279 | ACUAUAUC A ACAACUGA | 596 | UCAGUUGA CUGAUGAG GCCGUUAGGC CGAA IAUAUAGU | 2018 |
| 1282 | AUAUCAAC A ACUGAUAG | 597 | CUAUCAGU CUGAUGAG GCCGUUAGGC CGAA IUUGAUAU | 2019 |
| 1285 | UCAACAAC U GAUAGGAG | 598 | CUCCUAUC CUGAUGAG GCCGUUAGGC CGAA IUUGUUGA | 2020 |
| 1298 | GGAGAAAC A AUAAACUC | 599 | GAGUUUAU CUGAUGAG GCCGUUAGGC CGAA IUUUCUCC | 2021 |
| 1305 | CAAUAAAC U CAUUUUCA | 600 | UGAAAAUG CUGAUGAG GCCGUUAGGC CGAA IUUUAUUG | 2022 |
| 1307 | AUAAACUC A UUUUCAAA | 601 | UUUGAAAA CUGAUGAG GCCGUUAGGC CGAA IAGUUUAU | 2023 |
| 1313 | UCAUUUUC A AAGUGAAU | 602 | AUUCACUU CUGAUGAG GCCGUUAGGC CGAA IAAAAUGA | 2024 |
| 1355 | UGGUUGAC U UCCGGCUU | 603 | AAGCCGGA CUGAUGAG GCCGUUAGGC CGAA IUCAACCA | 2025 |
| 1358 | UUGACUUC C GGCUUUCU | 604 | AGAAAGCC CUGAUGAG GCCGUUAGGC CGAA IAAGUCAA | 2026 |
| 1362 | CUUCCGGC U UUCUAAGG | 605 | CCUUAGAA CUGAUGAG GCCGUUAGGC CGAA ICCGGAAG | 2027 |
| 1366 | CGGCUUUC U AAGGGUGA | 606 | UCACCCUU CUGAUGAG GCCGUUAGGC CGAA IAAAGCCG | 2028 |
| 1388 | UGGAGUUC A AGAGACAC | 607 | GUGUCUCU CUGAUGAG GCCGUUAGGC CGAA IAACUCCA | 2029 |
| 1395 | CAAGAGAC A CUUCCUGA | 608 | UCAGGAAG CUGAUGAG GCCGUUAGGC CGAA IUCUCUUG | 2030 |
| 1397 | AGAGACAC U UCCUGAAG | 609 | CUUCAGGA CUGAUGAG GCCGUUAGGC CGAA IUGUCUCU | 2031 |
| 1400 | GACACUUC C UGAAGAUU | 610 | AAUCUUCA CUGAUGAG GCCGUUAGGC CGAA IAAGUGUC | 2032 |
| 1401 | ACACUUCC U GAAGAUUA | 611 | UAAUCUUC CUGAUGAG GCCGUUAGGC CGAA IGAAGUGU | 2033 |
| 1419 | AGGGAAGC U GAUUGAUA | 612 | UAUCAAUC CUGAUGAG GCCGUUAGGC CGAA ICUUCCCU | 2034 |
| 1436 | UUGUGAGC A GCCAGAAG | 613 | CUUCUGGC CUGAUGAG GCCGUUAGGC CGAA ICUCACAA | 2035 |
| 1439 | UGAGCAGC C AGAAGGUU | 614 | AACCUUCU CUGAUGAG GCCGUUAGGC CGAA ICUGCUCA | 2036 |
| 1440 | GAGCAGCC A GAAGGUUU | 615 | AAACCUUC CUGAUGAG GCCGUUAGGC CGAA IGCUGCUC | 2037 |
| 1452 | GGUUUGGC U UCCUGCCA | 616 | UGGCAGGA CUGAUGAG GCCGUUAGGC CGAA ICCAAACC | 2038 |
| 1455 | UUGGCUUC C UGCCACAU | 617 | AUGUGGCA CUGAUGAG GCCGUUAGGC CGAA IAAGCCAA | 2039 |
| 1456 | UGGCUUCC U GCCACAUG | 618 | CAUGUGGC CUGAUGAG GCCGUUAGGC CGAA IGAAGCCA | 2040 |
| 1459 | CUUCCUGC C ACAUGAUC | 619 | GAUCAUGU CUGAUGAG GCCGUUAGGC CGAA ICAGGAAG | 2041 |
| 1460 | UUCCUGCC A CAUGAUCG | 620 | CGAUCAUG CUGAUGAG GCCGUUAGGC CGAA IGCAGGAA | 2042 |
| 1462 | CCUGCCAC A UGAUCGGA | 621 | UCCGAUCA CUGAUGAG GCCGUUAGGC CGAA IUGGCAGG | 2043 |
| 1472 | GAUCGGAC C AUCGGCUC | 622 | GAGCCGAU CUGAUGAG GCCGUUAGGC CGAA IUCCGAUC | 2044 |
| 1473 | AUCGGACC A UCGGCUCU | 623 | AGAGCCGA CUGAUGAG GCCGUUAGGC CGAA IGUCCGAU | 2045 |
| 1479 | CCAUCGGC U CUGGGGAA | 624 | UUCCCCAG CUGAUGAG GCCGUUAGGC CGAA ICCGAUGG | 2046 |
| 1481 | AUCGGCUC U GGGGAAUC | 625 | GAUUCCCC CUGAUGAG GCCGUUAGGC CGAA IAGCCGAU | 2047 |
| 1490 | GGGGAAUC C UGGUGAAU | 626 | AUUCACCA CUGAUGAG GCCGUUAGGC CGAA IAUUCCCC | 2048 |
| 1491 | GGGAAUCC U GGUGAAUA | 627 | UAUUCACC CUGAUGAG GCCGUUAGGC CGAA IGAUUCCC | 2049 |
| 1506 | UAUAGUGC U GCUAUGUU | 628 | AACAUAGC CUGAUGAG GCCGUUAGGC CGAA ICACUAUA | 2050 |
| 1509 | AGUGCUGC U AUGUUGAC | 629 | GUCAACAU CUGAUGAG GCCGUUAGGC CGAA ICAGCACU | 2051 |
| 1518 | AUGUUGAC A UUAUUCUU | 630 | AAGAAUAA CUGAUGAG GCCGUUAGGC CGAA IUCAACAU | 2052 |
| 1525 | CAUUAUUC U UCCUAGAG | 631 | CUCUAGGA CUGAUGAG GCCGUUAGGC CGAA IAAUAAUG | 2053 |
| 1528 | UAUUCUUC C UAGAGAAG | 632 | CUUCUCUA CUGAUGAG GCCGUUAGGC CGAA IAAGAAUA | 2054 |
| 1529 | AUUCUUCC U AGAGAAGA | 633 | UCUUCUCU CUGAUGAG GCCGUUAGGC CGAA IGAAGAAU | 2055 |
| 1543 | AGAUUAUC C UGUCCUGC | 634 | GCAGGACA CUGAUGAG GCCGUUAGGC CGAA IAUAAUCU | 2056 |
| 1544 | GAUUAUCC U GUCCUGCA | 635 | UGCAGGAC CUGAUGAG GCCGUUAGGC CGAA IGAUAAUC | 2057 |
| 1548 | AUCCUGUC C UGCAAACU | 636 | AGUUUGCA CUGAUGAG GCCGUUAGGC CGAA IACAGGAU | 2058 |
| 1549 | UCCUGUCC U GCAAACUG | 637 | CAGUUUGC CUGAUGAG GCCGUUAGGC CGAA IGACAGGA | 2059 |
| 1552 | UGUCCUGC A AACUGCAA | 638 | UUGCAGUU CUGAUGAG GCCGUUAGGC CGAA ICAGGACA | 2060 |
| 1556 | CUGCAAAC U GCAAAUAG | 639 | CUAUUUGC CUGAUGAG GCCGUUAGGC CGAA IUUUGCAG | 2061 |
| 1559 | CAAACUGC A AAUAGUAG | 640 | CUACUAUU CUGAUGAG GCCGUUAGGC CGAA ICAGUUUG | 2062 |
| 1571 | AGUAGUUC C UGAAGUGU | 641 | ACACUUCA CUGAUGAG GCCGUUAGGC CGAA IAACUACU | 2063 |
| 1572 | GUAGUUCC U GAAGUGUU | 642 | AACACUUC CUGAUGAG GCCGUUAGGC CGAA IGAACUAC | 2064 |
| 1582 | AAGUGUUC A CUUCCCUG | 643 | CAGGGAAG CUGAUGAG GCCGUUAGGC CGAA IAACACUU | 2065 |
| 1584 | GUGUUCAC U UCCCUGUU | 644 | AACAGGGA CUGAUGAG GCCGUUAGGC CGAA IUGAACAC | 2066 |
| 1587 | UUCACUUC C CUGUUUAU | 645 | AUAAACAG CUGAUGAG GCCGUUAGGC CGAA IAAGUGAA | 2067 |
| 1588 | UCACUUCC C UGUUUAUC | 646 | GAUAAACA CUGAUGAG GCCGUUAGGC CGAA IGAAGUGA | 2068 |
| 1589 | CACUUCCC U GUUUAUCC | 647 | GGAUAAAC CUGAUGAG GCCGUUAGGC CGAA IGGAAGUG | 2069 |
| 1597 | UGUUUAUC C AAACAUCU | 648 | AGAUGUUU CUGAUGAG GCCGUUAGGC CGAA IAUAAACA | 2070 |
| 1598 | GUUUAUCC A AACAUCUU | 649 | AAGAUGUU CUGAUGAG GCCGUUAGGC CGAA IGAUAAAC | 2071 |
| 1602 | AUCCAAAC A UCUUCCAA | 650 | UUGGAAGA CUGAUGAG GCCGUUAGGC CGAA IUUUGGAU | 2072 |
| 1605 | CAAACAUC U UCCAAUUU | 651 | AAAUUGGA CUGAUGAG GCCGUUAGGC CGAA IAUGUUUG | 2073 |
| 1608 | ACAUCUUC C AAUUUAUU | 652 | AAUAAAUU CUGAUGAG GCCGUUAGGC CGAA IAAGAUGU | 2074 |
| 1609 | CAUCUUCC A AUUUAUUU | 653 | AAAUAAAU CUGAUGAG GCCGUUAGGC CGAA IGAAGAUG | 2075 |
| 1630 | UGUUCGGC A UACAAAUA | 654 | UAUUUGUA CUGAUGAG GCCGUUAGGC CGAA ICCGAACA | 2076 |
| 1634 | CGGCAUAC A AAUAAUAC | 655 | GUAUUAUU CUGAUGAG GCCGUUAGGC CGAA IUAUGCCG | 2077 |
| 1643 | AAUAAUAC C UAUAUCUU | 656 | AAGAUAUA CUGAUGAG GCCGUUAGGC CGAA IUAUUAUU | 2078 |
| 1644 | AUAAUACC U AUAUCUUA | 657 | UAAGAUAU CUGAUGAG GCCGUUAGGC CGAA IGUAUUAU | 2079 |
| 1650 | CCUAUAUC U UAAUUGUA | 658 | UACAAUUA CUGAUGAG GCCGUUAGGC CGAA IAUAUAGG | 2080 |
| 1662 | UUGUAAGC A AAACUUUG | 659 | CAAAGUUU CUGAUGAG GCCGUUAGGC CGAA ICUUACAA | 2081 |
| 1667 | AGCAAAAC U UUGGGGAA | 660 | UUCCCCAA CUGAUGAG GCCGUUAGGC CGAA IUUUUGCU | 2082 |
| 1692 | UAGAAUUC A UUUGAUUA | 661 | UAAUCAAA CUGAUGAG GCCGUUAGGC CGAA IAAUUCUA | 2083 |
| 1705 | AUUAUUUC U UCAUGUGU | 662 | ACACAUGA CUGAUGAG GCCGUUAGGC CGAA IAAAUAAU | 2084 |
| 1708 | AUUUCUUC A UGUGUGUU | 663 | AACACACA CUGAUGAG GCCGUUAGGC CGAA IAAGAAAU | 2085 |
| 1724 | UUAGUAUC U GAAUUUGA | 664 | UCAAAUUC CUGAUGAG GCCGUUAGGC CGAA IAUACUAA | 2086 |
| 1736 | UUUGAAAC U CAUCUGGU | 665 | ACCAGAUG CUGAUGAG GCCGUUAGGC CGAA IUUUCAAA | 2087 |
| 1738 | UGAAACUC A UCUGGUGG | 666 | CCACCAGA CUGAUGAG GCCGUUAGGC CGAA IAGUUUCA | 2088 |
| 1741 | AACUCAUC U GGUGGAAA | 667 | UUUCCACC CUGAUGAG GCCGUUAGGC CGAA IAUGAGUU | 2089 |
| 1751 | GUGGAAAC C AAGUUUCA | 668 | UGAAACUU CUGAUGAG GCCGUUAGGC CGAA IUUUCCAC | 2090 |
| 1752 | UGGAAACC A AGUUUCAG | 669 | CUGAAACU CUGAUGAG GCCGUUAGGC CGAA IGUUUCCA | 2091 |
| 1759 | CAAGUUUC A GGGGACAU | 670 | AUGUCCCC CUGAUGAG GCCGUUAGGC CGAA IAAACUUG | 2092 |
| 1766 | CAGGGGAC A UGAGUUUU | 671 | AAAACUCA CUGAUGAG GCCGUUAGGC CGAA IUCCCCUG | 2093 |
| 1776 | GAGUUUUC C AGCUUUUA | 672 | UAAAAGCU CUGAUGAG GCCGUUAGGC CGAA IAAAACUC | 2094 |
| 1777 | AGUUUUCC A GCUUUUAU | 673 | AUAAAAGC CUGAUGAG GCCGUUAGGC CGAA IGAAAACU | 2095 |
| 1780 | UUUCCAGC U UUUAUACA | 674 | UGUAUAAA CUGAUGAG GCCGUUAGGC CGAA ICUGGAAA | 2096 |
| 1788 | UUUUAUAC A CACGUAUC | 675 | GAUACGUG CUGAUGAG GCCGUUAGGC CGAA IUAUAAAA | 2097 |
| 1790 | UUAUACAC A CGUAUCUC | 676 | GAGAUACG CUGAUGAG GCCGUUAGGC CGAA IUGUAUAA | 2098 |
| 1797 | CACGUAUC U CAUUUUUA | 677 | UAAAAAUG CUGAUGAG GCCGUUAGGC CGAA IAUACGUG | 2099 |
| 1799 | CGUAUCUC A UUUUUAUC | 678 | GAUAAAAA CUGAUGAG GCCGUUAGGC CGAA IAGAUACG | 2100 |
| 1808 | UUUUUAUC A AAACAUUU | 679 | AAAUGUUU CUGAUGAG GCCGUUAGGC CGAA IAUAAAAA | 2101 |
| 1813 | AUCAAAAC A UUUUGUUU | 680 | AAACAAAA CUGAUGAG GCCGUUAGGC CGAA IUUUUGAU | 2102 |

Input Sequence = AF016582 Cut Site = CH/.
Stem Length = 8. Core Sequence = CUGAUGAG GCCGUUAGGC CGAA
AF016582 (Homo sapiens checkpoint kinase Chk1 (CHK1) mRNA; 1821 bp)

Underlined region can be any X sequence or linker as previously defined herein.

I=Inosine

5

TABLE V
Human Chk1 G-Cleaver Ribozyme and Substrate Sequence

| Pos | Substrate | Seq ID | Ribozyme | Rz Seq ID |
|---|---|---|---|---|
| 14 | GACAGUCC G CCGAGGUG | 681 | CACCUCGG UGAUGGCAUGCACUAUGCGCG GGACUGUC | 2103 |
| 17 | AGUCCGCC G AGGUGCUC | 682 | GAGCACCU UGAUGGCAUGCACUAUGCGCG GGCGGACU | 2104 |
| 22 | GCCGAGGU G CUCGGUGG | 683 | CCACCGAG UGAUGGCAUGCACUAUGCGCG ACCUCGGC | 2105 |
| 43 | AUGGCAGU G CCCUUUGU | 684 | ACAAAGGG UGAUGGCAUGCACUAUGCGCG ACUGCCAU | 2106 |
| 50 | UGCCCUUU G UGGAAGAC | 685 | GUCUUCCA UGAUGGCAUGCACUAUGCGCG AAAGGGCA | 2107 |
| 70 | GACUUGGU G CAAACCCU | 686 | AGGGUUUG UGAUGGCAUGCACUAUGCGCG ACCAAGUC | 2108 |
| 89 | GAGAAGGU G CCUAUGGA | 687 | UCCAUAGG UGAUGGCAUGCACUAUGCGCG ACCUUCUC | 2109 |
| 110 | UUCAACUU G CUGUGAAU | 688 | AUUCACAG UGAUGGCAUGCACUAUGCGCG AAGUUGAA | 2110 |
| 113 | AACUUGCU G UGAAUAGA | 689 | UCUAUUCA UGAUGGCAUGCACUAUGCGCG AGCAAGUU | 2111 |
| 115 | CUUGCUGU G AAUAGAGU | 690 | ACUCUAUU UGAUGGCAUGCACUAUGCGCG ACAGCAAG | 2112 |
| 128 | GAGUAACU G AAGAAGCA | 691 | UGCUUCUU UGAUGGCAUGCACUAUGCGCG AGUUACUC | 2113 |
| 140 | AAGCAGUC G CAGUGAAG | 692 | CUUCACUG UGAUGGCAUGCACUAUGCGCG GACUGCUU | 2114 |
| 145 | GUCGCAGU G AAGAUUGU | 693 | ACAAUCUU UGAUGGCAUGCACUAUGCGCG ACUGCGAC | 2115 |
| 152 | UGAAGAUU G UAGAUAUG | 694 | CAUAUCUA UGAUGGCAUGCACUAUGCGCG AAUCUUCA | 2116 |
| 160 | GUAGAUAU G AAGCGUGC | 695 | GCACGCUU UGAUGGCAUGCACUAUGCGCG AUAUCUAC | 2117 |
| 167 | UGAAGCGU G CCGUAGAC | 696 | GUCUACGG UGAUGGCAUGCACUAUGCGCG ACGCUUCA | 2118 |
| 177 | CGUAGACU G UCCAGAAA | 697 | UUUCUGGA UGAUGGCAUGCACUAUGCGCG AGUCUACG | 2119 |
| 204 | AGAGAUCU G UAUCAAUA | 698 | UAUUGAUA UGAUGGCAUGCACUAUGCGCG AGAUCUCU | 2120 |
| 217 | AAUAAAAU G CUAAAUCA | 699 | UGAUUUAG UGAUGGCAUGCACUAUGCGCG AUUUUAUU | 2121 |
| 227 | UAAAUCAU G AAAAUGUA | 700 | UACAUUUU UGAUGGCAUGCACUAUGCGCG AUGAUUUA | 2122 |
| 233 | AUGAAAAU G AUGUAAAA | 701 | UUUUACUA UGAUGGCAUGCACUAUGCGCG AUUUUCAU | 2123 |
| 294 | GGAGUACU G UAGUGGAG | 702 | CUCCACUA UGAUGGCAUGCACUAUGCGCG AGUACUCC | 2124 |
| 314 | AGCUUUUU G ACAGAAUA | 703 | UAUUCUGU UGAUGGCAUGCACUAUGCGCG AAAAAGCU | 2125 |
| 340 | AUAGGCAU G CCUGAACC | 704 | GGUUCAGG UGAUGGCAUGCACUAUGCGCG AUGCCUAU | 2126 |
| 344 | GCAUGCCU G AACCAGAU | 705 | AUCUGGUU UGAUGGCAUGCACUAUGCGCG AGGCAUGC | 2127 |
| 353 | AACCAGAU G CUCAGAGA | 706 | UCUCUGAG UGAUGGCAUGCACUAUGCGCG AUCUGGUU | 2128 |
| 397 | GUUUAUCU G CAUGGUAU | 707 | AUACCAUG UGAUGGCAUGCACUAUGCGCG AGAUAAAC | 2129 |
| 445 | AAUCUUCU G UUGGAUGA | 708 | UCAUCCAA UGAUGGCAUGCACUAUGCGCG AGAAGAUU | 2130 |
| 452 | UGUUGGAU G AAAGGGAU | 709 | AUCCCUUU UGAUGGCAUGCACUAUGCGCG AUCCAACA | 2131 |
| 515 | AUAAUCGU G AGCGUUUG | 710 | CAAACGCU UGAUGGCAUGCACUAUGCGCG ACGAUUAU | 2132 |
| 523 | GAGCGUUU G UUGAACAA | 711 | UUGUUCAA UGAUGGCAUGCACUAUGCGCG AAACGCUC | 2133 |
| 526 | CGUUUGUU G AACAAGAU | 712 | AUCUUGUU UGAUGGCAUGCACUAUGCGCG AACAAACG | 2134 |
| 535 | AACAAGAU G UGUGGUAC | 713 | GUACCACA UGAUGGCAUGCACUAUGCGCG AUCUUGUU | 2135 |
| 537 | CAAGAUGU G UGGUACUU | 714 | AAGUACCA UGAUGGCAUGCACUAUGCGCG ACAUCUUG | 2136 |
| 554 | UACCAUAU G UUGCUCCA | 715 | UGGAGCAA UGAUGGCAUGCACUAUGCGCG AUAUGGUA | 2137 |
| 557 | CAUAUGUU G CUCCAGAA | 716 | UUCUGGAG UGAUGGCAUGCACUAUGCGCG AACAUAUG | 2138 |
| 571 | GAACUUCU G AAGAGAAG | 717 | CUUCUCUU UGAUGGCAUGCACUAUGCGCG AGAAGUUC | 2139 |
| 590 | AAUUUCAU G CAGAACCA | 718 | UGGUUCUG UGAUGGCAUGCACUAUGCGCG AUGAAAUU | 2140 |
| 602 | AACCAGUU G AUGUUUGG | 719 | CCAAACAU UGAUGGCAUGCACUAUGCGCG AACUGGUU | 2141 |
| 605 | CAGUUGAU G UUUGGUCC | 720 | GGACCAAA UGAUGGCAUGCACUAUGCGCG AUCAACUG | 2142 |
| 615 | UUGGUCCU G UGGAAUAG | 721 | CUAUUCCA UGAUGGCAUGCACUAUGCGCG AGGACCAA | 2143 |
| 632 | UACUUACU G CAAUGCUC | 722 | GAGCAUUG UGAUGGCAUGCACUAUGCGCG AGUAAGUA | 2144 |
| 637 | ACUGCAAU G CUCGCUGG | 723 | CCAGCGAG UGAUGGCAUGCACUAUGCGCG AUUGCAGU | 2145 |
| 641 | CAAUGCUC G CUGGAGAA | 724 | UUCUCCAG UGAUGGCAUGCACUAUGCGCG GAGCAUUG | 2146 |
| 652 | GGAGAAUU G CCAUGGGA | 725 | UCCCAUGG UGAUGGCAUGCACUAUGCGCG AAUUCUCC | 2147 |
| 671 | AACCCAGU G ACAGCUGU | 726 | ACAGCUGU UGAUGGCAUGCACUAUGCGCG ACUGGGUU | 2148 |
| 678 | UGACAGCU G UCAGGAGU | 727 | ACUCCUGA UGAUGGCAUGCACUAUGCGCG AGCUGUCA | 2149 |
| 692 | AGUAUUCU G ACUGGAAA | 728 | UUUCCAGU UGAUGGCAUGCACUAUGCGCG AGAAUACU | 2150 |
| 737 | AAAAAAUC G AUUCUGCU | 729 | AGCAGAAU UGAUGGCAUGCACUAUGCGCG GAUUUUUU | 2151 |
| 732 | UCGAUUCU G CUCCUCUA | 730 | UAGAGGAG UGAUGGCAUGCACUAUGCGCG AGAAUCGA | 2152 |
| 757 | CUAGCUCU G CUGCAUAA | 731 | UUAUGCAG UGAUGGCAUGCACUAUGCGCG AGAGCUAG | 2153 |
| 760 | GCUCUGCU G CAUAAAAU | 732 | AUUUUAUG UGAUGGCAUGCACUAUGCGCG AGCAGAGC | 2154 |
| 776 | UCUUAGUU G AGAAUCCA | 733 | UGGAUUCU UGAUGGCAUGCACUAUGCGCG AACUAAGA | 2155 |
| 864 | AAGGCCCC G AGUCACUU | 734 | AAGUGACU UGAUGGCAUGCACUAUGCGCG GGGGCCUU | 2156 |
| 881 | CAGGUGGU G UGUCAGAG | 735 | CUCUGACA UGAUGGCAUGCACUAUGCGCG ACCACCUG | 2157 |
| 883 | GGUGGUGU G UCAGAGUC | 736 | GACUCUGA UGAUGGCAUGCACUAUGCGCG ACACCACC | 2158 |
| 950 | UAAACAGU G CUUCUAGU | 737 | ACUAGAAG UGAUGGCAUGCACUAUGCGCG ACUGUUUA | 2159 |
| 959 | CUUCUAGU G AAGAAAAU | 738 | AUUUUCUU UGAUGGCAUGCACUAUGCGCG ACUAGAAG | 2160 |
| 968 | AAGAAAAU G UGAAGUAC | 739 | GUACUUCA UGAUGGCAUGCACUAUGCGCG AUUUUCUU | 2161 |
| 970 | GAAAAUGU G AAGUACUC | 740 | GAGUACUU UGAUGGCAUGCACUAUGCGCG ACAUUUUC | 2162 |
| 999 | AGAACCCC G CACAGGUC | 741 | GACCUGUG UGAUGGCAUGCACUAUGCGCG GGGGUUCU | 2163 |
| 1040 | CAUACAUU G AUAAAUUG | 742 | CAAUUUAU UGAUGGCAUGCACUAUGCGCG AAUGUAUG | 2164 |
| 1080 | GCCCACAU G UCCUGAUC | 743 | GAUCAGGA UGAUGGCAUGCACUAUGCGCG AUGUGGGC | 2165 |
| 1085 | CAUGUCCU G AUCAUAUG | 744 | CAUAUGAU UGAUGGCAUGCACUAUGCGCG AGGACAUG | 2166 |
| 1093 | GAUCAUAU G CUUUUGAA | 745 | UUCAAAAG UGAUGGCAUGCACUAUGCGCG AUAUGAUC | 2167 |
| 1099 | AUGCUUUU G AAUAGUCA | 746 | UGACUAUU UGAUGGCAUGCACUAUGCGCG AAAAGCAU | 2168 |
| 1165 | AAAAGAAU G ACACGAUU | 747 | AAUCGUGU UGAUGGCAUGCACUAUGCGCG AUUCUUUU | 2169 |
| 1170 | AAUGACAC G AUUCUUUA | 748 | UAAAGAAU UGAUGGCAUGCACUAUGCGCG GUGUCAUU | 2170 |
| 1190 | AAUUGGAU G CAGACAAA | 749 | UUUGUCUG UGAUGGCAUGCACUAUGCGCG AUCCAAUU | 2171 |
| 1209 | UUAUCAAU G CCUGAAAG | 750 | CUUUCAGG UGAUGGCAUGCACUAUGCGCG AUUGAUAA | 2172 |
| 1213 | CAAUGCCU G AAAGAGAC | 751 | GUCUCUUU UGAUGGCAUGCACUAUGCGCG AGGCAUUG | 2173 |
| 1224 | AGAGACUU G UGAGAAGU | 752 | ACUUCUCA UGAUGGCAUGCACUAUGCGCG AAGUCUCU | 2174 |
| 1226 | AGACUUGU G AGAAGUUG | 753 | CAACUUCU UGAUGGCAUGCACUAUGCGCG ACAAGUCU | 2175 |
| 1257 | GAAAAGUU G UAUGAAUC | 754 | GAUUCAUA UGAUGGCAUGCACUAUGCGCG AACUUUUC | 2176 |
| 1261 | AGUUGUAU G AAUCAGGU | 755 | ACCUGAUU UGAUGGCAUGCACUAUGCGCG AUACAACU | 2177 |
| 1286 | CAACAACU G AUAGGAGA | 756 | UCUCCUAU UGAUGGCAUGCACUAUGCGCG AGUUGUUG | 2178 |
| 1318 | UUCAAAGU G AAUUUGUU | 757 | AACAAAUU UGAUGGCAUGCACUAUGCGCG ACUUUGAA | 2179 |
| 1324 | GUGAAUUU G UUAGAAAU | 758 | AUUUCUAA UGAUGGCAUGCACUAUGCGCG AAAUUCAC | 2180 |
| 1337 | AAAUGGAU G AUAAAAUA | 759 | UAUUUUAU UGAUGGCAUGCACUAUGCGCG AUCCAUUU | 2181 |
| 1352 | UAUUGGUU G ACUUCCGG | 760 | CCGGAAGU UGAUGGCAUGCACUAUGCGCG AACCAAUA | 2182 |
| 1373 | CUAAGGGU G AUGGAUUG | 761 | CAAUCCAU UGAUGGCAUGCACUAUGCGCG ACCCUUAG | 2183 |
| 1402 | CACUUCCU G AAGAUUAA | 762 | UUAAUCUU UGAUGGCAUGCACUAUGCGCG AGGAAGUG | 2184 |
| 1420 | GGGAAGCU G AUUGAUAU | 763 | AUAUCAAU UGAUGGCAUGCACUAUGCGCG AGCUUCCC | 2185 |
| 1424 | AGCUGAUU G AUAUUGUG | 764 | CACAAUAU UGAUGGCAUGCACUAUGCGCG AAUCAGCU | 2186 |
| 1430 | UUGAUAUU G UGAGCAGC | 765 | GCUGCUCA UGAUGGCAUGCACUAUGCGCG AAUAUCAA | 2187 |
| 1432 | GAUAUUGU G AGCAGCCA | 766 | UGGCUGCU UGAUGGCAUGCACUAUGCGCG ACAAUAUC | 2188 |
| 1457 | GGCUUCCU G CCACAUGA | 767 | UCAUGUGG UGAUGGCAUGCACUAUGCGCG AGGAAGCC | 2189 |
| 1464 | UGCCACAU G AUCGGACC | 768 | GGUCCGAU UGAUGGCAUGCACUAUGCGCG AUGUGGCA | 2190 |
| 1495 | AUCCUGGU G AAUAUAGU | 769 | ACUAUAUU UGAUGGCAUGCACUAUGCGCG ACCAGGAU | 2191 |
| 1504 | AAUAUAGU G CUGCUAUG | 770 | CAUAGCAG UGAUGGCAUGCACUAUGCGCG ACUAUAUU | 2192 |
| 1507 | AUAGUGCU G CUAUGUUG | 771 | CAACAUAG UGAUGGCAUGCACUAUGCGCG AGCACUAU | 2193 |
| 1512 | GCUGCUAU G UUGACAUU | 772 | AAUGUCAA UGAUGGCAUGCACUAUGCGCG AUAGCAGC | 2194 |
| 1515 | GCUAUGUU G ACAUUAUU | 773 | AAUAAUGU UGAUGGCAUGCACUAUGCGCG AACAUAGC | 2195 |
| 1545 | AUUAUCCU G UCCUGCAA | 774 | UUGCAGGA UGAUGGCAUGCACUAUGCGCG AGGAUAAU | 2196 |
| 1550 | CCUGUCCU G CAAACUGC | 775 | GCAGUUUG UGAUGGCAUGCACUAUGCGCG AGGACAGG | 2197 |
| 1557 | UGCAAACU G CAAAUAGU | 776 | ACUAUUUG UGAUGGCAUGCACUAUGCGCG AGUUUGCA | 2198 |
| 1573 | UAGUUCCU G AAGUGUUC | 777 | GAACACUU UGAUGGCAUGCACUAUGCGCG AGGAACUA | 2199 |
| 1578 | CCUGAAGU G UUCACUUC | 778 | GAAGUGAA UGAUGGCAUGCACUAUGCGCG ACUUCAGG | 2200 |
| 1590 | ACUUCCCU G UUUAUCCA | 779 | UGGAUAAA UGAUGGCAUGCACUAUGCGCG AGGGAAGU | 2201 |
| 1619 | UUUAUUUU G UUUGUUCG | 780 | CGAACAAA UGAUGGCAUGCACUAUGCGCG AAAAUAAA | 2202 |
| 1623 | UUUUGUUU G UUCGGCAU | 781 | AUGCCGAA UGAUGGCAUGCACUAUGCGCG AAACAAAA | 2203 |
| 1656 | UCUUAAUU G UAAGCAAA | 782 | UUUGCUUA UGAUGGCAUGCACUAUGCGCG AAUUAAGA | 2204 |
| 1681 | GAAAGGAU G AAUAGAAU | 783 | AUUCUAUU UGAUGGCAUGCACUAUGCGCG AUCCUUUC | 2205 |
| 1696 | AUUCAUUU G AUUAUUUC | 784 | GAAAUAAU UGAUGGCAUGCACUAUGCGCG AAAUGAAU | 2206 |
| 1710 | UUCUUCAU G UGUGUUUA | 785 | UAAACACA UGAUGGCAUGCACUAUGCGCG AUGAAGAA | 2207 |
| 1712 | CUUCAUGU G UGUUUAGU | 786 | ACUAAACA UGAUGGCAUGCACUAUGCGCG ACAUGAAG | 2208 |
| 1714 | UCAUGUGU G UUUAGUAU | 787 | AUACUAAA UGAUGGCAUGCACUAUGCGCG ACACAUGA | 2209 |
| 1725 | UAGUAUCU G AAUUUGAA | 788 | UUCAAAUU UGAUGGCAUGCACUAUGCGCG AGAUACUA | 2210 |
| 1731 | CUGAAUUU G AAACUCAU | 789 | AUGAGUUU UGAUGGCAUGCACUAUGCGCG AAAUUCAG | 2211 |
| 1768 | GGGGACAU G AGUUUUCC | 790 | GGAAAACU UGAUGGCAUGCACUAUGCGCG AUGUCCCC | 2212 |

Input Sequence = AF016582. Cut Site = YG/M or UG/U.
Stem Length = 8. Core Sequence = UGAUG GCAUGCACUAUGC GCG
AF016582 (Homo sapiens checkpoint kinase Chk1 (CHK1) mRNA; 1821 bp)

TABLE VI
Human Chk1 Zinzyme Ribozyme and Substrate Sequence

| | | | | Rz Seq |
|---|---|---|---|---|
| Pos | Substrate | Seq ID | Ribozyme | ID |
| 10 | GCCGGACA G UCCGCCGA | 791 | UCGGCGGA GCCGAAAGGCGAGUCAAGGUCU UGUCCGGC | 2213 |
| 14 | GACAGUCC G CCGAGGUG | 792 | CACCUCGG GCCGAAAGGCGAGUCAAGGUCU GGACUGUC | 2214 |
| 20 | CCGCCGAG G UGCUCGGU | 793 | ACCGAGCA GCCGAAAGGCGAGUCAAGGUCU CUCGGCGG | 2215 |
| 22 | GCCGAGGU G CUCGGUGG | 794 | CCACCGAG GCCGAAAGGCGAGUCAAGGUCU ACCUCGGC | 2216 |
| 27 | GGUGCUCG G UGGAGUCA | 795 | UGACUCCA GCCGAAAGGCGAGUCAAGGUCU CGAGCACC | 2217 |
| 32 | UCGGUGGA G UCAUGGCA | 796 | UGCCAUGA GCCGAAAGGCGAGUCAAGGUCU UCCACCGA | 2218 |
| 38 | GAGUCAUG G CAGUGCCC | 797 | GGGCACUG GCCGAAAGGCGAGUCAAGGUCU CAUGACUC | 2219 |
| 41 | UCAUGGCA G UGCCCUUU | 798 | AAAGGGCA GCCGAAAGGCGAGUCAAGGUCU UGCCAUGA | 2220 |
| 43 | AUGGCAGU G CCCUUUGU | 799 | ACAAAGGG GCCGAAAGGCGAGUCAAGGUCU ACUGCCAU | 2221 |
| 50 | UGCCCUUU G UGGAAGAC | 800 | GUCUUCCA GCCGAAAGGCGAGUCAAGGUCU AAAGGGCA | 2222 |
| 68 | GGGACUUG G UGCAAACC | 801 | GGUUUGCA GCCGAAAGGCGAGUCAAGGUCU CAAGUCCC | 2223 |
| 70 | GACUUGGU G CAAACCCU | 802 | AGGGUUUG GCCGAAAGGCGAGUCAAGGUCU ACCAAGUC | 2224 |
| 87 | GGGAGAAG G UGCCUAUG | 803 | CAUAGGCA GCCGAAAGGCGAGUCAAGGUCU CUUCUCCC | 2225 |
| 89 | GAGAAGGU G CCUAUGGA | 804 | UCCAUAGG GCCGAAAGGCGAGUCAAGGUCU ACCUUCUC | 2226 |
| 101 | AUGGAGAA G UUCAACUU | 805 | AAGUUGAA GCCGAAAGGCGAGUCAAGGUCU UUCUCCAU | 2227 |
| 110 | UUCAACUU G CUGUGAAU | 806 | AUUCACAG GCCGAAAGGCGAGUCAAGGUCU AAGUUGAA | 2228 |
| 113 | AACUUGCU G UGAAUAGA | 807 | UCUAAUCA GCCGAAAGGCGAGUCAAGGUCU AGCAAGUU | 2229 |
| 122 | UGAAUAGA G UAACUGAA | 808 | UUCAGUUA GCCGAAAGGCGAGUCAAGGUCU UCUAUUCA | 2230 |
| 134 | CUGAAGAA G CAGUCGCA | 809 | UGCGACUG GCCGAAAGGCGAGUCAAGGUCU UUCUUCAG | 2231 |
| 137 | AAGAAGCA G UCGCAGUG | 810 | CACUGCGA GCCGAAAGGCGAGUCAAGGUCU UGCUUCUU | 2232 |
| 140 | AAGCAGUC G CAGUGAAG | 811 | CUUCACUG GCCGAAAGGCGAGUCAAGGUCU GACUGCUU | 2233 |
| 143 | CAGUCGCA G UGAAGAUU | 812 | AAUCUUCA GCCGAAAGGCGAGUCAAGGUCU UGCGACUG | 2234 |
| 152 | UGAAGAUU G UAGAUAUG | 813 | CAUAUCUA GCCGAAAGGCGAGUCAAGGUCU AAUCUUCA | 2235 |
| 163 | GAUAUGAA G CGUGCCGU | 814 | ACGGCACG GCCGAAAGGCGAGUCAAGGUCU UUCAUAUC | 2236 |
| 165 | UAUGAAGC G UGCCGUAG | 815 | CUACGGCA GCCGAAAGGCGAGUCAAGGUCU GCUUCAUA | 2237 |
| 167 | UGAAGCGU G CCGUAGAC | 816 | GUCUACGG GCCGAAAGGCGAGUCAAGGUCU ACGCUUCA | 2238 |
| 170 | AGCGUGCC G UAGACUGU | 817 | ACAGUCUA GCCGAAAGGCGAGUCAAGGUCU GGCACGCU | 2239 |
| 177 | CGUAGACU G UCCAGAAA | 818 | UUUCUGGA GCCGAAAGGCGAGUCAAGGUCU AGUCUACG | 2240 |
| 204 | AGAGAUCU G UAUCAAUA | 819 | UAUUGAUA GCCGAAAGGCGAGUCAAGGUCU AGAUCUCU | 2241 |
| 217 | AAUAAAAU G CUAAAUCA | 820 | UGAUUUAG GCCGAAAGGCGAGUCAAGGUCU AUUUUAUU | 2242 |
| 233 | AUGAAAAU G UAGUAAAA | 821 | UUUUACUA GCCGAAAGGCGAGUCAAGGUCU AUUUUCAU | 2243 |
| 236 | AAAAUGUA G UAAAAUUC | 822 | GAAUUUUA GCCGAAAGGCGAGUCAAGGUCU UACAUUUU | 2244 |
| 249 | AUUCUAUG G UCACAGGA | 823 | UCCUGUGA GCCGAAAGGCGAGUCAAGGUCU CAUAGAAU | 2245 |
| 264 | GAGAGAAG G CAAUAUCC | 824 | GGAUAUUG GCCGAAAGGCGAGUCAAGGUCU CUUCUCUC | 2246 |
| 289 | UUUCUGGA G UACUGUAG | 825 | CUACAGUA GCCGAAAGGCGAGUCAAGGUCU UCCAGAAA | 2247 |
| 294 | GGAGUACU G UAGUGGAG | 826 | CUCCACUA GCCGAAAGGCGAGUCAAGGUCU AGUACUCC | 2248 |
| 297 | GUACUGUA G UGGAGGAG | 827 | CUCCUCCA GCCGAAAGGCGAGUCAAGGUCU UACAGUAC | 2249 |
| 307 | GGAGGAGA G CUUUUUGA | 828 | UCAAAAAG GCCGAAAGGCGAGUCAAGGUCU UCUCCUCC | 2250 |
| 325 | AGAAUAGA G CCAGACAU | 829 | AUGUCUGG GCCGAAAGGCGAGUCAAGGUCU UCUAUUCU | 2251 |
| 336 | AGACAUAG G CAUGCCUG | 830 | CAGGCAUG GCCGAAAGGCGAGUCAAGGUCU CUAUGUCU | 2252 |
| 340 | AUAGGCAU G CCUGAACC | 831 | GGUUCAGG GCCGAAAGGCGAGUCAAGGUCU AUGCCUAU | 2253 |
| 353 | AACCAGAU G CUCAGAGA | 832 | UCUCUGAG GCCGAAAGGCGAGUCAAGGUCU AUCUGGUU | 2254 |
| 380 | AACUCAUG G CAGGGGUG | 833 | CACCCCUG GCCGAAAGGCGAGUCAAGGUCU CAUGAGUU | 2255 |
| 386 | UGGCAGGG G UGGUUUAU | 834 | AUAAACCA GCCGAAAGGCGAGUCAAGGUCU CCCUGCCA | 2256 |
| 389 | CAGGGGUG G UUUAUCUG | 835 | CAGAUAAA GCCGAAAGGCGAGUCAAGGUCU CACCCCUG | 2257 |
| 397 | GUUUAUCU G CAUGGUAU | 836 | AUACCAUG GCCGAAAGGCGAGUCAAGGUCU AGAUAAAC | 2258 |
| 402 | UCUGCAUG G UAUUGGAA | 837 | UUCCAAUA GCCGAAAGGCGAGUCAAGGUCU CAUGCAGA | 2259 |
| 445 | AAUCUUCU G UUGGAUGA | 838 | UCAUCCAA GCCGAAAGGCGAGUCAAGGUCU AGAAGAUU | 2260 |
| 483 | AGACUUUG G CUUGGCAA | 839 | UUGCCAAG GCCGAAAGGCGAGUCAAGGUCU CAAAGUCU | 2261 |
| 488 | UUGGCUUG G CAACAGUA | 840 | UACUGUUG GCCGAAAGGCGAGUCAAGGUCU CAAGCCAA | 2262 |
| 494 | UGGCAACA G UAUUUCGG | 841 | CCGAAAUA GCCGAAAGGCGAGUCAAGGUCU UGUUGCCA | 2263 |
| 502 | GUAUUUCG G UAUAAUAA | 842 | UUAUUAUA GCCGAAAGGCGAGUCAAGGUCU CGAAAUAC | 2264 |
| 513 | UAAUAAUC G UGAGCGUU | 843 | AACGCUCA GCCGAAAGGCGAGUCAAGGUCU GAUUAUUA | 2265 |
| 517 | AAUCGUGA G CGUUUGUU | 844 | AACAAACG GCCGAAAGGCGAGUCAAGGUCU UCACGAUU | 2266 |
| 519 | UCGUGAGC G UUUGUUGA | 845 | UCAACAAA GCCGAAAGGCGAGUCAAGGUCU GCUCACGA | 2267 |
| 523 | GAGCGUUU G UUGAACAA | 846 | UUGUUCAA GCCGAAAGGCGAGUCAAGGUCU AAACGCUC | 2268 |
| 535 | AACAAGAU G UGUGGUAC | 847 | GUACCACA GCCGAAAGGCGAGUCAAGGUCU AUCUUGUU | 2269 |
| 537 | CAAGAUGU G UGGUACUU | 848 | AAGUACCA GCCGAAAGGCGAGUCAAGGUCU ACAUCUUG | 2270 |
| 540 | GAUGUGUG G UACUUUAC | 849 | GUAAAGUA GCCGAAAGGCGAGUCAAGGUCU CACACAUC | 2271 |
| 554 | UACCAUAU G UUGCUCCA | 850 | UGGAGCAA GCCGAAAGGCGAGUCAAGGUCU AUAUGGUA | 2272 |
| 557 | CAUAUGUU G CUCCAGAA | 851 | UUCUGGAG GCCGAAAGGCGAGUCAAGGUCU AACAUAUG | 2273 |
| 590 | AAUUUCAU G CAGAACCA | 852 | UGGUUCUG GCCGAAAGGCGAGUCAAGGUCU AUGAAAUU | 2274 |
| 599 | CAGAACCA G UUGAUGUU | 853 | AACAUCAA GCCGAAAGGCGAGUCAAGGUCU UGGUUCUG | 2275 |
| 605 | CAGUUGAU G UUUGGUCC | 854 | GGACCAAA GCCGAAAGGCGAGUCAAGGUCU AUCAACUG | 2276 |
| 610 | GAUGUUUG G UCCUGUGG | 855 | CCACAGGA GCCGAAAGGCGAGUCAAGGUCU CAAACAUC | 2277 |
| 615 | UUGGUCCU G UGGAAUAG | 856 | CUAUUCCA GCCGAAAGGCGAGUCAAGGUCU AGGACCAA | 2278 |
| 623 | GUGGAAUA G UACUUACU | 857 | AGUAAGUA GCCGAAAGGCGAGUCAAGGUCU UAUUCCAC | 2279 |
| 632 | UACUUACU G CAAUGCUC | 858 | GAGCAUUG GCCGAAAGGCGAGUCAAGGUCU AGUAAGUA | 2280 |
| 637 | ACUGCAAU G CUCGCUGG | 859 | CCAGCGAG GCCGAAAGGCGAGUCAAGGUCU AUUGCAGU | 2281 |
| 641 | CAAUGCUC G CUGGAGAA | 860 | UUCUCCAG GCCGAAAGGCGAGUCAAGGUCU GAGCAUUG | 2282 |
| 652 | GGAGAAUU G CCAUGGGA | 861 | UCCCAUGG GCCGAAAGGCGAGUCAAGGUCU AAUUCUCC | 2283 |
| 669 | CCAACCCA G UGACAGCU | 862 | AGCUGUCA GCCGAAAGGCGAGUCAAGGUCU UGGGUUGG | 2284 |
| 675 | CAGUGACA G CUGUCAGG | 863 | CCUGACAG GCCGAAAGGCGAGUCAAGGUCU UGUCACUG | 2285 |
| 678 | UGACAGCU G UCAGGAGU | 864 | ACUCCUGA GCCGAAAGGCGAGUCAAGGUCU AGCUGUCA | 2286 |
| 685 | UGUCAGGA G UAUUCUGA | 865 | UCAGAAUA GCCGAAAGGCGAGUCAAGGUCU UCCUGACA | 2287 |
| 743 | UCGAUUCU G CUCCUCUA | 866 | UAGAGGAG GCCGAAAGGCGAGUCAAGGUCU AGAAUCGA | 2288 |
| 752 | CUCCUCUA G CUCUGCUG | 867 | CAGCAGAG GCCGAAAGGCGAGUCAAGGUCU UAGAGGAG | 2289 |
| 757 | CUAGCUCU G CUGCAUAA | 868 | UUAUGCAG GCCGAAAGGCGAGUCAAGGUCU AGAGCUAG | 2290 |
| 760 | GCUCUGCU G CAUAAAAU | 869 | AUUUUAUG GCCGAAAGGCGAGUCAAGGUCU AGCAGAGC | 2291 |
| 773 | AAAUCUUA G UUGAGAAU | 870 | AUUCUCAA GCCGAAAGGCGAGUCAAGGUCU UAAGAUUU | 2292 |
| 788 | AUCCAUCA G CAAGAAUU | 871 | AAUUCUUG GCCGAAAGGCGAGUCAAGGUCU UGAUGGAU | 2293 |
| 826 | GAUAGAUG G UACAACAA | 872 | UUGUUGUA GCCGAAAGGCGAGUCAAGGUCU CAUCUAUC | 2294 |
| 851 | AGAAAGGG G CAAAAAGG | 873 | CCUUUUUG GCCGAAAGGCGAGUCAAGGUCU CCCUUUCU | 2295 |
| 859 | GCAAAAAG G CCCCGAGU | 874 | ACUCGGGG GCCGAAAGGCGAGUCAAGGUCU CUUUUUGC | 2296 |
| 866 | GGCCCCGA G UCACUUCA | 875 | UGAAGUGA GCCGAAAGGCGAGUCAAGGUCU UCGGGGCC | 2297 |
| 876 | CACUUCAG G UGGUGUGU | 876 | ACACACCA GCCGAAAGGCGAGUCAAGGUCU CUGAAGUG | 2298 |
| 879 | UUCAGGUG G UGUGUCAG | 877 | CUGACACA GCCGAAAGGCGAGUCAAGGUCU CACCUGAA | 2299 |
| 881 | CAGGUGGU G UGUCAGAG | 878 | CUCUGACA GCCGAAAGGCGAGUCAAGGUCU ACCACCUG | 2300 |
| 883 | GGUGGUGU G UCAGAGUC | 879 | GACUCUGA GCCGAAAGGCGAGUCAAGGUCU ACACCACC | 2301 |
| 889 | GUGUCAGA G UCUCCCAG | 880 | CUGGGAGA GCCGAAAGGCGAGUCAAGGUCU UCUGACAC | 2302 |
| 897 | GUCUCCCA G UGGAUUUU | 881 | AAAAUCCA GCCGAAAGGCGAGUCAAGGUCU UGGGAGAC | 2303 |
| 910 | UUUUCUAA G CACAUUCA | 882 | UGAAUGUG GCCGAAAGGCGAGUCAAGGUCU UUAGAAAA | 2304 |
| 941 | UCUCUCCA G UAAACAGU | 883 | ACUGUUUA GCCGAAAGGCGAGUCAAGGUCU UGGAGAGA | 2305 |
| 948 | AGUAAACA G UGCUUCUA | 884 | UAGAAGCA GCCGAAAGGCGAGUCAAGGUCU UGUUUACU | 2306 |
| 950 | UAAACAGU G CUUCUAGU | 885 | ACUAGAAG GCCGAAAGGCGAGUCAAGGUCU ACUGUUUA | 2307 |
| 957 | UGCUUCUA G UGAAGAAA | 886 | UUUCUUCA GCCGAAAGGCGAGUCAAGGUCU UAGAAGCA | 2308 |
| 968 | AAGAAAAU G UGAAGUAC | 887 | GUACUUCA GCCGAAAGGCGAGUCAAGGUCU AUUUUCUU | 2309 |
| 973 | AAUGUGAA G UACUCCAG | 888 | CUGGAGUA GCCGAAAGGCGAGUCAAGGUCU UUCACAUU | 2310 |
| 981 | GUACUCCA G UUCUCAGC | 889 | GCUGAGAA GCCGAAAGGCGAGUCAAGGUCU UGGAGUAC | 2311 |
| 988 | AGUUCUCA G CCAGAACC | 890 | GGUUCUGG GCCGAAAGGCGAGUCAAGGUCU UGAGAACU | 2312 |
| 999 | AGAACCCC G CACAGGUC | 891 | GACCUGUG GCCGAAAGGCGAGUCAAGGUCU GGGGUUCU | 2313 |
| 1005 | CCGCACAG G UCUUUCCU | 892 | AGGAAAGA GCCGAAAGGCGAGUCAAGGUCU CUGUGCGG | 2314 |
| 1026 | GGAUACCA G CCCCUCAU | 893 | AUGAGGGG GCCGAAAGGCGAGUCAAGGUCU UGGUAUCC | 2315 |
| 1049 | AUAAAUUG G UACAAGGG | 894 | CCCUUGUA GCCGAAAGGCGAGUCAAGGUCU CAAUUUAU | 2316 |
| 1062 | AGGGAUCA G CUUUUCCC | 895 | GGGAAAAG GCCGAAAGGCGAGUCAAGGUCU UGAUCCCU | 2317 |
| 1072 | UUUUCCCA G CCCACAUG | 896 | CAUGUGGG GCCGAAAGGCGAGUCAAGGUCU UGGGAAAA | 2318 |
| 1080 | GCCCACAU G UCCUGAUC | 897 | GAUCAGGA GCCGAAAGGCGAGUCAAGGUCU AUGUGGGC | 2319 |
| 1093 | GAUCAUAU G CUUUUGAA | 898 | UUCAAAAG GCCGAAAGGCGAGUCAAGGUCU AUAUGAUC | 2320 |
| 1104 | UUUGAAUA G UCAGUUAC | 899 | GUAACUGA GCCGAAAGGCGAGUCAAGGUCU UAUUCAAA | 2321 |
| 1108 | AAUAGUCA G UUACUUGG | 900 | CCAAGUAA GCCGAAAGGCGAGUCAAGGUCU UGACUAUU | 2322 |
| 1116 | GUUACUUG G CACCCCAG | 901 | CUGGGGUG GCCGAAAGGCGAGUCAAGGUCU CAAGUAAC | 2323 |
| 1144 | AACCCCUG G CAGCGGUU | 902 | AACCGCUG GCCGAAAGGCGAGUCAAGGUCU CAGGGGUU | 2324 |
| 1147 | CCCUGGCA G CGGUUGGU | 903 | ACCAACCG GCCGAAAGGCGAGUCAAGGUCU UGCCAGGG | 2325 |
| 1150 | UGGCAGCG G UUGGUCAA | 904 | UUGACCAA GCCGAAAGGCGAGUCAAGGUCU CGCUGCCA | 2326 |
| 1154 | AGCGGUUG G UCAAAAGA | 905 | UCUUUUGA GCCGAAAGGCGAGUCAAGGUCU CAACCGCU | 2327 |
| 1190 | AAUUGGAU G CAGACAAA | 906 | UUUGUCUG GCCGAAAGGCGAGUCAAGGUCU AUCCAAUU | 2328 |
| 1209 | UUAUCAAU G CCUGAAAG | 907 | CUUUCAGG GCCGAAAGGCGAGUCAAGGUCU AUUGAUAA | 2329 |
| 1224 | AGAGACUU G UGAGAAGU | 908 | ACUUCUCA GCCGAAAGGCGAGUCAAGGUCU AAGUCUCU | 2330 |
| 1231 | UGUGAGAA G UUGGGCUA | 909 | UAGCCCAA GCCGAAAGGCGAGUCAAGGUCU UUCUCACA | 2331 |
| 1236 | GAAGUUGG G CUAUCAAU | 910 | AUUGAUAG GCCGAAAGGCGAGUCAAGGUCU CCAACUUC | 2332 |
| 1254 | GAAGAAAA G UUGUAUGA | 911 | UCAUACAA GCCGAAAGGCGAGUCAAGGUCU UUUUCUUC | 2333 |
| 1257 | GAAAAGUU G UAUGAAUC | 912 | GAUUCAUA GCCGAAAGGCGAGUCAAGGUCU AACUUUUC | 2334 |
| 1268 | UGAAUCAG G UUACUAUA | 913 | UAUAGUAA GCCGAAAGGCGAGUCAAGGUCU CUGAUUCA | 2335 |
| 1316 | UUUUCAAA G UGAAUUUG | 914 | CAAAUUCA GCCGAAAGGCGAGUCAAGGUCU UUUGAAAA | 2336 |
| 1324 | GUGAAUUU G UUAGAAAU | 915 | AUUUCUAA GCCGAAAGGCGAGUCAAGGUCU AAAUUCAC | 2337 |
| 1349 | AAAUAUUG G UUGACUUC | 916 | GAAGUCAA GCCGAAAGGCGAGUCAAGGUCU CAAUAUUU | 2338 |
| 1360 | GACUUCCG G CUUUCUAA | 917 | UUAGAAAG GCCGAAAGGCGAGUCAAGGUCU CGGAAGUC | 2339 |
| 1371 | UUCUAAGG G UGAUGGAU | 918 | AUCCAUCA GCCGAAAGGCGAGUCAAGGUCU CCUUAGAA | 2340 |
| 1384 | GGAUUGGA G UUCAAGAG | 919 | CUCUUGAA GCCGAAAGGCGAGUCAAGGUCU UCCAAUCC | 2341 |
| 1417 | AAAGGGAA G CUGAUUGA | 920 | UCAAUCAG GCCGAAAGGCGAGUCAAGGUCU UUCCCUUU | 2342 |
| 1430 | UUGAUAUU G UGAGCAGC | 921 | GCUGCUCA GCCGAAAGGCGAGUCAAGGUCU AAUAUCAA | 2343 |
| 1434 | UAUUGUGA G CAGCCAGA | 922 | UCUGGCUG GCCGAAAGGCGAGUCAAGGUCU UCACAAUA | 2344 |
| 1437 | UGUGAGCA G CCAGAAGG | 923 | CCUUCUGG GCCGAAAGGCGAGUCAAGGUCU UGCUCACA | 2345 |
| 1445 | GCCAGAAG G UUUGGCUU | 924 | AAGCCAAA GCCGAAAGGCGAGUCAAGGUCU CUUCUGGC | 2346 |
| 1450 | AAGGUUUG G CUUCCUGC | 925 | GCAGGAAG GCCGAAAGGCGAGUCAAGGUCU CAAACCUU | 2347 |
| 1457 | GGCUUCCU G CCACAUGA | 926 | UCAUGUGG GCCGAAAGGCGAGUCAAGGUCU AGGAAGCC | 2348 |
| 1477 | GACCAUCG G CUCUGGGG | 927 | CCCCAGAG GCCGAAAGGCGAGUCAAGGUCU CGAUGGUC | 2349 |
| 1493 | GAAUCCUG G UGAAUAUA | 928 | UAUAUUCA GCCGAAAGGCGAGUCAAGGUCU CAGGAUUC | 2350 |
| 1502 | UGAAUAUA G UGCUGCUA | 929 | UAGCAGCA GCCGAAAGGCGAGUCAAGGUCU UAUAUUCA | 2351 |
| 1504 | AAUAUAGU G CUGCUAUG | 930 | CAUAGCAG GCCGAAAGGCGAGUCAAGGUCU ACUAUAUU | 2352 |
| 1507 | AUAGUGCU G CUAUGUUG | 931 | CAACAUAG GCCGAAAGGCGAGUCAAGGUCU AGCACUAU | 2353 |
| 1512 | GCUGCUAU G UUGACAUU | 932 | AAUGUCAA GCCGAAAGGCGAGUCAAGGUCU AUAGCAGC | 2354 |
| 1545 | AUUAUCCU G UCCUGCAA | 933 | UUGCAGGA GCCGAAAGGCGAGUCAAGGUCU AGGAUAAU | 2355 |
| 1550 | CCUGUCCU G CAAACUGC | 934 | GCAGUUUG GCCGAAAGGCGAGUCAAGGUCU AGGACAGG | 2356 |
| 1557 | UGCAAACU G CAAAUAGU | 935 | ACUAUUUG GCCGAAAGGCGAGUCAAGGUCU AGUUUGCA | 2357 |
| 1564 | UGCAAAUA G UAGUUCCU | 936 | AGGAACUA GCCGAAAGGCGAGUCAAGGUCU UAUUUGCA | 2358 |
| 1567 | AAAUAGUA G UUCCUGAA | 937 | UUCAGGAA GCCGAAAGGCGAGUCAAGGUCU UACUAUUU | 2359 |
| 1576 | UUCCUGAA G UGUUCACU | 938 | AGUGAACA GCCGAAAGGCGAGUCAAGGUCU UUCAGGAA | 2360 |
| 1578 | CCUGAAGU G UUCACUUC | 939 | GAAGUGAA GCCGAAAGGCGAGUCAAGGUCU ACUUCAGG | 2361 |
| 1590 | ACUUCCCU G UUUAUCCA | 940 | UGGAUAAA GCCGAAAGGCGAGUCAAGGUCU AGGGAAGU | 2362 |
| 1619 | UUUAUUUU G UUUGUUCG | 941 | CGAACAAA GCCGAAAGGCGAGUCAAGGUCU AAAAUAAA | 2363 |
| 1623 | UUUUGUUU G UUCGGCAU | 942 | AUGCCGAA GCCGAAAGGCGAGUCAAGGUCU AAACAAAA | 2364 |
| 1628 | UUUGUUCG G CAUACAAA | 943 | UUUGUAUG GCCGAAAGGCGAGUCAAGGUCU CGAACAAA | 2365 |
| 1656 | UCUUAAUU G UAAGCAAA | 944 | UUUGCUUA GCCGAAAGGCGAGUCAAGGUCU AAUUAAGA | 2366 |
| 1660 | AAUUGUAA G CAAAACUU | 945 | AAGUUUUG GCCGAAAGGCGAGUCAAGGUCU UUACAAUU | 2367 |
| 1710 | UUCUUCAU G UGUGUUUA | 946 | UAAACACA GCCGAAAGGCGAGUCAAGGUCU AUGAAGAA | 2368 |
| 1712 | CUUCAUGU G UGUUUAGU | 947 | ACUAAACA GCCGAAAGGCGAGUCAAGGUCU ACAUGAAG | 2369 |
| 1714 | UCAUGUGU G UUUAGUAU | 948 | AUACUAAA GCCGAAAGGCGAGUCAAGGUCU ACACAUGA | 2370 |
| 1719 | UGUGUUUA G UAUCUGAA | 949 | UUCAGAUA GCCGAAAGGCGAGUCAAGGUCU UAAACACA | 2371 |
| 1743 | CUCAUCUG G UGGAAACC | 950 | GGUUUCCA GCCGAAAGGCGAGUCAAGGUCU CAGAUGAG | 2372 |
| 1754 | GAAACCAA G UUUCAGGG | 951 | CCCUGAAA GCCGAAAGGCGAGUCAAGGUCU UUGGUUUC | 2373 |
| 1770 | GGACAUGA G UUUUCCAG | 952 | CUGGAAAA GCCGAAAGGCGAGUCAAGGUCU UCAUGUCC | 2374 |
| 1778 | GUUUUCCA G CUUUUAUA | 953 | UAUAAAAG GCCGAAAGGCGAGUCAAGGUCU UGGAAAAC | 2375 |
| 1792 | AUACACAC G UAUCUCAU | 954 | AUGAGAUA GCCGAAAGGCGAGUCAAGGUCU GUGUGUAU | 2376 |

Input Sequence = AF016582. Cut Site = G/Y
Stem Length = 8. Core Sequence = GCcgaaagGCGaGuCaaGGuCu
AF016582 (Homo sapiens checkpoint kinase Chk1 (CHK1) mRNA; 1821 bp)

TABLE VII
Human Chk1 DNAzyme and Substrate Sequence

| Pos | Substrate | Seq ID | DNAzyme | Seq ID |
|---|---|---|---|---|
| 10 | GCCGGACA G UCCGCCGA | 791 | TCGGCGGA GGCTAGCTACAACGA TGTCCGGC | 2377 |
| 14 | GACAGUCC G CCGAGGUG | 792 | CACCTCGG GGCTAGCTACAACGA GGACTGTC | 2378 |
| 20 | CCGCCGAG G UGCUCGGU | 793 | ACCGAGCA GGCTAGCTACAACGA CTCGGCGG | 2379 |
| 22 | GCCGAGGU G CUCGGUGG | 794 | CCACCGAG GGCTAGCTACAACGA ACCTCGGC | 2380 |
| 27 | GGUGCUCG G UGGAGUCA | 795 | TGACTCCA GGCTAGCTACAACGA CGAGCACC | 2381 |
| 32 | UCGGUGGA G UCAUGGCA | 796 | TGCCATGA GGCTAGCTACAACGA TCCACCGA | 2382 |
| 38 | GAGUCAUG G CAGUGCCC | 797 | GGGCACTG GGCTAGCTACAACGA CATGACTC | 2383 |
| 41 | UCAUGGCA G UGCCCUUU | 798 | AAAGGGCA GGCTAGCTACAACGA TGCCATGA | 2384 |
| 43 | AUGGCAGU G CCCUUUGU | 799 | ACAAAGGG GGCTAGCTACAACGA ACTGCCAT | 2385 |
| 50 | UGCCCUUU G UGGAAGAC | 800 | GTCTTCCA GGCTAGCTACAACGA AAAGGGCA | 2386 |
| 68 | GGGACUUG G UGCAAACC | 801 | GGTTTGCA GGCTAGCTACAACGA CAAGTCCC | 2387 |
| 70 | GACUUGGU G CAAACCCU | 802 | AGGGTTTG GGCTAGCTACAACGA ACCAAGTC | 2388 |
| 87 | GGGAGAAG G UGCCUAUG | 803 | CATAGGCA GGCTAGCTACAACGA CTTCTCCC | 2389 |
| 89 | GAGAAGGU G CCUAUGGA | 804 | TCCATAGG GGCTAGCTACAACGA ACCTTCTC | 2390 |
| 101 | AUGGAGAA G UUCAACUU | 805 | AAGTTGAA GGCTAGCTACAACGA TTCTCCAT | 2391 |
| 110 | UUCAACUU G CUGUGAAU | 806 | ATTCACAG GGCTAGCTACAACGA AAGTTGAA | 2392 |
| 113 | AACUUGCU G UGAAUAGA | 807 | TCTATTCA GGCTAGCTACAACGA AGCAAGTT | 2393 |
| 122 | UGAAUAGA G UAACUGAA | 808 | TTCAGTTA GGCTAGCTACAACGA TCTATTCA | 2394 |
| 134 | CUGAAGAA G CAGUCGCA | 809 | TGCGACTG GGCTAGCTACAACGA TTCTTCAG | 2395 |
| 137 | AAGAAGCA G UCGCAGUG | 810 | CACTGCGA GGCTAGCTACAACGA TGCTTCTT | 2396 |
| 140 | AAGCAGUC G CAGUGAAG | 811 | CTTCACTG GGCTAGCTACAACGA GACTGCTT | 2397 |
| 143 | CAGUCGCA G UGAAGAUU | 812 | AATCTTCA GGCTAGCTACAACGA TGCGACTG | 2398 |
| 152 | UGAAGAUU G UAGAUAUG | 813 | CATATCTA GGCTAGCTACAACGA AATCTTCA | 2399 |
| 163 | GAUAUGAA G CGUGCCGU | 814 | ACGGCACG GGCTAGCTACAACGA TTCATATC | 2400 |
| 165 | UAUGAAGC G UGCCGUAG | 815 | CTACGGCA GGCTAGCTACAACGA GCTTCATA | 2401 |
| 167 | UGAAGCGU G CCGUAGAC | 816 | GTCTACGG GGCTAGCTACAACGA ACGCTTCA | 2402 |
| 170 | AGCGUGCC G UAGACUGU | 817 | ACAGTCTA GGCTAGCTACAACGA GGCACGCT | 2403 |
| 177 | CGUAGACU G UCCAGAAA | 818 | TTTCTGGA GGCTAGCTACAACGA AGTCTACG | 2404 |
| 204 | AGAGAUCU G UAUCAAUA | 819 | TATTGATA GGCTAGCTACAACGA AGATCTCT | 2405 |
| 217 | AAUAAAAU G CUAAAUCA | 820 | TGATTTAG GGCTAGCTACAACGA ATTTTATT | 2406 |
| 233 | AUGAAAAU G UAGUAAAA | 821 | TTTTACTA GGCTAGCTACAACGA ATTTTCAT | 2407 |
| 236 | AAAAUGUA G UAAAAUUC | 822 | GAATTTTA GGCTAGCTACAACGA TACATTTT | 2408 |
| 249 | AUUCUAUG G UCACAGGA | 823 | TCCTGTGA GGCTAGCTACAACGA CATAGAAT | 2409 |
| 264 | GAGAGAAG G CAAUAUCC | 824 | GGATATTG GGCTAGCTACAACGA CTTCTCTC | 2410 |
| 289 | UUUCUGGA G UACUGUAG | 825 | CTACAGTA GGCTAGCTACAACGA TCCAGAAA | 2411 |
| 294 | GGAGUACU G UAGUGGAG | 826 | CTCCACTA GGCTAGCTACAACGA AGTACTCC | 2412 |
| 297 | GUACUGUA G UGGAGGAG | 827 | CTCCTCCA GGCTAGCTACAACGA TACAGTAC | 2413 |
| 307 | GGAGGAGA G CUUUUUGA | 828 | TCAAAAAG GGCTAGCTACAACGA TCTCCTCC | 2414 |
| 325 | AGAAUAGA G CCAGACAU | 829 | ATGTCTGG GGCTAGCTACAACGA TCTATTCT | 2415 |
| 336 | AGACAUAG G CAUGCCUG | 830 | CAGGCATG GGCTAGCTACAACGA CTATGTCT | 2416 |
| 340 | AUAGGCAU G CCUGAACC | 831 | GGTTCAGG GGCTAGCTACAACGA ATGCCTAT | 2417 |
| 353 | AACCAGAU G CUCAGAGA | 832 | TCTCTGAG GGCTAGCTACAACGA ATCTGGTT | 2418 |
| 380 | AACUCAUG G CAGGGGUG | 833 | CACCCCTG GGCTAGCTACAACGA CATGAGTT | 2419 |
| 386 | UGGCAGGG G UGGUUUAU | 834 | ATAAACCA GGCTAGCTACAACGA CCCTGCCA | 2420 |
| 389 | CAGGGGUG G UUUAUCUG | 835 | CAGATAAA GGCTAGCTACAACGA CACCCCTG | 2421 |
| 397 | GUUUAUCU G CAUGGUAU | 836 | ATACCATG GGCTAGCTACAACGA AGATAAAC | 2422 |
| 402 | UCUGCAUG G UAUUGGAA | 837 | TTCCAATA GGCTAGCTACAACGA CATGCAGA | 2423 |
| 445 | AAUCUUCU G UUGGAUGA | 838 | TCATCCAA GGCTAGCTACAACGA AGAAGATT | 2424 |
| 483 | AGACUUUG G CUUGGCAA | 839 | TTGCCAAG GGCTAGCTACAACGA CAAAGTCT | 2425 |
| 488 | UUGGCUUG G CAACAGUA | 840 | TACTGTTG GGCTAGCTACAACGA CAAGCCAA | 2426 |
| 494 | UGGCAACA G UAUUUCGG | 841 | CCGAAATA GGCTAGCTACAACGA TGTTGCCA | 2427 |
| 502 | GUAUUUCG G UAUAAUAA | 842 | TTATTATA GGCTAGCTACAACGA CGAAATAC | 2428 |
| 513 | UAAUAAUC G UGAGCGUU | 843 | AACGCTCA GGCTAGCTACAACGA GATTATTA | 2429 |
| 517 | AAUCGUGA G CGUUUGUU | 844 | AACAAACG GGCTAGCTACAACGA TCACGATT | 2430 |
| 519 | UCGUGAGC G UUUGUUGA | 845 | TCAACAAA GGCTAGCTACAACGA GCTCACGA | 2431 |
| 523 | GAGCGUUU G UUGAACAA | 846 | TTGTTCAA GGCTAGCTACAACGA AAACGCTC | 2432 |
| 535 | AACAAGAU G UGUGGUAC | 847 | GTACCACA GGCTAGCTACAACGA ATCTTGTT | 2433 |
| 537 | CAAGAUGU G UGGUACUU | 848 | AAGTACCA GGCTAGCTACAACGA ACATCTTG | 2434 |
| 540 | GAUGUGUG G UACUUUAC | 849 | GTAAAGTA GGCTAGCTACAACGA CACACATC | 2435 |
| 554 | UACCAUAU G UUGCUCCA | 850 | TGGAGCAA GGCTAGCTACAACGA ATATGGTA | 2436 |
| 557 | CAUAUGUU G CUCCAGAA | 851 | TTCTGGAG GGCTAGCTACAACGA AACATATG | 2437 |
| 590 | AAUUUCAU G CAGAACCA | 852 | TGGTTCTG GGCTAGCTACAACGA ATGAAATT | 2438 |
| 599 | CAGAACCA G UUGAUGUU | 853 | AACATCAA GGCTAGCTACAACGA TGGTTCTG | 2439 |
| 605 | CAGUUGAU G UUUGGUCC | 854 | GGACCAAA GGCTAGCTACAACGA ATCAACTG | 2440 |
| 610 | GAUGUUUG G UCCUGUGG | 855 | CCACAGGA GGCTAGCTACAACGA CAAACATC | 2441 |
| 615 | UUGGUCCU G UGGAAUAG | 856 | CTATTCCA GGCTAGCTACAACGA AGGACCAA | 2442 |
| 623 | GUGGAAUA G UACUUACU | 857 | AGTAAGTA GGCTAGCTACAACGA TATTCCAC | 2443 |
| 632 | UACUUACU G CAAUGCUC | 858 | GAGCATTG GGCTAGCTACAACGA AGTAAGTA | 2444 |
| 637 | ACUGCAAU G CUCGCUGG | 859 | CCAGCGAG GGCTAGCTACAACGA ATTGCAGT | 2445 |
| 641 | CAAUGCUC G CUGGAGAA | 860 | TTCTCCAG GGCTAGCTACAACGA GAGCATTG | 2446 |
| 652 | GGAGAAUU G CCAUGGGA | 861 | TCCCATGG GGCTAGCTACAACGA AATTCTCC | 2447 |
| 669 | CCAACCCA G UGACAGCU | 862 | AGCTGTCA GGCTAGCTACAACGA TGGGTTGG | 2448 |
| 675 | CAGUGACA G CUGUCAGG | 863 | CCTGACAG GGCTAGCTACAACGA TGTCACTG | 2449 |
| 678 | UGACAGCU G UCAGGAGU | 864 | ACTCCTGA GGCTAGCTACAACGA AGCTGTCA | 2450 |
| 685 | UGUCAGGA G UAUUCUGA | 865 | TCAGAATA GGCTAGCTACAACGA TCCTGACA | 2451 |
| 743 | UCGAUUCU G CUCCUCUA | 866 | TAGAGGAG GGCTAGCTACAACGA AGAATCGA | 2452 |
| 752 | CUCCUCUA G CUCUGCUG | 867 | CAGCAGAG GGCTAGCTACAACGA TAGAGGAG | 2453 |
| 757 | CUAGCUCU G CUGCAUAA | 868 | TTATGCAG GGCTAGCTACAACGA AGAGCTAG | 2454 |
| 760 | GCUCUGCU G CAUAAAAU | 869 | ATTTTATG GGCTAGCTACAACGA AGCAGAGC | 2455 |
| 773 | AAAUCUUA G UUGAGAAU | 870 | ATTCTCAA GGCTAGCTACAACGA TAAGATTT | 2456 |
| 788 | AUCCAUCA G CAAGAAUU | 871 | AATTCTTG GGCTAGCTACAACGA TGATGGAT | 2457 |
| 826 | GAUAGAUG G UACAACAA | 872 | TTGTTGTA GGCTAGCTACAACGA CATCTATC | 2458 |
| 851 | AGAAAGGG G CAAAAAGG | 873 | CCTTTTTG GGCTAGCTACAACGA CCCTTTCT | 2459 |
| 859 | GCAAAAAG G CCCCGAGU | 874 | ACTCGGGG GGCTAGCTACAACGA CTTTTTGC | 2460 |
| 866 | GGCCCCGA G UCACUUCA | 875 | TGAAGTGA GGCTAGCTACAACGA TCGGGGCC | 2461 |
| 876 | CACUUCAG G UGGUGUGU | 876 | ACACACCA GGCTAGCTACAACGA CTGAAGTG | 2462 |
| 879 | UUCAGGUG G UGUGUCAG | 877 | CTGACACA GGCTAGCTACAACGA CACCTGAA | 2463 |
| 881 | CAGGUGGU G UGUCAGAG | 878 | CTCTGACA GGCTAGCTACAACGA ACCACCTG | 2464 |
| 883 | GGUGGUGU G UCAGAGUC | 879 | GACTCTGA GGCTAGCTACAACGA ACACCACC | 2465 |
| 889 | GUGUCAGA G UCUCCCAG | 880 | CTGGGAGA GGCTAGCTACAACGA TCTGACAC | 2466 |
| 897 | GUCUCCCA G UGGAUUUU | 881 | AAAATCCA GGCTAGCTACAACGA TGGGAGAC | 2467 |
| 910 | UUUUCUAA G CACAUUCA | 882 | TGAATGTG GGCTAGCTACAACGA TTAGAAAA | 2468 |
| 941 | UCUCUCCA G UAAACAGU | 883 | ACTGTTTA GGCTAGCTACAACGA TGGAGAGA | 2469 |
| 948 | AGUAAACA G UGCUUCUA | 884 | TAGAAGCA GGCTAGCTACAACGA TGTTTACT | 2470 |
| 950 | UAAACAGU G CUUCUAGU | 885 | ACTAGAAG GGCTAGCTACAACGA ACTGTTTA | 2471 |
| 957 | UGCUUCUA G UGAAGAAA | 886 | TTTCTTCA GGCTAGCTACAACGA TAGAAGCA | 2472 |
| 968 | AAGAAAAU G UGAAGUAC | 887 | GTACTTCA GGCTAGCTACAACGA ATTTTCTT | 2473 |
| 973 | AAUGUGAA G UACUCCAG | 888 | CTGGAGTA GGCTAGCTACAACGA TTCACATT | 2474 |
| 981 | GUACUCCA G UUCUCAGC | 889 | GCTGAGAA GGCTAGCTACAACGA TGGAGTAC | 2475 |
| 988 | AGUUCUCA G CCAGAACC | 890 | GGTTCTGG GGCTAGCTACAACGA TGAGAACT | 2476 |
| 999 | AGAACCCC G CACAGGUC | 891 | GACCTGTG GGCTAGCTACAACGA GGGGTTCT | 2477 |
| 1005 | CCGCACAG G UCUUUCCU | 892 | AGGAAAGA GGCTAGCTACAACGA CTGTGCGG | 2478 |
| 1026 | GGAUACCA G CCCCUCAU | 893 | ATGAGGGG GGCTAGCTACAACGA TGGTATCC | 2479 |
| 1049 | AUAAAUUG G UACAAGGG | 894 | CCCTTGTA GGCTAGCTACAACGA CAATTTAT | 2480 |
| 1062 | AGGGAUCA G CUUUUCCC | 895 | GGGAAAAG GGCTAGCTACAACGA TGATCCCT | 2481 |
| 1072 | UUUUCCCA G CCCACAUG | 896 | CATGTGGG GGCTAGCTACAACGA TGGGAAAA | 2482 |
| 1080 | GCCCACAU G UCCUGAUC | 897 | GATCAGGA GGCTAGCTACAACGA ATGTGGGC | 2483 |
| 1093 | GAUCAUAU G CUUUUGAA | 898 | TTCAAAAG GGCTAGCTACAACGA ATATGATC | 2484 |
| 1104 | UUUGAAUA G UCAGUUAC | 899 | GTAACTGA GGCTAGCTACAACGA TATTCAAA | 2485 |
| 1108 | AAUAGUCA G UUACUUGG | 900 | CCAAGTAA GGCTAGCTACAACGA TGACTATT | 2486 |
| 1116 | GUUACUUG G CACCCCAG | 901 | CTGGGGTG GGCTAGCTACAACGA CAAGTAAC | 2487 |
| 1144 | AACCCCUG G CAGCGGUU | 902 | AACCGCTG GGCTAGCTACAACGA CAGGGGTT | 2488 |
| 1147 | CCCUGGCA G CGGUUGGU | 903 | ACCAACCG GGCTAGCTACAACGA TGCCAGGG | 2489 |
| 1150 | UGGCAGCG G UUGGUCAA | 904 | TTGACCAA GGCTAGCTACAACGA CGCTGCCA | 2490 |
| 1154 | AGCGGUUG G UCAAAAGA | 905 | TCTTTTGA GGCTAGCTACAACGA CAACCGCT | 2491 |
| 1190 | AAUUGGAU G CAGACAAA | 906 | TTTGTCTG GGCTAGCTACAACGA ATCCAATT | 2492 |
| 1209 | UUAUCAAU G CCUGAAAG | 907 | CTTTCAGG GGCTAGCTACAACGA ATTGATAA | 2493 |
| 1224 | AGAGACUU G UGAGAAGU | 908 | ACTTCTCA GGCTAGCTACAACGA AAGTCTCT | 2494 |
| 1231 | UGUGAGAA G UUGGGCUA | 909 | TAGCCCAA GGCTAGCTACAACGA TTCTCACA | 2495 |
| 1236 | GAAGUUGG G CUAUCAAU | 910 | ATTGATAG GGCTAGCTACAACGA CCAACTTC | 2496 |
| 1254 | GAAGAAAA G UUGUAUGA | 911 | TCATACAA GGCTAGCTACAACGA TTTTCTTC | 2497 |
| 1257 | GAAAAGUU G UAUGAAUC | 912 | GATTCATA GGCTAGCTACAACGA AACTTTTC | 2498 |
| 1268 | UGAAUCAG G UUACUAUA | 913 | TATAGTAA GGCTAGCTACAACGA CTGATTCA | 2499 |
| 1316 | UUUUCAAA G UGAAUUUG | 914 | CAAATTCA GGCTAGCTACAACGA TTTGAAAA | 2500 |
| 1324 | GUGAAUUU G UUAGAAAU | 915 | ATTTCTAA GGCTAGCTACAACGA AAATTCAC | 2501 |
| 1349 | AAAUAUUG G UUGACUUC | 916 | GAAGTCAA GGCTAGCTACAACGA CAATATTT | 2502 |
| 1360 | GACUUCCG G CUUUCUAA | 917 | TTAGAAAG GGCTAGCTACAACGA CGGAAGTC | 2503 |
| 1371 | UUCUAAGG G UGAUGGAU | 918 | ATCCATCA GGCTAGCTACAACGA CCTTAGAA | 2504 |
| 1384 | GGAUUGGA G UUCAAGAG | 919 | CTCTTGAA GGCTAGCTACAACGA TCCAATCC | 2505 |
| 1417 | AAAGGGAA G CUGAUUGA | 920 | TCAATCAG GGCTAGCTACAACGA TTCCCTTT | 2506 |
| 1430 | UUGAUAUU G UGAGCAGC | 921 | GCTGCTCA GGCTAGCTACAACGA AATATCAA | 2507 |
| 1434 | UAUUGUGA G CAGCCAGA | 922 | TCTGGCTG GGCTAGCTACAACGA TCACAATA | 2508 |
| 1437 | UGUGAGCA G CCAGAAGG | 923 | CCTTCTGG GGCTAGCTACAACGA TGCTCACA | 2509 |
| 1445 | GCCAGAAG G UUUGGCUU | 924 | AAGCCAAA GGCTAGCTACAACGA CTTCTGGC | 2510 |
| 1450 | AAGGUUUG G CUUCCUGC | 925 | GCAGGAAG GGCTAGCTACAACGA CAAACCTT | 2511 |
| 1457 | GGCUUCCU G CCACAUGA | 926 | TCATGTGG GGCTAGCTACAACGA AGGAAGCC | 2512 |
| 1477 | GACCAUCG G CUCUGGGG | 927 | CCCCAGAG GGCTAGCTACAACGA CGATGGTC | 2513 |
| 1493 | GAAUCCUG G UGAAUAUA | 928 | TATATTCA GGCTAGCTACAACGA CAGGATTC | 2514 |
| 1502 | UGAAUAUA G UGCUGCUA | 929 | TAGCAGCA GGCTAGCTACAACGA TATATTCA | 2515 |
| 1504 | AAUAUAGU G CUGCUAUG | 930 | CATAGCAG GGCTAGCTACAACGA ACTATATT | 2516 |
| 1507 | AUAGUGCU G CUAUGUUG | 931 | CAACATAG GGCTAGCTACAACGA AGCACTAT | 2517 |
| 1512 | GCUGCUAU G UUGACAUU | 932 | AATGTCAA GGCTAGCTACAACGA ATAGCAGC | 2518 |
| 1545 | AUUAUCCU G UCCUGCAA | 933 | TTGCAGGA GGCTAGCTACAACGA AGGATAAT | 2519 |
| 1550 | CCUGUCCU G CAAACUGC | 934 | GCAGTTTG GGCTAGCTACAACGA AGGACAGG | 2520 |
| 1557 | UGCAAACU G CAAAUAGU | 935 | ACTATTTG GGCTAGCTACAACGA AGTTTGCA | 2521 |
| 1564 | UGCAAAUA G UAGUUCCU | 936 | AGGAACTA GGCTAGCTACAACGA TATTTGCA | 2522 |
| 1567 | AAAUAGUA G UUCCUGAA | 937 | TTCAGGAA GGCTAGCTACAACGA TACTATTT | 2523 |
| 1576 | UUCCUGAA G UGUUCACU | 938 | AGTGAACA GGCTAGCTACAACGA TTCAGGAA | 2524 |
| 1578 | CCUGAAGU G UUCACUUC | 939 | GAAGTGAA GGCTAGCTACAACGA ACTTCAGG | 2525 |
| 1590 | ACUUCCCU G UUUAUCCA | 940 | TGGATAAA GGCTAGCTACAACGA AGGGAAGT | 2526 |
| 1619 | UUUAUUUU G UUUGUUCG | 941 | CGAACAAA GGCTAGCTACAACGA AAAATAAA | 2527 |
| 1623 | UUUUGUUU G UUCGGCAU | 942 | ATGCCGAA GGCTAGCTACAACGA AAACAAAA | 2528 |
| 1628 | UUUGUUCG G CAUACAAA | 943 | TTTGTATG GGCTAGCTACAACGA CGAACAAA | 2529 |
| 1656 | UCUUAAUU G UAAGCAAA | 944 | TTTGCTTA GGCTAGCTACAACGA AATTAAGA | 2530 |
| 1660 | AAUUGUAA G CAAAACUU | 945 | AAGTTTTG GGCTAGCTACAACGA TTACAATT | 2531 |
| 1710 | UUCUUCAU G UGUGUUUA | 946 | TAAACACA GGCTAGCTACAACGA ATGAAGAA | 2532 |
| 1712 | CUUCAUGU G UGUUUAGU | 947 | ACTAAACA GGCTAGCTACAACGA ACATGAAG | 2533 |
| 1714 | UCAUGUGU G UUUAGUAU | 948 | ATACTAAA GGCTAGCTACAACGA ACACATGA | 2534 |
| 1719 | UGUGUUUA G UAUCUGAA | 949 | TTCAGATA GGCTAGCTACAACGA TAAACACA | 2535 |
| 1743 | CUCAUCUG G UGGAAACC | 950 | GGTTTCCA GGCTAGCTACAACGA CAGATGAG | 2536 |
| 1754 | GAAACCAA G UUUCAGGG | 951 | CCCTGAAA GGCTAGCTACAACGA TTGGTTTC | 2537 |
| 1770 | GGACAUGA G UUUUCCAG | 952 | CTGGAAAA GGCTAGCTACAACGA TCATGTCC | 2538 |
| 1778 | GUUUUCCA G CUUUUAUA | 953 | TATAAAAG GGCTAGCTACAACGA TGGAAAAC | 2539 |
| 1792 | AUACACAC G UAUCUCAU | 954 | ATGAGATA GGCTAGCTACAACGA GTGTGTAT | 2540 |
| 35 | GUGGAGUC A UGGCAGUG | 955 | CACTGCCA GGCTAGCTACAACGA GACTCCAC | 2541 |
| 57 | UGUGGAAG A CUGGGACU | 956 | AGTCCCAG GGCTAGCTACAACGA CTTCCACA | 2542 |
| 63 | AGACUGGG A CUUGGUGC | 957 | GCACCAAG GGCTAGCTACAACGA CCCAGTCT | 2543 |
| 74 | UGGUGCAA A CCCUGGGA | 958 | TCCCAGGG GGCTAGCTACAACGA TTGCACCA | 2544 |
| 93 | AGGUGCCU A UGGAGAAG | 959 | CTTCTCCA GGCTAGCTACAACGA AGGCACCT | 2545 |
| 106 | GAAGUUCA A CUUGCUGU | 960 | ACAGCAAG GGCTAGCTACAACGA TGAACTTC | 2546 |
| 117 | UGCUGUGA A UAGAGUAA | 961 | TTACTCTA GGCTAGCTACAACGA TCACAGCA | 2547 |
| 125 | AUAGAGUA A CUGAAGAA | 962 | TTCTTCAG GGCTAGCTACAACGA TACTCTAT | 2548 |
| 149 | CAGUGAAG A UUGUAGAU | 963 | ATCTACAA GGCTAGCTACAACGA CTTCACTG | 2549 |
| 156 | GAUUGUAG A UAUGAAGC | 964 | GCTTCATA GGCTAGCTACAACGA CTACAATC | 2550 |
| 158 | UUGUAGAU A UGAAGCGU | 965 | ACGCTTCA GGCTAGCTACAACGA ATCTACAA | 2551 |
| 174 | UGCCGUAG A CUGUCCAG | 966 | CTGGACAG GGCTAGCTACAACGA CTACGGCA | 2552 |
| 186 | UCCAGAAA A UAUUAAGA | 967 | TCTTAATA GGCTAGCTACAACGA TTTCTGGA | 2553 |
| 188 | CAGAAAAU A UUAAGAAA | 968 | TTTCTTAA GGCTAGCTACAACGA ATTTTCTG | 2554 |
| 200 | AGAAAGAG A UCUGUAUC | 969 | GATACAGA GGCTAGCTACAACGA CTCTTTCT | 2555 |
| 206 | AGAUCUGU A UCAAUAAA | 970 | TTTATTGA GGCTAGCTACAACGA ACAGATCT | 2556 |
| 210 | CUGUAUCA A UAAAAUGC | 971 | GCATTTTA GGCTAGCTACAACGA TGATACAG | 2557 |
| 215 | UCAAUAAA A UGCUAAAU | 972 | ATTTAGCA GGCTAGCTACAACGA TTTATTGA | 2558 |
| 222 | AAUGCUAA A UCAUGAAA | 973 | TTTCATGA GGCTAGCTACAACGA TTAGCATT | 2559 |
| 225 | GCUAAAUC A UGAAAAUG | 974 | CATTTTCA GGCTAGCTACAACGA GATTTAGC | 2560 |
| 231 | UCAUGAAA A UGUAGUAA | 975 | TTACTACA GGCTAGCTACAACGA TTTCATGA | 2561 |
| 241 | GUAGUAAA A UUCUAUGG | 976 | CCATAGAA GGCTAGCTACAACGA TTTACTAC | 2562 |
| 246 | AAAAUUCU A UGGUCACA | 977 | TGTGACCA GGCTAGCTACAACGA AGAATTTT | 2563 |
| 252 | CUAUGGUC A CAGGAGAG | 978 | CTCTCCTG GGCTAGCTACAACGA GACCATAG | 2564 |
| 267 | AGAAGGCA A UAUCCAAU | 979 | ATTGGATA GGCTAGCTACAACGA TGCCTTCT | 2565 |
| 269 | AAGGCAAU A UCCAAUAU | 980 | ATATTGGA GGCTAGCTACAACGA ATTGCCTT | 2566 |
| 274 | AAUAUCCA A UAUUUAUU | 981 | AATAAATA GGCTAGCTACAACGA TGGATATT | 2567 |
| 276 | UAUCCAAU A UUUAUUUC | 982 | GAAATAAA GGCTAGCTACAACGA ATTGGATA | 2568 |
| 280 | CAAUAUUU A UUUCUGGA | 983 | TCCAGAAA GGCTAGCTACAACGA AAATATTG | 2569 |
| 291 | UCUGGAGU A CUGUAGUG | 984 | CACTACAG GGCTAGCTACAACGA ACTCCAGA | 2570 |
| 315 | GCUUUUUG A CAGAAUAG | 985 | CTATTCTG GGCTAGCTACAACGA CAAAAAGC | 2571 |
| 320 | UUGACAGA A UAGAGCCA | 986 | TGGCTCTA GGCTAGCTACAACGA TCTGTCAA | 2572 |
| 330 | AGAGCCAG A CAUAGGCA | 987 | TGCCTATG GGCTAGCTACAACGA CTGGCTCT | 2573 |
| 332 | AGCCAGAC A UAGGCAUG | 988 | CATGCCTA GGCTAGCTACAACGA GTCTGGCT | 2574 |
| 338 | ACAUAGGC A UGCCUGAA | 989 | TTCAGGCA GGCTAGCTACAACGA GCCTATGT | 2575 |
| 346 | AUGCCUGA A CCAGAUGC | 990 | GCATCTGG GGCTAGCTACAACGA TCAGGCAT | 2576 |
| 351 | UGAACCAG A UGCUCAGA | 991 | TCTGAGCA GGCTAGCTACAACGA CTGGTTCA | 2577 |
| 361 | GCUCAGAG A UUCUUCCA | 992 | TGGAAGAA GGCTAGCTACAACGA CTCTGAGC | 2578 |
| 369 | AUUCUUCC A UCAACUCA | 993 | TGAGTTGA GGCTAGCTACAACGA GGAAGAAT | 2579 |
| 373 | UUCCAUCA A CUCAUGGC | 994 | GCCATGAG GGCTAGCTACAACGA TGATGGAA | 2580 |
| 377 | AUCAACUC A UGGCAGGG | 995 | CCCTGCCA GGCTAGCTACAACGA GAGTTGAT | 2581 |
| 393 | GGUGGUUU A UCUGCAUG | 996 | CATGCAGA GGCTAGCTACAACGA AAACCACC | 2582 |
| 399 | UUAUCUGC A UGGUAUUG | 997 | CAATACCA GGCTAGCTACAACGA GCAGATAA | 2583 |
| 404 | UGCAUGGU A UUGGAAUA | 998 | TATTCCAA GGCTAGCTACAACGA ACCATGCA | 2584 |
| 410 | GUAUUGGA A UAACUCAC | 999 | GTGAGTTA GGCTAGCTACAACGA TCCAATAC | 2585 |
| 413 | UUGGAAUA A CUCACAGG | 1000 | CCTGTGAG GGCTAGCTACAACGA TATTCCAA | 2586 |
| 417 | AAUAACUC A CAGGGAUA | 1001 | TATCCCTG GGCTAGCTACAACGA GAGTTATT | 2587 |
| 423 | UCACAGGG A UAUUAAAC | 1002 | GTTTAATA GGCTAGCTACAACGA CCCTGTGA | 2588 |
| 425 | ACAGGGAU A UUAAACCA | 1003 | TGGTTTAA GGCTAGCTACAACGA ATCCCTGT | 2589 |
| 430 | GAUAUUAA A CCAGAAAA | 1004 | TTTTCTGG GGCTAGCTACAACGA TTAATATC | 2590 |
| 438 | ACCAGAAA A UCUUCUGU | 1005 | ACAGAAGA GGCTAGCTACAACGA TTTCTGGT | 2591 |
| 450 | UCUGUUGG A UGAAAGGG | 1006 | CCCTTTCA GGCTAGCTACAACGA CCAACAGA | 2592 |
| 459 | UGAAAGGG A UAACCUCA | 1007 | TGAGGTTA GGCTAGCTACAACGA CCCTTTCA | 2593 |
| 462 | AAGGGAUA A CCUCAAAA | 1008 | TTTTGAGG GGCTAGCTACAACGA TATCCCTT | 2594 |
| 470 | ACCUCAAA A UCUCAGAC | 1009 | GTCTGAGA GGCTAGCTACAACGA TTTGAGGT | 2595 |
| 477 | AAUCUCAG A CUUUGGCU | 1010 | AGCCAAAG GGCTAGCTACAACGA CTGAGATT | 2596 |
| 491 | GCUUGGCA A CAGUAUUU | 1011 | AAATACTG GGCTAGCTACAACGA TGCCAAGC | 2597 |
| 496 | GCAACAGU A UUUCGGUA | 1012 | TACCGAAA GGCTAGCTACAACGA ACTGTTGC | 2598 |
| 504 | AUUUCGGU A UAAUAAUC | 1013 | GATTATTA GGCTAGCTACAACGA ACCGAAAT | 2599 |
| 507 | UCGGUAUA A UAAUCGUG | 1014 | CACGATTA GGCTAGCTACAACGA TATACCGA | 2600 |
| 510 | GUAUAAUA A UCGUGAGC | 1015 | GCTCACGA GGCTAGCTACAACGA TATTATAC | 2601 |
| 528 | UUUGUUGA A CAAGAUGU | 1016 | ACATCTTG GGCTAGCTACAACGA TCAACAAA | 2602 |
| 533 | UGAACAAG A UGUGUGGU | 1017 | ACCACACA GGCTAGCTACAACGA CTTGTTCA | 2603 |
| 542 | UGUGUGGU A CUUUACCA | 1018 | TGGTAAAG GGCTAGCTACAACGA ACCACACA | 2604 |
| 547 | GGUACUUU A CCAUAUGU | 1019 | ACATATGG GGCTAGCTACAACGA AAAGTACC | 2605 |
| 550 | ACUUUACC A UAUGUUGC | 1020 | GCAACATA GGCTAGCTACAACGA GGTAAAGT | 2606 |
| 552 | UUUACCAU A UGUUGCUC | 1021 | GAGCAACA GGCTAGCTACAACGA ATGGTAAA | 2607 |
| 565 | GCUCCAGA A CUUCUGAA | 1022 | TTCAGAAG GGCTAGCTACAACGA TCTGGAGC | 2608 |
| 583 | AGAAGAGA A UUUCAUGC | 1023 | GCATGAAA GGCTAGCTACAACGA TCTCTTCT | 2609 |
| 588 | AGAAUUUC A UGCAGAAC | 1024 | GTTCTGCA GGCTAGCTACAACGA GAAATTCT | 2610 |
| 595 | CAUGCAGA A CCAGUUGA | 1025 | TCAACTGG GGCTAGCTACAACGA TCTGCATG | 2611 |
| 603 | ACCAGUUG A UGUUUGGU | 1026 | ACCAAACA GGCTAGCTACAACGA CAACTGGT | 2612 |
| 620 | CCUGUGGA A UAGUACUU | 1027 | AAGTACTA GGCTAGCTACAACGA TCCACAGG | 2613 |
| 625 | GGAAUAGU A CUUACUGC | 1028 | GCAGTAAG GGCTAGCTACAACGA ACTATTCC | 2614 |
| 629 | UAGUACUU A CUGCAAUG | 1029 | CATTGCAG GGCTAGCTACAACGA AAGTACTA | 2615 |
| 635 | UUACUGCA A UGCUCGCU | 1030 | AGCGAGCA GGCTAGCTACAACGA TGCAGTAA | 2616 |
| 649 | GCUGGAGA A UUGCCAUG | 1031 | CATGGCAA GGCTAGCTACAACGA TCTCCAGC | 2617 |
| 655 | GAAUUGCC A UGGGACCA | 1032 | TGGTCCCA GGCTAGCTACAACGA GGCAATTC | 2618 |
| 660 | GCCAUGGG A CCAACCCA | 1033 | TGGGTTGG GGCTAGCTACAACGA CCCATGGC | 2619 |
| 664 | UGGGACCA A CCCAGUGA | 1034 | TCACTGGG GGCTAGCTACAACGA TGGTCCCA | 2620 |
| 672 | ACCCAGUG A CAGCUGUC | 1035 | GACAGCTG GGCTAGCTACAACGA CACTGGGT | 2621 |
| 687 | UCAGGAGU A UUCUGACU | 1036 | AGTCAGAA GGCTAGCTACAACGA ACTCCTGA | 2622 |
| 693 | GUAUUCUG A CUGGAAAG | 1037 | CTTTCCAG GGCTAGCTACAACGA CAGAATAC | 2623 |
| 710 | AAAAAAAA A CAUACCUC | 1038 | GAGGTATG GGCTAGCTACAACGA TTTTTTTT | 2624 |
| 712 | AAAAAAAC A UACCUCAA | 1039 | TTGAGGTA GGCTAGCTACAACGA GTTTTTTT | 2625 |
| 714 | AAAAACAU A CCUCAACC | 1040 | GGTTGAGG GGCTAGCTACAACGA ATGTTTTT | 2626 |
| 720 | AUACCUCA A CCCUUGGA | 1041 | TCCAAGGG GGCTAGCTACAACGA TGAGGTAT | 2627 |
| 734 | GGAAAAAA A UCGAUUCU | 1042 | AGAATCGA GGCTAGCTACAACGA TTTTTTCC | 2628 |
| 738 | AAAAAUCG A UUCUGCUC | 1043 | GAGCAGAA GGCTAGCTACAACGA CGATTTTT | 2629 |
| 762 | UCUGCUGC A UAAAAUCU | 1044 | AGATTTTA GGCTAGCTACAACGA GCAGCAGA | 2630 |
| 767 | UGCAUAAA A UCUUAGUU | 1045 | AACTAAGA GGCTAGCTACAACGA TTTATGCA | 2631 |
| 780 | AGUUGAGA A UCCAUCAG | 1046 | CTGATGGA GGCTAGCTACAACGA TCTCAACT | 2632 |
| 784 | GAGAAUCC A UCAGCAAG | 1047 | CTTGCTGA GGCTAGCTACAACGA GGATTCTC | 2633 |
| 794 | CAGCAAGA A UUACCAUU | 1048 | AATGGTAA GGCTAGCTACAACGA TCTTGCTG | 2634 |
| 797 | CAAGAAUU A CCAUUCCA | 1049 | TGGAATGG GGCTAGCTACAACGA AATTCTTG | 2635 |
| 800 | GAAUUACC A UUCCAGAC | 1050 | GTCTGGAA GGCTAGCTACAACGA GGTAATTC | 2636 |
| 807 | CAUUCCAG A CAUCAAAA | 1051 | TTTTGATG GGCTAGCTACAACGA CTGGAATG | 2637 |
| 809 | UUCCAGAC A UCAAAAAA | 1052 | TTTTTTGA GGCTAGCTACAACGA GTCTGGAA | 2638 |
| 819 | CAAAAAAG A UAGAUGGU | 1053 | ACCATCTA GGCTAGCTACAACGA CTTTTTTG | 2639 |
| 823 | AAAGAUAG A UGGUACAA | 1054 | TTGTACCA GGCTAGCTACAACGA CTATCTTT | 2640 |
| 828 | UAGAUGGU A CAACAAAC | 1055 | GTTTGTTG GGCTAGCTACAACGA ACCATCTA | 2641 |
| 831 | AUGGUACA A CAAACCCC | 1056 | GGGGTTTG GGCTAGCTACAACGA TGTACCAT | 2642 |
| 835 | UACAACAA A CCCCUCAA | 1057 | TTGAGGGG GGCTAGCTACAACGA TTGTTGTA | 2643 |
| 869 | CCCGAGUC A CUUCAGGU | 1058 | ACCTGAAG GGCTAGCTACAACGA GACTCGGG | 2644 |
| 901 | CCCAGUGG A UUUUCUAA | 1059 | TTAGAAAA GGCTAGCTACAACGA CCACTGGG | 2645 |
| 912 | UUCUAAGC A CAUUCAAU | 1060 | ATTGAATG GGCTAGCTACAACGA GCTTAGAA | 2646 |
| 914 | CUAAGCAC A UUCAAUCC | 1061 | GGATTGAA GGCTAGCTACAACGA GTGCTTAG | 2647 |
| 919 | CACAUUCA A UCCAAUUU | 1062 | AAATTGGA GGCTAGCTACAACGA TGAATGTG | 2648 |
| 924 | UCAAUCCA A UUUGGACU | 1063 | AGTCCAAA GGCTAGCTACAACGA TGGATTGA | 2649 |
| 930 | CAAUUUGG A CUUCUCUC | 1064 | GAGAGAAG GGCTAGCTACAACGA CCAAATTG | 2650 |
| 945 | UCCAGUAA A CAGUGCUU | 1065 | AAGCACTG GGCTAGCTACAACGA TTACTGGA | 2651 |
| 966 | UGAAGAAA A UGUGAAGU | 1066 | ACTTCACA GGCTAGCTACAACGA TTTCTTCA | 2652 |
| 975 | UGUGAAGU A CUCCAGUU | 1067 | AACTGGAG GGCTAGCTACAACGA ACTTCACA | 2653 |
| 994 | CAGCCAGA A CCCCGCAC | 1068 | GTGCGGGG GGCTAGCTACAACGA TCTGGCTG | 2654 |
| 1001 | AACCCCGC A CAGGUCUU | 1069 | AAGACCTG GGCTAGCTACAACGA GCGGGGTT | 2655 |
| 1015 | CUUUCCUU A UGGGAUAC | 1070 | GTATCCCA GGCTAGCTACAACGA AAGGAAAG | 2656 |
| 1020 | CUUAUGGG A UACCAGCC | 1071 | GGCTGGTA GGCTAGCTACAACGA CCCATAAG | 2657 |
| 1022 | UAUGGGAU A CCAGCCCC | 1072 | GGGGCTGG GGCTAGCTACAACGA ATCCCATA | 2658 |
| 1033 | AGCCCCUC A UACAUUGA | 1073 | TCAATGTA GGCTAGCTACAACGA GAGGGGCT | 2659 |
| 1035 | CCCCUCAU A CAUUGAUA | 1074 | TATCAATG GGCTAGCTACAACGA ATGAGGGG | 2660 |
| 1037 | CCUCAUAC A UUGAUAAA | 1075 | TTTATCAA GGCTAGCTACAACGA GTATGAGG | 2661 |
| 1041 | AUACAUUG A UAAAUUGG | 1076 | CCAATTTA GGCTAGCTACAACGA CAATGTAT | 2662 |
| 1045 | AUUGAUAA A UUGGUACA | 1077 | TGTACCAA GGCTAGCTACAACGA TTATCAAT | 2663 |
| 1051 | AAAUUGGU A CAAGGGAU | 1078 | ATCCCTTG GGCTAGCTACAACGA ACCAATTT | 2664 |
| 1058 | UACAAGGG A UCAGCUUU | 1079 | AAAGCTGA GGCTAGCTACAACGA CCCTTGTA | 2665 |
| 1076 | CCCAGCCC A CAUGUCCU | 1080 | AGGACATG GGCTAGCTACAACGA GGGCTGGG | 2666 |
| 1078 | CAGCCCAC A UGUCCUGA | 1081 | TCAGGACA GGCTAGCTACAACGA GTGGGCTG | 2667 |
| 1086 | AUGUCCUG A UCAUAUGC | 1082 | GCATATGA GGCTAGCTACAACGA CAGGACAT | 2668 |
| 1089 | UCCUGAUC A UAUGCUUU | 1083 | AAAGCATA GGCTAGCTACAACGA GATCAGGA | 2669 |
| 1091 | CUGAUCAU A UGCUUUUG | 1084 | CAAAAGCA GGCTAGCTACAACGA ATGATCAG | 2670 |
| 1101 | GCUUUUGA A UAGUCAGU | 1085 | ACTGACTA GGCTAGCTACAACGA TCAAAAGC | 2671 |
| 1111 | AGUCAGUU A CUUGGCAC | 1086 | GTGCCAAG GGCTAGCTACAACGA AACTGACT | 2672 |
| 1118 | UACUUGGC A CCCCAGGA | 1087 | TCCTGGGG GGCTAGCTACAACGA GCCAAGTA | 2673 |
| 1126 | ACCCCAGG A UCCUCACA | 1088 | TGTGAGGA GGCTAGCTACAACGA CCTGGGGT | 2674 |
| 1132 | GGAUCCUC A CAGAACCC | 1089 | GGGTTCTG GGCTAGCTACAACGA GAGGATCC | 2675 |
| 1137 | CUCACAGA A CCCCUGGC | 1090 | GCCAGGGG GGCTAGCTACAACGA TCTGTGAG | 2676 |
| 1163 | UCAAAAGA A UGACACGA | 1091 | TCGTGTCA GGCTAGCTACAACGA TCTTTTGA | 2677 |
| 1166 | AAAGAAUG A CACGAUUC | 1092 | GAATCGTG GGCTAGCTACAACGA CATTCTTT | 2678 |
| 1168 | AGAAUGAC A CGAUUCUU | 1093 | AAGAATCG GGCTAGCTACAACGA GTCATTCT | 2679 |
| 1171 | AUGACACG A UUCUUUAC | 1094 | GTAAAGAA GGCTAGCTACAACGA CGTGTCAT | 2680 |
| 1178 | GAUUCUUU A CCAAAUUG | 1095 | CAATTTGG GGCTAGCTACAACGA AAAGAATC | 2681 |
| 1183 | UUUACCAA A UUGGAUGC | 1096 | GCATCCAA GGCTAGCTACAACGA TTGGTAAA | 2682 |
| 1188 | CAAAUUGG A UGCAGACA | 1097 | TGTCTGCA GGCTAGCTACAACGA CCAATTTG | 2683 |
| 1194 | GGAUGCAG A CAAAUCUU | 1098 | AAGATTTG GGCTAGCTACAACGA CTGCATCC | 2684 |
| 1198 | GCAGACAA A UCUUAUCA | 1099 | TGATAAGA GGCTAGCTACAACGA TTGTCTGC | 2685 |
| 1203 | CAAAUCUU A UCAAUGCC | 1100 | GGCATTGA GGCTAGCTACAACGA AAGATTTG | 2686 |
| 1207 | UCUUAUCA A UGCCUGAA | 1101 | TTCAGGCA GGCTAGCTACAACGA TGATAAGA | 2687 |
| 1220 | UGAAAGAG A CUUGUGAG | 1102 | CTCACAAG GGCTAGCTACAACGA CTCTTTCA | 2688 |
| 1239 | GUUGGGCU A UCAAUGGA | 1103 | TCCATTGA GGCTAGCTACAACGA AGCCCAAC | 2689 |
| 1243 | GGCUAUCA A UGGAAGAA | 1104 | TTCTTCCA GGCTAGCTACAACGA TGATAGCC | 2690 |
| 1259 | AAAGUUGU A UGAAUCAG | 1105 | CTGATTCA GGCTAGCTACAACGA ACAACTTT | 2691 |
| 1263 | UUGUAUGA A UCAGGUUA | 1106 | TAACCTGA GGCTAGCTACAACGA TCATACAA | 2692 |
| 1271 | AUCAGGUU A CUAUAUCA | 1107 | TGATATAG GGCTAGCTACAACGA AACCTGAT | 2693 |
| 1274 | AGGUUACU A UAUCAACA | 1108 | TGTTGATA GGCTAGCTACAACGA AGTAACCT | 2694 |
| 1276 | GUUACUAU A UCAACAAC | 1109 | GTTGTTGA GGCTAGCTACAACGA ATAGTAAC | 2695 |
| 1280 | CUAUAUCA A CAACUGAU | 1110 | ATCAGTTG GGCTAGCTACAACGA TGATATAG | 2696 |
| 1283 | UAUCAACA A CUGAUAGG | 1111 | CCTATCAG GGCTAGCTACAACGA TGTTGATA | 2697 |
| 1287 | AACAACUG A UAGGAGAA | 1112 | TTCTCCTA GGCTAGCTACAACGA CAGTTGTT | 2698 |
| 1296 | UAGGAGAA A CAAUAAAC | 1113 | GTTTATTG GGCTAGCTACAACGA TTCTCCTA | 2699 |
| 1299 | GAGAAACA A UAAACUCA | 1114 | TGAGTTTA GGCTAGCTACAACGA TGTTTCTC | 2700 |
| 1303 | AACAAUAA A CUCAUUUU | 1115 | AAAATGAG GGCTAGCTACAACGA TTATTGTT | 2701 |
| 1307 | AUAAACUC A UUUUCAAA | 1116 | TTTGAAAA GGCTAGCTACAACGA GAGTTTAT | 2702 |
| 1320 | CAAAGUGA A UUUGUUAG | 1117 | CTAACAAA GGCTAGCTACAACGA TCACTTTG | 2703 |
| 1331 | UGUUAGAA A UGGAUGAU | 1118 | ATCATCCA GGCTAGCTACAACGA TTCTAACA | 2704 |
| 1335 | AGAAAUGG A UGAUAAAA | 1119 | TTTTATCA GGCTAGCTACAACGA CCATTTCT | 2705 |
| 1338 | AAUGGAUG A UAAAAUAU | 1120 | ATATTTTA GGCTAGCTACAACGA CATCCATT | 2706 |
| 1343 | AUGAUAAA A UAUUGGUU | 1121 | AACCAATA GGCTAGCTACAACGA TTTATCAT | 2707 |
| 1345 | GAUAAAAU A UUGGUUGA | 1122 | TCAACCAA GGCTAGCTACAACGA ATTTTATC | 2708 |
| 1353 | AUUGGUUG A CUUCCGGC | 1123 | GCCGGAAG GGCTAGCTACAACGA CAACCAAT | 2709 |
| 1374 | UAAGGGUG A UGGAUUGG | 1124 | CCAATCCA GGCTAGCTACAACGA CACCCTTA | 2710 |
| 1378 | GGUGAUGG A UUGGAGUU | 1125 | AACTCCAA GGCTAGCTACAACGA CCATCACC | 2711 |
| 1393 | UUCAAGAG A CACUUCCU | 1126 | AGGAAGTG GGCTAGCTACAACGA CTCTTGAA | 2712 |
| 1395 | CAAGAGAC A CUUCCUGA | 1127 | TCAGGAAG GGCTAGCTACAACGA GTCTCTTG | 2713 |
| 1406 | UCCUGAAG A UUAAAGGG | 1128 | CCCTTTAA GGCTAGCTACAACGA CTTCAGGA | 2714 |
| 1421 | GGAAGCUG A UUGAUAUU | 1129 | AATATCAA GGCTAGCTACAACGA CAGCTTCC | 2715 |
| 1425 | GCUGAUUG A UAUUGUGA | 1130 | TCACAATA GGCTAGCTACAACGA CAATCAGC | 2716 |
| 1427 | UGAUUGAU A UUGUGAGC | 1131 | GCTCACAA GGCTAGCTACAACGA ATCAATCA | 2717 |
| 1460 | UUCCUGCC A CAUGAUCG | 1132 | CGATCATG GGCTAGCTACAACGA GGCAGGAA | 2718 |
| 1462 | CCUGCCAC A UGAUCGGA | 1133 | TCCGATCA GGCTAGCTACAACGA GTGGCAGG | 2719 |
| 1465 | GCCACAUG A UCGGACCA | 1134 | TGGTCCGA GGCTAGCTACAACGA CATGTGGC | 2720 |
| 1470 | AUGAUCGG A CCAUCGGC | 1135 | GCCGATGG GGCTAGCTACAACGA CCGATCAT | 2721 |
| 1473 | AUCGGACC A UCGGCUCU | 1136 | AGAGCCGA GGCTAGCTACAACGA GGTCCGAT | 2722 |
| 1487 | UCUGGGGA A UCCUGGUG | 1137 | CACCAGGA GGCTAGCTACAACGA TCCCCAGA | 2723 |
| 1497 | CCUGGUGA A UAUAGUGC | 1138 | GCACTATA GGCTAGCTACAACGA TCACCAGG | 2724 |
| 1499 | UGGUGAAU A UAGUGCUG | 1139 | CAGCACTA GGCTAGCTACAACGA ATTCACCA | 2725 |
| 1510 | GUGCUGCU A UGUUGACA | 1140 | TGTCAACA GGCTAGCTACAACGA AGCAGCAC | 2726 |
| 1516 | CUAUGUUG A CAUUAUUC | 1141 | GAATAATG GGCTAGCTACAACGA CAACATAG | 2727 |
| 1518 | AUGUUGAC A UUAUUCUU | 1142 | AAGAATAA GGCTAGCTACAACGA GTCAACAT | 2728 |
| 1521 | UUGACAUU A UUCUUCCU | 1143 | AGGAAGAA GGCTAGCTACAACGA AATGTCAA | 2729 |
| 1537 | UAGAGAAG A UUAUCCUG | 1144 | CAGGATAA GGCTAGCTACAACGA CTTCTCTA | 2730 |
| 1540 | AGAAGAUU A UCCUGUCC | 1145 | GGACAGGA GGCTAGCTACAACGA AATCTTCT | 2731 |
| 1554 | UCCUGCAA A CUGCAAAU | 1146 | ATTTGCAG GGCTAGCTACAACGA TTGCAGGA | 2732 |
| 1561 | AACUGCAA A UAGUAGUU | 1147 | AACTACTA GGCTAGCTACAACGA TTGCAGTT | 2733 |
| 1582 | AAGUGUUC A CUUCCCUG | 1148 | CAGGGAAG GGCTAGCTACAACGA GAACACTT | 2734 |
| 1594 | CCCUGUUU A UCCAAACA | 1149 | TGTTTGGA GGCTAGCTACAACGA AAACAGGG | 2735 |
| 1600 | UUAUCCAA A CAUCUUCC | 1150 | GGAAGATG GGCTAGCTACAACGA TTGGATAA | 2736 |
| 1602 | AUCCAAAC A UCUUCCAA | 1151 | TTGGAAGA GGCTAGCTACAACGA GTTTGGAT | 2737 |
| 1610 | AUCUUCCA A UUUAUUUU | 1152 | AAAATAAA GGCTAGCTACAACGA TGGAAGAT | 2738 |
| 1614 | UCCAAUUU A UUUUGUUU | 1153 | AAACAAAA GGCTAGCTACAACGA AAATTGGA | 2739 |
| 1630 | UGUUCGGC A UACAAAUA | 1154 | TATTTGTA GGCTAGCTACAACGA GCCGAACA | 2740 |
| 1632 | UUCGGCAU A CAAAUAAU | 1155 | ATTATTTG GGCTAGCTACAACGA ATGCCGAA | 2741 |
| 1636 | GCAUACAA A UAAUACCU | 1156 | AGGTATTA GGCTAGCTACAACGA TTGTATGC | 2742 |
| 1639 | UACAAAUA A UACCUAUA | 1157 | TATAGGTA GGCTAGCTACAACGA TATTTGTA | 2743 |
| 1641 | CAAAUAAU A CCUAUAUC | 1158 | GATATAGG GGCTAGCTACAACGA ATTATTTG | 2744 |
| 1645 | UAAUACCU A UAUCUUAA | 1159 | TTAAGATA GGCTAGCTACAACGA AGGTATTA | 2745 |
| 1647 | AUACCUAU A UCUUAAUU | 1160 | AATTAAGA GGCTAGCTACAACGA ATAGGTAT | 2746 |
| 1653 | AUAUCUUA A UUGUAAGC | 1161 | GCTTACAA GGCTAGCTACAACGA TAAGATAT | 2747 |
| 1665 | UAAGCAAA A CUUUGGGG | 1162 | CCCCAAAG GGCTAGCTACAACGA TTTGCTTA | 2748 |
| 1679 | GGGAAAGG A UGAAUAGA | 1163 | TCTATTCA GGCTAGCTACAACGA CCTTTCCC | 2749 |
| 1683 | AAGGAUGA A UAGAAUUC | 1164 | GAATTCTA GGCTAGCTACAACGA TCATCCTT | 2750 |
| 1688 | UGAAUAGA A UUCAUUUG | 1165 | CAAATGAA GGCTAGCTACAACGA TCTATTCA | 2751 |
| 1692 | UAGAAUUC A UUUGAUUA | 1166 | TAATCAAA GGCTAGCTACAACGA GAATTCTA | 2752 |
| 1697 | UUCAUUUG A UUAUUUCU | 1167 | AGAAATAA GGCTAGCTACAACGA CAAATGAA | 2753 |
| 1700 | AUUUGAUU A UUUCUUCA | 1168 | TGAAGAAA GGCTAGCTACAACGA AATCAAAT | 2754 |
| 1708 | AUUUCUUC A UGUGUGUU | 1169 | AACACACA GGCTAGCTACAACGA GAAGAAAT | 2755 |
| 1721 | UGUUUAGU A UCUGAAUU | 1170 | AATTCAGA GGCTAGCTACAACGA ACTAAACA | 2756 |
| 1727 | GUAUCUGA A UUUGAAAC | 1171 | GTTTCAAA GGCTAGCTACAACGA TCAGATAC | 2757 |
| 1734 | AAUUUGAA A CUCAUCUG | 1172 | CAGATGAG GGCTAGCTACAACGA TTCAAATT | 2758 |
| 1738 | UGAAACUC A UCUGGUGG | 1173 | CCACCAGA GGCTAGCTACAACGA GAGTTTCA | 2759 |
| 1749 | UGGUGGAA A CCAAGUUU | 1174 | AAACTTGG GGCTAGCTACAACGA TTCCACCA | 2760 |
| 1764 | UUCAGGGG A CAUGAGUU | 1175 | AACTCATG GGCTAGCTACAACGA CCCCTGAA | 2761 |
| 1766 | CAGGGGAC A UGAGUUUU | 1176 | AAAACTCA GGCTAGCTACAACGA GTCCCCTG | 2762 |
| 1784 | CAGCUUUU A UACACACG | 1177 | CGTGTGTA GGCTAGCTACAACGA AAAAGCTG | 2763 |
| 1786 | GCUUUUAU A CACACGUA | 1178 | TACGTGTG GGCTAGCTACAACGA ATAAAAGC | 2764 |
| 1788 | UUUUAUAC A CACGUAUC | 1179 | GATACGTG GGCTAGCTACAACGA GTATAAAA | 2765 |
| 1790 | UUAUACAC A CGUAUCUC | 1180 | GAGATACG GGCTAGCTACAACGA GTGTATAA | 2766 |
| 1794 | ACACACGU A UCUCAUUU | 1181 | AAATGAGA GGCTAGCTACAACGA ACGTGTGT | 2767 |
| 1799 | CGUAUCUC A UUUUUAUC | 1182 | GATAAAAA GGCTAGCTACAACGA GAGATACG | 2768 |
| 1805 | UCAUUUUU A UCAAAACA | 1183 | TGTTTTGA GGCTAGCTACAACGA AAAAATGA | 2769 |
| 1811 | UUAUCAAA A CAUUUUGU | 1184 | ACAAAATG GGCTAGCTACAACGA TTTGATAA | 2770 |
| 1813 | AUCAAAAC A UUUUGUUU | 1185 | AAACAAAA GGCTAGCTACAACGA GTTTTGAT | 2771 |

Input Sequence = AF016582 Cut Site = R/Y
Stem Length = 8. Core Sequence = GGCTAGCTACAACGA
AF016582 (Homo sapiens checkpoint kinase Chk1 (CHK1) mRNA; 1821 bp)

TABLE VIII
Human Chk1 Amberzyme Ribozyme and Substrate Sequence

| Pos | Substrate | Seq ID | Ribozyme | Rz Seq ID |
|---|---|---|---|---|
| 10 | GCCGGACA G UCCGCCGA | 791 | UCGGCGGA GGAGGAAACUCC CU UCAAGGACAUCGUCCGGG UGUCCGGC | 2772 |
| 14 | GACAGUCC G CCGAGGUG | 792 | CACCUCGG GGAGGAAACUCC CU UCAAGGACAUCGUCCGGG GGACUGUC | 2773 |
| 20 | CCGCCGAG G UGCUCGGU | 793 | ACCGAGCA GGAGGAAACUCC CU UCAAGGACAUCGUCCGGG CUCGGCGG | 2774 |
| 22 | GCCGAGGU G CUCGGUGG | 794 | CCACCGAG GGAGGAAACUCC CU UCAAGGACAUCGUCCGGG ACCUCGGC | 2775 |
| 27 | GGUGCUCG G UGGAGUCA | 795 | UGACUCCA GGAGGAAACUCC CU UCAAGGACAUCGUCCGGG CGAGCACC | 2776 |
| 32 | UCGGUGGA G UCAUGGCA | 796 | UGCCAUGA GGAGGAAACUCC CU UCAAGGACAUCGUCCGGG UCCACCGA | 2777 |
| 38 | GAGUCAUG G CAGUGCCC | 797 | GGGCACUG GGAGGAAACUCC CU UCAAGGACAUCGUCCGGG CAUGACUC | 2778 |
| 41 | UCAUGGCA G UGCCCUUU | 798 | AAAGGGCA GGAGGAAACUCC CU UCAAGGACAUCGUCCGGG UGCCAUGA | 2779 |
| 43 | AUGGCAGU G CCCUUUGU | 799 | ACAAAGGG GGAGGAAACUCC CU UCAAGGACAUCGUCCGGG ACUGCCAU | 2780 |
| 50 | UGCCCUUU G UGGAAGAC | 800 | GUCUUCCA GGAGGAAACUCC CU UCAAGGACAUCGUCCGGG AAAGGGCA | 2781 |
| 68 | GGGACUUG G UGCAAACC | 801 | GGUUUGCA GGAGGAAACUCC CU UCAAGGACAUCGUCCGGG CAAGUCCC | 2782 |
| 70 | GACUUGGU G CAAACCCU | 802 | AGGGUUUG GGAGGAAACUCC CU UCAAGGACAUCGUCCGGG ACCAAGUC | 2783 |
| 87 | GGGAGAAG G UGCCUAUG | 803 | CAUAGGCA GGAGGAAACUCC CU UCAAGGACAUCGUCCGGG CUUCUCCC | 2784 |
| 89 | GAGAAGGU G CCUAUGGA | 804 | UCCAUAGG GGAGGAAACUCC CU UCAAGGACAUCGUCCGGG ACCUUCUC | 2785 |
| 101 | AUGGAGAA G UUCAACUU | 805 | AAGUUGAA GGAGGAAACUCC CU UCAAGGACAUCGUCCGGG UUCUCCAU | 2786 |
| 110 | UUCAACUU G CUGUGAAU | 806 | AUUCACAG GGAGGAAACUCC CU UCAAGGACAUCGUCCGGG AAGUUGAA | 2787 |
| 113 | AACUUGCU G UGAAUAGA | 807 | UCUAUUCA GGAGGAAACUCC CU UCAAGGACAUCGUCCGGG AGCAAGUU | 2788 |
| 122 | UGAAUAGA G UAACUGAA | 808 | UUCAGUUA GGAGGAAACUCC CU UCAAGGACAUCGUCCGGG UCUAUUCA | 2789 |
| 134 | CUGAAGAA G CAGUCGCA | 809 | UGCGACUG GGAGGAAACUCC CU UCAAGGACAUCGUCCGGG UUCUUCAG | 2790 |
| 137 | AAGAAGCA G UCGCAGUG | 810 | CACUGCGA GGAGGAAACUCC CU UCAAGGACAUCGUCCGGG UGCUUCUU | 2791 |
| 140 | AAGCAGUC G CAGUGAAG | 811 | CUUCACUG GGAGGAAACUCC CU UCAAGGACAUCGUCCGGG GACUGCUU | 2792 |
| 143 | CAGUCGCA G UGAAGAUU | 812 | AAUCUUCA GGAGGAAACUCC CU UCAAGGACAUCGUCCGGG UGCGACUG | 2793 |
| 152 | UGAAGAUU G UAGAUAUG | 813 | CAUAUCUA GGAGGAAACUCC CU UCAAGGACAUCGUCCGGG AAUCUUCA | 2794 |
| 163 | GAUAUGAA G CGUGCCGU | 814 | ACGGCACG GGAGGAAACUCC CU UCAAGGACAUCGUCCGGG UUCAUAUC | 2795 |
| 165 | UAUGAAGC G UGCCGUAG | 815 | CUACGGCA GGAGGAAACUCC CU UCAAGGACAUCGUCCGGG UUCAUAUC | 2796 |
| 167 | UGAAGCGU G CCGUAGAC | 816 | GUCUACGG GGAGGAAACUCC CU UCAAGGACAUCGUCCGGG ACGCUUCA | 2797 |
| 170 | AGCGUGCC G UAGACUGU | 817 | ACAGUCUA GGAGGAAACUCC CU UCAAGGACAUCGUCCGGG GGCACGCU | 2798 |
| 177 | CGUAGACU G UCCAGAAA | 818 | UUUCUGGA GGAGGAAACUCC CU UCAAGGACAUCGUCCGGG AGUCUACG | 2799 |
| 204 | AGAGAUCU G UAUCAAUA | 819 | UAUUGAUA GGAGGAAACUCC CU UCAAGGACAUCGUCCGGG AGAUCUCU | 2800 |
| 217 | AAUAAAAU G CUAAAUCA | 820 | UGAUUUAG GGAGGAAACUCC CU UCAAGGACAUCGUCCGGG AUUUUAUU | 2801 |
| 233 | AUGAAAAU G UAGUAAAA | 821 | UUUUACUA GGAGGAAACUCC CU UCAAGGACAUCGUCCGGG AUUUUCAU | 2802 |
| 236 | AAAAUGUA G UAAAAUUC | 822 | GAAUUUUA GGAGGAAACUCC CU UCAAGGACAUCGUCCGGG UACAUUUU | 2803 |
| 249 | AUUCUAUG G UCACAGGA | 823 | UCCUGUGA GGAGGAAACUCC CU UCAAGGACAUCGUCCGGG CAUAGAAU | 2804 |
| 264 | GAGAGAAG G CAAUAUCC | 824 | GGAUAUUG GGAGGAAACUCC CU UCAAGGACAUCGUCCGGG CUUCUCUC | 2805 |
| 289 | UUUCUGGA G UACUGUAG | 825 | CUACAGUA GGAGGAAACUCC CU UCAAGGACAUCGUCCGGG UCCAGAAA | 2806 |
| 294 | GGAGUACU G UAGUGGAG | 826 | CUCCACUA GGAGGAAACUCC CU UCAAGGACAUCGUCCGGG AGUACUCC | 2807 |
| 297 | GUACUGUA G UGGAGGAG | 827 | CUCCUCCA GGAGGAAACUCC CU UCAAGGACAUCGUCCGGG UACAGUAC | 2808 |
| 307 | GGAGGAGA G CUUUUUGA | 828 | UCAAAAAG GGAGGAAACUCC CU UCAAGGACAUCGUCCGGG UCUCCUCC | 2809 |
| 325 | AGAAUAGA G CCAGACAU | 829 | AUGUCUGG GGAGGAAACUCC CU UCAAGGACAUCGUCCGGG UCUAUUCU | 2810 |
| 336 | AGACAUAG G CAUGCCUG | 830 | CAGGCAUG GGAGGAAACUCC CU UCAAGGACAUCGUCCGGG CUAUGUCU | 2811 |
| 340 | AUAGGCAU G CCUGAACC | 831 | GGUUCAGG GGAGGAAACUCC CU UCAAGGACAUCGUCCGGG AUGCCUAU | 2812 |
| 353 | AACCAGAU G CUCAGAGA | 832 | UCUCUGAG GGAGGAAACUCC CU UCAAGGACAUCGUCCGGG AUCUGGUU | 2813 |
| 380 | AACUCAUG G CAGGGGUG | 833 | CACCCCUG GGAGGAAACUCC CU UCAAGGACAUCGUCCGGG CAUGAGUU | 2814 |
| 386 | UGGCAGGG G UGGUUUAU | 834 | AUAAACCA GGAGGAAACUCC CU UCAAGGACAUCGUCCGGG CCCUGCCA | 2815 |
| 389 | CAGGGGUG G UUUAUCUG | 835 | CAGAUAAA GGAGGAAACUCC CU UCAAGGACAUCGUCCGGG CACCCCUG | 2816 |
| 397 | GUUUAUCU G CAUGGUAU | 836 | AUACCAUG GGAGGAAACUCC CU UCAAGGACAUCGUCCGGG AGAUAAAC | 2817 |
| 402 | UCUGCAUG G UAUUGGAA | 837 | UUCCAAUA GGAGGAAACUCC CU UCAAGGACAUCGUCCGGG CAUGCAGA | 2818 |
| 445 | AAUCUUCU G UUGGAUGA | 838 | UCAUCCAA GGAGGAAACUCC CU UCAAGGACAUCGUCCGGG AGAAGAUU | 2819 |
| 483 | AGACUUUG G CUUGGCAA | 839 | UUGCCAAG GGAGGAAACUCC CU UCAAGGACAUCGUCCGGG CAAAGUCU | 2820 |
| 488 | UUGGCUUG G CAACAGUA | 840 | UACUGUUG GGAGGAAACUCC CU UCAAGGACAUCGUCCGGG CAAGCCAA | 2821 |
| 494 | UGGCAACA G UAUUUCGG | 841 | CCGAAAUA GGAGGAAACUCC CU UCAAGGACAUCGUCCGGG UGUUGCCA | 2822 |
| 502 | GUAUUUCG G UAUAAUAA | 842 | UUAUUAUA GGAGGAAACUCC CU UCAAGGACAUCGUCCGGG CGAAAUAC | 2823 |
| 513 | UAAUAAUC G UGAGCGUU | 843 | AACGCUCA GGAGGAAACUCC CU UCAAGGACAUCGUCCGGG GAUUAUUA | 2824 |
| 517 | AAUCGUGA G CGUUUGUU | 844 | AACAAACG GGAGGAAACUCC CU UCAAGGACAUCGUCCGGU UCACGAUU | 2825 |
| 519 | UCGUGAGC G UUUGUUGA | 845 | UCAACAAA GGAGGAAACUCC CU UCAAGGACAUCGUCCGGG GCUCACGA | 2826 |
| 523 | GAGCGUUU G UUGAACAA | 846 | UUGUUCAA GGAGGAAACUCC CU UCAAGGACAUCGUCCGGG AAACGCUC | 2827 |
| 535 | AACAAGAU G UGUGGUAC | 847 | GUACCACA GGAGGAAACUCC CU UCAAGGACAUCGUCCGGG AUCUUGUU | 2828 |
| 537 | CAAGAUGU G UGGUACUU | 848 | AAGUACCA GGAGGAAACUCC CU UCAAGGACAUCGUCCGGG ACAUCUUG | 2829 |
| 540 | GAUGUGUG G UACUUUAC | 849 | GUAAAGUA GGAGGAAACUCC CU UCAAGGACAUCGUCCGGG CACACAUC | 2830 |
| 554 | UACCAUAU G UUGCUCCA | 850 | UGGAGCAA GGAGGAAACUCC CU UCAAGGACAUCGUCCGGG AUAUGGUA | 2831 |
| 557 | CAUAUGUU G CUCCAGAA | 851 | UUCUGGAG GGAGGAAACUCC CU UCAAGGACAUCGUCCGGG AACAUAUG | 2832 |
| 590 | AAUUUCAU G CAGAACCA | 852 | UGGUUCUG GGAGGAAACUCC CU UCAAGGACAUCGUCCGGG AUGAAAUU | 2833 |
| 599 | CAGAACCA G UUGAUGUU | 853 | AACAUCAA GGAGGAAACUCC CU UCAACGACAUCGUCCGGG UGGUUCUG | 2834 |
| 605 | CAGUUGAU G UUUGGUCC | 854 | GGACCAAA GGAGGAAACUCC CU UCAAGGACAUCGUCCGGG AUCAACUG | 2835 |
| 610 | GAUGUUUG G UCCUGUGG | 855 | CCACAGGA GGAGGAAACUCC CU UCAAGGACAUCGUCCGGG CAAACAUC | 2836 |
| 615 | UUGGUCCU G UGGAAUAG | 856 | CUAUUCCA GGAGGAAACUCC CU UCAAGGACAUCGUCCGGG AGGACCAA | 2837 |
| 623 | GUGGAAUA G UACUUACU | 857 | AGUAAGUA GGAGGAAACUCC CU UCAAGGACAUCGUCCGGG UAUUCCAC | 2838 |
| 632 | UACUUACU G CAAUGCUC | 858 | GAGCAUUG GGAGGAAACUCC CU UCAAGGACAUCGUCCGGG AGUAAGUA | 2839 |
| 637 | ACUGCAAU G CUCGCUGG | 859 | CCAGCGAG GGAGGAAACUCC CU UCAAGGACAUCGUCCGGG AUUGCAGU | 2840 |
| 641 | CAAUGCUC G CUGGAGAA | 860 | UUCUCCAG GGAGGAAACUCC CU UCAAGGACAUCGUCCGGG GAGCAUUG | 2841 |
| 652 | GGAGAAUU G CCAUGGGA | 861 | UCCCAUGG GGAGGAAACUCC CU UCAAGGACAUCGUCCGGG AAUUCUCC | 2842 |
| 669 | CCAACCCA G UGACAGCU | 862 | AGCUGUCA GGAGGAAACUCC CU UCAAGGACAUCGUCCGGG UGGGUUGG | 2843 |
| 675 | CAGUGACA G CUGUCAGG | 863 | CCUGACAG GGAGGAAACUCC CU UCAAGGACAUCGUCCGGG UGUCACUG | 2844 |
| 676 | UGACAGCU G UCAGGAGU | 864 | ACUCCUGA GGAGGAAACUCC CU UCAAGGACAUCGUCCGGG AGCUGUCA | 2845 |
| 685 | UGUCAGGA G UAUUCUGA | 865 | UCAGAAUA GGAGGAAACUCC CU UCAAGGACAUCGUCCGGG UCCUGACA | 2846 |
| 743 | UCGAUUCU G CUCCUCCA | 866 | UAGAGGAG GGAGGAAACUCC CU UCAAGGACAUCGUCCGGG AGAAUCGA | 2847 |
| 752 | CUCCUCUA G CUCUGCUG | 867 | CAGCAGAG GGAGGAAACUCC CU UCAAGGACAUCGUCCGGG UAGAGGAG | 2848 |
| 757 | CUAGCUCU G CUGCAUAA | 868 | UUAUGCAG GGAGGAAACUCC CU UCAAGGACAUCGUCCGGG AGAGUAGC | 2849 |
| 760 | GCUCUGCU G CAUAAAAU | 869 | AUUUUAUG GGAGGAAACUCC CU UCAAGGACAUCGUCCGGG AGCAGAGC | 2850 |
| 773 | AAAUCUUA G UUGAGAAU | 870 | AUUCUCAA GGAGGAAACUCC CU UCAAGGACAUCGUCCGGG UAAGAUUU | 2851 |
| 788 | AUCCAUCA G CAAGAAUU | 871 | AAUUCUUG GGAGGAAACUCC CU UCAAGGACAUCGUCCGGG UGAUGGAU | 2852 |
| 826 | GAUAGAUG G UACAACAA | 872 | UUGUUGUA GGAGGAAACUCC CU UCAAGGACAUCGUCCGGG CAUCUAUC | 2853 |
| 851 | AGAAAGGG G CAAAAAGG | 873 | CCUUUUUG GGAGGAAACUCC CU UCAAGGACAUCGUCCGGG CCCUUUCU | 2854 |
| 859 | GCAAAAAG G CCCCGAGU | 874 | ACUCGGGG GGAGGAAACUCC CU UCAAGGACAUCGUCCGGG CUUUUUGC | 2855 |
| 866 | GGCCCCGA G UCACUUCA | 875 | UGAAGUGA GGAGGAAACUCC CU UCAAGGACAUCGUCCGGG UCGGGGCC | 2856 |
| 876 | CACUUCAG G UGGUGUGU | 876 | ACACACCA GGAGGAAACUCC CU UCAAGGACAUCGUCCGGG CUGAAGUG | 2857 |
| 879 | UUCAGGUG G UGUGUCAG | 877 | CUGACACA GGAGGAAACUCC CU UCAAGGACAUCGUCCGGG CACCUGAA | 2858 |
| 881 | CAGGUGGU G UGUCAGAG | 878 | CUCUGACA GGAGGAAACUCC CU UCAAGGACAUCGUCCGGG ACCACCUG | 2859 |
| 883 | GGUGGUGU G UCAGAGUC | 879 | GACUCUGA GGAGGAAACUCC CU UCAAGGACAUCGUCCGGG ACACCACC | 2860 |
| 889 | GUGUCAGA G UCUCCCAG | 880 | CUGGGAGA GGAGGAAACUCC CU UCAAGGACAUCGUCCGGG UCUGACAC | 2861 |
| 897 | GUCUCCCA G UGGAUUUU | 881 | AAAAUCCA GGAGGAAACUCC CU UCAAGGACAUCGUCCGGG UGGGAGAC | 2862 |
| 910 | UUUUCUAA G CACAUUCA | 882 | UGAAUGUG GGAGGAAACUCC CU UCAAGGACAUCGUCCGGG UUAGAAAA | 2863 |
| 941 | UCUCUCCA G UAAACAGU | 883 | ACUGUUUA GGAGGAAACUCC CU UCAAGGACAUCGUCCGGG UGGAGAGA | 2864 |
| 948 | AGUAAACA G UGCUUCUA | 884 | UAGAAGCA GGAGGAAACUCC CU UCAAGGACAUCGUCCGGG UGUUUACU | 2865 |
| 950 | UAAACAGU G CUUCUAGU | 885 | ACUAGAAG GGAGGAAACUCC CU UCAAGGACAUCGUCCGGG ACUGUUUA | 2866 |
| 957 | UGCUUCUA G UGAAGAAA | 886 | UUUCUUCA GGAGGAAACUCC CU UCAAGGACAUCGUCCGGG UAGAAGCA | 2867 |
| 968 | AAGAAAAU G UGAAGUAC | 887 | GUACUUCA GGAGGAAACUCC CU UCAAGGACAUCGUCCGGG AUUUUCUU | 2868 |
| 973 | AAUGUGAA G UACUCCAG | 888 | CUGGAGUA GGAGGAAACUCC CU UCAACGACAUCGUCCGGG UUCACAUU | 2869 |
| 981 | GUACUCCA G UUCUCAGC | 889 | GCUGAGAA GGAGGAAACUCC CU UCAAGGACAUCGUCCGGG UGGAGUAC | 2870 |
| 988 | AGUUCUCA G CCAGAACC | 890 | GGUUCUGG GGAGGAAACUCC CU UCAAGGACAUCGUCCGGG UGAGAACU | 2871 |
| 999 | AGAACCCC G CACAGGUC | 891 | GACCUGUG GGAGGAAACUCC CU UCAAGGACAUCGUCCGGG GGGGUUCU | 2872 |
| 1005 | CCGCACAG G UCUUUCCU | 892 | AGGAAAGA GGAGGAAACUCC CU UCAAGGACAUCGUCCGGG CUGUGCGG | 2873 |
| 1026 | GGAUACCA G CCCCUCAU | 893 | AUGAGGGG GGAGGAAACUCC CU UCAAGGACAUCGUCCGGG UGGUAUCC | 2874 |
| 1049 | AUAAAUUG G UACAAGGG | 894 | CCCUUGUA GGAGGAAACUCC CU UCAAGGACAUCGUCCGGG CAAUUUAU | 2875 |
| 1062 | AGGGAUCA G CUUUUCCC | 895 | GGGAAAAG GGAGGAAACUCC CU UCAAGGACAUCGUCCGGG UGAUCCCU | 2876 |
| 1072 | UUUUCCCA G CCCACAUG | 896 | CAUGUGGG GGAGGAAACUCC CU UCAAGGACAUCGUCCGGG UGGGAAAA | 2877 |
| 1080 | GCCCACAU G UCCUGAUC | 897 | GAUCAGGA GGAGGAAACUCC CU UCAAGGACAUCGUCCGGG AUGUGGGC | 2878 |
| 1093 | GAUCAUAU G CUUUUGAA | 898 | UUCAAAAG GGAGGAAACUCC CU UCAAGGACAUCGUCCGGG AUAUGAUC | 2879 |
| 1104 | UUUGAAUA G UCAGUUAC | 899 | GUAACUGA GGAGGAAACUCC CU UCAAGGACAUCGUCCGGG UAUUCAAA | 2880 |
| 1108 | AAUAGUCA G UUACUUGG | 900 | CCAAGUAA GGAGGAAACUCC CU UCAAGGACAUCGUCCGGG UGACUAUU | 2881 |
| 1116 | GUUACUUG G CACCCCAG | 901 | CUGGGGUG GGAGGAAACUCC CU UCAAGGACAUCGUCCGGG CAAGUAAC | 2882 |
| 1144 | AACCCCUG G CAGCGGUU | 902 | AACCGCCG GGAGGAAACUCC CU UCAAGGACAUCGUCCGGG CAGGGGUU | 2883 |
| 1147 | CCCUGGCA G CGGUUGGU | 903 | ACCAACCG GGAGGAAACUCC CU UCAAGGACAUCGUCCGGG UGCCAGGG | 2884 |
| 1150 | UGGCAGCG G UUGGUCAA | 904 | UUGACCAA GGAGGAAACUCC CU UCAAGGACAUCGUCCGGG CGCUGCCA | 2885 |
| 1154 | AGCGGUUG G UCAAAAGA | 905 | UCUUUUGA GGAGGAAACUCC CU UCAAGGACAUCGUCCGGG CAACCGCU | 2886 |
| 1190 | AAUUGGAU G CAGACAAA | 906 | UUUGUCUG GGAGGAAACUCC CU UCAAGGACAUCGUCCGGG AUCCAAUU | 2887 |
| 1209 | UUAUCAAU G CCUGAAAG | 907 | CUUUCAGG GGAGGAAACUCC CU UCAAGGACAUCGUCCGGG AUUGAUAA | 2888 |
| 1224 | AGAGACUU G UGAGAAGU | 908 | ACUUCUCA GGAGGAAACUCC CU UCAAGGACAUCGUCCGGG AAGUCUCU | 2889 |
| 1231 | UGUGAGAA G UUGGGCUA | 909 | UAGCCCAA GGAGGAAACUCC CU UCAAGGACAUCGUCCGGG UUCUCACA | 2890 |
| 1236 | GAAGUUGG G CUAUCAAU | 910 | AUUGAUAG GGAGGAAACUCC CU UCAAGGACAUCGUCCGGG CCAACUUC | 2891 |
| 1254 | GAAGAAAA G UUGUAUGA | 911 | UCAUACAA GGAGGAAACUCC CU UCAAGGACAUCGUCCGGG UUUUCUUC | 2892 |
| 1257 | GAAAAGUU G UAUGAAUC | 912 | GAUUCAUA GGAGGAAACUCC CU UCAAGGACAUCGUCCGGG AACUUUUC | 2893 |
| 1268 | UGAAUCAG G UUACUAUA | 913 | UAUAGUAA GGAGGAAACUCC CU UCAAGGACAUCGUCCGGG CUGAUUCA | 2894 |
| 1316 | UUUUCAAA G UGAAUUUG | 914 | CAAAUUCA GGAGGAAACUCC CU UCAAGGACAUCGUCCGGG UUUGAAAA | 2895 |
| 1324 | GUGAAUUU G UUAGAAAU | 915 | AUUUCUAA GGAGGAAACUCC CU UCAAGGACAUCGUCCGGG AAAUUCAC | 2896 |
| 1349 | AAAUAUUG G UUGACUUC | 916 | GAAGUCAA GGAGGAAACUCC CU UCAAGGACAUCGUCCGGG CAAUAUUU | 2897 |
| 1360 | GACUUCCG G CUUUCUAA | 917 | UUAGAAAG GGAGGAAACUCC CU UCAAGGACAUCGUCCGGG CGGAAGUC | 2898 |
| 1371 | UUCUAAGG G UGAUGGAU | 918 | AUCCAUCA GGAGGAAACUCC CU UCAAGGACAUCGUCCGGG CCUUAGAA | 2899 |
| 1384 | GGAUUGGA G UUCAAGAG | 919 | CUCUUGAA GGAGGAAACUCC CU UCAAGGACAUCGUCCGGG UCCAAUCC | 2900 |
| 1417 | AAAGGGAA G CUGAUUGA | 920 | UCAAUCAG GGAGGAAACUCC CU UCAAGGACAUCGUCCGGG UUCCCUUU | 2901 |
| 1430 | UUGAUAUU G UGAGCAGC | 921 | GCUGCUCA GGAGGAAACUCC CU UCAAGGACAUCGUCCGGG AAUAUCAA | 2902 |
| 1434 | UAUUGUGA G CAGCCAGA | 922 | UCUGGCUG GGAGGAAACUCC CU UCAAGGACAUCGUCCGGG UCACAAUA | 2903 |
| 1437 | UGUGAGCA G CCAGAAGG | 923 | CCUUCUGG GGAGGAAACUCC CU UCAAGGACAUCGUCCGGG UGCUCACA | 2904 |
| 1445 | GCCAGAAG G UUUGGCUU | 924 | AAGCCAAA GGAGGAAACUCC CU UCAAGGACAUCGUCCGGG CUUCUGGC | 2905 |
| 1450 | AAGGUUUG G CUUCCUGC | 925 | CCAGGAAG GGAGGAAACUCC CU UCAAGGACAUCGUCCGGG CAAACCUU | 2906 |
| 1457 | GGCUUCCU G CCACAUGA | 926 | UCAUGUGG GGAGGAAACUCC CU UCAAGGACAUCGUCCGGG AGGAAGCC | 2907 |
| 1477 | GACCAUCG G CUCUGGGG | 927 | CCCCAGAG GGAGGAAACUCC CU UCAAGGACAUCGUCCGGG CGAUGGUC | 2908 |
| 1493 | GAAUCCUG G UGAAUAUA | 928 | UAUAUUCA GGAGGAAACUCC CU UCAAGGACAUCGUCCGGG CAGGAUUC | 2909 |
| 1502 | UGAAUAUA G UGCUGCUA | 929 | UAGCAGCA GGAGGAAACUCC CU UCAAGGACAUCGUCCGGG UAUAUUCA | 2910 |
| 1504 | AAUAUAGU G CUGCUAUG | 930 | CAUAGCAG GGAGGAAACUCC CU UCAAGGACAUCGUCCGGG ACUAUAUU | 2911 |
| 1507 | AUAGUGCU G CUAUGUUG | 931 | CAACAUAG GGAGGAAACUCC CU UCAAGGACAUCGUCCGGG AGCACUAU | 2912 |
| 1512 | GCUGCUAU G UUGACAUU | 932 | AAUGUCAA GGAGGAAACUCC CU UCAAGGACAUCGUCCGGG AUAGCAGC | 2913 |
| 1545 | AUUAUCCU G UCCUGCAA | 933 | UUGCAGGA GGAGGAAACUCC CU UCAAGGACAUCGUCCGGG AGGAUAAU | 2914 |
| 1550 | CCUGUCCU G CAAACUGC | 934 | GCAGUUUG GGAGGAAACUCC CU UCAAGGACAUCGUCCCGG AGGACAGG | 2915 |
| 1557 | UGCAAACU G CAAAUAGU | 935 | ACUAUUUG GGAGGAAACUCC CU UCAAGGACAUCGUCCCGG AGUUUGCA | 2916 |
| 1564 | UGCAAAUA G UAGUUCCU | 936 | AGGAACUA GGAGGAAACUCC CU UCAAGGACAUCGUCCCGG UAUUUGCA | 2917 |
| 1567 | AAAUAGUA G UUCCUGAA | 937 | UUCAGGAA GGAGGAAACUCC CU UCAAGGACAUCGUCCGGG UACUAUUU | 2918 |
| 1576 | UUCCUGAA G UGUUCACU | 938 | AGUGAACA GGAGGAAACUCC CU UCAAGGACAUCGUCCGGG UUCAGGAA | 2919 |
| 1578 | CCUGAAGU G UUCACUUC | 939 | GAAGUGAA GGAGGAAACUCC CU UCAAGGACAUCGUCCGGG ACUUCAGG | 2920 |
| 1590 | ACUUCCCU G UUUAUCCA | 940 | UGGAUAAA GGAGGAAACUCC CU UCAAGGACAUCGUCCGGG AGGGAAGU | 2921 |
| 1619 | UUUAUUUU G UUUGUUCG | 941 | CGAACAAA GGAGGAAACUCC CU UCAAGGACAUCGUCCGGG AAAAUAAA | 2922 |
| 1623 | UUUUGUUU G UUCGGCAU | 942 | AUGCCGAA GGAGGAAACUCC CU UCAAGGACAUCGUCCGGG AAACAAAA | 2923 |
| 1628 | UUUGUUCG G CAUACAAA | 943 | UUUGUAUG GGAGGAAACUCC CU UCAAGGACAUCGUCCGGG CGAACAAA | 2924 |
| 1656 | UCUUAAUU G UAAGCAAA | 944 | UUUGCUUA GGAGGAAACUCC CU UCAAGGACAUCGUCCGGG AAUUAAGA | 2925 |
| 1660 | AAUUGUAA G CAAAACUU | 945 | AAGUUUUG GGAGGAAACUCC CU UCAAGGACAUCGUCCGGG UUACAAUU | 2926 |
| 1710 | UUCUUCAU G UGUGUUUA | 946 | UAAACACA GGAGGAAACUCC CU UCAAGGACAUCGUCCGGG AUGAAGAA | 2927 |
| 1712 | CUUCAUGU G UGUUUAGU | 947 | ACUAAACA GGAGGAAACUCC CU UCAAGGACAUCGUCCGGG ACAUGAAG | 2928 |
| 1714 | UCAUGUGU G UUUAGUAU | 948 | AUACUAAA GGAGGAAACUCC CU UCAAGGACAUCGUCCGGG ACACAUGA | 2929 |
| 1719 | UGUGUUUA G UAUCUGAA | 949 | UUCAGAUA GGAGGAAACUCC CU UCAAGGACAUCGUCCGGG UAAACACA | 2930 |
| 1743 | CUCAUCUG G UGGAAACC | 950 | GGUUUCCA GGAGGAAACUCC CU UCAAGGACAUCGUCCGGG CAGAUGAG | 2931 |
| 1754 | GAAACCAA G UUUCAGGG | 951 | CCCUGAAA GGAGGAAACUCC CU UCAAGGACAUCGUCCGGG UUGGUUUC | 2932 |
| 1770 | GGACAUGA G UUUUCCAG | 952 | CUGGAAAA GGAGGAAACUCC CU UCAAGGACAUCGUCCGGG UCAUGUCC | 2933 |
| 1778 | GUUUUCCA G CUUUUAUA | 953 | UAUAAAAG GGAGGAAACUCC CU UCAAGGACAUCGUCCGGG UGGAAAAC | 2934 |
| 1792 | AUACACAC G UAUCUCAU | 954 | AUGAGAUA GGAGGAAACUCC CU UCAAGGACAUCGUCCGGG GUGUGUAU | 2935 |
| 17 | AGUCCGCC G AGGUGCUC | 1186 | GAGCACCU GGAGGAAACUCC CU UCAAGGACAUCGUCCGGG GGCGACUG | 2936 |
| 19 | UCCGCCGA G GUGCUCGG | 1187 | CCGAGCAC GGAGGAAACUCC CU UCAAGGACAUCGUCCGGG UCGGCGGA | 2937 |
| 26 | AGGUGCUC G GUGGAGUC | 1188 | GACUCCAC GGAGGAAACUCC CU UCAAGGACAUCGUCCGGG GAGCACCU | 2938 |
| 29 | UGCUCGGU G GAGUCAUG | 1189 | CAUGACUC GGAGGAAACUCC CU UCAAGGACAUCGUCCGGG ACCGAGCA | 2939 |
| 30 | GCUCGGUG G AGUCAUGG | 1190 | CCAUGACU GGAGGAAACUCC CU UCAAGGACAUCGUCCGGG CACCGAGC | 2940 |
| 37 | GGAGUCAU G GCAGUGCC | 1191 | GGCACUGC GGAGGAAACUCC CU UCAAGGACAUCGUCCGGG AUGACUCC | 2941 |
| 52 | CCCUUUGU G GAAGACUG | 1192 | CAGUCUUC GGAGGAAACUCC CU UCAAGGACAUCGUCCGGG ACAAAGGG | 2942 |
| 53 | CCUUUGUG G AAGACUGG | 1193 | CCAGUCUU GGAGGAAACUCC CU UCAAGGACAUCGUCCGGG CACAAAGG | 2943 |
| 56 | UUGUGGAA G ACUGGGAC | 1194 | GUCCCAGU GGAGGAAACUCC CU UCAAGGACAUCGUCCGGG UUCCACAA | 2944 |
| 60 | GGAAGACU G GGACUUGG | 1195 | CCAAGUCC GGAGGAAACUCC CU UCAAGGACAUCGUCCGGG AGUCUUCC | 2945 |
| 61 | GAAGACUG G GACUUGGU | 1196 | ACCAAGUC GGAGGAAACUCC CU UCAAGGACAUCGUCCGGG CAGUCUUC | 2946 |
| 62 | AAGACUGG G ACUUGGUG | 1197 | CACCAAGU GGAGGAAACUCC CU UCAAGGACAUCGUCCGGG CCAGUCUU | 2947 |
| 67 | UGGGACUU G GUGCAAAC | 1198 | GUUUGCAC GGAGGAAACUCC CU UCAAGGACAUCGUCCGGG AAGUCCCA | 2948 |
| 79 | CAAACCCU G GGAGAAGG | 1199 | CCUUCUCC GGAGGAAACUCC CU UCAAGGACAUCGUCCGGG AGGGUUUG | 2949 |
| 80 | AAACCCUG G GAGAAGGU | 1200 | ACCUUCUC GGAGGAAACUCC CU UCAAGGACAUCGUCCGGG CAGGGUUU | 2950 |
| 81 | AACCCUGG G AGAAGGUG | 1201 | CACCUUCU GGAGGAAACUCC CU UCAAGGACAUCGUCCGGG CCAGGGUU | 2951 |
| 83 | CCCUGGGA G AAGGUGCC | 1202 | GGCACCUU GGAGGAAACUCC CU UCAAGGACAUCGUCCGGG UCCCAGGG | 2952 |
| 86 | UGGGAGAA G GUGCCUAU | 1203 | AUAGGCAC GGAGGAAACUCC CU UCAAGGACAUCGUCCGGG UUCUCCCA | 2953 |
| 95 | GUGCCUAU G GAGAAGUU | 1204 | AACUUCUC GGAGGAAACUCC CU UCAAGGACAUCGUCCGGG AUAGGCAC | 2954 |
| 96 | UGCCUAUG G AGAAGUUC | 1205 | GAACUUCU GGAGGAAACUCC CU UCAAGGACAUCGUCCGGG CAUAGGCA | 2955 |
| 98 | CCUAUGGA G AAGUUCAA | 1206 | UUGAACUU GGAGGAAACUCC CU UCAAGGACAUCGUCCGGG UCCAUAGG | 2956 |
| 115 | CUUGCUGU G AAUAGAGU | 1207 | ACUCUAUU GGAGGAAACUCC CU UCAAGGACAUCGUCCGGG ACAGCAAG | 2957 |
| 120 | UGUGAAUA G AGUAACUG | 1208 | CAGUUACU GGAGGAAACUCC CU UCAAGGACAUCGUCCGGG UAUUCACA | 2958 |
| 128 | GAGUAACU G AAGAAGCA | 1209 | UGCUUCUU GGAGGAAACUCC CU UCAAGGACAUCGUCCGGG AGUUACUC | 2959 |
| 131 | UAACUGAA G AAGCAGUC | 1210 | GACUGCUU GGAUUAAACUCC CU UCAAGGACAUCGUCCGGG UUCAGUUA | 2960 |
| 145 | GUCGCAGU G AAGAUUGU | 1211 | ACAAUCUU GGAGGAAACUCC CU UCAAGGACAUCGUCCGGG ACUGCGAC | 2961 |
| 148 | GCAGUGAA G AUUGUAGA | 1212 | UCUACAAU GGAGGAAACUCC CU UCAAGGACAUCGUCCGGG UUCACUGC | 2962 |
| 155 | AGAUUGUA G AUAUGAAG | 1213 | CUUCAUAU GGAGGAAACUCC CU UCAAGGACAUCGUCCGGG UACAAUCU | 2963 |
| 160 | GUAGAUAU G AAGCGUGC | 1214 | GCACGCUU GGAGGAAACUCC CU UCAAGGACAUCGUCCGCG AUAUCUAC | 2964 |
| 173 | GUGCCGUA G ACUGUCCA | 1215 | UGGACAGU GGAGGAAACUCC CU UCAAGGACAUCGUCCGGG UACGGCAC | 2965 |
| 182 | ACUGUCCA G AAAAUAUU | 1216 | AAUAUUUU GGAGGAAACUCC CU UCAAGGACAUCGUCCGGG UGGACAGU | 2966 |
| 193 | AAUAUUAA G AAAGAGAU | 1217 | AUCUCUUU GGAGGAAACUCC CU UCAAGGACAUCGUCCGGG UUAAUAUU | 2967 |
| 197 | UUAAGAAA G AGAUCUGU | 1218 | ACAGAUCU GGAGGAAACUCC CU UCAAGGACAUCGUCCGGG UUUCUUAA | 2968 |
| 199 | AAGAAAGA G AUCUGUAU | 1219 | AUACAGAU GGAGGAAACUCC CU UCAAGGACAUCGUCCGGG UCUUUCUU | 2969 |
| 227 | UAAAUCAU G AAAAUGUA | 1220 | UACAUUUU GGAGGAAACUCC CU UCAAGGACAUCGUCCGGG AUGAUUUA | 2970 |
| 248 | AAUUCUAU G GUCACAGG | 1221 | CCUGUGAC GGAGGAAACUCC CU UCAAGGACAUCGUCCGGG AUAGAAUU | 2971 |
| 255 | UGGUCACA G GAGAGAAG | 1222 | CUUCUCUC GGAGGAAACUCC CU UCAAGGACAUCGUCCGGG UGUGACCA | 2972 |
| 256 | GGUCACAG G AGAGAAGG | 1223 | CCUUCUCU GGAGGAAACUCC CU UCAAGGACAUCGUCCGGG CUGUGACC | 2973 |
| 258 | UCACAGGA G AGAAGGCA | 1224 | UGCCUUCU GGAGGAAACUCC CU UCAAGGACAUCGUCCGGG UCCUGUGA | 2974 |
| 260 | ACAGGAGA G AAGGCAAU | 1225 | AUUGCCUU GGAGGAAACUCC CU UCAAGGACAUCGUCCGGG UCUCCUGU | 2975 |
| 263 | GGAGAGAA G GCAAUAUC | 1226 | GAUAUUGC GGAGGAAACUCC CU UCAAGGACAUCGUCCGGG UUCUCUCC | 2976 |
| 286 | UUAUUUCU G GAGUACUG | 1227 | CAGUACUC GGAGGAAACUCC CU UCAAGGACAUCGUCCGGG AGAAAUAA | 2977 |
| 287 | UAUUUCUG G AGUACUGU | 1228 | ACAGUACU GGAGGAAACUCC CU UCAAGGACAUCGUCCGGG CAGAAAUA | 2978 |
| 299 | ACUGUAGU G GAGGAGAG | 1229 | CUCUCCUC GGAGGAAACUCC CU UCAAGGACAUCGUCCGGG ACUACAGU | 2979 |
| 300 | CUGUAGUG G AGGAGAGC | 1230 | GCUCUCCU GGAGGAAACUCC CU UCAAGGACAUCGUCCGGG CACUACAG | 2980 |
| 302 | GUAGUGGA G GAGAGCUU | 1231 | AAGCUCUC GGAGGAAACUCC CU UCAAGGACAUCGUCCGGG UCCACUAC | 2981 |
| 303 | UAGUGGAG G AGAGCUUU | 1232 | AAAGCUCU GGAGGAAACUCC CU UCAAGGACAUCGUCCGGG CUCCACUA | 2982 |
| 305 | GUGGAGGA G AGCUUUUU | 1233 | AAAAAGCU GGAGGAAACUCC CU UCAAGGACAUCGUCCGGG UCCUCCAC | 2983 |
| 314 | AGCUUUUU G ACAGAAUA | 1234 | UAUUCUGU GGAGGAAACUCC CU UCAAGGACAUCGUCCGGG AAAAAGCU | 2984 |
| 318 | UUUUGACA G AAUAGAGC | 1235 | GCUCUAUU GGAGGAAACUCC CU UCAAGGACAUCGUCCGGG UGUCAAAA | 2985 |
| 323 | ACAGAAUA G AGCCAGAC | 1236 | GUCUGGCU GGAGGAAACUCC CU UCAAGGACAUCGUCCGGG UAUUCUGU | 2986 |
| 329 | UAGAGCCA G ACAUAGGC | 1237 | GCCUAUGU GGAGGAAACUCC CU UCAAGGACAUCGUCCGGG UGGCUCUA | 2987 |
| 335 | CAGACAUA G GCAUGCCU | 1238 | AGGCAUGC GGAGGAAACUCC CU UCAAGGACAUCGUCCGGG UAUGUCUG | 2988 |
| 344 | GCAUGCCU G AACCAGAU | 1239 | AUCUGGUU GGAGGAAACUCC CU UCAAGGACAUCGUCCGGG AGGCAUGC | 2989 |
| 350 | CUGAACCA G AUGCUCAG | 1240 | CUGAGCAU GGAGGAAACUCC CU UCAAGGACAUCGUCCGGG UGGUUCAG | 2990 |
| 358 | GAUGCUCA G AGAUUCUU | 1241 | AAGAAUCU GGAGGAAACUCC CU UCAAGGACAUCGUCCGGG UGAGCAUC | 2991 |
| 360 | UGCUCAGA G AUUCUUCC | 1242 | GGAAGAAU GGAGGAAACUCC CU UCAAGGACAUCGUCCGGG UCUGAGCA | 2992 |
| 379 | CAACUCAU G GCAGGGGU | 1243 | ACCCCUGC GGAGGAAACUCC CU UCAAGGACAUCGUCCGGG AUGAGUUG | 2993 |
| 383 | UCAUGGCA G GGGUGGUU | 1244 | AACCACCC GGAGGAAACUCC CU UCAAGGACAUCGUCCGGG UGCCAUGA | 2994 |
| 384 | CAUGGCAG G GGUGGUUU | 1245 | AAACCACC GGAGGAAACUCC CU UCAAGGACAUCGUCCGGG CUGCCAUG | 2995 |
| 385 | AUGGCAGG G GUGGUUUA | 1246 | UAAACCAC GGAGGAAACUCC CU UCAAGGACAUCGUCCGGG CCUGCCAU | 2996 |
| 388 | GCAGGGGU G GUUUAUCU | 1247 | AGAUAAAC GGAGGAAACUCC CU UCAAGGACAUCGUCCGGG ACCCCUGC | 2997 |
| 401 | AUCUGCAU G GUAUUGGA | 1248 | UCCAAUAC GGAGGAAACUCC CU UCAAGGACAUCGUCCGGG AUGCAGAU | 2998 |
| 407 | AUGGUAUU G GAAUAACU | 1249 | AGUUAUUC GGAGGANXCUCC CU UCAAGGACAUCGUCCGGG AAUACCAU | 2999 |
| 408 | UGGUAUUG G AAUAACUC | 1250 | GAGUUAUU GGAGGAAACUCC CU UCAAGGACAUCGUCCGGG CAAUACCA | 3000 |
| 420 | AACUCACA G GGAUAUUA | 1251 | UAAUAUCC GGAGGAAACUCC CU UCAAGGACAUCGUCCGGG UGUGAGUU | 3001 |
| 421 | ACUCACAG G GAUAUUAA | 1252 | UUAAUAUC GGAGGAAACUCC CU UCAAGGACAUCGUCCGGG CUGUGAGU | 3002 |
| 422 | CUCACAGG G AUAUUAAA | 1253 | UUUAAUAU GGAGGAAACUCC CU UCAAGGACAUCGUCCGGG CCUGUGAG | 3003 |
| 434 | UUAAACCA G AAAAUCUU | 1254 | AAGAUUUU GGAGGAAACUCC CU UCAAGGACAUCGUCCGGG UGGUUUAA | 3004 |
| 448 | CUUCUGUU G GAUGAAAG | 1255 | CUUUCAUC GGAGGAAACUCC CU UCAAGGACAUCGUCCGGG AACAGAAG | 3005 |
| 449 | UUCUGUUG G AUGAAAGG | 1256 | CCUUUCAU GGAGGAAACUCC CU UCAAGGACAUCGUCCGGG CAACAGAA | 3006 |
| 452 | UGUUGGAU G AAAGGGAU | 1257 | AUCCCUUU GGAGGAAACUCC CU UCAAGGACAUCGUCCGGG AUCCAACA | 3007 |
| 456 | GGAUGAAA G GGAUAACC | 1258 | GGUUAUCC GGAGGAAACUCC CU UCAAGGACAUCGUCCGGG UUUCAUCC | 3008 |
| 457 | GAUGAAAG G GAUAACCU | 1259 | AGGUUAUC GGAGGAAACUCC CU UCAAGGACAUCGUCCGGG CUUUCAUC | 3009 |
| 458 | AUGAAAGG G AUAACCUC | 1260 | GAGGUUAU GGAGGAAACUCC CU UCAAGGACAUCGUCCGGG CCUUUCAU | 3010 |
| 476 | AAAUCUCA G ACUUUGGC | 1261 | GCCAAAGU GGAGGAAACUCC CU UCAAGGACAUCGUCCGGG UGAGAUUU | 3011 |
| 482 | CAGACUUU G GCUUGGCA | 1262 | UGCCAAGC GGAGGAAACUCC CU UCAAGGACAUCGUCCGGG AAAGUCUG | 3012 |
| 487 | UUUGGCUU G GCAACAGU | 1263 | ACUGUUGC GGAGGAAACUCC CU UCAAGGACAUCGUCCGGG AAGCCAAA | 3013 |
| 501 | AGUAUUUC G GUAUAAUA | 1264 | UAUUAUAC GGAGGAAACUCC CU UCAAGGACAUCGUCCGGG GAAAUACU | 3014 |
| 515 | AUAAUCGU G AGCGUUUG | 1265 | CAAACGCU GGAGGAAACUCC CU UCAAGGACAUCGUCCGGG ACGAUUAU | 3015 |
| 526 | CGUUUGUU G AACAAGAU | 1266 | AUCUUGUU GGAGGAAACUCC CU UCAAGGACAUCGUCCGGG AACAAACG | 3016 |
| 532 | UUGAACAA G AUGUGUGG | 1267 | CCACACAU GGAGGAAACUCC CU UCAAGGACAUCGUCCGGG UUGUUCAA | 3017 |
| 539 | AGAUGUGU G GUACUUUA | 1268 | UAAAGUAC GGAGGAAACUCC CU UCAAGGACAUCGUCCGGG ACACAUCU | 3018 |
| 563 | UUGCUCCA G AACUUCUG | 1269 | CAGAAGUU GGAGGAAACUCC CU UCAAGGACAUCGUCCGGG UGGAGCAA | 3019 |
| 571 | GAACUUCU G AAGAGAAG | 1270 | CUUCUCUU GGAGGAAACUCC CU UCAAGGACAUCGUCCGGG AGAAGUUC | 3020 |
| 574 | CUUCUGAA G AGAAGAGA | 1271 | UCUCUUCU GGAGGAAACUCC CU UCAAGGACAUCGUCCGGG UUCAGAAG | 3021 |
| 576 | UCUGAAGA G AAGAGAAU | 1272 | AUUCUCUU GGAGGAAACUCC CU UCAAGGACAUCGUCCGGG UCUUCAGA | 3022 |
| 579 | GAAGAGAA G AGAAUUUC | 1273 | GAAAUUCU GGAGGAAACUCC CU UCAACGACAUCGUCCGGG UUCUCUUC | 3023 |
| 581 | AGAGAAGA G AAUUUCAU | 1274 | AUGAAAUU GGAGGAAACUCC CU UCAAGGACAUCGUCCGGG UCUUCUCU | 3024 |
| 593 | UUCAUGCA G AACCAGUU | 1275 | AACUGGUU GGAGGAAACUCC CU UCAAGGACAUCGUCCGGG UGCAUGAA | 3025 |
| 602 | AACCAGUU G AUGUUUGG | 1276 | CCAAACAU GGAGGAAACUCC CU UCAAGGACAUCGUCCGGG AACUGGUU | 3026 |
| 609 | UGAUGUUU G GUCCUGUG | 1277 | CACAGGAC GGAGGAAACUCC CU UCAAGGACAUCGUCCGGG AAACAUCA | 3027 |
| 617 | GGUCCUGU G GAAUAGUA | 1278 | UACUAUUC GGAGGAAACUCC CU UCAAGGACAUCGUCCGGG ACAGGACC | 3028 |
| 618 | GUCCUGUG G AAUAGUAC | 1279 | GUACUAUU GGAGGAAACUCC CU UCAAGGACAUCGUCCGGG CACAGGAC | 3029 |
| 644 | UGCUCGCU G GAGAAUUG | 1280 | CAAUUCUC GGAGGAAACUCC CU UCAAGGACAUCGUCCGGG AGCGAGCA | 3030 |
| 645 | GCUCGCUG G AGAAUUGC | 1281 | GCAAUUCU GGAGGAAACUCC CU UCAAGGACAUCGUCCGGG CAGCGAGC | 3031 |
| 647 | UCGCUGGA G AAUUGCCA | 1282 | UGGCAAUU GGAGGAAACUCC CU UCAAGGACAUCGUCCGGG UCCAGCGA | 3032 |
| 657 | AUUGCCAU G GGACCAAC | 1283 | GUUGGUCC GGAGGAAACUCC CU UCAAGGACAUCGUCCGGG AUGGCAAU | 3033 |
| 658 | UUGCCAUG G GACCAACC | 1284 | GGUUGGUC GGAGGAAACUCC CU UCAAGGACAUCGUCCGGG CAUGGCAA | 3034 |
| 659 | UGCCAUGG G ACCAACCC | 1285 | GGGUUGGU GGAGGAAACUCC CU UCAAGGACAUCGUCCGGG CCAUGGCA | 3035 |
| 671 | AACCCAGU G ACAGCUGU | 1286 | ACAGCUGU GGAGGAAACUCC CU UCAAGGACAUCGUCCGGG ACUGGGUU | 3036 |
| 682 | AGCUGUCA G GAGUAUUC | 1287 | GAAUACUC GGAGGAAACUCC CU UCAAGGACAUCGUCCGGG UGACAGCU | 3037 |
| 683 | GCUGUCAG G AGUAUUCU | 1288 | AGAAUACU GGAGGAAACUCC CU UCAAGGACAUCGUCCGGG CUGACAGC | 3038 |
| 692 | AGUAUUCU G ACUGGAAA | 1289 | UUUCCAGU GGAGGAAACUCC CU UCAAGGACAUCGUCCGGG AGAAUACU | 3039 |
| 696 | UUCUGACU G GAAAGAAA | 1290 | UUUCUUUC GGAGGAAACUCC CU UCAAGGACAUCGUCCGGG AGUCAGAA | 3040 |
| 697 | UCUGACUG G AAAGAAAA | 1291 | UUUUCUUU GGAGGAAACUCC CU UCAAGGACAUCGUCCGGG CAGUCAGA | 3041 |
| 701 | ACUGGAAA G AAAAAAAA | 1292 | UUUUUUUU GGAGGAAACUCC CU UCAAGGACAUCGUCCGGG UUUCCAGU | 3042 |
| 726 | CAACCCUU G GAAAAAAA | 1293 | UUUUUUUC GGAGGAAACUCC CU UCAAGGACAUCGUCCGGG AAGGGUUG | 3043 |
| 727 | AACCCUUG G AAAAAAAU | 1294 | AUUUUUUU GGAGGAAACUCC CU UCAAGGACAUCGUCCGGG CAAGGGUU | 3044 |
| 737 | AAAAAAUC G AUUCUGCU | 1295 | AGCAGAAU GGAGGAAACUCC CU UCAAGGACAUCGUCCGGG GAUUUUUU | 3045 |
| 776 | UCUUAGUU G AGAAUCCA | 1296 | UGGAUUCU GGAGGAAACUCC CU UCAAGGACAUCGUCCGGG AACUAAGA | 3046 |
| 778 | UUAGUUGA G AAUCCAUC | 1297 | GAUGGAUU GGAGGAAACUCC CU UCAAGGACAUCGUCCGGG UCAACUAA | 3047 |
| 792 | AUCAGCAA G AAUUACCA | 1298 | UGGUAAUU GGAGGAAACUCC CU UCAAGGACAUCGUCCGGG UUGCUGAU | 3048 |
| 806 | CCAUUCCA G ACAUCAAA | 1299 | UUUGAUGU GGAGGAAACUCC CU UCAAGGACAUCGUCCGGG UGGAAUGG | 3049 |
| 818 | UCAAAAAA G AUAGAUGG | 1300 | CCAUCUAU GGAGGAAACUCC CU UCAAGGACAUCGUCCGGG UUUUUUGA | 3050 |
| 822 | AAAAGAUA G AUGGUACA | 1301 | UGUACCAU GGAGGAAACUCC CU UCAAGGACAUCGUCCGGG UAUCUUUU | 3051 |
| 825 | AGAUAGAU G GUACAACA | 1302 | UGUUGUAC GGAGGAAACUCC CU UCAAGGACAUCGUCCGGG AUCUAUCU | 3052 |
| 844 | CCCCUCAA G AAAGGGGC | 1303 | GCCCCUUU GGAGGAAACUCC CU UCAAGGACAUCGUCCGGG UUGAGGGG | 3053 |
| 848 | UCAAGAAA G GGGCAAAA | 1304 | UUUUGCCC GGAGGAAACUCC CU UCAAGGACAUCGUCCGGG UUUCUUGA | 3054 |
| 849 | CAAGAAAG G GGCAAAAA | 1305 | UUUUUGCC GGAGGAAACUCC CU UCAAGGACAUCGUCCGGG CUUUCUUG | 3055 |
| 850 | AAGAAAGG G GCAAAAAG | 1306 | CUUUUUGC GGAGGAAACUCC CU UCAAGGACAUCGUCCGGG CCUUUCUU | 3056 |
| 858 | GGCAAAAA G GCCCCGAG | 1307 | CUCGGGGC GGAGGAAACUCC CU UCAAGGACAUCGUCCGGG UUUUUGCC | 3057 |
| 864 | AAGGCCCC G ACUCACUU | 1308 | AAGUGACU GGAGGAAACUCC CU UCAAGGACAUCGUCCGGG GGGGCCUU | 3058 |
| 875 | UCACUUCA G GUGGUGUG | 1309 | CACACCAC GGAGGAAACUCC CU UCAAGGACAUCGUCCGGG UGAAGUGA | 3059 |
| 878 | CUUCAGGU G GUGUGUCA | 1310 | UGACACAC GGAGGAAACUCC CU UCAAGGACAUCGUCCGGG ACCUGAAG | 3060 |
| 887 | GUGUGUCA G AGUCUCCC | 1311 | GGGAGACU GGAGGAAACUCC CU UCAAGGACAUCGUCCGGG UGACACAC | 3061 |
| 899 | CUCCCAGU G GAUUUUCU | 1312 | AGAAAAUC GGAGGAAACUCC CU UCAAGGACAUCGUCCGGG ACUGGGAG | 3062 |
| 900 | UCCCAGUG G AUUUUCUA | 1313 | UAGAAAAU GGAGGAAACUCC CU UCAAGGACAUCGUCCGGG CACUGGGA | 3063 |
| 928 | UCCAAUUU G GACUUCUC | 1314 | GAGAAGUC GGAGGAAACUCC CU UCAAGGACAUCGUCCGGG AAAUUGGA | 3064 |
| 929 | CCAAUUUG G ACUUCUCU | 1315 | AGAGAAGU GGAGGAAACUCC CU UCAAGGACAUCGUCCGGG CAAAUUGG | 3065 |
| 959 | CUUCUAGU G AAGAAAAU | 1316 | AUUUUCUU GGAGGAAACUCC CU UCAAGGACAUCGUCCGGG ACUAGAAG | 3066 |
| 962 | CUAGUGAA G AAAAUGUG | 1317 | CACAUUUU GGAGGAAACUCC CU UCAAGGACAUCGUCCGGG UUCACUAG | 3067 |
| 970 | GAAAAUGU G AAGUACUC | 1318 | GAGUACUU GGAGGAAACUCC CU UCAAGGACAUCGUCCGGG ACAUUUUC | 3068 |
| 992 | CUCAGCCA G AACCCCGC | 1319 | GCGGGGUU GGAGGAAACUCC CU UCAAGGACAUCGUCCGGG UGGCUGAG | 3069 |
| 1004 | CCCGCACA G GUCUUUCC | 1320 | GGAAAGAC GGAGGAAACUCC CU UCAAGGACAUCGUCCGGG UGUGCGGG | 3070 |
| 1017 | UUCCUUAU G GGAUACCA | 1321 | UGGUAUCC GGAGGAAACUCC CU UCAAGGACAUCGUCCGGG AUAAGGAA | 3071 |
| 1018 | UCCUUAUG G GAUACCAG | 1322 | CUGGUAUC GGAGGAAACUCC CU UCAAGGACAUCGUCCGGG CAUAAGGA | 3072 |
| 1019 | CCUUAUGG G AUACCAGC | 1323 | GCUGGUAU GGAGGAAACUCC CU UCAAGGACAUCGUCCGGG CCAUAAGG | 3073 |
| 1040 | CAUACAUU G AUAAAUUG | 1324 | CAAUUUAU GGAGGAAACUCC CU UCAAGGACAUCGUCCGGG AAUGUAUG | 3074 |
| 1048 | GAUAAAUU G GUACAAGG | 1325 | CCUUGUAC GGAGGAAACUCC CU UCAAGGACAUCGUCCGGG AAUUUAUC | 3075 |
| 1055 | UGGUACAA G GGAUCAGC | 1326 | GCUGAUCC GGAGGAAACUCC CU UCAAGGACAUCGUCCGGG UUGUACCA | 3076 |
| 1056 | GGUACAAG G GAUCAGCU | 1327 | AGCUGAUC GGAGGAAACUCC CU UCAAGGACAUCGUCCGGG CUUGUACC | 3077 |
| 1057 | GUACAAGG G AUCAGCUU | 1328 | AAGCUGAU GGAGGAAACUCC CU UCAAGGACAUCGUCCGGG CCUUGUAC | 3078 |
| 1085 | CAUGUCCU G AUCAUAUG | 1329 | CAUAUGAU GGAGGAAACUCC CU UCAAGGACAUCGUCCGGG AGGACAUG | 3079 |
| 1099 | AUGCUUUU G AAUAGUCA | 1330 | UGACUAUU GGAGGAAACUCC CU UCAAGGACAUCGUCCGGG AAAAGCAU | 3080 |
| 1115 | AGUUACUU G GCACCCCA | 1331 | UGGGGUGC GGAGGAAACUCC CU UCAAGGACAUCGUCCGGG AAGUAACU | 3081 |
| 1124 | GCACCCCA G GAUCCUCA | 1332 | UGAGGAUC GGAGGAAACUCC CU UCAAGGACAUCGUCCGGG UGGGGUGC | 3082 |
| 1125 | CACCCCAG G AUCCUCAC | 1333 | GUGAGGAU GGAGGAAACUCC CU UCAAGGACAUCGUCCGGG CUGGGGUG | 3083 |
| 1135 | UCCUCACA G AACCCCUG | 1334 | CAGGGGUU GGAGGAAACUCC CU UCAAGGACAUCGUCCGGG UGUGAGGA | 3084 |
| 1143 | GAACCCCU G GCAGCGGU | 1335 | ACCGCUGC GGAGGAAACUCC CU UCAAGGACAUCGUCCGGG AGGGGUUC | 3085 |
| 1149 | CUGGCAGC G GUUGGUCA | 1336 | UGACCAAC GGAGGAAACUCC CU UCAAGGACAUCGUCCGCG GCUGCCAG | 3086 |
| 1153 | CAGCGGUU G GUCAAAAG | 1337 | CUUUUGAC GGAGGAAACUCC CU UCAAGGACAUCGUCCGGG AACCGCUG | 3087 |
| 1161 | GGUCAAAA G AAUGACAC | 1338 | GUGUCAUU GGAGGAAACUCC CU UCAAGGACAUCGUCCGGG UUUUGACC | 3088 |
| 1165 | AAAAGAAU G ACACGAUU | 1339 | AAUCGUGU GGAGGAAACUCC CU UCAAGGACAUCGUCCGCG AUUCUUUU | 3089 |
| 1170 | AAUGACAC G AUUCUUUA | 1340 | UAAAGAAU GGAGGAAACUCC CU UCAAGGACAUCGUCCGGG GUGUCAUU | 3090 |
| 1186 | ACCAAAUU G GAUGCAGA | 1341 | UCUGCAUC GGAGGAAACUCC CU UCAAGGACAUCGUCCGGG AAUUUGGU | 3091 |
| 1187 | CCAAAUUG G AUGCAGAC | 1342 | GUCUGCAU GGAGGAAACUCC CU UCAAGGACAUCGUCCGGG CAAUUUGG | 3092 |
| 1193 | UGGAUGCA G ACAAAUCU | 1343 | AGAUUUGU GGAGGAAACUCC CU UCAAGGACAUCGUCCGGG UGCAUCCA | 3093 |
| 1213 | CAAUGCCU G AAAGAGAC | 1344 | GUCUCUUU GGAGGAAACUCC CU UCAAGGACAUCGUCCGGG AGGCAUUG | 3094 |
| 1217 | GCCUGAAA G AGACUUGU | 1345 | ACAAGUCU GGAGGAAACUCC CU UCAAGGACAUCGUCCGGG UUUCAGGC | 3095 |
| 1219 | CUGAAAGA G ACUUGUGA | 1346 | UCACAAGU GGAGGAAACUCC CU UCAAGGACAUCGUCCGGG UCUUUCAG | 3096 |
| 1226 | AGACUUGU G AGAAGUUG | 1347 | CAACUUCU GGAGGAAACUCC CU UCAAGGACAUCGUCCGGG ACAAGUCU | 3097 |
| 1228 | ACUUGUGA G AAGUUGGG | 1348 | CCCAACUU GGAGGAAACUCC CU UCAAGGACAUCGUCCGGG UCACAAGU | 3098 |
| 1234 | GAGAAGUU G GGCUAUCA | 1349 | UGAUAGCC GGAGGAAACUCC CU UCAAGGACAUCGUCCGGG AACUUCUC | 3099 |
| 1235 | AGAAGUUG G GCUAUCAA | 1350 | UUGAUAGC GGAGGAAACUCC CU UCAAGGACAUCGUCCGGG CAACUUCU | 3100 |
| 1245 | CUAUCAAU G GAAGAAAA | 1351 | UUUUCUUC GGAGGAAACUCC CU UCAAGGACAUCGUCCGGG AUUGAUAG | 3101 |
| 1246 | UAUCAAUG G AAGAAAAG | 1352 | CUUUUCUU GGAGGAAACUCC CU UCAAGGACAUCGUCCGGG CAUUGAUA | 3102 |
| 1249 | CAAUGGAA G AAAAGUUG | 1353 | CAACUUUU GGAGGAAACUCC CU UCAAGGACAUCGUCCGGG UUCCAUUG | 3103 |
| 1261 | AGUUGUAU G AAUCAGGU | 1354 | ACCUGAUU GGAGGAAACUCC CU UCAAGGACAUCGUCCGGG AUACAACU | 3104 |
| 1267 | AUGAAUCA G GUUACUAU | 1355 | AUAGUAAC GGAGGAAACUCC CU UCAAGGACAUCGUCCGGG UGAUUCAU | 3105 |
| 1286 | CAACAACU G AUAGGAGA | 1356 | UCUCCUAU GGAGGAAACUCC CU UCAAGGACAUCGUCCGGG AGUUGUUG | 3106 |
| 1290 | AACUGAUA G GAGAAACA | 1357 | UGUUUCUC GGAGGAAACUCC CU UCAAGGACAUCGUCCGGG UAUCAGUU | 3107 |
| 1291 | ACUGAUAG G AGAAACAA | 1358 | UUGUUUCU GGAGGAAACUCC CU UCAAGGACAUCGUCCGGG CUAUCAGU | 3108 |
| 1293 | UGAUACGA G AAACAAUA | 1359 | UAUUGUUU GGAGGAAACUCC CU UCAAGGACAUCGUCCGGG UCCUAUCA | 3109 |
| 1318 | UUCAAAGU G AAUUUGUU | 1360 | AACAAAUU GGAGGAAACUCC CU UCAAGGACAUCGUCCGGG ACUUUGAA | 3110 |
| 1328 | AUUUGUUA G AAAUGGAU | 1361 | AUCCAUUU GGAGGAAACUCC CU UCAAGGACAUCGUCCGGG UAACAAAU | 3111 |
| 1333 | UUAGAAAU G GAUGAUAA | 1362 | UUAUCAUC GGAGGAAACUCC CU UCAAGGACAUCGUCCGGG AUUUCUAA | 3112 |
| 1334 | UAGAAAUG G AUGAUAAA | 1363 | UUUAUCAU GGAGGAAACUCC CU UCAAGGACAUCGUCCGGG CAUUUCUA | 3113 |
| 1337 | AAAUGGAU G AUAAAAUA | 1364 | UAUUUUAU GGAGGAAACUCC CU UCAAGGACAUCGUCCGGG AUCCAUUU | 3114 |
| 1348 | AAAAUAUU G GUUGACUU | 1365 | AAGUCAAC GGAGGAAACUCC CU UCAAGGACAUCGUCCGGG AAUAUUUU | 3115 |
| 1352 | UAUUGGUU G ACUUCCGG | 1366 | CCGGAAGU GGAGGAAACUCC CU UCAAGGACAUCGUCCGGG AACCAAUA | 3116 |
| 1359 | UGACUUCC G GCUUUCUA | 1367 | UAGAAAGC GGAGGAAACUCC CU UCAAGGACAUCGUCCGGG GGAAGUCA | 3117 |
| 1369 | CUUUCUAA G GGUGAUGG | 1368 | CCAUCACC GGAGGAAACUCC CU UCAAGGACAUCGUCCGGG UUAGAAAG | 3118 |
| 1370 | UUUCUAAG G GUGAUGGA | 1369 | UCCAUCAC GGAGGAAACUCC CU UCAAGGACAUCGUCCGGG CUUAGAAA | 3119 |
| 1373 | CUAAGGGU G AUGGAUUG | 1370 | CAAUCCAU GGAGGAAACUCC CU UCAAGGACAUCGUCCGGG ACCCUUAG | 3120 |
| 1376 | AGGGUGAU G GAUUGGAG | 1371 | CUCCAAUC GGAGGAAACUCC CU UCAAGGACAUCGUCCGGG AUCACCCU | 3121 |
| 1377 | GGGUGAUG G AUUGGAGU | 1372 | ACUCCAAU GGAGGAAACUCC CU UCAAGGACAUCGUCCGGG CAUCACCC | 3122 |
| 1381 | GAUGGAUU G GAGUUCAA | 1373 | UUGAACUC GGAGGAAACUCC CU UCAAGGACAUCGUCCGGG AAUCCAUC | 3123 |
| 1382 | AUGGAUUG G AGUUCAAG | 1374 | CUUGAACU GGAGGAAACUCC CU UCAAGGACAUCGUCCGGG CAAUCCAU | 3124 |
| 1390 | GAGUUCAA G AGACACUU | 1375 | AAGUGUCU GGAGGAAACUCC CU UCAAGGACAUCGUCCGGG UUGAACUC | 3125 |
| 1392 | GUUCAAGA G ACACUUCC | 1376 | GGAAGUGU GGAGGAAACUCC CU UCAAGGACAUCGUCCGGG UCUUGAAC | 3126 |
| 1402 | CACUUCCU G AAGAUUAA | 1377 | UUAAUCUU GGAGGAAACUCC CU UCAAGGACAUCGUCCGGG AGGAAGUG | 3127 |
| 1405 | UUCCUGAA G AUUAAAGG | 1378 | CCUUUAAU GGAGGAAACUCC CU UCAAGGACAUCGUCCGGG UUCAGGAA | 3128 |
| 1412 | AGAUUAAA G GGAAGCUG | 1379 | CAGCUUCC GGAGGAAACUCC CU UCAAGGACAUCGUCCGGG UUUAAUCU | 3129 |
| 1413 | GAUUAAAG G GAAGCUGA | 1380 | UCAGCUUC GGAGGAAACUCC CU UCAAGGACAUCGUCCGGG CUUUAAUC | 3130 |
| 1414 | AUUAAAGG G AAGCUGAU | 1381 | AUCAGCUU GGAGGAAACUCC CU UCAAGGACAUCGUCCGGG CCUUUAAU | 3131 |
| 1420 | GGGAAGCU G AUUGAUAU | 1382 | AUAUCAAU GGAGGAAACUCC CU UCAAGGACAUCGUCCGGG AGCUUCCC | 3132 |
| 1424 | AGCUGAUU G AUAUUGUG | 1383 | CACAAUAU GGAGGAAACUCC CU UCAAGGACAUCGUCCGGG AAUCAGCU | 3133 |
| 1432 | GAUAUUGU G AGCAGCCA | 1384 | UGGCUGCU GGAGGAAACUCC CU UCAAGGACAUCGUCCGGG ACAAUAUC | 3134 |
| 1441 | AGCAGCCA G AAGGUUUG | 1385 | CAAACCUU GGAGGAAACUCC CU UCAAGGACAUCGUCCGGG UGGCUGCU | 3135 |
| 1444 | AGCCAGAA G GUUUGGCU | 1386 | AGCCAAAC GGAGGAAACUCC CU UCAAGGACAUCGUCCGGG UUCUGGCU | 3136 |
| 1449 | GAAGGUUU G GCUUCCUG | 1387 | CAGGAAGC GGAGGAAACUCC CU UCAAGGACAUCGUCCGGG AAACCUUC | 3137 |
| 1464 | UGCCACAU G AUCGGACC | 1388 | GGUCCGAU GGAGGAAACUCC CU UCAAGGACAUCGUCCGGG AUGUGGCA | 3138 |
| 1468 | ACAUGAUC G GACCAUCG | 1389 | CGAUGGUC GGAGGAAACUCC CU UCAAGGACAUCGUCCGGG GAUCAUGU | 3139 |
| 1469 | CAUGAUCG G ACCAUCGG | 1390 | CCGAUGGU GGAGGAAACUCC CU UCAAGGACAUCGUCCGGG CGAUCAUG | 3140 |
| 1476 | GGACCAUC G GCUCUGGG | 1391 | CCCAGAGC GGAGGAAACUCC CU UCAAGGACAUCGUCCGGG GAUGGUCC | 3141 |
| 1482 | UCGGCUCU G GGGAAUCC | 1392 | GGAUUCCC GGAGGAAACUCC CU UCAAGGACAUCGUCCGGG AGAGCCGA | 3142 |
| 1483 | CGGCUCUG G GGAAUCCU | 1393 | AGGAUUCC GGAGGAAACUCC CU UCAAGGACAUCGUCCGGG CAGAGCCG | 3143 |
| 1484 | GGCUCUGG G GAAUCCUG | 1394 | CAGGAUUC GGAGGAAACUCC CU UCAAGGACAUCGUCCGGG CCAGAGCC | 3144 |
| 1485 | GCUCUGGG G AAUCCUGG | 1395 | CCAGGAUU GGAGGAAACUCC CU UCAAGGACAUCGUCCGGG CCCAGAGC | 3145 |
| 1492 | GGAAUCCU G GUGAAUAU | 1396 | AUAUUCAC GGAGGAAACUCC CU UCAAGGACAUCGUCCGGG AGGAUUCC | 3146 |
| 1495 | AUCCUGGU G AAUAUAGU | 1397 | ACUAUAUU GGAGGAAACUCC CU UCAAGGACAUCGUCCGGG ACCAGGAU | 3147 |
| 1515 | GCUAUGUU G ACAUUAUU | 1398 | AAUAAUGU GGAGGAAACUCC CU UCAAGGACAUCGUCCGGG AACAUAGC | 3148 |
| 1531 | UCUUCCUA G AGAAGAUU | 1399 | AAUCUUCU GGAGGAAACUCC CU UCAAGGACAUCGUCCGGG UAGGAAGA | 3149 |
| 1533 | UUCCUAGA G AAGAUUAU | 1400 | AUAAUCUU GGAGGAAACUCC CU UCAAGGACAUCGUCCGGG UCUAGGAA | 3150 |
| 1536 | CUAGAGAA G AUUAUCCU | 1401 | AGGAUAAU GGAGGAAACUCC CU UCAAGGACAUCGUCCGGG UUCUCUAG | 3151 |
| 1573 | UAGUUCCU G AAGUGUUC | 1402 | GAACACUU GGAGGAAACUCC CU UCAAGGACAUCGUCCGGG AGGAACUA | 3152 |
| 1627 | GUUUGUUC G GCAUACAA | 1403 | UUGUAUGC GGAGGAAACUCC CU UCAAGGACAUCGUCCGGG GAACAAAC | 3153 |
| 1670 | AAAACUUU G GGGAAAGG | 1404 | CCUUUCCC GGAGGAAACUCC CU UCAAGGACAUCGUCCGGG AAAGUUUU | 3154 |
| 1671 | AAACUUUG G GGAAAGGA | 1405 | UCCUUUCC GGAGGAAACUCC CU UCAAGGACAUCGUCCGGG CAAAGUUU | 3155 |
| 1672 | AACUUUGG G GAAAGGAU | 1406 | AUCCUUUC GGAGGAAACUCC CU UCAAGGACAUCGUCCGGG CCAAAGUU | 3156 |
| 1673 | ACUUUGGG G AAAGGAUG | 1407 | CAUCCUUU GGAGGAAACUCC CU UCAAGGACAUCGUCCGGG CCCAAAGU | 3157 |
| 1677 | UGGGGAAA G GAUGAAUA | 1408 | UAUUCAUC GGAGGAAACUCC CU UCAAGGACAUCGUCCGGG UUUCCCCA | 3158 |
| 1678 | GGGGAAAG G AUGAAUAG | 1409 | CUAUUCAU GGAGGAAACUCC CU UCAAGGACAUCGUCCGGG CUUUCCCC | 3159 |
| 1681 | GAAAGGAU G AAUAGAAU | 1410 | AUUCUAUU GGAGGAAACUCC CU UCAAGGACAUCGUCCGGG AUCCUUUC | 3160 |
| 1686 | GAUGAAUA G AAUUCAUU | 1411 | AAUGAAUU GGAGGAAACUCC CU UCAAGGACAUCGUCCGGG UAUUCAUC | 3161 |
| 1696 | AUUCAUUU G AUUAUUUC | 1412 | GAAAUAAU GGAGGAAACUCC CU UCAAGGACAUCGUCCGGG AAAUGAAU | 3162 |
| 1725 | UAGUAUCU G AAUUUGAA | 1413 | UUCAAAUU GGAGGAAACUCC CU UCAAGGACAUCGUCCGGG AGAUACUA | 3163 |
| 1731 | CUGAAUUU G AAACUCAU | 1414 | AUGAGUUU GGAGGAAACUCC CU UCAAGGACAUCGUCCGGG AAAUUCAG | 3164 |
| 1742 | ACUCAUCU G GUGGAAAC | 1415 | GUUUCCAC GGAGGAAACUCC CU UCAAGGACAUCGUCCGGG AGAUGAGU | 3165 |
| 1745 | CAUCUGGU G GAAACCAA | 1416 | UUGGUUUC GGAGGAAACUCC CU UCAAGGACAUCGUCCGGG ACCAGAUG | 3166 |
| 1746 | AUCUGGUG G AAACCAAG | 1417 | CUUGGUUU GGAGGAAACUCC CU UCAAGGACAUCGUCCGGG CACCAGAU | 3167 |
| 1760 | AAGUUUCA G GGGACAUG | 1418 | CAUGUCCC GGAGGAAACUCC CU UCAAGGACAUCGUCCGGG UGAAACUU | 3168 |
| 1761 | AGUUUCAG G GGACAUGA | 1419 | UCAUGUCC GGAGGAAACUCC CU UCAAGGACAUCGUCCGGG CUGAAACU | 3169 |
| 1762 | GUUUCAGG G GACAUGAG | 1420 | CUCAUGUC GGAGGAAACUCC CU UCAAGGACAUCGUCCGGG CCUGAAAC | 3170 |
| 1763 | UUUCAGGG G ACAUGAGU | 1421 | ACUCAUGU GGAGGAAACUCC CU UCAAGGACAUCGUCCGGG CCCUGAAA | 3171 |
| 1768 | GGGGACAU G AGUUUUCC | 1422 | GGAAAACU GGAGGAAACUCC CU UCAAGGACAUCGUCCGGG AUGUCCCC | 3172 |

Input Sequence = AF016582. Cut Site = G/.
Stem Length = 8. Core Sequence = GGAGGAAACUCC CU UCAAGGACAUCGUCCGGG
AF016582 (Homo sapiens checkpoint kinase Chk1 (CHK1) mRNA; 1821 bp)

What is claimed is:

1. A nucleic acid molecule which down regulates expression of a Chk1 gene.

2. The nucleic acid of claim 1, wherein said nucleic acid molecule is used to treat cancer.

3. The nucleic acid molecule of claim 1, wherein said nucleic acid molecule is an enzymatic nucleic acid molecule.

4. The nucleic acid of claim 3, wherein a binding arm of said enzymatic nucleic acid molecule comprise sequences complementary to any of sequences defined as Sequence ID Nos. 1-1422.

5. The nucleic acid molecule of claim 3, wherein said enzymatic nucleic acid molecule comprises any of sequences defined as sequence ID Nos. 1423-3172.

6. The nucleic acid molecule of claim 1, wherein said nucleic acid molecule is an antisense nucleic acid molecule.

7. The nucleic acid molecule of claim 6, wherein said antisense nucleic acid molecule comprises sequence complementary to any of sequence defined as Sequence ID Nos. 1-1422 and 3173-3180.

8. The nucleic acid molecule of claim 6, wherein said antisense nucleic acid molecule comprise any of sequences defined as sequence ID Nos. 3181-3188.

9. The nucleic acid molecule of claim 3, wherein said enzymatic nucleic acid molecule is in a hammerhead (HH) motif.

10. The nucleic acid molecule of claim 3, wherein said enzymatic nucleic acid molecule is in a hairpin, hepatitis Delta virus, group I intron, VS nucleic acid, amberzyme, zinzyme or RNAse P nucleic acid motif.

11. The nucleic acid molecule of claim 3, wherein said enzymatic nucleic acid molecule is in a NCH motif.

12. The nucleic acid molecule of claim 3, wherein said enzymatic nucleic acid molecule is in a G-cleaver motif.

13. The nucleic acid molecule of claim 3, wherein said enzymatic nucleic acid molecule is a DNAzyme.

14. The nucleic acid molecule of claim 3, wherein said enzymatic nucleic acid molecule comprises between 12 and 100 bases complementary to the RNA of Chk1 gene.

15. The nucleic acid of claim 3, wherein said enzymatic nucleic acid molecule comprises between 14 and 24 bases complementary to the RNA of Chk1 gene.

16. The nucleic acid molecule of claim 1, wherein said nucleic acid is chemically synthesized.

17. The nucleic acid molecule of claim 1, wherein said nucleic acid comprises at least one 2′-sugar modification.

18. The nucleic acid molecule of claim 1, wherein said nucleic acid comprises at least one nucleic acid base modification.

19. The nucleic acid molecule of claim 1, wherein said nucleic acid comprises at least one phosphate backbone modification.

20. A mammalian cell including the nucleic acid molecule of claim 1.

21. The mammalian cell of claim 20, wherein said mammalian cell is a human cell.

22. A method of reducing Chk1 activity in a cell, comprising the step of contacting said cell with the nucleic acid molecule of claim 1, under conditions suitable for said reduction of Chk1 activity.

23. A method of treatment of a patient having a condition associated with the level of Chk1, comprising contacting cells of said patient with the nucleic acid molecule of claim 1, under conditions suitable for said treatment.

24. The method of claim 23 further comprising the use of one or more therapies under conditions suitable for said treatment.

25. A method of cleaving RNA of Chk1 gene, comprising, contacting the nucleic acid molecule of claim 1, with said RNA under conditions suitable for the cleavage of said RNA.

26. The method of claim 25, wherein said cleavage is carried out in the presence of a divalent cation.

27. The method of claim 26, wherein said divalent cation is Mg²⁺.

28. The nucleic acid molecule of claim 1, wherein said nucleic acid comprises a cap structure, wherein the cap structure is at the 5′-end or 3′-end or both the 5′-end and the 3′-end.

29. The enzymatic nucleic acid molecule of claim 9, wherein said hammerhead motif comprises sequences complementary to any of sequences shown as Seq ID Nos 1-358.

30. The enzymatic nucleic acid molecule of claim 11, wherein said NCH motif comprises sequences complementary to any of sequences shown as Seq ID Nos 359-680.

31. The enzymatic nucleic acid molecule of claim 12, wherein said G-cleaver motif comprises sequences complementary to any of sequences shown as Seq ID Nos 681-790.

32. The enzymatic nucleic acid molecule of claim 13, wherein said DNAzyme comprises sequences complementary to any of substrate sequences shown as Seq. ID Nos 791-1185.

33. The enzymatic nucleic acid molecule of claim 10, wherein said zinzyme comprises sequences complementary to any of substrate sequences shown as Seq. ID Nos 791-954.

34. The enzymatic nucleic acid molecule of claim 10, wherein said amberzyme comprises sequences complementary to any of substrate sequences shown as Seq. ID Nos 791-1422.

35. An expression vector comprising nucleic acid sequence encoding at least one nucleic acid molecule of claim 1, in a manner which allows expression of that nucleic acid molecule.

36. A mammalian cell including an expression vector of claim 35.

37. The mammalian cell of claim 36, wherein said mammalian cell is a human cell.

38. The expression vector of claim 35, wherein said nucleic acid molecule is an enzymatic nucleic acid molecule.

39. The expression vector of claim 35, wherein said expression vector further comprises a sequence for an antisense nucleic acid molecule complementary to the RNA of Chk1 gene.

40. The expression vector of claim 35, wherein said expression vector comprises sequence encoding at least two said nucleic acid molecules, which may be same or different.

41. The expression vector of claim 40, wherein one said expression vector further comprises sequence encoding antisense nucleic acid molecule complementary to the RNA of Chk1 gene.

42. The expression vector of claim 40, wherein one said expression vector further comprises sequence encoding enzymatic nucleic acid molecule complementary to the RNA of Chk1 gene.

43. A method for treatment of cancer comprising the step of administering to a patient the nucleic acid molecule of claim 1 under conditions suitable for said treatment.

44. The method of claim 43, wherein said cancer is colorectal cancer.

45. The method of claim 43, wherein said cancer is lung cancer.

46. The method of claim 43, wherein said cancer is breast cancer.

47. The method of claim 43, wherein said cancer is prostate cancer.

48. A method for treatment of cancer comprising the step of administering to a patient the antisense nucleic acid molecule of claim 7 under conditions suitable for said treatment.

49. The method of claim 45, wherein said method further comprises administering to said patient the nucleic acid molecule of claim 1 in conjunction with one or more of other therapies.

50. The method of claim 49, wherein said “other therapies” are therapies selected from the group consisting of radiation and chemotherapy treatment.

51. The nucleic acid molecule of claim 7, wherein said nucleic acid molecule comprises at least five ribose residues; at least ten 2′-O-methyl modifications, and a 3′-end modification.

52. The nucleic acid molecule of claim 51, wherein said nucleic acid molecule further comprises phosphorothioate linkages on at least three of the 5′ terminal nucleotides.

53. The nucleic acid molecule of claim 51, wherein said 3′-end modification is 3′-3′ inverted abasic moiety.

54. The nucleic acid molecule of claim 3, wherein said nucleic acid molecule comprises at least five ribose residues; at least ten 2′-O-methyl modifications, and a 3′-end modification.

55. The nucleic acid molecule of claim 54, wherein said nucleic acid molecule further comprises phosphorothioate linkages on at least three of the 5′ terminal nucleotides.

56. The nucleic acid molecule of claim 54, wherein said 3′- end modification is 3′-3′ inverted abasic moiety.

57. The enzymatic nucleic acid molecule of claim 13, wherein said DNAzyme comprises at least ten 2′-O-methyl modifications and a 3′-end modification.

58. The enzymatic nucleic acid molecule of claim 57, wherein said DNAzyme further comprises phosphorothioate linkages on at least three of the 5′ terminal nucleotides.

59. The enzymatic nucleic acid molecule of claim 57, wherein said 3′-end modification is 3′-3′ inverted abasic moiety.
